(19) United States
(12) Patent Application Publication
(10) Pub. No.: US 2016/0060687 A1
(43) Pub. Date: Mar. 3, 2016

(54) METHODS AND COMPOSITIONS TO IDENTIFY, QUANTIFY, AND CHARACTERIZE TARGET ANALYTES AND BINDING MOIETIES

(71) Applicant: CDI Laboratories, Inc., Mayaguez, PR (US)

(72) Inventors: Heng ZHU, Towson, MD (US); Jiang QIAN, Lutherville, MD (US); Ignacio PINO, Mayaguez, PR (US)

(21) Appl. No.: 14/802,417

(22) Filed: Jul. 17, 2015

Related U.S. Application Data
(60) Provisional application No. 62/134,171, filed on Mar. 17, 2015.
(60) Provisional application No. 62/091,920, filed on Dec. 15, 2014.
(60) Provisional application No. 62/062,511, filed on Oct. 10, 2014.
(60) Provisional application No. 62/026,601, filed on Jul. 18, 2014.

(51) Int. Cl. C12Q 1/68

(52) U.S. Cl. CPC C12Q 1/6837

(57) ABSTRACT

Proximity coupling and sequencing methods to screen identify, validate and/or characterize interactions between analytes and binding moieties are disclosed. Also disclosed herein are proximity coupling methods and sequencing methods to determine or quantify levels of target analytes. The disclosed methods can be multiplexed in two dimensions, and can be used to determine the affinity and specificity of each of a plurality of binding moieties for each of a plurality of target analytes. Also disclosed herein are substrates, arrays, and reagents for use in the methods, and methods of their preparation.

### CROSS-REFERENCE

This application claims priority to U.S. Provisional Application No. 62/026,601, filed Jul. 18, 2014; U.S. Provisional Application No. 62/062,511, filed Oct. 10, 2014; U.S. Provisional Application No. 62/091,920, filed Dec. 15, 2014; and U.S. Provisional Application No. 62/134,171, filed Mar. 17, 2015; each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 15, 2015, is named 38759-709.201_SL.txt and is 27,245 bytes in size.

### BACKGROUND

Formation of complexes of analytes and binding moieties plays a central role in various disease states. Binding moieties (e.g., polypeptides, nucleic acids, and small molecules) can be central to the progression or treatment of these diseases. In an autoimmune reaction, binding moieties (e.g., antibodies) can locate and bind to target analytes, and signal the body to attack the tissues where these target analytes are located. Diseases can be treated by identifying a binding moiety's target analyte and blocking the binding moiety's recognition of its target analyte. Thus, screening to identify binding moieties and their target analytes is an important tool and can be used to combat disease progression.

Traditional means of screening binding moieties, such as monoclonal antibodies (mAbs), aptamers, and small molecules, for a disease or condition are inefficient and limited by the current state of the art. A multiplex assay that allows for screening hundreds to thousands of binding moieties simultaneously can greatly reduce cost and improve throughput. In addition, such highly multiplexed screens allow a precise measurement of the specificity of a given analyte, because the counter screens are performed simultaneously. Thus, there exists a critical need for faster and cheaper screening of binding moieties to identify their respective target analyte, determine the affinities of the binding moieties for target analytes, and determine the binding specificity of binding moieties for target analytes. To date, most drug screenings have focused on a single target at a time because of limitations of current technologies. A significant drawback of these approaches is their inability to determine specificities of binding moieties, i.e., whether an identified candidate compound would also cross-react with other analytes, which may cause unwanted side effects.

Proximity-probe based detection assays and particularly proximity ligation assays, have proved very useful in the specific and sensitive detection of proteins in a number of different applications, e.g. the detection of weakly expressed or low abundance proteins. However, such assays are not without their problems and room for improvement exists, with respect to both the sensitivity and specificity of the assay.

### SUMMARY

Generally, proximity probes comprising a binding moiety and a proximity polynucleotide are contacted to a solid support comprising a plurality of target analytes and a plurality of address polynucleotides each barcoded to a target analyte. When a binding moiety is bound to a target analyte, the proximity polynucleotide is coupled to the address polynucleotide. The coupled products are then amplified and sequenced. When a binding moiety can recognize a particular target analyte on the address polynucleotide array, the binding moiety's barcode and the address polynucleotide barcode will appear in the same sequence. By counting numbers of the reads for the same sequences, the relative strength of the binding moiety can be determined. By counting the number of different proximity barcode coexisting with a given address polynucleotide, binding specificity of the binding moiety can be determined.

Disclosed herein are proximity coupling and sequencing methods to detect a level of an analyte, and to screen, identify, validate and characterize interactions between analytes and binding moieties. Also disclosed herein are two-dimensional multiplexed methods to determine the affinity and specificity of each of a plurality of binding moieties for each of a plurality of target analytes. Also disclosed herein are substrates, arrays, and reagents for use in the methods, methods of making the substrates, arrays, and reagents are also disclosed herein. Also disclosed herein are applications of reagents under various biological conditions.

An exemplary method described herein is performed by mixing, e.g., one or more, such as 100-10,000, binding moiety-proximity polynucleotide probes to a protein array that has been barcoded with address polynucleotides, followed by addition of a splint polynucleotide, ligation, amplification of the ligation products, and sequencing of the resulting amplified products to reveal the combinations of the antibody barcodes and protein address barcodes.

In one aspect, provided herein is a solid support comprising a discrete address region comprising a first discrete location comprising an address polynucleotide coupled thereto, and a second discrete location comprising a target analyte coupled thereto; wherein the address polynucleotide is barcoded to the target analyte and in proximity to the target analyte, and wherein the target analyte does not base pair with the address polynucleotide.

In some embodiments, the solid support comprises a plurality of discrete address regions, each comprising a first discrete location comprising an address polynucleotide coupled thereto, and a second discrete location comprising a target analyte is barcoded to the address polynucleotide; wherein each address polynucleotide is in proximity to the target analyte in the same discrete address region.

In one aspect, provided herein is an array comprising a plurality of discrete address regions, a plurality of address polynucleotides, and a plurality of target analytes, wherein each discrete address region of the plurality of discrete address regions comprises: a first discrete location coupled to an address polynucleotide of the plurality, and a second discrete location coupled to a target analyte of the plurality; wherein each address polynucleotide of the plurality of address polynucleotides identifies the discrete address region to which the address polynucleotide is coupled, or the target analyte coupled to the same discrete address region, wherein each target analyte of the plurality is a polypeptide or a small molecule.

In some embodiments, the target analyte in each discrete region of the plurality is different. In some embodiments, each address polynucleotide of the plurality comprises a unique address barcode that identifies the discrete address region, identity, or both of the corresponding target analyte. In some embodiments, the target analyte is barcoded to an address barcode sequence of the address polynucleotide. In some embodiments, the address barcode is unique. In some embodiments, the address polynucleotide of a first discrete address region is not in proximity to an address polynucleotide of a second discrete address region. In some embodiments, the address polynucleotide is in proximity to a proximity probe when the proximity probe is bound to the target analyte. In some embodiments, the proximity probe comprises a binding moiety. In some embodiments, the proximity probe is bound to the target analyte. In some embodiments, the binding moiety is bound to the target analyte. In some embodiments, the proximity probe comprises a proximity polynucleotide. In some embodiments, the binding moiety is coupled to the proximity polynucleotide. In some embodiments, the binding moiety is barcoded to the proximity polynucleotide to which it is coupled. In some embodiments, the proximity polynucleotide comprises a proximity barcode. In some embodiments, the proximity barcode identifies the binding moiety to which it is coupled. In some embodiments, the proximity barcode is unique.

In some embodiments, the proximity polynucleotide further comprises a proximity linker sequence. In some embodiments, the proximity polynucleotide further comprises a proximity primer binding sequence. In some embodiments, the proximity polynucleotide further comprises a proximity spacer sequence. In some embodiments, the proximity polynucleotide is arranged in the order of the proximity linker sequence, the proximity barcode, the proximity primer binding sequence, and the proximity spacer propagating toward the binding moiety. In some embodiments, the proximity polynucleotide is arranged in the order of the proximity linker sequence, the proximity barcode, the proximity primer binding sequence, and the proximity spacer from a 5′ end of the proximity polynucleotide to a 3′ end of the proximity polynucleotide. In some embodiments, an end of the proximity polynucleotide comprises a functional group.

In some embodiments, a 5′ end of the proximity polynucleotide comprises a functional group. In some embodiments, the functional group of the proximity polynucleotide is selected from the group consisting of an amino group, a carboxyl group, a maleimide group, biotin, avidin, and a phosphate group. In some embodiments, the functional group of the proximity polynucleotide is a maleimide group.

In some embodiments, the target analyte is not purified or isolated. In some embodiments, the target analyte is purified or isolated.

In some embodiments, the target analyte is from a biological sample. In some embodiments, the target analyte is from a sample from a host. In some embodiments, the target analyte is in a cell lysate or cell culture medium.

In some embodiments, the target analyte is a polypeptide. In some embodiments, the target analyte is synthesized. In some embodiments, the target analyte is synthesized in situ. In some embodiments, the target analyte is expressed in a cell-free system. In some embodiments, the target analyte is expressed in vitro. In some embodiments, the target analyte is translated in vitro. In some embodiments, the target analyte is expressed in a cell. In some embodiments, the target analyte is expressed naturally. In some embodiments, the target analyte is expressed recombinantly. In some embodiments, the target analyte is a membrane protein in an envelope of virus particle. In some embodiments, the target analyte is in a complex. In some embodiments, the target analyte is an antibody or fragment thereof. In some embodiments, the target analyte is a transcription factor. In some embodiments, the target analyte is a receptor. In some embodiments, the receptor is a transmembrane receptor. In some embodiments, the target analyte is a nuclear protein. In some embodiments, the target analyte is a cytoplasmic protein. In some embodiments, the target analyte is a nucleosome. In some embodiments, the target analyte is recombinant.

In some embodiments, the target analyte is immunoprecipitated. In some embodiments, the target analyte comprises a tag. In some embodiments, the target analyte comprises an affinity tag.

In some embodiments, the target analyte is a small molecule or a macrocycle. In some embodiments, the target analyte is a drug. In some embodiments, the target analyte is a compound. In some embodiments, the target analyte is an organic compound. In some embodiments, the target analyte has a molecular weight of 900 Daltons or less. In some embodiments, the target analyte has a molecular weight of 500 Daltons or more. In some embodiments, the target analyte does not comprise a phosphodiester linkage. In some embodiments, the target analyte comprises at least two amide bonds. In some embodiments, the target analyte is not DNA or RNA.

In some embodiments, the binding moiety is a polynucleotide. In some embodiments, the polynucleotide is single stranded. In some embodiments, the polynucleotide is double stranded. In some embodiments, the polynucleotide is RNA. In some embodiments, the polynucleotide is DNA. In some embodiments, the polynucleotide is an RNA-DNA hybrid.

In some embodiments, the polynucleotide is an aptamer. In some embodiments, the target analyte is a polypeptide comprising a interacting polynucleotide. In some embodiments, the interacting polynucleotide is genomic DNA. In some embodiments, the interacting polynucleotide is sheared. In some embodiments, the interacting polynucleotide comprises an adaptor on one or both ends. In some embodiments, the adaptor is a Y adaptor. In some embodiments, the adaptor comprises a primer binding site. In some embodiments, the interacting polynucleotide is not attached to the solid support directly. In some embodiments, the interacting polynucleotide comprises a sequence that interacts with the target analyte and is downstream of a primer binding site.

In some embodiments, the solid support is a bead. In some embodiments, solid support does not comprise an address polynucleotide.

In some embodiments, the polynucleotide comprises a hairpin structure.

In some embodiments, the binding moiety is methylated. In some embodiments, the binding moiety is unmethylated. In some embodiments, the binding moiety is a library of binding moieties. In some embodiments, the library of binding moieties that are polynucleotides comprises polynucleotides with sequences selected from the group consisting of NNNNCGNNNN, NNNNGCNNNN, NNNNCCGGNNNN (SEQ ID NO: 1), and combinations thereof, wherein N is any nucleotide. In some embodiments, the library of binding moieties that are polynucleotides comprises polynucleotides with sequences selected from the group consisting of NNNNCGNNNN, NNNNGCNNNN, NNNNCCGGNNNN (SEQ ID NO: 1), NNNNmCGNNNN, NNNNGmCNNNN, NNNNmCCGGNNNN (SEQ ID NO: 2), NNNNCmCGGNNNN (SEQ ID NO: 3), and combinations thereof, wherein mC is a methylated cytosine.

In some embodiments, the proximity polynucleotide is a 5′ overhang region of the binding moiety that is a polynucleotide. In some embodiments, a sequence of the binding moiety that is a polynucleotide comprises a proximity barcode. In some embodiments, the proximity polynucleotide that is a 5′ overhang region of the binding moiety that is a polynucleotide comprises a proximity linker sequence. In some embodiments, the proximity polynucleotide that is a 5′ overhang region of the binding moiety that is a polynucleotide does not comprise a proximity primer binding sequence. In some embodiments, the proximity polynucleotide that is a 5′ overhang region of the binding moiety that is a polynucleotide does not comprise a proximity barcode.

In some embodiments, the binding moiety that is a polynucleotide comprises a universal 3′ region. In some embodiments, the binding moiety that is a polynucleotide comprises a universal 3′ region that does not comprise a potential binding motif to a target analyte or a fragment thereof. In some embodiments, the universal 3′ region comprises a proximity primer binding sequence.

In some embodiments, the solid support comprises a primer set comprising a first primer that binds to a primer binding site upstream of the address barcode; and a second primer that binds to a 3′ region of the binding moiety that is a polynucleotide.

In some embodiments, the binding moiety is a polypeptide. In some embodiments, the polypeptide is an antibody or fragment thereof. In some embodiments, the polypeptide is a purified. In some embodiments, the polypeptide is recombinant. In some embodiments, the polypeptide comprises a variable heavy chain (V_{H}) or light chain (V_{L}) region. In some embodiments, the binding moiety is a library of binding moieties that are polypeptides. In some embodiments, the polypeptide is transcribed from a transcript encoding the polypeptide. In some embodiments, the polypeptide is linked to the transcript encoding the polypeptide.

In some embodiments, the polypeptide linked to the transcript encoding the polypeptide is attached to a molecule that enters the A site of a ribosome when the ribosome reaches a 3′ end of the template during translation. In some embodiments, the molecule that enters the A site of a ribosome when the ribosome reaches a 3′ end of the template during translation is puromycin. In some embodiments, the transcript encoding the polypeptide is ligated to a polynucleotide attached to the molecule that enters the A site of a ribosome when the ribosome reaches a 3′ end of the template during translation. In some embodiments, the polypeptide is attached to a cDNA of the transcript encoding the polypeptide.

In some embodiments, the address polynucleotide further comprises an address linker sequence. In some embodiments, the address polynucleotide further comprises an address primer binding sequence. In some embodiments, the address polynucleotide further comprises an address spacer sequence. In some embodiments, the address polynucleotide is arranged in the order of the address linker sequence, the address barcode, the address primer binding sequence, and the address spacer propagating toward the solid support. In some embodiments, the address polynucleotide is arranged in the order of the address linker sequence, the address barcode, the address primer binding sequence, and the address spacer from a 3′ end of the address polynucleotide to a 5′ end of the address polynucleotide.

In some embodiments, an end of the address polynucleotide comprises a functional group. In some embodiments, a 3′ end of the address polynucleotide comprises a functional group. In some embodiments, the functional group of the address polynucleotide is selected from the group consisting of an amino group, a carboxyl group, a hydroxyl group, biotin, avidin, and a phosphate group. In some embodiments, the functional group of the address polynucleotide is avidin.

In some embodiments, the address polynucleotide is coupled to the solid support covalently. In some embodiments, the address polynucleotide is coupled to the solid support non-covalently. In some embodiments, the address polynucleotide is coupled to the solid support by a linker. In some embodiments, the address polynucleotide is coupled to the solid support by the functional group.

In some embodiments, the target analyte is coupled to the solid support covalently. In some embodiments, the target analyte is coupled to the solid support non-covalently. In some embodiments, the target analyte is coupled to the solid support by a linker. In some embodiments, the linker is an antibody. In some embodiments, the linker is specific for the target analyte. In some embodiments, the linker is specific for a post-translational modification of the target analyte. In some embodiments, the linker comprises a plurality of linkers, each specific for a target analyte or modification thereof.

In some embodiments, the target analyte is coupled to the solid support by a tag. In some embodiments, the tag is a universal tag. In some embodiments, the tag is selected from the group consisting of a His tag, a GST tag, a FLAG tag, a maltose binding protein (MBP) tag, and combinations thereof.

In some embodiments, the proximity linker sequence is coupled to the address linker sequence. In some embodiments, an end of the proximity polynucleotide is adjacent to an end of the address polynucleotide. In some embodiments, the end of the proximity polynucleotide adjacent to an end of the address polynucleotide is a 3′ end of the proximity polynucleotide. In some embodiments, the end of the address polynucleotide adjacent to an end of the proximity polynucleotide is a 5′ end of the address polynucleotide.

In some embodiments, the proximity polynucleotide is hybridized to a splint polynucleotide. In some embodiments, the address polynucleotide is hybridized to a splint polynucleotide. In some embodiments, the proximity polynucleotide and the address polynucleotide are coupled together. In some embodiments, the proximity polynucleotide and the address polynucleotide are coupled non-covalently together. In some embodiments, the proximity polynucleotide and the address polynucleotide are hybridized. In some embodiments, the proximity polynucleotide and the address polynucleotide are not directly hybridized together. In some embodiments, the proximity polynucleotide and the address polynucleotide are not complimentary to each other. In some embodiments, the address polynucleotide and the proximity polynucleotide are hybridized to a same splint polynucleotide. In some embodiments, the proximity polynucleotide and the address polynucleotide are coupled covalently together.

In some embodiments, the proximity barcode and the address barcode are on a same polynucleotide molecule. In some embodiments, the proximity polynucleotide and the address polynucleotide are not directly hybridized together. In some embodiments, the address polynucleotide and the proximity polynucleotide are ligated together.

In some embodiments, the solid support further comprises a DNA ligase. In some embodiments, the solid support further comprises a polymerase. In some embodiments, the solid support further comprises a reverse transcriptase. In some embodiments, the solid support further comprises a splint polynucleotide. In some embodiments, the solid support further comprises an amplified product of a polynucleotide comprising a proximity barcode and an address barcode.

In some embodiments, the proximity polynucleotide and the address polynucleotide are from a same discrete address region of the solid support. In some embodiments, the proximity barcode and the address barcode are from the same discrete address region of the solid support. In some embodiments, the solid support comprises a plurality of target analytes, wherein each target analyte of the plurality is different. In some embodiments, the solid support comprises a plurality of target analytes, wherein each target analyte of the plurality is located within a different discrete address region of the solid support.

In some embodiments, the proximity probe comprises a plurality of proximity probes, where each proximity probe of the plurality is different. In some embodiments, the proximity probe comprises a plurality of proximity probes, where each proximity probe of the plurality is different. In some embodiments, the binding moiety comprises a plurality of binding moieties, wherein each binding moiety of the plurality is different. In some embodiments, the binding moiety comprises a plurality of binding moieties, wherein two or more binding moieties of the plurality are bound to a target analyte within a different discrete address region of the solid support. In some embodiments, the solid support comprises a plurality of address polynucleotides, wherein each address polynucleotide of the plurality is different. In some embodiments, the solid support comprises a plurality of address polynucleotides, wherein each address polynucleotide of the plurality is located within a different discrete address region of the solid support.

In some embodiments, the solid support comprises primer set comprising a first primer that binds to a primer binding site upstream of the address barcode; and a second primer that binds to a primer binding site upstream of the proximity barcode. In some embodiments, the first primer comprises a 5′ overhang region. In some embodiments, the 5′ overhang region of the first primer comprises a first universal sequencing primer binding site. In some embodiments, the second primer comprises a 5′ overhang region. In some embodiments, the 5′ overhang region of the second primer comprises a second universal sequencing primer binding site.

In some embodiments, the solid support comprises at least at least 2, or at least about 5, 10, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more discrete address regions. In some embodiments, the solid support comprises from 100 to about 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the solid support comprises from 2 to about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the proteins of an organism's proteome. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors of an organism's proteome. In some embodiments, the organism is selected from the group consisting of mouse, rat, rabbit, cat, dog, bird, horse, pig, monkey, goat, cow, and human. In some embodiments, the organism is human.

In some embodiments, the target analyte comprises a plurality of target analytes comprising at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more target analytes. In some embodiments, the address polynucleotide comprises a plurality of address polynucleotides comprising at least at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more address polynucleotides. In some embodiments, each target analyte of the plurality is different. In some embodiments, each address polynucleotide of the plurality comprises a unique address barcode. In some embodiments, each address polynucleotide of the plurality comprises a same address linker sequence. In some embodiments, each address polynucleotide of the plurality comprises a same address primer binding site.

In some embodiments, the proximity probe comprises a plurality of proximity probes comprising of at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more proximity probes. In some embodiments, each proximity probe of the plurality comprises a unique proximity barcode. In some embodiments, each proximity probe of the plurality comprises a same proximity linker sequence. In some embodiments, each proximity probe of the plurality comprises a same proximity primer binding site. In some embodiments, each proximity probe of the plurality comprises a different binding moiety. In some embodiments, the concentration of the target analyte in a discrete address region is known.

In some embodiments, the solid support is an array.

In one aspect, provided herein is a method of manufacturing comprising producing any of the solid supports described herein.

In one aspect, provided herein is a method comprising: contacting to a solid support a proximity probe, wherein the solid support comprises a discrete address region comprising a first discrete location comprising an address polynucleotide, and a second discrete location comprising a target analyte; wherein the address polynucleotide is barcoded to the target analyte, and wherein the target analyte is incapable of base pairing with the address polynucleotide.

In one aspect, provided herein is a method comprising: forming a complex between a target analyte and a proximity probe, wherein the target analyte is coupled to a solid support, the solid support comprising a discrete address region, the discrete address region comprising: a first discrete location comprising an address polynucleotide, and a second discrete location comprising the target analyte; wherein the address polynucleotide is barcoded to the target analyte; and wherein the target analyte does not base pair with the address polynucleotide.

In some embodiments, the proximity probe comprises a proximity polynucleotide coupled to a binding moiety. In some embodiments, the method further comprises coupling the proximity probe in the discrete address region to the address polynucleotide in the discrete address region. In some embodiments, the method further comprises amplifying a coupled product. In some embodiments, the method further comprises detecting a coupled product or an amplified product thereof.

In some embodiments, the solid support comprises a plurality of discrete address regions, each discrete address region of the plurality comprising a first discrete location comprising an address polynucleotide, and a second discrete location comprising a target analyte barcoded to the address polynucleotide. In some embodiments, the first and second discrete locations are in proximity. In some embodiments, each address polynucleotide is in proximity to the target analyte in the same discrete address region. In some embodiments, the target analyte is barcoded to an address barcode sequence of the address polynucleotide. In some embodiments, the address barcode is unique. In some embodiments, the address polynucleotide of a first discrete address region is not in proximity to a target analyte of a second discrete address region. In some embodiments, the address polynucleotide is in proximity to a proximity probe when the proximity probe is bound to the target analyte.

In some embodiments, the proximity probe comprises a binding moiety. In some embodiments, the method comprises binding the proximity probe to the target analyte. In some embodiments, the binding moiety is bound to the target analyte. In some embodiments, the proximity probe comprises a proximity polynucleotide. In some embodiments, the binding moiety is coupled to the proximity polynucleotide. In some embodiments, the binding moiety is barcoded to the proximity polynucleotide to which it is coupled. In some embodiments, the proximity polynucleotide comprises a proximity barcode sequence. In some embodiments, the binding moiety barcoded to the proximity barcode sequence of the proximity polynucleotide to which it is coupled. In some embodiments, the proximity barcode is unique. In some embodiments, the proximity polynucleotide further comprises a proximity linker sequence. In some embodiments, the proximity polynucleotide further comprises a proximity primer binding sequence. In some embodiments, the proximity polynucleotide further comprises a proximity spacer sequence. In some embodiments, the proximity polynucleotide is arranged in the order of the proximity linker sequence, the proximity barcode, the proximity primer binding sequence, and the proximity spacer propagating toward the binding moiety. In some embodiments, the proximity polynucleotide is arranged in the order of the proximity linker sequence, the proximity barcode, the proximity primer binding sequence, and the proximity spacer from a 5′ end of the proximity polynucleotide to a 3′ end of the proximity polynucleotide.

In some embodiments, an end of the proximity polynucleotide comprises a functional group. In some embodiments, a 5′ end of the proximity polynucleotide comprises a functional group. In some embodiments, the functional group of the proximity polynucleotide is selected from the group consisting of an amino group, a carboxyl group, a hydroxyl group, a maleimide group, biotin, avidin, and a phosphate group. In some embodiments, the functional group of the proximity polynucleotide is a maleimide group.

In some embodiments, the target analyte is a polypeptide. In some embodiments, the polypeptide is within a virus particle. In some embodiments, the polypeptide is within a virus particle membrane. In some embodiments, the polypeptide is an antibody or fragment thereof. In some embodiments, the polypeptide is a transcription factor. In some embodiments, the polypeptide is a receptor. In some embodiments, the receptor is a transmembrane receptor

In some embodiments, the target analyte is a small molecule. In some embodiments, the small molecule is a drug. In some embodiments, the small molecule is a compound. In some embodiments, the small molecule is an organic compound. In some embodiments, the small molecule has a molecular weight of 900 Daltons or less. In some embodiments, the small molecule has a molecular weight of 500 Daltons or more. In some embodiments, the target analyte does not comprise a phosphodiester linkage. In some embodiments, the target analyte comprises at least two amide bonds. In some embodiments, the target analyte is not DNA or RNA.

In some embodiments, the binding moiety is a polynucleotide. In some embodiments, the binding moiety that is a polynucleotide is single stranded. In some embodiments, the binding moiety that is a polynucleotide is double stranded. In some embodiments, the binding moiety that is a polynucleotide is RNA. In some embodiments, the binding moiety that is a polynucleotide is DNA. In some embodiments, the binding moiety that is a polynucleotide is an RNA-DNA hybrid. In some embodiments, the binding moiety that is a polynucleotide is an aptamer. In some embodiments, the binding moiety that is a polynucleotide comprises a hairpin structure.

In some embodiments, the binding moiety that is a polynucleotide is methylated. In some embodiments, the binding moiety that is a polynucleotide is unmethylated.

In some embodiments, the binding moiety that is a polynucleotide is a library of binding moieties that are polynucleotides. In some embodiments, the library of binding moieties that are polynucleotides comprises polynucleotides with sequences selected from the group consisting of NNNNCGNNNN, NNNNGCNNNN, NNNNCCGGNNNN (SEQ ID NO: 1), and combinations thereof, wherein N is any nucleotide. In some embodiments, the library of binding moieties that are polynucleotides comprises polynucleotides with sequences selected from the group consisting of NNNNCGNNNN, NNNNGCNNNN, NNNNCCGGNNNN (SEQ ID NO: 1), NNNNmCGNNNN, NNNNGmCNNNN, NNNNmCCGGNNNN (SEQ ID NO: 2), NNNNCmCGGNNNN (SEQ ID NO: 3), and combinations thereof, wherein mC is a methylated cytosine.

In some embodiments, the proximity polynucleotide is a 5′ overhang region of the binding moiety that is a polynucleotide. In some embodiments, a sequence of the binding moiety that is a polynucleotide comprises a proximity barcode. In some embodiments, the proximity polynucleotide that is a 5′ overhang region of the binding moiety that is a polynucleotide comprises a proximity linker sequence. In some embodiments, the proximity polynucleotide that is a 5′ overhang region of the binding moiety that is a polynucleotide does not comprise a proximity primer binding sequence. In some embodiments, the proximity polynucleotide that is a 5′ overhang region of the binding moiety that is a polynucleotide does not comprise a proximity barcode. In some embodiments, the 5′ overhang region is generated using an enzyme with 3′→5′ exonuclease activity. In some embodiments, the enzyme with 3′→5′ exonuclease activity is T4 DNA polymerase. In some embodiments, the binding moiety that is a polynucleotide comprises a universal 3′ region. In some embodiments, the binding moiety that is a polynucleotide comprises a universal 3′ region that does not comprise a potential binding motif to a target analyte or a fragment thereof. In some embodiments, the universal 3′ region comprises a proximity primer binding sequence.

In some embodiments, the amplifying comprises adding a primer set comprising a first primer that binds to a primer binding site upstream of the address barcode of the coupled product or an amplified product thereof; and a second primer that binds to a 3′ region of the binding moiety that is a polynucleotide of the coupled product or an amplified product thereof.

In some embodiments, the binding moiety is a polypeptide. In some embodiments, the polypeptide is an antibody or fragment thereof. In some embodiments, the polypeptide is a purified protein. In some embodiments, the binding moiety that is a polypeptide is a library of binding moieties that are polypeptides.

In some embodiments, the method further comprises transcribing the polypeptide. In some embodiments, the method further comprises linking the transcribed polypeptide to a transcript encoding the polypeptide. In some embodiments, the linking the transcribed polypeptide to a transcript encoding the polypeptide comprises attaching the transcript encoding the polypeptide to a molecule that enters the A site of a ribosome when the ribosome reaches a 3′ end of the template during translation. In some embodiments, the molecule that enters the A site of a ribosome when the ribosome reaches a 3′ end of the template during translation is puromycin. In some embodiments, the attaching the transcript encoding the polypeptide to a molecule that enters the A site of a ribosome when the ribosome reaches a 3′ end of the template during translation comprises ligating the transcript encoding the polypeptide to a polynucleotide attached to the molecule that enters the A site of a ribosome when the ribosome reaches a 3′ end of the template during translation.

In some embodiments, the method further comprises translating the polypeptide from a transcript encoding the polypeptide, wherein the transcript encoding the polypeptide is attached to a molecule that enters the A site of a ribosome when the ribosome reaches a 3′ end of the template during translation. In some embodiments, the method further comprises reverse transcribing a transcript encoding the polypeptide, wherein the transcript encoding the polypeptide is linked to the polypeptide. In some embodiments, the amplifying a coupled product comprises error prone PCR.

In some embodiments, the method further comprises selecting one or more nucleotide sequences encoding polypeptides with a high affinity for a target analyte.

In some embodiments, the method further comprises performing a selection round, wherein a selection round comprises: transcribing the selected one or more nucleotide sequences encoding the polypeptides with a high affinity for a target analyte, attaching a transcript to a molecule that enters the A site of a ribosome when the ribosome reaches a 3′ end of the template during translation, translating a transcript, reverse transcribing a transcript, repeating these steps, and selecting one or more nucleotide sequences encoding polypeptides with a high affinity for a target analyte. In some embodiments, the method further comprises performing 1, 2, 3, 4, 5, 6, 7, 8, 9, 10 or more further selection rounds.

In some embodiments, the polypeptide is attached to a transcript encoding the binding moiety that is a polypeptide. In some embodiments, the polypeptide is attached to a molecule that enters the A site of a ribosome when the ribosome reaches a 3′ end of the template during translation. In some embodiments, the polypeptide is attached to a cDNA of a transcript encoding the binding moiety that is a polypeptide.

In some embodiments, the binding moiety is a small molecule. In some embodiments, the small molecule is cyclic.

In some embodiments, the address polynucleotide further comprises an address linker sequence. In some embodiments, the address polynucleotide further comprises an address primer binding sequence. In some embodiments, the address polynucleotide further comprises an address spacer sequence.

In some embodiments, the address polynucleotide is arranged in the order of the address linker sequence, the address barcode, the address primer binding sequence, and the address spacer propagating toward the solid support. In some embodiments, the address polynucleotide is arranged in the order of the address linker sequence, the address barcode, the address primer binding sequence, and the address spacer from a 5′ end of the address polynucleotide to a 3′ end of the address polynucleotide. In some embodiments, an end of the address polynucleotide comprises a functional group. In some embodiments, a 3′ end of the address polynucleotide comprises a functional group. In some embodiments, the functional group of the address polynucleotide is selected from the group consisting of an amino group, a carboxyl group, a hydroxyl group, biotin, avidin, and a phosphate group. In some embodiments, the functional group of the address polynucleotide is avidin.

In some embodiments, the address polynucleotide is coupled to the solid support covalently. In some embodiments, the address polynucleotide is coupled to the solid support non-covalently. In some embodiments, the address polynucleotide is coupled to the solid support by a linker. In some embodiments, the address polynucleotide is coupled to the solid support by the functional group. In some embodiments, the target analyte is coupled to the solid support covalently. In some embodiments, the target analyte is coupled to the solid support non-covalently. In some embodiments, the target analyte is coupled to the solid support by a linker. In some embodiments, the linker is an antibody. In some embodiments, the linker is specific for the target analyte.

In some embodiments, the linker is specific for a post-translational modification of the target analyte. In some embodiments, the linker comprises a plurality of linkers, each specific for a target analyte or modification thereof. In some embodiments, the target analyte is coupled to the solid support by a tag. In some embodiments, the target analyte is coupled to the solid support by a universal tag.

In some embodiments, the coupling comprises coupling the proximity linker sequence to the address linker sequence. In some embodiments, the coupling comprises bringing an end of the proximity polynucleotide to a position adjacent to an end of the address polynucleotide.

In some embodiments, the end of the proximity polynucleotide adjacent to an end of the address polynucleotide is a 3′ end of the proximity polynucleotide. In some embodiments, the end of the address polynucleotide adjacent to an end of the proximity polynucleotide is a 5′ end of the address polynucleotide. In some embodiments, the coupling comprises hybridizing the proximity polynucleotide to a splint polynucleotide.

In some embodiments, the coupling comprises hybridizing the address polynucleotide to a splint polynucleotide. In some embodiments, the coupling comprises hybridizing the address polynucleotide and the proximity polynucleotide and the address polynucleotide to a same splint polynucleotide. In some embodiments, the coupling comprises coupling a plurality of proximity probes to a plurality of address polynucleotides, wherein the coupled proximity probes and address polynucleotides are in the same discrete address region. In some embodiments, the coupling comprises coupling a plurality of proximity probes to a plurality of address polynucleotides simultaneously, wherein the coupled proximity probes and address polynucleotides are in the same discrete address region. In some embodiments, the coupling comprises coupling a plurality of proximity probes to a plurality of address polynucleotides in a same reaction, wherein the coupled proximity probes and address polynucleotides are in the same discrete address region. In some embodiments, the coupling comprises non-covalently attaching the proximity probe to the address polynucleotide. In some embodiments, the coupling comprises hybridizing the address polynucleotide to the proximity polynucleotide. In some embodiments, the coupling comprises indirectly hybridizing the address polynucleotide to the proximity polynucleotide. In some embodiments, the proximity polynucleotide and the address polynucleotide are not complimentary to each other. In some embodiments, the coupling comprises covalently attaching the proximity polynucleotide to the address polynucleotide. In some embodiments, the coupling comprises forming a polynucleotide molecule comprising the proximity barcode and the address barcode. In some embodiments, the coupling comprises indirectly hybridizing the address polynucleotide to the proximity polynucleotide. In some embodiments, the coupling comprises ligating the address polynucleotide to the proximity polynucleotide. In some embodiments, the ligating comprises hybridizing a splint polynucleotide to the proximity linker sequence and the address polynucleotide linker sequence. In some embodiments, the ligating comprises hybridizing an overhang region at an end of the proximity polynucleotide to an overhang region at an end of the address polynucleotide. In some embodiments, the ligating comprises adding a DNA ligase.

In some embodiments, method further comprises contacting a DNA ligase to the solid support. In some embodiments, method further comprises contacting a polymerase to the solid support. In some embodiments, method further comprises contacting a reverse transcriptase to the solid support. In some embodiments, method further comprises contacting a splint polynucleotide to the solid support.

In some embodiments, the solid support comprises a plurality of target analytes, wherein each target analyte of the plurality is different. In some embodiments, the solid support comprises a plurality of target analytes, wherein each target analyte of the plurality is located within a different discrete address region of the solid support. In some embodiments, the proximity probe comprises a plurality of proximity probes, where each proximity probe of the plurality is different. In some embodiments, the proximity probe comprises a plurality of proximity probes, where each proximity probe of the plurality is different. In some embodiments, the binding moiety comprises a plurality of binding moieties, wherein each binding moiety of the plurality is different. In some embodiments, the binding moiety comprises a plurality of binding moieties, wherein two or more binding moieties of the plurality are bound to a target analyte within a different discrete address region of the solid support. In some embodiments, the solid support comprises a plurality of address polynucleotides, wherein each address polynucleotide of the plurality is different. In some embodiments, the solid support comprises a plurality of address polynucleotides, wherein each address polynucleotide of the plurality is located within a different discrete address region of the solid support.

In some embodiments, the amplifying comprises adding a primer set comprising a first primer that binds to a primer binding site upstream of the address barcode of the coupled product or an amplified product thereof; and a second primer that binds to a primer binding site upstream of the proximity barcode of the coupled product or an amplified product thereof. In some embodiments, the first primer comprises a 5′ overhang region. In some embodiments, the 5′ overhang region of the first primer comprises a first universal sequencing primer binding site. In some embodiments, the second primer comprises a 5′ overhang region. In some embodiments, the 5′ overhang region of the second primer comprises a second universal sequencing primer binding site.

In some embodiments, the amplifying comprises PCR. In some embodiments, the amplifying comprises primer extension. In some embodiments, the amplifying comprises reverse transcription. In some embodiments, the amplifying comprises linear amplification. In some embodiments, the amplifying comprises non-linear amplification. In some embodiments, the amplifying is performed on the solid support. In some embodiments, the amplifying comprises amplifying a plurality of coupled products, wherein the plurality of coupled products is amplified simultaneously. In some embodiments, the amplifying comprises amplifying a plurality of coupled products, wherein the plurality of coupled products is amplified in a single reaction.

In some embodiments, the detecting comprises sequencing a coupled product or an amplified product thereof. In some embodiments, the detecting comprises detecting a plurality of different coupled products or amplified products thereof. In some embodiments, the detecting comprises detecting a plurality of different coupled products or amplified products thereof simultaneously. In some embodiments, the detecting comprises detecting a plurality of different coupled products or amplified products thereof in a same reaction.

In some embodiments, the method further comprises identifying a first target analyte as a specific binding partner of a binding moiety or fragment thereof. In some embodiments, the identifying comprises identifying the first target analyte as a specific binding partner of the binding moiety or fragment thereof when a K_{D} of the proximity probe for the target analyte is at most about 1×10⁻⁶, 1×10⁻⁷, 1×10⁻⁸, 1×10⁻⁹, 1×10⁻¹⁰, 1×10⁻¹¹, or less. In some embodiments, the identifying comprises identifying a first target analyte as a specific binding partner of the binding moiety or fragment thereof when a K_{D} of the proximity probe for the first target analyte is at least about 10, 50, 100, 500, 1,000, 5,000, 10,000, or more times less than the K_{D} of the proximity probe for a second target analyte.

In some embodiments, the second target analyte comprises a plurality of second target analytes. In some embodiments, the plurality of second target analytes comprises each second target analyte on the solid substrate to which the proximity probe was contacted.

In some embodiments, the method further comprises identifying a specific binding moiety or fragment thereof for each of a plurality of target analytes. In some embodiments, the method further comprises determining a relative binding affinity of the binding moiety to a first target analyte. In some embodiments, the determining a relative binding affinity comprises determining a number of sequence reads having a same proximity barcode sequence and a same address barcode sequence. In some embodiments, the number of sequence reads is proportional to the relative binding affinity. In some embodiments, the method further comprises determining a binding specificity of the binding moiety to the target analyte. In some embodiments, the determining a binding specificity comprises determining a number of sequence reads having a same proximity barcode and a different address barcode. In some embodiments, the number of sequence reads having a same proximity barcode and a different address barcode is inversely proportional to the binding specificity.

In some embodiments, the solid support comprises a solid support.

In some embodiments, the solid support comprises at least at least 2, or at least about 5, 10, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more discrete address regions. In some embodiments, the solid support comprises from 100 to about 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the solid support comprises from 2 to about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the proteins of an organism's proteome. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors of an organism's proteome. In some embodiments, the organism is selected from the group consisting of mouse, rat, rabbit, cat, dog, bird, horse, pig, monkey, goat, cow, and human. In some embodiments, the organism is human. In some embodiments, the target analyte comprises a plurality of target analytes comprising at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more target analytes. In some embodiments, the address polynucleotide comprises a plurality of address polynucleotides comprising at least at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more address polynucleotides.

In some embodiments, each target analyte of the plurality is different. In some embodiments, each address polynucleotide of the plurality comprises a unique address barcode. In some embodiments, each address polynucleotide of the plurality comprises a same address linker sequence. In some embodiments, each address polynucleotide of the plurality comprises a same address primer binding site.

In some embodiments, the proximity probe comprises a plurality of proximity probes comprising of at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more proximity probes. In some embodiments, each proximity probe of the plurality comprises a unique proximity barcode. In some embodiments, each proximity probe of the plurality comprises a same proximity linker sequence. In some embodiments, each proximity probe of the plurality comprises a same proximity primer binding site. In some embodiments, each proximity probe of the plurality comprises a different binding moiety. In some embodiments, the concentration of the target analyte in a discrete address region is known. In some embodiments, the method further comprises determining a concentration of the target analyte in a discrete address region.

In one aspect, provided herein is a library of binding moieties prepared according to any one of the methods described herein, wherein the library comprises a plurality of binding moieties selected based on one or more characteristics selected from the group consisting of affinity, selectivity, stability, and combinations thereof.

In one aspect, provided herein is a method of making an array, comprising coupling an address polynucleotide to a first discrete location within a discrete address region on a solid support; and coupling a target analyte to a second discrete location within the same discrete address region, wherein the address polynucleotide is barcoded to the target analyte, and wherein the target analyte does not base pair with the address polynucleotide.

In some embodiments, the method comprises coupling each of a plurality of address polynucleotides to a first discrete location of a plurality of first discrete locations, wherein each first discrete location is within a different discrete address region on the solid support; and coupling each of a plurality of target analytes to a second discrete location of a plurality of second discrete locations, wherein each second discrete location is within a different discrete address region on the solid support, wherein each target analyte is in proximity to an address polynucleotide, and wherein each address polynucleotide is barcoded to a different target analyte.

In one aspect, provided herein is a method of making a solid support, comprising coupling a first address polynucleotide to a first discrete location of a first discrete address region on the solid support; coupling a second address polynucleotide to a first discrete location of a second discrete address region on the solid support; coupling a first target analyte to a second discrete location of the first discrete address region on the solid support; and coupling a second target analyte to a second discrete location of the second discrete address region on the solid support; wherein the first address polynucleotide identifies the first discrete address region, an identity of the first target analyte, or both; wherein the second address polynucleotide identifies the second discrete address region, an identity of the second target analyte, or both; and wherein the first and second target analytes are polypeptides or small molecules

In some embodiments, the first and second discrete locations are in proximity. In some embodiments, the target analyte is barcoded to an address barcode sequence of the address polynucleotide. In some embodiments, the address barcode is unique. In some embodiments, the address polynucleotide of a first discrete address region is not in proximity to a target analyte of a second discrete address region. In some embodiments, the address polynucleotide is in proximity to a proximity probe when the proximity probe is bound to the target analyte.

In some embodiments, the proximity probe comprises a binding moiety coupled to a proximity polynucleotide. In some embodiments, the proximity polynucleotide comprises a proximity barcode, a proximity linker sequence, a proximity primer binding sequence, a proximity spacer sequence, or any combination thereof. In some embodiments, the proximity polynucleotide is arranged in the order of the proximity linker sequence, the proximity barcode, the proximity primer binding sequence, and the proximity spacer propagating toward the binding moiety. In some embodiments, the proximity polynucleotide is arranged in the order of the proximity linker sequence, the proximity barcode, the proximity primer binding sequence, and the proximity spacer from a 5′ end of the proximity polynucleotide to a 3′ end of the proximity polynucleotide. In some embodiments, the address polynucleotide is in proximity to the proximity linker sequence when the proximity probe is bound to the target analyte.

In some embodiments, the target analyte is a polypeptide. In some embodiments, the polypeptide is within a virus particle. In some embodiments, the polypeptide is within a virus particle membrane. In some embodiments, the polypeptide is an antibody or fragment thereof. In some embodiments, the polypeptide is a transcription factor. In some embodiments, the polypeptide is a receptor. In some embodiments, the receptor is a transmembrane receptor

In some embodiments, the target analyte is a small molecule. In some embodiments, the small molecule is a drug. In some embodiments, the small molecule is a compound. In some embodiments, the small molecule is an organic compound. In some embodiments, the small molecule has a molecular weight of 900 Daltons or less. In some embodiments, the small molecule has a molecular weight of 500 Daltons or more. In some embodiments, the target analyte does not comprise a phosphodiester linkage. In some embodiments, the target analyte comprises at least two amide bonds. In some embodiments, the target analyte is not DNA or RNA.

In some embodiments, the address polynucleotide further comprises an address linker sequence.

In some embodiments, the address polynucleotide further comprises an address primer binding sequence. In some embodiments, the address polynucleotide further comprises an address spacer sequence. In some embodiments, the address polynucleotide is arranged in the order of the address linker sequence, the address barcode, the address primer binding sequence, and the address spacer propagating toward the solid support. In some embodiments, the address polynucleotide is arranged in the order of the address linker sequence, the address barcode, the address primer binding sequence, and the address spacer from a 3′ end of the address polynucleotide to a 5′ end of the address polynucleotide. In some embodiments, the address linker sequence is in proximity to the proximity linker sequence when the proximity probe is bound to the target analyte. In some embodiments, an end of the address polynucleotide comprises a functional group. In some embodiments, a 3′ end of the address polynucleotide comprises a functional group. In some embodiments, the functional group of the address polynucleotide is selected from the group consisting of an amino group, a carboxyl group, a hydroxyl group, biotin, avidin, and a phosphate group. In some embodiments, the functional group of the address polynucleotide is avidin.

In some embodiments, the coupling the address polynucleotide comprises non-covalently attaching the address polynucleotide to the solid support. In some embodiments, the coupling the address polynucleotide comprises covalently attaching the address polynucleotide to the solid support. In some embodiments, the coupling the address polynucleotide or the target analyte comprises reactive plasma etching, corona discharge treatment, a plasma deposition process, spin coating, dip coating, spray painting, deposition, printing, stamping. In some embodiments, the coupling the address polynucleotide comprises coupling the address polynucleotide to a linker. In some embodiments, the coupling the target analyte comprises non-covalently attaching the target analyte to the solid support. In some embodiments, the coupling the target analyte comprises covalently attaching the target analyte to the solid support. In some embodiments, the coupling the target analyte comprises coupling the target analyte to a linker. In some embodiments, the coupling the first and second target analytes comprises coupling the first target analyte to a first linker and the second target analyte to a second linker.

In some embodiments, the first linker is a first antibody and the second linker is a second antibody. In some embodiments, the first linker is specific for the first target analyte or a post-translational modification thereof, and the second linker is specific for the second target analyte or a post-translational modification thereof. In some embodiments, the linker is an antibody. In some embodiments, the linker is specific for the target analyte. In some embodiments, the linker is specific for a post-translational modification of the target analyte. In some embodiments, the linker comprises a plurality of linkers, each specific for a target analyte or modification thereof.

In some embodiments, the solid support comprises a plurality of target analytes, wherein each target analyte of the plurality is different. In some embodiments, the solid support comprises a plurality of target analytes, wherein each target analyte of the plurality is located within a different discrete address region of the solid support. In some embodiments, the solid support comprises a plurality of address polynucleotides, wherein each address polynucleotide of the plurality is different. In some embodiments, the solid support comprises a plurality of address polynucleotides, wherein each address polynucleotide of the plurality is located within a different discrete address region of the solid support.

In some embodiments, the solid support comprises an array. In some embodiments, the array comprises at least 2, or at least about 5, 10, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more discrete address regions. In some embodiments, the array comprises from 100 to about 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the array comprises from 2 to about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 discrete address regions. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the proteins of an organism's proteome. In some embodiments, the target analyte comprises a plurality of proteins comprising at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the transcription factors of an organism's proteome. In some embodiments, the organism is selected from the group consisting of mouse, rat, rabbit, cat, dog, bird, horse, pig, monkey, goat, cow, and human. In some embodiments, the organism is human.

In some embodiments, the target analyte comprises a plurality of target analytes comprising at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more target analytes. In some embodiments, each target analyte of the plurality is different. In some embodiments, the address polynucleotide comprises a plurality of address polynucleotides comprising at least at least 2, or at least about 5, 10, 100, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000 or more address polynucleotides. In some embodiments, each address polynucleotide of the plurality comprises a unique address barcode. In some embodiments, each address polynucleotide of the plurality comprises a same address linker sequence. In some embodiments, each address polynucleotide of the plurality comprises a same address primer binding site. In some embodiments, a known concentration of the target analyte is coupled to the discrete address region.

In some embodiments, the target analyte is from a biological sample. In some embodiments, the target analyte is from a cell. In some embodiments, the target analyte is in a lysate of the cell. In some embodiments, the target analyte is an immunoprecipitation from the cell. In some embodiments, the cell is a single cell. In some embodiments, the cell is a plurality of cells.

In one aspect, provided herein is a method comprising: contacting a first plurality of aptamers to a sample comprising a plurality of target analytes; forming a first complex between an aptamer of the first plurality and a target analyte of the plurality; and detecting the aptamer of the first complex or an amplified product thereof, wherein the aptamer of the first complex comprises an aptamer barcode sequence that identifies the target analyte the first complex.

In some embodiments, the method further comprises determining a level of the target analyte of the first complex. In some embodiments, the target analyte of the first complex is coupled to a solid support. In some embodiments, the method further comprises washing the solid support. In some embodiments, the method further comprises amplifying the aptamer barcode sequence of the aptamer of the first complex. In some embodiments, the detecting comprises sequencing the aptamer barcode sequence of the aptamer of the first complex or an amplified product thereof. In some embodiments, the determining a level of the target analyte of the first complex comprises determining a number of sequence reads comprising the aptamer barcode sequence of the aptamer of the first complex. In some embodiments, the target analyte of the first complex is a polypeptide comprising an interacting polynucleotide. In some embodiments, the interacting polynucleotide is genomic DNA. In some embodiments, the interacting polynucleotide comprises an adaptor on one or both ends. In some embodiments, the method further comprises amplifying a sequence of the interacting polynucleotide. In some embodiments, the method further comprises determining a sequence of the interacting polynucleotide that interacts with the target analyte of the first complex. In some embodiments, the solid support is a bead.

In some embodiments, the method further comprises contacting a second plurality of aptamers to the sample, and forming a second complex comprising an aptamer of the second plurality and a binding moiety of the plurality; wherein the aptamer of the second complex comprises an aptamer barcode that identifies the binding moiety of the second complex. In some embodiments, the method further comprises coupling an aptamer of the first complex to an aptamer of the second complex, wherein the target analyte of the first complex interacts with the binding moiety of the second complex to form a third complex. In some embodiments, the coupling comprises ligating.

In some embodiments, the method further comprises identifying the target analyte of the first complex as a binding partner of the binding moiety of the second complex. In some embodiments, the method further comprises determining an affinity of the binding moiety of the second complex for the target analyte of the first complex. In some embodiments, the method further comprises determining a selectivity of the binding moiety of the second complex for the target analyte of the first complex. In some embodiments, the method further comprises determining a level of the third complex in the sample.

In one aspect, provided herein is a solid support comprising a plurality of first complexes, wherein each complex of the plurality of first complexes comprises an aptamer of a first plurality of aptamers bound to a target analyte, wherein each aptamer of the first plurality of aptamers comprises an aptamer barcode sequence that identifies the target analyte to which it is bound.

In some embodiments, the solid support further comprises a plurality of second complexes, wherein each complex of the plurality of second complexes comprises an aptamer of a second plurality of aptamers bound to a binding moiety, wherein each aptamer of the second plurality of aptamers comprises an aptamer barcode sequence that identifies the binding moiety to which it is bound, and wherein a target analyte of a complex of the plurality of first complexes interacts with a binding moiety of a complex of the plurality of second complexes to form a third complex.

In some embodiments, the plurality of first complexes or the plurality of second complexes are coupled to the solid support covalently, non-covalently, by a functional group, or by a linker.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A depicts a schematic of the various compositions used in the methods described herein. An array comprising a solid support is depicted, wherein an address polynucleotide comprising an address barcode is in proximity to a target analyte which is bound/complexed to a binding moiety comprising a proximity polynucleotide comprising a proximity barcode, wherein the target analyte is coupled to the solid support and the address polynucleotide is coupled to the solid support.

FIG. 1B depicts a schematic of the various compositions used in the methods described herein to determine protein-target analyte interactions, such as protein-protein interactions (pDAPPL).

FIG. 1C depicts a schematic of the various compositions used in the methods described herein to determine antibody-target analyte interactions, such as antibody-protein interactions (aDAPPL).

FIG. 1D depicts a schematic of the various compositions used in the methods described herein to determine DNA-target analyte interactions, such as DNA-transcription factor (TF) interactions, using a proximity probe containing a proximity barcode.

FIG. 1E depicts a schematic of the various compositions used in the methods described herein to determine DNA-target analyte interactions, such as DNA-TF interactions, where the proximity barcode is a sequence of the DNA.

FIG. 1F depicts a schematic of the various compositions used in the methods described herein to determine RNA-target analyte interactions (rDAPPL), such as RNA-RNA-binding protein (RBP) interactions, using a proximity probe containing a proximity barcode.

FIG. 1G depicts a schematic of the various compositions used in the methods described herein to determine RNA-target analyte interactions (rDAPPL) such as RNA-RBP interactions, where the proximity barcode is a sequence of the RNA.

FIG. 1H depicts a schematic of the various compositions used in the methods described herein to determine aptamer-target analyte interactions using a proximity probe containing a proximity barcode.

FIG. 1I depicts a schematic of the various compositions used in the methods described herein to determine aptamer-target analyte interactions where the proximity barcode is a sequence of the aptamer.

FIG. 1J depicts a schematic of the various compositions used in the methods described herein to determine small molecule-target analyte interactions.

FIG. 1K depicts a schematic of the various compositions used in the methods described herein. An array comprising a solid support is depicted, wherein an address polynucleotide comprising an address barcode is in proximity to a target analyte which is bound/complexed to a binding moiety comprising a proximity polynucleotide comprising a proximity barcode, wherein the target analyte is coupled to the solid support via a linker and the address polynucleotide is coupled to the solid support.

FIG. 1L depicts a schematic of the various compositions used in the methods described herein. An array comprising a solid support is depicted, wherein an address polynucleotide comprising an address barcode is in proximity to a target analyte which is bound/complexed to a binding moiety comprising a proximity polynucleotide comprising a proximity barcode, wherein the target analyte is coupled to the solid support via an antibody linker and the address polynucleotide is attached to the solid support.

FIG. 1M depicts a schematic of the various compositions used in the methods described herein. An array comprising a solid support is depicted, wherein an address polynucleotide comprising an address barcode is in proximity to a target macrocycle/small molecule which is bound/complexed to a binding moiety comprising a proximity polynucleotide comprising a proximity barcode, wherein the target macrocycle/small molecule is coupled to the solid support and the address polynucleotide is attached to the solid support.

FIG. 2A depicts a schematic of the oligonucleotides of the compositions used in the methods described herein where the proximity probe contains a proximity barcode.

FIG. 2B depicts a schematic of the oligonucleotides of the compositions used in the methods described herein where the binding moiety is a polynucleotide, such as a DNA, RNA, or aptamer that contains a proximity barcode.

FIG. 3A depicts a schematic of a protein array, a magnification of a portion of the array, and a magnification of one well of the portion of the array.

FIG. 3B depicts a schematic of a virion array comprising the depicted exemplary membrane proteins within virion particles.

FIG. 4A depicts a schematic of the ligation of the methods described herein.

FIG. 4B depicts a schematic of the amplification and sequencing steps of the methods described herein.

FIG. 5A depicts a solid support with an address polynucleotide comprising an address barcode in proximity to a target analyte which is bound/complexed to a binding moiety comprising a proximity polynucleotide comprising a proximity barcode, wherein the target analyte is attached to the solid support and the address polynucleotide is attached to the solid support.

FIG. 5B depicts a solid support, with an address polynucleotide comprising an address barcode in proximity to a target analyte which is bound/complexed to a binding moiety comprising a proximity polynucleotide which comprises a proximity barcode, wherein the target analyte is attached to the solid support and the address polynucleotide is attached to the solid support, wherein the proximity polynucleotide and the address polynucleotide are ligated.

FIG. 6 depicts a schematic of the step of library preparation for sequencing of the ligated products produced by the methods described herein.

FIG. 7A depicts a schematic of the methods described herein to determine aptamer-protein interactions.

FIG. 7B depicts a schematic of the methods described herein to determine DNA-protein interactions.

FIG. 7C depicts a schematic of the methods described herein to determine DNA-protein interactions.

FIG. 8A depicts a schematic of the methods described herein to apply previously identified aptamers that specifically bind to a target protein to enable proteome-wide detection of protein abundance inside a cell or tissue (WB-omix). Briefly, after a pool of the aptamers is mixed with biotinylated lysates, streptavidin beads are added to the mixture and washed. In the example shown, each of the aptamers in the pool is known to specifically interact with a particular protein Bound aptamers are then PCR amplified. The number of sequence reads for a particular aptamer sequence can be used to determine the approximate level or relative abundance of a particular protein.

FIG. 8B depicts a schematic of a bead coupled via an avidin-biotin linker to a complex of a target analyte bound to an aptamer containing a pair of adapter sequences for PCR amplification.

FIG. 8C depicts a schematic of a complex of a target analyte bound to an aptamer containing a pair of adapter sequences and coupled to a bead via hybridization with the adapter sequences.

FIG. 8D depicts a schematic of a bead coupled to a plurality of complexes each containing a target analyte bound to an aptamer containing a unique sequence corresponding to the target analyte to which the aptamer is bound.

FIG. 9A depicts a schematic of the methods described herein to apply previously identified aptamers that specifically bind to a target protein to determine protein-protein interactions by testing multiple combinations using lysates from cells (e.g., a cell line) and/or tissue (PPI-omix). A mixture of DNA/RNA aptamers that each recognizes a unique human protein is utilized to examine all possible combinations of protein-protein interactions inside a cell or tissue.

FIG. 9B depicts a schematic of a bead coupled to one target analyte of a protein complex, comprised of either two different proteins (i.e., a heterodimer) or the same proteins (i.e., a homodimer), wherein each target analyte is bound to an aptamer and the two aptamers are ligated via a splint polynucleotide hybridized to the adapter sequences of each aptamer. When two different aptamers are found in the same sequence read based on the sequencing results, it indicates formation of a heterodimer. When the same aptamers are found on the same read sequence based on the sequencing results, it indicates formation of a homodimer.

FIG. 9C depicts a schematic of a bead coupled via hybridization to a protein complex, wherein each target analyte is bound to an aptamer and the two aptamers are ligated via a splint polynucleotide hybridized to the adapter sequences of each aptamer. When two different aptamers are found in the same sequence read based on the sequencing results, it indicates formation of a heterodimer. When the same aptamers are found on the same read sequence based on the sequencing results, it indicates formation of a homodimer.

FIG. 9D depicts a schematic of a bead coupled to a plurality of protein complexes, wherein each target analyte of a complex is bound to an aptamer and the two aptamers of each complex are ligated via a splint polynucleotide hybridized to the adapter sequences of each aptamer. When two different aptamers are found in the same sequence read based on the sequencing results, it indicates formation of a heterodimer. When the same aptamers are found on the same read sequence based on the sequencing results, it indicates formation of a homodimer.

FIG. 10 depicts a schematic of the methods described herein to perform screens for disease-specific aptamers.

FIG. 11 depicts a schematic exemplary methods and uses of dimeric and multimeric aptamer scaffolds.

FIG. 12A depicts a schematic of the methods described herein to perform aptamer screens against human transmembrane proteins on virion arrays (VirD arrays). Briefly, a library of DNA or RNA aptamers containing ends with fixed sequences are incubated on an array of virus particles (i.e., virions) containing transmembrane proteins of interest and bound aptamers are then recovered and amplified. Asymmetric amplification of the amplified products is then performed to regenerate the aptamers. In the case of using an RNA aptamer library, in vitro transcription is then performed to regenerate the RNA aptamers. This process is repeated for 4 cycles. During the 5^{th} cycle, bound aptamers are ligated to address polynucleotides, amplified, and sequenced to identify transmembrane proteins recognized by the aptamers. This process is repeated for a 6^{th} and 7^{th} cycle. Sequencing data from cycles 5, 6, and 7 can be compared to identify high affinity aptamers specific to transmembrane proteins.

FIG. 12B depicts a schematic of the methods described herein to perform peptide ligand screens for human G-protein coupled receptors (GPCRs) on VirD arrays.

FIG. 13 depicts a graph of the number of transcription factor binding motifs as a function of the number of binding proteins.

FIG. 14 depicts a schematic for DNA probe preparation.

FIG. 15A depicts a schematic of the methods described herein using a transcription factor array and a library of DNA probes with potential transcription factor binding motifs (“N₄CCGGN₄” as SEQ ID NO: 1).

FIG. 15B depicts unmethylated and methylated DNA probes (unmethylated probes 3 and 6 disclosed as SEQ ID NO: 1, methylated probes 9 and 13 disclosed as SEQ ID NO: 2, and methylated probes 10 and 14 disclosed as SEQ ID NO: 3, respectively, in order of appearance) used in the methods described herein for determining binding of proteins to methylated and unmethylated DNA binding sites.

FIG. 16 depicts an agarose gel of three polynucleotide libraries (SEQ ID NOS 6-8, respectively, in order of appearance) used for determining binding of proteins to methylated and unmethylated DNA binding sites.

FIG. 17 depicts a schematic of the methods described herein using a transcription factor array and a library of methylated DNA probes with potential protein binding motifs.

FIG. 18 depicts a heat map generated from the methods in FIG. 17 used to determine the transcription factors that bind to the probes.

FIG. 19A depicts a schematic of the methods described herein using a transcription factor array and a library of methylated DNA probes with potential protein binding motifs. In the example shown, the library covers the entire 8-mer DNA space, wherein ˜104 million binding events (=1,620 TF proteins×65,536 DNA sequences) are surveyed simultaneously. Also depicted is the recovery of a methylated DNA motif 5′-ACGGmCGGATAat (SEQ ID NO: 4) KLF4 protein spots (“AAAGmCGTTCA” disclosed as SEQ ID NO: 9).

FIG. 19B depicts a schematic of the methods described herein using a transcription factor array and a library of methylated DNA probes with potential protein binding motifs. hTF=human transcription factor.

FIG. 20 depicts agarose gels demonstrating detection of ligated DNA after performing protein-DNA binding assays on arrays to detect interactions between a methylated motif and a human TF, such as those depicted in FIG. 19A and FIG. 19B, and amplification by PCR with the indicated number of PCR cycles. Controls are depicted on the left. The right panel shows that MPG2 captures a methylated motif M62 (SEQ ID NO: 10).

FIG. 21 depicts Sanger sequencing results confirming the expected sequences (SEQ ID NOS 11-15, respectively, in order of appearance) of the ligated products after performing protein-DNA binding assays on arrays to detect interactions between a methylated motif and a human TF, such as those depicted in FIG. 20.

FIG. 22 depicts agarose gels demonstrating detection of ligated DNA after performing protein-DNA binding assays on arrays with two different probe sets and the indicated methylated polynucleotide probe libraries.

FIG. 23 depicts an agarose gel of input DNA probes loaded on the gel at the indicated concentrations. Ligated products were analyzed by gel electrophoresis. Competitor (double stranded DNA) was analyzed separately.

FIG. 24 depicts agarose gels of PCR amplified ligated products at the indicated number of PCR cycles.

FIG. 25A depicts a schematic of the methods described herein to determine RNA-protein interactions (rDAPPL). RBP=RNA binding protein.

FIG. 25B depicts an agarose gel of PCR ligation products in an RNA DAPPL (rDAPPL) assay to detect interactions between MSI1 (SEQ ID NOS 16 and 16, respectively, in order of appearance) and Qk1 with their known RNA sequences.

FIG. 25C depicts Sanger sequencing confirming the expected sequences (SEQ ID NOS 17-25, respectively, in order of appearance) of the ligated products after performing an RNA DAPPL (rDAPPL) assay to detect interactions between MSI1 and Qk1 with their known RNA sequences, such as those depicted in FIG. 25A and FIG. 25B.

FIG. 26A depicts a schematic of the methods described herein to determine antibody-protein interactions (aDAPPL). Ag=Antigen (target analyte), Ab=Antibody.

FIG. 26B depicts a schematic of the preparation and conjugation of an antibody with a polynucleotide for use in the methods described herein to determine antibody-protein interactions (aDAPPL).

FIG. 26C depicts a polyacrylamide gel stained with Coommaisse (top) and visualization of the fluorophore Cy5 on the same polyacrylamide gel demonstrating coupling of a polynucleotide to an antibody, at different ratios between the antibody and polynucleotides.

FIG. 26D depicts a schematic of a design for an exemplary aDAPPL assay and corresponding chip (left) and an agarose gel of PCR ligation products in an aDAPPL assay

FIG. 26E depicts labeling of antibodies with maleimide proximity oligos (POs) and labeling of address oligos with biotin and streptavidin.

FIG. 26F depicts an exemplary process for generating cohesive sticky ends of address oligos and proximity oligos for aDAPPL.

FIG. 26G depicts an exemplary process and design for probing macrocycle and protein binding partners of FKBP1A (GST tagged) (SEQ ID NOS 26-31, respectively, in order of appearance).

FIG. 26H depicts a stained agarose gel of primary PCR products.

FIG. 26I depicts a stained agarose gel of the primary PCR products after a cleaning step (left) and a stained agarose gel of secondary PCR products using AO_F1 and PO_R2-2 primers as shown in FIG. 26G.

FIG. 27A depicts a schematic of the methods described herein to determine protein-protein interactions (pDAPPL).

FIG. 27B depicts a schematic of the preparation and conjugation of a protein with a polynucleotide for use in the methods described herein to determine protein-protein interactions (pDAPPL). In the schematic shown, proteins are expressed with a glutathione S-transferase (GST) tag. Conjugation of the proteins to a polynucleotide is predominantly through a thiol group on the GST tag.

FIG. 27C depicts a polyacrylamide gel stained with Coommaisse (top) and visualization of the fluorophore Cy5 on the same polyacrylamide gel demonstrating coupling of a polynucleotide with a protein.

FIG. 28 depicts a schematic of the methods described herein to perform a multiplexed chromatin-immunoprecipitation coupled deep-sequencing (ChIP-seq) method to enable simultaneous detection of transcription factor (TF) binding sites in chromatin. Briefly, multiple monoclonal antibodies against different transcription factors are spotted onto a nitrocellulose-coated slide with address polynucleotides. Cells are then processed to crosslink the TFs to genomic DNA; the genomic DNA is then sheared, and the sheered ends are ligated to a “Y” shaped DNA adapter. These chromatin preps are then incubated on the array to capture chromatin-DNA complexes by the Abs spotted on the array. A splint polynucleotide is then added to facilitate ligation between captured genomic DNA fragments to the address polynucleotides. The ligated products are then amplified and sequenced. Bioinformatics analyses are performed to deconvolute the sequencing data and identify TF binding sites. When the sequence reads are binned by each address polynucleotide, a given TF as represented by a particular address polynucleotide is connected to the genomic sequences that it binds to, and hence, resulted in mapping of its chromatin binding sites.

FIG. 29 depicts a schematic of the methods described herein for quantification of proteins of interest and/or posttranslational modifications of proteins of interest from single cells. Direct detection of phosphoproteins is depicted on the left. Briefly, a group of single cells (e.g., ˜100-1,000) or a group of lysates each extracted from a single cell thereof is placed into separate spots on an array or into a well of a 384- or 1536-well titer dish with a unique address polynucleotide (AO). A library of proximity probes comprising a plurality of binding moieties (e.g., Abs) specific to proteins of interest or posttranslational modifications of proteins of interest is then added to each well, the proximity polynucleotides are then ligated to address polynucleotides on the array or in each well, and the ligated products are amplified and sequenced. Identity of each single cell is represented by the address polynucleotides and identity of the proteins of interest or posttranslational modifications of proteins of interest is represented by the proximity polynucleotide sequences of the proximity probes. Detection of the phospho-tyrosine phosphorylome in a sandwich format is depicted on the right. Briefly, an antibody that detects a desired posttranslational modification (e.g., phospho-tyrosine) is coated on multiple spots on an array or in a well of a 384- or 1536-well titer dish with a unique address polynucleotide. A group of lysate extracted from single cells is then place into separate spots on the array or a particular well in the titer dish. A library of proximity probes comprising a plurality of binding moieties (e.g., Abs) specific to proteins of interest is then added to the array or to each well; the proximity polynucleotides are then ligated to address polynucleotides, and the ligated products are amplified and sequenced. Identity of each single cell is represented by the address polynucleotides and identity of the tyrosine-phosphorylated proteins is represented by the proximity polynucleotide sequences of the proximity probes.

FIG. 30 depicts a schematic of the methods described herein for detection and quantification of specific posttranslational modifications (PTMs) on histone proteins from single cells. Briefly, a generic anti-histone antibody is coated on bottom of each well in a titer dish. A group of lysates extracted from single cells is then placed into separate wells in the titer dish. A library of proximity probes comprising a plurality of binding moieties (e.g., Abs) specific to a desired histone PTM is then added to each well, the proximity polynucleotides are then ligated to address polynucleotides, and the ligated products are amplified and sequenced.

FIG. 31A depicts a schematic of the methods described herein to screen and identify DNA or RNA aptamers that specifically bind transcription factors and protein kinases with mono-specificity and high affinity. Briefly, a library of DNA or RNA aptamers is incubated on an array of containing transcription factors and/or protein kinases of interest. Bound aptamers are then recovered and amplified. Asymmetric amplification of the amplified products is then performed to regenerate the DNA aptamers. In the case of using an RNA aptamer library, in vitro transcription is then performed to regenerate the RNA aptamers. This process is repeated for 4 cycles. During the 5^{th} cycle, bound aptamers are ligated to address polynucleotides, amplified, and sequenced to identify transcription factors and/or protein kinases recognized by the aptamers. This process is repeated for a 6^{th} and 7^{th} cycle. Sequencing data from cycles 5, 6, and 7 can be compared to identify high affinity aptamers specific to transcription factors and/or protein kinases.

FIG. 31B depicts graphs comparing aptamer-protein interactions from the indicated cycles using the method depicted in FIG. 31A.

FIG. 31C depicts an alignment of aptamer sequences (SEQ ID NOS 32-77, respectively, in order of appearance) identified using the method depicted in FIG. 31A and aptamer consensus sequences for binding to IKZF1. Identification of significant consensus aptamer sequences are is a good indicator of aptamer sequence enrichment. Bioinformatics analysis also indicated clear enrichments observed in later rounds of selection.

FIG. 31D depicts exemplary structural consensus sequences (SEQ ID NOS 78 and 79, respectively, in order of appearance) from aptamers identified using the method depicted in FIG. 31A.

FIG. 31E depicts agarose gels of DNA template (left), transcribed RNA probes (middle), and PCR amplified ligated rDAPPL products from the 3^{rd} cycle using the method depicted in FIG. 31A.

FIG. 32A depicts a schematic of the methods described herein to screen and identify synthetic heavy chain variable region (V_{H}) and light chain variable region (V_{L}) single domains that can specifically recognize human proteins with high affinity (e.g., recDAPPL). Briefly, a synthetic pool of single chain V_{H} or V_{L} cDNA sequences are in vitro transcribed and tethered with a puromycin-labeled DNA polynucleotide to the 3′-ends of the transcribed RNA species. In vitro translation reaction is then performed and the RNA templates are tethered to the translated protein products (e.g., a library of V_{H} or V_{L} single domains). The corresponding cDNAs are reverse-transcribed with a primer complementary to the puromycin-labeled DNA moiety, followed by an RNase treatment to remove the RNA moieties. If necessary, translated single domains can be purified with the HisX6 tag (6-His) (SEQ ID NO: 5) using a nickel column to ensure full-length products. After being incubated on an array of target proteins, tethered cDNAs are amplified and this process is repeated for 6 cycles. During the 7^{th} cycle, tethered cDNAs are ligated to address polynucleotides, amplified, and sequenced to identify proteins recognized single chain V_{H} or V_{L} domains. If necessary, tethered cDNAs can be ligated to the address polynucleotides in cycles 4, 5, or 6.

FIG. 32B depicts an exemplary design schematic for the methods depicted in FIG. 32A to screen affinity reagents, e.g., heavy chain variable region (V_{H}) and light chain variable region (V_{L}) single domains, against the human proteome (“His×6” disclosed as SEQ ID NO: 5).

FIG. 32C depicts an exemplary schematic of the methods described herein to screen and identify synthetic V_{H} single domains that can specifically recognize human RAS with high affinity (SEQ ID NO: 80).

FIG. 32D depicts agarose gels stained for DNA and visualization on the same agarose gels demonstrating in vitro transcription of RNA and DNA-puromycin linker ligation.

FIG. 32E depicts a polyacrylamide gel stained with Coommaisse (right) showing in vitro translation of anti-RAS V_{H} and a Western blot with an anti-FLAG antibody (left).

FIG. 33 depicts a schematic of the methods described herein to apply previously identified aptamers that specifically bind to a transcription factor to perform a comprehensive chromatin-IP assay against a plurality of human TFs simultaneously with a mixture of their corresponding aptamers (ChIP-omix). Briefly, a library of identified aptamers that contain adaptor sequences on their ends, and that specifically bind to a target protein, are biotinylated and mixed with chromatin preparations in which genomic DNA from cells has been sheered and modified to contain “Y” adapters on their ends. The ends of the aptamers are then ligated to the “Y” adapter ends of the sheered genomic DNA. Beads coated in streptavidin are added to the mixture. After washing, bound ligation products are amplified and sequenced. The sequence of an aptamer can be used to identify the TF and the genomic DNA sequences ligated to this aptamer can be used to identify chromatin sequences to which that TF binds.

FIG. 34 depicts a schematic of the methods described herein to perform small molecule inhibitor screens against ion channels using an array of virion particles containing transmembrane proteins of interest. Virion-displayed ion channels (e.g., 64 recombinant virions) are spotted in duplicate at bottom of every well in a 96-well plate. After virions in the plates are loaded with the dyes (e.g., ANG-2 for sodium channel imaging), a collection of compounds (e.g., Sigma LOPAC and Microsource Spectrum) will be robotically added to each well to a final concentration of 10 μM. After incubation, the plates will be loaded onto a BD Pathway Imager to establish a baseline, followed by adding stimulus buffer. The fluorescence signals are continuously detected for 180 sec. Signals obtained from WT virions will sever as baseline of fluorescent signal detection. Compared to the WT virions, a compound that causes a signal reduction >3 standard deviations in activity will be scored as a hit.

FIG. 35A depicts an exemplary design of address oligos, a 40-mer DNA aptamer library, and sequences reads designated as indicating a positive interaction for a dDAPPL method as described herein.

FIG. 35B depicts a graph of the distribution of aptamer sequences (SEQ ID NOS 81-90, respectively, in order of appearance) identified using a 40-mer DNA aptamer library in a dDAPPL method as described herein.

FIG. 36A depicts an agarose gel showing unexpected DAPPL ligation products at higher than expected molecular weights when an aptamer-protein DAPPL screen was performed according to the accompanying schematic (left).

FIG. 36B depicts an agarose gel showing a single clean band corresponding to a DAPPL ligation product when a modified aptamer-protein DAPPL screen was performed according to the accompanying schematic (left). The redesigned DAPPL produced a clean, single-banded ligation product of 119 bp.

FIG. 37A depicts exemplary macrocycles utilized to screen and identify macrocycle-protein interactions (SEQ ID NOS 91 and 92, respectively, in order of appearance).

FIG. 37B depicts a schematic of the methods described herein to screen and identify macrocycle-protein interactions.

FIG. 37C depicts an agarose gel of eluted DAPPL products that were amplified using PCR for 40 cycles. 10 colonies were chosen for Sanger sequencing; 7 were confirmed correctly ligated.

FIG. 37D depicts Sanger sequencing confirming the expected sequences (SEQ ID NOS 93 and 94, respectively, in order of appearance) of the ligated products after performing a macrocycle-protein DAPPL assay to detect interactions between Src macrocycle 1 and IDE macrocycle 6 with Src1 or IDE6.

FIG. 38A depicts an exemplary DAPPL-PPI method schematic. FK506, GST and printing buffer were mixed with different address oligos and then printed on an array.

FIG. 38B depicts labeling of FKBP1A (GST tagged) and GST with different 5′ maleimide proximity oligos (POs).

FIG. 38C depicts an exemplary process for generating cohesive sticky ends of address oligos and proximity oligos.

FIG. 38D depicts an exemplary process for probing macrocycle and protein binding partners of FKBP1A (GST tagged). FKBP1A binds to FK506 and rapamycin. Proximity oligo #1 is close to address oligo #1 and #2 and ready for ligation. GST with proximity oligo #2 is washed away.

FIG. 38E depicts an exemplary process and design for probing macrocycle and protein binding partners of FKBP1A (GST tagged) (SEQ ID NOS 26-31, respectively, in order of appearance).

FIG. 38F depicts a stained agarose gel of primary PCR products.

FIG. 38G depicts a stained agarose gel of the primary PCR products after a cleaning step (left) and a stained agarose gel of secondary PCR products using AO_F1 and PO_R2-2 primers as shown in FIG. 38E.

FIG. 39A depicts a schematic of the methods described herein to perform RNA aptamer screening screens against human kinases to identify phospho-specific RNA aptamers potential or application as therapies. RNA aptamers can be induced to express in cells and phospho-specific RNA aptamers can serve as a unique set of molecular tools for dissecting protein kinase functions in cells.

FIG. 39A depicts a stained agarose gel showing DAPPL product detection using the RNA aptamer screening methodology as shown in FIG. 39B.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

### DEFINITIONS

The terms “attach”, “bind”, “couple”, and “link” are used interchangeably and refer to covalent interactions (e.g., by chemically coupling), or non-covalent interactions (e.g., ionic interactions, hydrophobic interactions, hydrogen bonds, hybridization, etc.). The terms “specific”, “specifically”, or specificity” refer to the preferential recognition, contact, and formation of a stable complex between a first molecule and a second molecule compared to that of the first molecule with any one of a plurality of other molecules (e.g., substantially less to no recognition, contact, or formation of a stable complex between the first molecule and any one of the plurality of other molecules). For example, two molecules may be specifically attached, specifically bound, specifically coupled, or specifically linked. For example, specific hybridization between a first polynucleotide and a second polynucleotide can refer to the binding, duplexing, or hybridizing of the first polynucleotide preferentially to a particular nucleotide sequence of the second polynucleotide under stringent conditions. A sufficient number complementary base pairs in a polynucleotide sequence may be required to specifically hybridize with a target nucleic acid sequence. A high degree of complementarity may be needed for specificity and sensitivity involving hybridization, although it need not be 100%.

The term “barcoded to” refers to a relationship between molecules where a first molecule contains a barcode that can be used to identify a second molecule.

“Proximity” or “in proximity to” refers to a distance between two locations or molecules relative to each other that allows a reaction to take place. The distance, can be a length that permits the address polynucleotide of a first discrete location of a discrete region to be coupled, such as through ligation, to a proximity probe when the proximity probe is bound to a target analyte at a second discrete location of the discrete region.

### Digital Affinity Profiling Via Proximity Ligation (DAPPL)

The present invention relates to methods, kits, and compositions for Digital Affinity Profiling via Proximity Ligation (DAPPL) that can be used to screen multiple binding moieties against multiple target analytes using proximity coupling and deep sequencing techniques in an extremely high-throughput manner. The present invention relates to a proximity-probe based detection assay, (e.g., a proximity ligation assay (PLA)), for detecting binding of a binding moiety to an analyte in a sample, such as a target analyte on a solid support. For example, by screening mAbs against a proteome array (e.g., human proteome microarray) using the methods and compositions described herein, corresponding antigen(s) recognized by a given mAb can be directly identified.

Proximity ligation assays rely on proximal binding of proximity probes to an analyte to generate a signal from a ligation reaction involving or mediated by (e.g. between and/or templated by) nucleic acid domains of the proximity assays. Proximity-probe based detection assays, permit sensitive, rapid, and convenient detection and/or quantification of one or more analytes in a sample by converting the presence of such an analyte into a readily detectable or quantifiable nucleic acid-based signal, and can be performed in homogeneous or heterogeneous formats. Proximity probes of the art are generally used in pairs, and individually consist of an analyte-binding domain with specificity to the target analyte, and a functional domain, e.g. a nucleic acid domain coupled thereto.

The methods, kits, and compositions described herein rely on the principle of proximity probing, wherein a binding moiety's interaction with an analyte is detected through the coupling of multiple (e.g., two or three or four or more) polynucleotide probes, which when brought into proximity by interaction of a binding moiety to an analyte, allow a signal to be generated. Typically, at least one of the proximity probes comprises a nucleic acid domain (e.g., a polynucleotide) linked to an analyte-binding domain (e.g., a binding moiety). Generation of a signal can involve an interaction between a nucleic acid moiety of the proximity probe and a nucleic acid domain (e.g., a polynucleotide) comprised on another probe comprising a polynucleotide, such as an address polynucleotide. Generation of a signal can depend on or indicate an interaction between the probes exists. Thus, because binding of binding moiety of a proximity probe to a target analyte brings a nucleic acid domain of the proximity probe into proximity to a nucleic acid domain (e.g., a polynucleotide) comprised on another probe, such as an address polynucleotide, generation of a signal can depend on, indicate, or be used to determine, for example, an interaction, affinity, specificity or a combination thereof, between the binding moiety and the target analyte. For example, an affinity, or strength of an interaction between a binding moiety and a target analyte can be determined. Furthermore, lack of signal generation can indicate or be used to determine an interaction between an analyte-binding domain and a target analyte does not exist.

Thus, use of proximity probes, which bind to a target analyte, and address polynucleotides, which interact with one or more proximity probes in a proximity-dependent manner, can be used in the methods described herein, for example, to determine binding partners, affinities of one or more binding moieties to one or more target analytes, and/or specificities of one or more binding moieties for one or more target analytes.

The multiplex assays described herein utilize a unique label (or barcode) to be attached to each analyte and probe such that positive hits can be deconvoluted at the end using high-throughput methodologies. Hundreds or thousands mAbs can be simultaneously screened for their binding partners in a multiplex format (e.g., on a human proteome (HuProt) array, harboring over 17,000 full-length human proteins). Using proximity ligation coupled with deep sequencing, multiple interacting target analyte-binding moiety pairs (e.g., mAb-polypeptide pairs) can be identified in a single experiment. Binding specificity of a given binding moiety (e.g., a mAb) can be determined by analyzing sequencing results. The methods and compositions can reveal the wide or narrow spectrum of target analytes to which one or more binding moieties interact. Because the methods and compositions described herein provide unique digital sequencing reads barcoded to each binding moiety and target analyte, the total number of resulting sequencing reads can serve as a unique determinant for the affinity of each binding moiety-target analyte pair.

The applications and potential of the invention described herein is large. For example, the DAPPL methods and compositions can be used to perform functional genome wide associations of SNPs, map transcription factors to DNase I hypersensitive sites (DHSs), determine protein and RNA (coding or non-coding) interactions, determine antigen and antibody interactions, determine protein to protein interactions, determine peptide to protein interactions, screen for aptamer binding partners, determine protein-DNA interactions (e.g., transcription factor-DNA interactions), determine small molecule to protein interactions, perform serum profiling, and much more.

### Target Analytes

An analyte, or target analyte, can be, but is not limited to, a polypeptide, a protein, a protein fragment, a tagged protein, an antibody, an antibody fragment, a small molecule, a virus particle (e.g., a virus particle comprising a transmembrane protein), or a cell. A target analyte does not base pair with an address polynucleotide in proximity thereto. In some instances, a target analyte comprises at least two amide bonds. In some instances, a target analyte does not comprise a phosphodiester linkage. In some instances, a target analyte is not DNA or RNA.

In some instances, a target analyte comprises a polypeptide, protein, or fragment thereof “Polypeptide” and “protein” are used interchangeably and refer to a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics).

For example, a target analyte can comprise an isolated polypeptide, a purified polypeptide, or a polypeptide within a virus particle. For example, a target analyte can comprise a polypeptide is within a virus particle membrane. A virus particle refers to a fully or partially assembled capsid of a virus surrounded by a lipid envelope. A viral particle may or may not contain nucleic acids.

For example, a target analyte can comprise an antibody or fragment thereof. For example, a target analyte can comprise a transcription factor. For example, a target analyte can comprise a receptor. For example, a target analyte can comprise a transmembrane receptor.

Target analytes include isolated, purified, and/or recombinant polypeptides. Target analytes include target analytes present in a mixture of analytes (e.g., a lysate). For example, target analytes include target analytes present in a lysate from a plurality of cells or from a lysate of a single cell.

In some instances, a target analyte comprises a small molecule. For example, a target analyte can comprise a drug. For example, a target analyte can comprise a compound. For example, a target analyte can comprise an organic compound. In some instances, a target analyte comprises a small molecule with a molecular weight of 900 Daltons or less. In some instances, a target analyte comprises a small molecule with a molecular weight of 500 Daltons or more. Small molecules may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A target analyte can comprise a member of a specific binding pair (e.g., a ligand). A target analyte can be monovalent (monoepitopic) or polyvalent (polyepitopic). A target analyte can be antigenic or haptenic. A target analyte can be a single molecule or a plurality of molecules that share at least one common epitope or determinant site. A target analyte can be a part of a cell (e.g., a bacteria cell, a plant cell, or an animal cell). A target cell can be either in a natural environment (e.g., tissue), a cultured cell, or a microorganism (e.g., a bacterium, fungus, protozoan, or virus), or a lysed cell. A target analyte can be further modified (e.g. chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

A target analyte comprises at least one potential binding site for a binding moiety. In some instances, a target analyte comprises one binding site. In some instances, a target analyte comprises at least two binding sites. For example, a target analyte can comprise 2, 3, 4, 5, 6, 7, 8, 9, 10, 11, 12, 13, 14, 15, 16, 17, 18, 19, 20, or more binding sites.

In some instances, a target analyte is a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render the target analyte more readily detectible. Samples include a quantity of a substance from a living thing or formerly living things. A sample can be natural, recombinant, synthetic, or not naturally occurring. A target analyte can be expressed from a cell naturally or recombinantly, in a cell lysate or cell culture medium, an in vitro translated sample, or an immunoprecipitation from a sample (e.g., a cell lysate).

In some instances, a target analyte is expressed in a cell-free system or in vitro. For example, a target analyte can be in a cell extract containing a nucleotide template and raw materials for translation of the target analyte. In some instances, a target analyte can be in a cell extract containing a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and μIP-microintaglio printing, and pMAC—protein microarray copying (See Kilb et al., Eng. Life Sci. 2014, 14, 352-364).

In some instances, a target analyte is synthesized in situ (e.g., on a solid substrate of an array) from a DNA template. In some instances, a plurality of target analytes is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, target analyte polypeptide synthesis is carried out on a solid surface (e.g., an array surface) coated with a protein-capturing reagent or antibody. In some instances, the target polypeptides comprise a tag (e.g., polyhistidine or GST) that is bound by the capture reagent or antibody, thus coupling the target polypeptides to the solid surface (e.g., a nucleic acid programmable protein array (NAPPA)). In some instances, the DNA template is immobilized onto the same protein-capture surface. For example, the DNA template can be biotinylated and bound to avidin pre-coated onto the protein capture surface. In some instances, the DNA template is not coupled to the solid support. In some instances, the DNA template is added as a free molecule in the reaction synthesis mixture (e.g., a protein in situ array (PISA)).

In some instances, in situ puromycin-capture methods can be used to express target polypeptides. For example, the template DNA can be transcribed to mRNA, and a single-stranded DNA oligonucleotide modified with biotin and puromycin on each end can be hybridized to the 3′-end of the mRNA. The mRNAs can be coupled to the surface e.g., by the binding of biotin to streptavidin that is pre-coated on the surface. Cell extract can then be added to initiate in situ translation. When the ribosome reaches the hybridized oligonucleotide, it stalls and incorporates the puromycin molecule to the nascent polypeptide chain, thereby attaching the newly synthesized protein to the surface via the DNA oligonucleotide. Purified target polypeptides may be obtained after the mRNA is removed (e.g., digested with RNase).

In some instances, DNA array to protein array (DAPA) methods can be used to repeatedly produce protein arrays by printing them from a single DNA template array, on demand. An array of immobilized DNA templates on a substrate is assembled face-to-face with a second substrate pre-coated with a protein-capturing reagent, and a membrane soaked with a cell extract is placed between the two substrates for transcription and translation. The synthesized target polypeptides are then immobilized onto a substrate to form the array.

A target analyte can comprise a plurality of target analytes. A target analyte can comprise a plurality of target analytes representing a substantial portion or an entire organism's proteome, such as a bacterial, viral, fungal, plant, or animal proteome. A target analyte can comprise a plurality of target analytes representing a substantial portion or an entire proteome of an insect or mammal, such as a mouse, rat, rabbit, cat, dog, monkey, goat, or human. For example, a target analyte can comprise a plurality of target analytes representing at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of an organism's proteome.

A target analyte can comprise a plurality of target analytes comprising at least 2 different target analytes. For example, a target analyte can comprise a plurality of target analytes comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different target analytes.

In some instances, target analytes can comprise a tag. For example, a proteinaceous target analyte can comprise a fusion tag. For example, a proteinaceous target analyte can comprise a GST-tag, His-tag, FLAG-tag, T7 tag, S tag, PKA tag, HA tag, c-Myc tag, Trx tag, Hsv tag, CBD tag, Dsb tag, pelB/ompT, KSI, MBP tag, VSV-G tag, 3-Gal tag, GFP tag, or a combination thereof, or other similar tags. In some instances, the protein tag binder is a group which binds an endogenous protein tag (e.g., an epitope on the protein). In this group of embodiments, the protein tag binder will typically be an antibody or antibody fragment which is sufficient to form a non-covalent association complex with the protein tag or epitope. In some embodiments, the polypeptide target analytes comprise PTMs including, but not limited to, glycosylation, phosphorylation, acetylation, methylation, myristoylation, prenylation, or proteolytic processing. In some embodiments, a polypeptide target analyte is homologous to a native polypeptide.

In some instances, a target analyte comprises a contiguous span of at least 6 amino acids, for example, least 8, 9, 10, 12, 15, 20, 25, 30, 40, 50, or 100 amino acids of a reference sequence. In some instances, a target analyte comprises a contiguous stretch of amino acids comprising a site of a mutation or functional mutation, including a deletion, addition, swap, or truncation of the amino acids in a polypeptide sequence. Polypeptides may be isolated from human or mammalian tissue samples or expressed from human or mammalian genes. Polypeptides may be made using routine expression methods known in the art. A polynucleotide encoding a desired polypeptide may be inserted into an expression vector suitable for any convenient host. Both eukaryotic and prokaryotic host systems can be used in forming recombinant polypeptides. A polypeptide may be isolated from lysed cells or from the culture medium and purified to the extent needed for its intended use. (See, e.g., WO2012103260 and WO2011159959). Purification may be by any technique known in the art, for example, differential extraction, salt fractionation, chromatography, centrifugation, and the like (See, e.g., Abbondanzo et al., (1993) Methods in Enzymology, Academic Press, New York. pp. 803-23).

In addition, shorter protein fragments may be produced by chemical synthesis. Alternatively proteins of the presently disclosed subject matter are extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis, for example. Reference cDNA may be used to express polypeptides. A nucleic acid encoding a polypeptide to be expressed can be operably linked to a promoter in an expression vector using conventional cloning technology. For example, a polypeptide in an expression vector may comprise the full coding sequence for the polypeptide or a portion thereof.

In some embodiments, a target analyte is a membrane bound protein. In one embodiment, the membrane bound protein is CD4, a classical type I membrane protein with a single transmembrane (TM) domain. (Carr et al., (1989) J. Biol. Chem. 264:21286-95). In another embodiment, the membrane bound protein is GPR77, a multi-spanning, G-protein coupled receptor (GPCR) membrane protein. (Cain & Monk, (2002) J. Biol. Chem. 277:7165-69).

Additional exemplary membrane bound proteins include, but are not limited to, GPCRs (e.g. adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), receptor tyrosine kinases, receptor serine/threonine kinases, receptor guanylate cyclases, growth factor and hormone receptors (e.g., epidermal growth factor (EGF) receptor), and others. Mutant or modified variants of membrane-bound proteins may also be used. For example, some single or multiple point mutations of GPCRs retain function and are involved in disease (See, e.g., Stadel et al., (1997) Trends in Pharmacological Review 18:430-37).

### Target Analyte Libraries

In some embodiments, a target analyte can comprise a plurality of target analytes that are specific to a common pathway. Target analytes belong to a common pathway when they share one or more attributes in common in a gene ontology, a collection that assigns defined characteristics to a set of genes and their products. The ontology administered by the Gene Ontology (“GO”) Consortium is particularly useful in this regard. Target analytes belonging to common pathways can be identified by searching gene ontology, such as GO, for genes sharing one or more attributes. The common attribute could be, for example, a common structural feature, a common location, a common biological process or a common molecular function.

The wealth of information that exists in published, peer-reviewed literature concerning the function of human genes and proteins has been organized and curated using a coordinated system of controlled vocabulary that is administered by the Gene Ontology (GO) Consortium. Of the approximately 40,000 transcribed units in the human genome, approximately 20,000 code for annotated proteins, and approximately 14,000 of those proteins have a functional annotation in the GO database. The functional annotations contained in the GO database are organized in a hierarchical manner, and it is possible to access this information from the GO database and search for all of the genes in the human genome that are annotated to be involved in the same biological process, reside in the same cellular component, or perform the same molecular function.

In some embodiments, target analytes in a common pathway are the expression product of genes involved in the same biological process or molecular function as annotated by gene ontology. (e.g., genes involved in the response to DNA damage or gene products of transcription factors, such as of a particular tissue, cell type or organ, such as the brain). In some embodiments, target analytes in a common pathway are small molecules that affect the expression product of genes involved in the same biological process or molecular function as annotated by gene ontology.

In some embodiments, target analytes in a common pathway are the gene products of genes whose transcript levels or proteins levels change upon treatment or exposure to the same stimulus and are thus co-regulated (e.g., target analytes that are induced or repressed upon treatment to UV radiation). In some embodiments, target analytes in a common pathway are small molecules that affect the gene products of genes whose transcript levels or proteins levels change upon treatment or exposure to the same small molecule and are thus co-regulated.

In some embodiments, target analytes in a common pathway are target analytes that contain similar sequence features. These features may be a DNA sequence motif, collection of DNA sequence motifs, or enrichment of higher order sequence features that are distinguishable from a background model of random genomic sequences. A DNA sequence motif can either be defined by a consensus sequence or a probability matrix where the identity of each base at each position of a motif is defined as a probability. In some embodiments, the members of the pathway share a common structural or functional attribute. For example, the target analytes could share a common sequence motif, such as a zinc finger or a transmembrane region.

In some embodiments, target analytes in a common pathway could be the gene products of genes whose sequences, transcripts or proteins are connected via metabolic transformations and/or physical protein-protein, protein-DNA and protein-compound interactions. Enzymes catalyze these reactions, and often require dietary minerals, vitamins and other cofactors in order to function properly. Because of the many chemicals that may be involved, pathways can be quite elaborate. In some embodiments, target analytes in a common pathway are gene products of genes which are all bound by the same transcription factor protein, complex of transcription factor proteins, other nucleic acid binding proteins, or other molecules. These interactions may occur in a living cell (in vivo) or in a solution of purified molecules (in vitro).

In some embodiments, the target analytes in a common pathway belong to the same signal transduction pathway. Typically, in biology signal transduction refers to any process by which a cell converts one kind of signal or stimulus into another, most often involving ordered sequences of biochemical reactions inside the cell that are carried out by enzymes, activated by second messengers resulting in what is thought of as a signal transduction pathway. Usually, signal transduction involves the binding of extracellular signaling molecules (or ligands) to cell-surface receptors that face outwards from the plasma membrane and trigger events inside the cell. Additionally, intracellular signaling cascades can be triggered through cell-substratum interactions, as in the case of integrins which bind ligands found within the extracellular matrix. Steroids represent another example of extracellular signaling molecules that may cross the plasma membrane due to their lipophilic or hydrophobic nature. Many steroids, but not all, have receptors within the cytoplasm and usually act by stimulating the binding of their receptors to the promoter region of steroid responsive genes. Within multicellular organisms there are a diverse number of small molecules and polypeptides that serve to coordinate a cell's individual biological activity within the context of the organism as a whole. Examples include hormones (e.g. melatonin), growth factors (e.g. epidermal growth factor), extra-cellular matrix components (e.g. fibronectin), cytokines (e.g. interferon-gamma), chemokines (e.g. RANTES), neurotransmitters (e.g. acetylcholine), and neurotrophins (e.g. nerve growth factor).

In addition to many of the regular signal transduction stimuli listed above, in complex organisms, there are also examples of additional environmental stimuli that initiate signal transduction processes. Environmental stimuli may also be molecular in nature or more physical, such as, light striking cells in the retina of the eye, odorants binding to odorant receptors in the nasal epithelium, bitter and sweet tastes stimulating taste receptors in the taste buds, UV light altering DNA in a cell, and hypoxia activating a series of events in cells. Certain microbial molecules e.g. viral nucleotides, bacterial lipopolysaccharides, or protein target analytes are able to elicit an immune system response against invading pathogens, mediated via signal transduction processes.

Activation of genes, alterations in metabolism, the continued proliferation and death of the cell, and the stimulation or suppression of locomotion, are some of the cellular responses to extracellular stimulation that require signal transduction. Gene activation leads to further cellular effects, since the protein products of many of the responding genes include enzymes and transcription factors themselves. Transcription factors produced as a result of a signal transduction cascade can in turn activate yet more genes. Therefore an initial stimulus can trigger the expression of an entire cohort of genes, and this in turn can lead to the activation of any number of complex physiological events. These events include, for example, the increased uptake of glucose from the blood stream stimulated by insulin and the migration of neutrophils to sites of infection stimulated by bacterial products.

Most mammalian cells require stimulation to control not only cell division, but also survival. In the absence of growth factor stimulation, programmed cell death ensues in most cells. Such requirements for extra-cellular stimulation are necessary for controlling cell behavior in both the context of unicellular and multi-cellular organisms. Signal transduction pathways are so central to biological processes that it is not surprising that a large number of diseases have been attributed to their dysregulation.

In some embodiments, target analytes in a common pathway are part of an oncology pathway. Target analytes in an oncology pathway are those gene products of genes involve in the development of hyperplasia, neoplasia and/or cancer. Examples of oncology pathways include, but are not limited to, hypoxia, DNA damage, apoptosis, cell cycle, and p53 pathway. In some embodiments, target analytes in a common pathway are part of a membrane pathway. Examples of membrane pathways include, but are not limited to, transport protein, G-coupled receptor, ion channel, cell-adhesion protein, and receptor pathways. In some embodiments, target analytes in a common pathway are part of a nuclear receptor pathway. Examples of target analytes in a nuclear receptor pathway include, but are not limited to, gene products regulated by the glucocorticoid receptor protein, estrogen receptor proteins, peroxisome proliferator-activated receptor proteins, androgen receptor proteins, and transporter proteins, including ABC and SLC transporters. In some embodiments, target analytes in a common pathway are part of a neuronal pathway. Examples of target analytes in a neuronal pathway include, but not limited to, gene products of genes expressed in neurons such as neurotransmitters and cell adhesion proteins. In some embodiments, target analytes in a common pathway are part of a vascular pathway. Examples of target analytes in a vascular pathway include, but not limited to, target analytes involved in angiogenesis, lipid metabolism, and inflammation. In some embodiments, target analytes in a common pathway are part of a signaling pathway. Examples of target analytes in a signaling pathway include, but are not limited to, gene products involved in cell-to-cell signaling, hormones, hormone receptors, cAMP response, and cytokines. In some embodiments, target analytes in a common pathway are part of an enzymatic pathway. Examples of target analytes in a enzymatic pathway include, but are not limited to, gene products of genes involved in glycolysis, anaerobic respiration, Krebs cycle/Citric acid cycle, Oxidative phosphorylation, fatty acid oxidation (β-oxidation), gluconeogenesis, HMG-CoA reductase pathway, pentose phosphate pathway, porphyrin synthesis (or heme synthesis) pathway, urea cycle, photosynthesis (plants, algae, cyanobacteria) and chemosynthesis (some bacteria).

The present invention also provides a library of target analytes comprising a plurality of target analytes in which at least 5%, 10%, 20%, 30%, 40%, 50%, 60%, 70%, 80%, 90%, 99% or 100% of the target analytes are part of a common pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of an oncology pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a hypoxia pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a DNA-damage pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of an apoptosis pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a cell cycle pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a p53 pathway. In some embodiments, a library of target analytes comprises a plurality of target analytes in which at least 5%, 10%, 20%, 30%, 40%, 50%, 60%, 70%, 80%, 90%, 99% or 100% of the target analytes are differently selected from the group consisting of hypoxia pathway, DNA-damage pathway, apoptosis pathway, cell cycle pathway, and p53 pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a membrane bound pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a nuclear receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a glucocorticoid receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a peroxisome proliferator-activated receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of an estrogen receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of an androgen receptor pathway In some embodiments, a library of target analytes comprises a plurality of target analytes in which at least 5%, 10%, 20%, 30%, 40%, 50%, 60%, 70%, 80%, 90%, 99% or 100% of the target analytes are part of a cytochrome P450 receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a transporter receptor pathway. In some embodiments, a library of target analytes comprises a plurality of target analytes in which at least 5%, 10%, 20%, 30%, 40%, 50%, 60%, 70%, 80%, 90%, 99% or 100% of the target analytes are differently selected from the group consisting of glucocorticoid receptor pathway, peroxisome proliferator-activated receptor pathway, estrogen receptor pathway, androgen receptor pathway, cytochrome P450 pathway, and transporter pathways In some embodiments, a library of target analytes comprises a plurality of target analytes in which at least 5%, 10%, 20%, 30%, 40%, 50%, 60%, 70%, 80%, 90%, 99% or 100% of the target analytes are part of a vascular pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a neuronal pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a transcription factor pathway. In some embodiments, a library of target analytes comprises a plurality of target analytes in which at least 5%, 10%, 20%, 30%, 40%, 50%, 60%, 70%, 80%, 90%, 99% or 100% of the target analytes are part of a signaling pathway.

The present invention also provides a library of target analytes in which the library represents at least 5%, 10%, 20%, 30%, 40%, 50%, 60%, 70%, 80%, 90%, 99% or 100% of all the target analytes that are part of a common pathway in the genome. In some embodiments, the library represents at least 5%, 10%, 20%, 30%, 40%, 50%, 60%, 70%, 80%, 90%, 99% or 100% of all the target analytes that are part of an oncology pathway in the genome. In some embodiments, the library represents at least 5%, 10%, 20%, 30%, 40%, 50%, 60%, 70%, 80%, 90%, 99% or 100% of all the target analytes that are part of a hypoxia pathway in the genome. In some embodiments, the library represents at least 5%, 10%, 20%, 30%, 40%, 50%, 60%, 70%, 80%, 90%, 99% or 100% of all the target analytes that are part of a DNA-damage pathway in the genome. In some embodiments, the library represents at least 5%, 10%, 20%, 30%, 40%, 50%, 60%, 70%, 80%, 90%, 99% or 100% of all the target analytes that are part of an apoptosis pathway in the genome. In some embodiments, the library represents at least 5%, 10%, 20%, 30%, 40%, 50%, 60%, 70%, 80%, 90%, 99% or 100% of all the target analytes that are part of a cell cycle pathway in the genome. In some embodiments, the library represents at least 5%, 10%, 20%, 30%, 40%, 50%, 60%, 70%, 80%, 90%, 99% or 100% of all the target analytes that are part of a p53 pathway in the genome. In some embodiments, the library represents at least 5%, 10%, 20%, 30%, 40%, 50%, 60%, 70%, 80%, 90%, 99% or 100% of all the target analytes that are part of a membrane bound pathway in the genome. In some embodiments, the library represents at least 5%, 10%, 20%, 30%, 40%, 50%, 60%, 70%, 80%, 90%, 99% or 100% of all the target analytes that are part of a nuclear receptor pathway in the genome. In some embodiments, the library represents at least 5%, 10%, 20%, 30%, 40%, 50%, 60%, 70%, 80%, 90%, 99% or 100% of all the target analytes that are part of a glucocorticoid receptor pathway in the genome. In some embodiments, the library represents at least 5%, 10%, 20%, 30%, 40%, 50%, 60%, 70%, 80%, 90%, 99% or 100% of all the target analytes that are part of a peroxisome proliferator-activated receptor pathway in the genome. In some embodiments, the library represents at least 5%, 10%, 20%, 30%, 40%, 50%, 60%, 70%, 80%, 90%, 99% or 100% of all the target analytes that are part of an estrogen receptor pathway in the genome. In some embodiments, the library represents at least 5%, 10%, 20%, 30%, 40%, 50%, 60%, 70%, 80%, 90%, 99% or 100% of all the target analytes that are part of an androgen receptor pathway in the genome. In some embodiments, the library represents at least 5%, 10%, 20%, 30%, 40%, 50%, 60%, 70%, 80%, 90%, 99% or 100% of all the target analytes that are part of a cytochrome P450 receptor pathway in the genome. In some embodiments, the library represents at least 5%, 10%, 20%, 30%, 40%, 50%, 60%, 70%, 80%, 90%, 99% or 100% of all the target analytes that are part of a transporter receptor pathway in the genome. In some embodiments, the library represents at least 5%, 10%, 20%, 30%, 40%, 50%, 60%, 70%, 80%, 90%, 99% or 100% of all the target analytes that are part of a neuronal pathway in the genome. In some embodiments, the library represents at least 5%, 10%, 20%, 30%, 40%, 50%, 60%, 70%, 80%, 90%, 99% or 100% of all the target analytes that are part of a signaling pathway in the genome. n some embodiments, the library represents at least 5%, 10%, 20%, 30%, 40%, 50%, 60%, 70%, 80%, 90%, 99% or 100% of all the target analytes that are part of a vascular pathway in the genome. In some embodiments, the library represents at least 5%, 10%, 20%, 30%, 40%, 50%, 60%, 70%, 80%, 90%, 99% or 100% of all the target analytes that are part of a transcription factor pathway in the genome.

### Coupling of Target Analytes to a Solid Support

A target analyte can be coupled to a solid support (e.g., an array). In some instances, a target analyte is non-covalently coupled to a solid support. For example, a non-covalent interaction can be an ionic interaction or a van der Waals interaction. In some instances, a target analyte is covalently coupled to a solid support. In some instances, a target analyte is reversibly coupled to a solid support. In some instances, a target analyte is irreversibly coupled to a solid support.

A surface of a solid support can be coated with a functional group and a target analyte can be attached to the solid support through the functional group. For example, a solid support can be coated with a first functional group and a target analyte comprising a second functional group can be attached to the solid support by reacting the first functional group with the second functional group. For example, a surface of a solid support can be coated with streptavidin and a biotinylated target analyte can be attached thereto. Exemplary couplings of a target analyte include streptavidin- or avidin- to biotin interactions; hydrophobic interactions; magnetic interactions; polar interactions, (e.g., associations between two polar surfaces); formation of a covalent bond (e.g., an amide bond, disulfide bond, thioether bond, or via crosslinking agents; and via an acid-labile linker.

In some instances, a target analyte is coupled to a solid surface through a linker. For example, a first functional group of a linker attached to a solid surface can be coupled to a target analyte, thereby coupling the target analyte to the solid surface. For example, a first functional group of a linker can be coupled to a target analyte and a second functional group of the linker can be coupled to a solid support, thereby coupling the target analyte to the solid surface. target analyte can be coupled to a solid surface through a linker. In some instances, a linker comprising a first and a second functional group can be attached to the solid support via the second functional group after the first functional group is coupled to the target analyte. In some instances, a linker comprising a first and a second functional group can be attached to the solid support via the second functional group before the first functional group is coupled to the target analyte.

In some instances, a target analyte is coupled to a solid surface via an antibody. For example, an antibody linker can be attached to a solid surface and a target analyte to which the antibody specifically bind can be linked to the solid support by binding to the antibody linker. In some instances, the coupling is photocleavable. In some instances, target analytes can comprise a tag that is directly coupled to a solid surface. For example, a proteinaceous target analyte can comprise a fusion tag that is directly conjugated to the solid surface. For example, a proteinaceous target analyte can comprise a GST-tag, His-tag, FLAG-tag, or other similar tags and the tag can be directly coupled to the solid surface instead of the proteinaceous target analyte itself.

Depending on the type of target analyte (e.g., a polypeptide, a protein, an antibody, an antibody fragment, a small molecule, a virus particle, or a cell) different suitable coupling chemistries can be employed to couple the target analyte to a solid surface.

There are many known methods for covalently immobilizing polypeptides and antibodies onto a solid support. For example, MacBeath et al., (1999) J Am Chem Soc 121: 7967-68) use the Michael addition to link thiol-containing compounds to maleimide-derivatized glass slides to form a microarray of small molecules. (See also, Lam & Renil (2002) Current Opin Chemical Biol 6:353-58). Non-covalent coupling may be by any suitable secondary interaction, including but not limited to hydrophobic bonding, hydrogen bonding, Van der Waals interactions, ionic bonding, etc.

Amine chemistry can be used to couple or immobilize target analytes to a solid surface. For example, a covalent amide bond can be formed between a target analyte and a solid support. For example, a covalent amide bond can be formed by reacting a carboxyl-functionalized target analyte with an amino-functionalized solid support. For example, a covalent amide bond can be formed by reacting an amide-functionalized target analyte with a carboxyl-functionalized solid support. Amine-terminated target agents may be immobilized using amine/cyanuric chloride coupling; amide bonding through reactions with N-hydroxysuccinimide (NHS)-ester-, carboxylic acid-, carbonate-, anhydride- or acyl group-functionalized surfaces; amidine formation through reaction with imidoester-functionalized surfaces; sulphonamide formation through reactions with sulfonyl halide-functionalized surfaces; aniline formation through reactions with surface presenting aryl groups; imine formation through reactions with aldehyde-functionalized surfaces; amino ketone formation through Mannich reactions with aldehyde-functionalized surfaces; guanidine formation through reactions with carbodiimide-functionalized surfaces; urea formation through reactions with isocyanate-functionalized surfaces; thiourea formation through reactions with isothiocyanate-functionalized surfaces, or; amino alcohol formation through reactions with epoxide-functionalized surfaces. Hydrazine- or oxyamine-terminated binding agents may be immobilized in the same way.

Thiol groups can be used to couple or immobilize target analytes to a solid surface. For example, target analytes having or functionalized with thiol groups with may be immobilized on surfaces presenting, e.g., maleimide, aryl- or carbon-carbon double-bond-containing groups through formation of stable carbon-sulfur bonds, or through interactions with aziridine-functionalized surfaces. Disulfide exchange reactions with thiol-functionalized surfaces may also be used. Target analytes having or functionalized with thiol groups may be immobilized on gold surfaces through semi-covalent interactions between gold and sulphur groups.

Carboxylic acid-functionalized surfaces may also be used to immobilize target analytes functionalized with carbodiimide and diazoalkane groups. Solid surfaces presenting hydroxyl groups may be used to immobilize isocyanate- and epoxide-functionalized target analytes.

Functionalized target analytes may also be immobilized through cycloaddition reactions between functional groups having a conjugated diene and groups having a substituted alkene through Diels-Alder chemistry, or using “click” chemistry, through reactions between nitrile and azine groups. In any of the above described covalent couplings, the target analytes-surface orientation of functional groups may be reversed. An alternative means of covalent attachment not utilizing a derivatized binding agent utilizes array surfaces having photoreactive groups such as benzophenone, diazo, diazirine, phthalamido and arylazide groups.

Non-covalent immobilization may involve electrostatic interactions between target analytes and surfaces modified to contain positively- or negatively-charged groups, such as amine or carboxy groups, respectively. Target analytes may be non-covalently immobilized in a defined orientation, for example, using fluorophilic, biotin-streptavidin, histidine-Ni, histidine-Co, and complementary single-stranded DNA interactions between tagged target analytes and binding partner-coated surfaces, in either orientation.

Appropriate agents for coupling of target analytes to a solid surface include a variety of agents that are capable of reacting with a functional group present on a surface of the target analyte and with a functional group present on the solid surface. Reagents capable of such reactivity include homo- and hetero-bifunctional reagents, many of which are known in the art. Exemplary bifunctional cross-linking agents include is N-succinimidyl(4-iodoacetyl) aminobenzoate (SIAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl) cyclohexane-1-carboxylate (SMCC) and 6-hydrazinonicotimide (HYNIC). Any suitable nucleophile reactive group can be used including —NR₁—NH₂ (hydrazide), —NR₁(C═O)NR₂NH₂ (semicarbazide), —NR₁(C═S)NR₂NH₂(thiosemicarbazide), (C═O)NR₁NH₂ (carbonylhydrazide), —(C═S)NR₁NH₂ (thiocarbonylhydrazide), —(SO₂)NR₁NH₂ (sulfonylhydrazide), —NR₁NR₂(C)NR₃NH₂ (carbazide), —NR₁NR₂(C═S)NR₃NH₂ (thiocarbazide), and —O—NH₂ (hydroxylamine), where each R₁, R₂, and R₃ is independently H, or alkyl having 1-6 carbons. The nucleophilic moiety can include any suitable nucleophile, e.g., hydrazide, hydroxylamine, semicarbazide, or carbonylhydrazide.

In addition to those described above, other covalent and non-covalent means of attachment may be employed and are well known to those skilled in the art. A target analyte may be deposited onto a substrate or support by any suitable technique. For example, a target analyte may be deposited as a monolayer (e.g., a self-assembled monolayer), a continuous layer or as a discontinuous (e.g., patterned) layer. A target analyte may be deposited or coupled to a support or substrate by modification of the substrate or support by chemical reaction (See, e.g., U.S. Pat. No. 6,444,254), reactive plasma etching, corona discharge treatment, a plasma deposition process, spin coating, dip coating, spray painting, deposition, printing, stamping, diffusion, adsorption/absorption, covalent cross-linking, or combinations thereof. The target analytes may be directly spotted onto a surface (e.g., a planar glass surface). In some instances, when necessary or beneficial to keep target analytes (e.g., Abs) in a wet environment during the printing process, glycerol (30-40%) may be employed, and/or spotting can be carried out in a humidity-controlled environment.

### Address Polynucleotides

An address polynucleotide is a polynucleotide containing a sequence barcoded to a target analyte or discrete region containing a target analyte. “Polynucleotide”, “nucleic acid sequence”, and “nucleic acid” are used interchangeably and refer to deoxyribonucleotides or ribonucleotides and polymers thereof in either single-stranded or double-stranded form. Nucleic acid sequences can contain known nucleotide analogs or modified backbone residues or linkages. Nucleic acid sequences implicitly encompass conservatively modified variants thereof (e.g., degenerate codon substitutions/mutations) and complementary sequences. Polynucleotides include, among others, single-stranded DNA, double-stranded DNA, DNA that is a mixture of single- and double-stranded regions, single-stranded RNA, double-stranded RNA, and RNA that is mixture of single- and double-stranded regions, and hybrid molecules comprising DNA and RNA.

In preferred embodiments, an address polynucleotide does not substantially interact with a target analyte. In preferred embodiments, an address polynucleotide interacts with, or can be coupled to, a proximity polynucleotide when the address polynucleotide is in proximity to the proximity polynucleotide. In a preferred embodiment, an address polynucleotide can be coupled to a proximity polynucleotide when the binding moiety of a proximity probe binds to a target analyte in proximity to the address polynucleotide.

An address polynucleotide can be coupled directly or indirectly to a solid support. An address polynucleotide can be coupled covalently or non-covalently to a solid support. An address polynucleotide can be coupled to a solid support. An address polynucleotide can be coupled to a solid surface at a particular address (e.g., a discrete region), of the solid support. An address polynucleotide can be coupled to a solid surface at a discrete location within a particular address of the solid support. An address polynucleotide can be coupled to a solid support at a first location within a discrete region of the solid support comprising a target analyte. An address polynucleotide can be coupled to a solid support at a first location within a discrete region of the solid support and a target analyte can be coupled to the solid support at a second location within the discrete region of the solid support. An address polynucleotide coupled to a solid support within a first discrete region of the solid support can be different than an address polynucleotide within a second discrete region of the solid support. An address polynucleotide coupled to a solid support at a first location within a first discrete region of the solid support can be different than an address polynucleotide coupled to the solid support at a first location within a second discrete region of the solid support.

An address polynucleotide can comprise a plurality of address polynucleotides, each barcoded to a particular target analyte. For example, a first address polynucleotide can be barcoded to a first target analyte and a second address polynucleotide can be barcoded to a second target analyte. An address polynucleotide of the invention can be used to identify a target analyte to which it is barcoded. For example, a barcode of an address polynucleotide can correspond to a target analyte. For example, a barcode of a first address polynucleotide can correspond to a first target analyte and a barcode of a second address polynucleotide can correspond to a second target analyte. Thus, a sequence of an address polynucleotide can be used to identify a target polynucleotide.

An address polynucleotide can comprise a plurality of segments. An address polynucleotide can comprise an address barcode sequence. An address polynucleotide can comprise an address linker sequence. An address polynucleotide can comprise an address primer binding sequence. An address polynucleotide can comprise an address spacer sequence. An address polynucleotide can comprise an address polynucleotide linker sequence, an address barcode, an address primer binding sequence, an address spacer, or any combination thereof, arranged in a particular order. For example, an address polynucleotide can be arranged in the order of the address polynucleotide linker sequence, the address barcode, the address primer binding sequence, and the address spacer propagating toward the solid support.

An address polynucleotide can comprise an address barcode sequence, an address linker sequence, an address primer binding sequence an address spacer sequence, or any combination thereof. An address polynucleotide can be arranged in an order such that an address linker sequence is located at one end of the address polynucleotide. An address polynucleotide can be arranged in an order such that it contains an address barcode upstream of the address linker sequence. An address polynucleotide can comprise an address spacer sequence between the address barcode and the address linker sequence. An address polynucleotide can be arranged in an order such that it contains an address primer binding sequence upstream of the address barcode. An address polynucleotide can comprise an address spacer sequence between the address barcode and the address primer binding sequence. An address polynucleotide can comprise an address spacer sequence between the address barcode and the address primer binding sequence. An address polynucleotide can be arranged in an order such that an address spacer sequence is located upstream or downstream of the proximity primer binding sequence. An address polynucleotide can be arranged in an order such that an address spacer sequence is located upstream of the proximity barcode sequence. An address polynucleotide can be arranged in an order such that an address spacer sequence is located at one end of the address polynucleotide, for example, an end of the address polynucleotide that does not contain the address linker sequence. For example, an address polynucleotide can be arranged in an order of the address linker sequence, the address barcode, the address primer binding sequence, and the address spacer sequence. For example, an address polynucleotide can be arranged in an order of the address linker sequence, the address barcode, the address primer binding sequence, and the address spacer sequence propagating toward the solid surface. For example, an address polynucleotide can be arranged in the order of the address linker sequence, the address barcode, the address primer binding sequence, and the address spacer sequence from the 5′ end to the 3′ end. For example, an address polynucleotide can comprise a 5′ end address linker sequence, a unique address barcode sequence, a reverse address primer binding sequence, and a 3′ address spacer sequence attached to a solid support directly or indirectly through a linker (e.g., via a primary amine group attached to the 3′ end) in that order. For example, an address polynucleotide attached to a solid support can be arranged, propagating toward the solid support, in the order of the address linker sequence, the address barcode sequence, the address proximity primer binding sequence, and the address spacer sequence.

An address polynucleotide can comprise a plurality of address polynucleotides. The plurality of address polynucleotides can be comprised by a plurality of discrete regions on a solid support. For example, an address polynucleotide can comprise a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 address polynucleotides. For example, a plurality of address polynucleotide can comprise a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 address polynucleotides comprised by a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 discrete regions of a solid support.

### Address Polynucleotide Linker

An address polynucleotide can comprise an address linker sequence. An address linker sequence is a sequence or end of an address polynucleotide that can be coupled to a proximity polynucleotide, (e.g., a proximity address linker sequence). For example, an address linker sequence can be indirectly hybridized to a proximity polynucleotide through use of a splint polynucleotide. For example, an address linker sequence can be hybridized to a proximity polynucleotide directly. For example, an end of an address linker sequence can be ligated to an end of a proximity polynucleotide. For example, 3′ end of an address polynucleotide comprising an address linker sequence can be ligated to a 5′ end of a proximity polynucleotide (e.g., a proximity linker sequence). An address linker sequence can be located at a terminus or an end of an address polynucleotide. For example, an address linker sequence can be a 3′ terminus or end of an address polynucleotide. An address linker sequence can be interposed between an end of an address polynucleotide and an address primer binding sequence of an address polynucleotide. An address linker sequence can be located downstream of an address primer binding sequence. For example, an address linker sequence can be located 3′ to an address primer binding sequence. An address linker sequence can be located downstream of an address barcode sequence of an address polynucleotide. For example, an address linker sequence can be located 3′ to an address barcode sequence. An address linker sequence can be located downstream of an address spacer sequence of an address polynucleotide. For example, an address linker sequence can be located 3′ to an address spacer sequence.

An address linker sequence can be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more consecutive nucleotides. An address linker sequence can be a sequence of known length.

An address linker sequence of each address proximity polynucleotide of a plurality of address polynucleotides can be a unique or a same linker sequence. For example, any one address linker sequence of a plurality of address linker sequences can be a unique linker sequence. In preferred embodiments, each address linker sequence of a plurality of address linker sequences is the same sequence. For example, each address linker sequence of a plurality of address linker sequences can comprise a same sequence that hybridizes to a splint polynucleotide. For example, each address linker sequence of a plurality of address linker sequences can comprise a same sequence that hybridizes to a same sequence of a splint polynucleotide. For example, each address linker sequence of a plurality of address linker sequences can comprise a same sequence that hybridizes to a same sequence of a splint polynucleotide, wherein the splint polynucleotide can hybridize to a proximity linker sequence (thus, coupling the address polynucleotide and the proximity polynucleotide). For example, each address polynucleotide can comprise the same address linker sequence. Thus, an address linker sequence can be a universal address linker sequence.

An address linker sequence can comprise a randomly assembled sequence of nucleotides. An address linker sequence can be a sequence of known length. An address linker sequence can be a known sequence. An address linker sequence can be a predefined sequence. An address linker sequence can be an unknown sequence of known length. An address linker sequence can be an unknown sequence of known length. In a preferred embodiment, an address linker sequence is a universal sequence such that coupling of each address linker sequence of a plurality of address polynucleotides to a proximity polynucleotide can be carried out with a universal splint polynucleotide. Thus, a universal splint polynucleotide that hybridizes to each of the address linker sequences can be utilized to couple all address polynucleotides to a proximity polynucleotide in a single reaction, simultaneously, and/or without bias for the coupling reaction.

### Address Polynucleotide Barcode

An address polynucleotide can comprise an address barcode sequence or compliment thereof. A barcode or barcode sequence relates to a natural or synthetic nucleic acid sequence comprised by a polynucleotide allowing for unambiguous identification of the polynucleotide and other sequences comprised by the polynucleotide having said barcode sequence. For example, an address barcode comprised by an address polynucleotide can allow for identification of the address polynucleotide. The number of different barcode sequences theoretically possible can be directly dependent on the length of the barcode sequence; e.g., if a DNA barcode with randomly assembled adenine, thymidine, guanosine and cytidine nucleotides can be used, the theoretical maximal number of barcode sequences possible can be 1,048,576 for a length of ten nucleotides, and can be 1,073,741,824 for a length of fifteen nucleotides. An address barcode sequence can be used to identify a specific target analyte. An address barcode sequence can be barcoded to a target analyte in proximity to the address polynucleotide containing the address barcode. An address barcode sequence can be barcoded to a discrete region of a solid support, such as a discrete region comprising a target analyte. For example, an address barcode sequence can be barcoded to a discrete region comprising a target analyte in proximity to the address polynucleotide, wherein the address polynucleotide is in the same discrete region as the target analyte.

An address barcode can be a unique barcode sequence. For example, any one address barcode of a plurality of address barcodes can be a unique barcode sequence. An address barcode can be used to identify the target analyte to which it is barcoded from a plurality of target analytes (e.g., a plurality of different target analytes or same target analytes from different samples or sources). An address barcode can be used to identify the region, location, or position of a target analyte on a solid support from a plurality of discrete regions, locations, or positions on the solid support. An address barcode can be used to identify a target analyte on a solid support to which the address polynucleotide is in proximity from a plurality of target analytes on the solid support to which the address polynucleotide is not in proximity. An address barcode can be used identify a target analyte that interacts with a binding moiety from a plurality of target analytes. Together with a proximity barcode, an address barcode can be used identify a target analyte from a plurality of target analytes and a binding moiety that interacted with the identified target analyte. An address barcode can be barcoded to a target analyte exclusively. An address barcode can be barcoded to a discrete region on a solid support exclusively.

An address barcode sequence can comprise a sequence of at least 3, 4, 5, 6, 7, 8, 9, 10, 11, 12, 13, 14, 15, 16, 17, 18, 19, 20, 25, 30, 40, 45, or 50 or more consecutive nucleotides. An address polynucleotide can comprise two or more address barcode sequences or compliments thereof. An address barcode sequence can comprise a randomly assembled sequence of nucleotides. An address barcode sequence can be a degenerate sequence. An address barcode sequence can be a known sequence. An address barcode sequence can be a predefined sequence.

In a preferred embodiment, an address barcode sequence is a known, unique sequence that is barcoded a target analyte in a discrete region of a solid support such that a signal containing the address barcode sequence (e.g., a sequence read) or compliment thereof can be used to identify a target analyte of a plurality of target analytes that interacted with a binding moiety of a plurality of binding moieties.

### Address Polynucleotide Primer Binding Site

An address primer binding sequence can be used as a primer binding site for a reaction, such as amplification or sequencing. A primer binding sequence relates to a nucleic acid sequence that specifically hybridizes to a predefined amplification primer under conditions typicality used in PCR or other nucleic acid amplifying methods. An address primer binding sequence can be a first primer binding sequence of a primer pair used for a reaction (e.g., amplification or sequencing). For example, an address primer binding sequence can be a forward or reverse primer binding site. For example, an address primer binding site can be a forward primer binding site and a proximity primer binding sequence can be a reverse primer binding sequence. In some embodiments, an address primer binding sequence is a universal primer binding sequence.

An address primer binding sequence and a proximity primer binding sequence (e.g., of a proximity polynucleotide of a proximity probe) can comprise melting temperatures that differ by no more than 6, 5, 4, 3, 2, or 1 degree Celsius. The nucleotide sequence of a proximity primer binding sequence and an address primer binding sequence of an address polynucleotide can differ such that a polynucleotide that hybridizes to one does not hybridize to the other.

An address primer binding sequence can be located upstream of an address barcode. For example, an address primer binding sequence can be 3′ to an address barcode or compliment thereof. An address primer binding sequence can be located upstream of an address linker sequence. For example, an address primer binding sequence can be 3′ to an address linker sequence. An address primer binding sequence can be located upstream of a proximity linker sequence when the address polynucleotide is coupled to a proximity polynucleotide. For example, an address primer binding sequence can be 3′ to a proximity linker sequence when the address polynucleotide is coupled to a proximity polynucleotide. An address primer binding sequence can be located upstream of a proximity barcode sequence when the address polynucleotide is coupled to a proximity polynucleotide. For example, an address primer binding sequence can be 3′ to a proximity barcode sequence when the address polynucleotide is coupled to a proximity polynucleotide.

### Address Polynucleotide Spacer

A spacer (e.g., a spacer sequence) can include natural or synthetic nucleic acid sequences, peptides, or other chemical entities, interposed between two sequences of a polynucleotide, interposed between a sequence of a polynucleotide and a binding moiety to which the polynucleotide is attached, or interposed between a sequence of a polynucleotide and a solid support to which the polynucleotide is attached. A spacer can also include natural or synthetic nucleic acid sequences, peptides, or other chemical entities, interposed between two amino acid sequences that do not naturally link the two polypeptide domains in nature.

An address polynucleotide can comprise an address spacer. An address spacer sequence is a sequence used to increase the length of the address polynucleotide or to separate one or more of an address barcode, an address linker, an address primer binding site, and a solid support to which the address polynucleotide is attached, from each other. For example, an address spacer sequence can be interposed between an address primer binding sequence of an address polynucleotide and a solid support to which an end or other portion of the address polynucleotide is attached. For example, a spacer can be interposed between a primer binding sequence and a binding moiety of an address polynucleotide. In some embodiments, an address polynucleotide does not comprise a spacer sequence. For example, an address polynucleotide can be coupled to a solid support at an end of the address polynucleotide comprising an address primer binding site.

In some embodiments, an address spacer is attached to a solid support. In some embodiments, an address spacer is located upstream of an address primer binding sequence. For example, an address spacer can be located 3′ to an address primer binding sequence. In some embodiments, an address spacer is located downstream of an address primer binding sequence. For example, an address spacer can be located 5′ to an address primer binding sequence. In some embodiments, an address spacer is located upstream of an address barcode. For example, an address spacer can be located 3′ to an address barcode. In some embodiments, an address spacer is located downstream of an address barcode. For example, an address spacer sequence can be located 5′ to an address barcode. In some embodiments, an address spacer is located upstream of an address linker sequence. For example, an address spacer sequence can be located 3′ to an address linker sequence.

In some embodiments, an address spacer is interposed between an address primer binding sequence and a solid support to which the address polynucleotide is attached. For example, an address spacer sequence can be located 3′ to an address primer binding sequence and a 3′ end of the address polynucleotide attached to a solid support. In some embodiments, an address spacer is interposed between an address primer binding sequence and an address barcode. For example, an address spacer sequence can be located 5′ to an address primer binding sequence and 3′ to an address barcode. In some embodiments, an address spacer is interposed between an address linker sequence and an address barcode. For example, an address spacer sequence can be located 5′ to a proximity barcode and 3′ to a proximity linker sequence.

An address spacer sequence can be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 250, 300, 400, 500 or more consecutive nucleotides. An address spacer sequence can comprise a randomly assembled sequence of nucleotides. An address spacer sequence can be a sequence of known length. An address spacer sequence can be a known sequence. An address spacer sequence can be a predefined sequence. An address spacer sequence can be an unknown sequence of known length. An address spacer sequence can be a known sequence of known length.

### Coupling of Address Polynucleotide to a Solid Support

An address polynucleotide can be coupled to a solid support. For example, an address polynucleotide can be immobilized on a solid substrate. An address polynucleotide can be coupled to the solid support through covalent or non-covalent interactions. For example, address polynucleotide can be coupled to the solid support non-covalently through hydrophobic bonding, hydrogen bonding, Van der Waals interactions, ionic bonding, etc. In some instances, an address polynucleotide is coupled reversibly. In some instances, an address polynucleotide is coupled irreversibly.

An address polynucleotide can be coupled to a solid support at any internal position along its length or at either the 5′ or 3′ position. A solid support-coupled address polynucleotide is then able to undergo interactions at positions distant from the solid support. In preferred embodiments, the coupling allows removal of undesired molecules on the solid support (e.g., molecules that non-specifically interact with the solid support or components on the solid support) by washing.

An address polynucleotide can be coupled a solid support through a functional group (e.g., a reactive group). An address polynucleotide can comprise any suitable functional group for coupling to a solid support. Any suitable methods and reagent for modifying the ends of polynucleotides and/or for incorporating bases modified with functional groups into polynucleotides can be used. (See, e.g., Atherton et al., (1989) Tet Lett 30(15):1927-30; Bannworth & Knorr, (1991) Tet Lett 32(9): 1157-60; Wilchek et al., (1994) Bioconjugate Chem 5(5):491-92; Solid Phase Peptide Synthesis, (1989) IRL Press, Oxford, England; and Lloyd-Williams et al., Chemical Approaches to the Synthesis of Peptides and Proteins, (1997) CRC Press).

For example, an address polynucleotide can be phosphorylated at the 5′-terminus (e.g., with phosphokinase) and covalently attached to an amino-activated substrate through a phosphoramidate or phosphodiester linkage. In some embodiments, an address polynucleotides modified is modified at its 3′- or 5′-termini with a primary amino group and coupled to a carboxy-activated substrate. The functional group may be selected to covalently or non-covalently couple the address polynucleotide to the surface. Coupling can be at an internal position or at either the 5′ or 3′ position of an address polynucleotide. For example, a surface of a solid support can be coated with a functional group and an address polynucleotide can be attached to the solid support through the functional group. For example, a solid support can be coated with a first functional group and an address polynucleotide comprising a second functional group can be attached to the solid support by binding or reacting the first and second functional groups. For example, a surface of a solid support can be coated with streptavidin and a biotinylated address polynucleotide can be attached thereto.

In some embodiments, address polynucleotides are synthesized directly on a solid substrate (e.g., a hydroxy-activated solid surface), such as by using phosphoramidite synthesis reagents, photoprotected phosphoramidites, or photolithographic methods (See, e.g., U.S. Pat. No. 5,744,305). For example, address polynucleotides can be covalently attached to a substrate via its 3′-terminus via a phosphodiester linkage.

An address polynucleotide or functional group for attachment of the address polynucleotide can be deposited on a solid surface (e.g., an array or bead) by any suitable technique. For example, an address polynucleotide or functional group may be deposited as a self-assembled monolayer, modification of the solid substrate by chemical reaction (See, e.g., U.S. Pat. No. 6,444,254), reactive plasma etching, corona discharge treatment, a plasma deposition process, spin coating, dip coating, spray painting, deposition, printing, stamping, etc. An address polynucleotide or functional group may be deposited as a continuous layer or as a discontinuous (e.g., patterned) layer. An address polynucleotide or functional group may be deposited via diffusion, adsorption/absorption, or covalent cross-linking. In some embodiments, address polynucleotides or functional groups are spotted onto a glass surface. In some instances, a solid support is modified to achieve better binding capacity. For example, a glass surface may be coated with a thin nitrocellulose membrane or poly-L-lysine such that an address polynucleotide can be passively adsorbed onto the modified surface via non-specific interactions. In some instances, a surface of the solid substrate is coated with streptavidin and a biotinylated address polynucleotide can be coupled thereto.

Examples of solid surface materials and corresponding functional groups include gold, silver, copper, cadmium, zinc, palladium, platinum, mercury, lead, iron, chromium, manganese, tungsten, and any alloys thereof. Exemplary functional groups of solid surfaces include sulfur-containing functional groups such as thiols, sulfides, disulfides (e.g., —SR or —SSR where R is H, alkyl, or aryl), and the like; doped or undoped silicon with silanes and chlorosilanes (e.g., —SiR₂Cl where R is H, alkyl, or aryl); metal oxides (e.g., silica, alumina, quartz, glass, and the like) with carboxylic acids; platinum and palladium with nitrites and isonitriles; copper with hydroxamic acids; benzophenones; acid chlorides; anhydrides; epoxides; sulfonyl groups; phosphoryl groups; hydroxyl groups; phosphonates; phosphonic acids; amino acid groups; amides; and the like (See, e.g., U.S. Pat. No. 6,413,587).

Address polynucleotides can optionally be coupled to a solid support through one or more bifunctional linkers (e.g., the linkers comprising one functional group capable of forming a linkage with a solid substrate and another functional group capable of forming a linkage with another linker molecule or the address polynucleotides. Depending on the particular application, linkers may be long or short, flexible or rigid, charged or uncharged, and/or hydrophobic or hydrophilic.

### Proximity Probes

A proximity probe comprises a binding moiety and a proximity polynucleotide. A proximity barcode of the proximity probe's proximity polynucleotide can be used to identify the one or more binding moieties that the proximity probe comprises. In some embodiments, the proximity polynucleotide is attached covalently or non-covalently to the binding moiety. In some embodiments, the proximity polynucleotide is an extension of the binding moiety, for example, when the binding moiety is a polynucleotide.

In some embodiments, a proximity probe comprises a single binding moiety. In some embodiments, proximity probes are multivalent proximity probes. Multivalent proximity probes comprise at least two analyte-binding domains conjugated to one or more nucleic acid(s). For example, multivalent proximity probes may comprise at least about 2, 3, 4, 5, 6, 7, 8, 9, 10, 20, 50, 100, 200, 500, or 1,000 analyte-binding domains conjugated to at least one, or more than one, nucleic acid (e.g., a proximity polynucleotide).

In some embodiments, a proximity probe comprises a single proximity polynucleotide. In some embodiments, a proximity probe comprises 2 or more proximity polynucleotides. For example, a proximity probe can comprise at least about 2, 3, 4, 5, 6, 7, 8, 9, 10, 20, 50, 100, 200, 500, or 1,000 proximity polynucleotides conjugated to at least one, or more than one, binding moiety. In some embodiments, a proximity probe comprises 2 or more proximity polynucleotides containing a same proximity barcode sequence.

### Binding Moieties

An analyte-binding moiety, also referred to as a binding moiety (or domain) is the region, molecule, domain, portion, fragment, or moiety of a proximity probe that binds to a target analyte. Thus, a binding moiety confers the ability to bind or specifically bind to a given target analyte.

In preferred embodiments, a binding moiety does not substantially interact with an address polynucleotide. In preferred embodiments, an analyte-binding moiety does not prevent coupling of the proximity polynucleotide to an address polynucleotide in proximity thereto. In preferred embodiments, a binding moiety does not substantially interact with an address polynucleotide. In preferred embodiments, a binding moiety is a molecule that can contain a nucleic acid, or to which a nucleic acid can be attached, without substantially abolishing the binding of the analyte-binding moiety to its target analyte.

An analyte-binding moiety can be a nucleic acid molecule or can be proteinaceous. Binding moieties include, but are not limited to, RNAs, DNAs, RNA-DNA hybrids, small molecules (e.g., drugs), aptamers, polypeptides, proteins, antibodies, viruses, virus particles, cells, fragments thereof, and combinations thereof (See, e.g., Fredriksson et al., (2002) Nat Biotech 20:473-77; Gullberg et al., (2004) PNAS, 101:8420-24). For example, a binding moiety can be a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, an antibody, an antibody fragment, a mixture of antibodies, a virus particle, a cell, or any combination thereof.

In some embodiments, a binding moiety is a polypeptide, a protein, or any fragment thereof. For example, a binding moiety can be a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a phosphatase, an aromatase, a helicase, a protease, an oxidoreductase, a reductase, a transferase, a hydrolase, a lyase, an isomerase, a glycosylase, a extracellular matrix protein, a ligase, an ion transporter, a channel, a pore, an apoptotic protein, a cell adhesion protein, a pathogenic protein, an aberrantly expressed protein, an transcription factor, a transcription regulator, a translation protein, a chaperone, a secreted protein, a ligand, a hormone, a cytokine, a chemokine, a nuclear protein, a receptor, a transmembrane receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, any fragment thereof, or any combination thereof. In some embodiments, a binding moiety is a heterologous polypeptide. In some embodiments, a binding moiety is a protein overexpressed in a cell using molecular techniques, such as transfection. In some embodiments, a binding moiety is recombinant polypeptide. For example, a binding moiety can comprise samples produced in bacterial (e.g., E. coli), yeast, mammalian, or insect cells (e.g., proteins overexpressed by the organisms). In some embodiments, a binding moiety is a polypeptide containing a mutation, insertion, deletion, or polymorphism. In some embodiments, a binding moiety is an antigen, such as a polypeptide used to immunize an organism or to generate an immune response in an organism, such as for antibody production.

In some embodiments, a binding moiety is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can be comprised of three domains, C_{H1}, C_{H2} and C_{H3}. Each light chain can be comprised of a light chain variable region (V_{L}) and a light chain constant region. The light chain constant region can be comprised of one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulins or fragments thereof. An antibody fragment can refer to one or more fragments of an antibody that retain the ability to specifically bind to a target analyte, such as an antigen. In addition, aggregates, polymers, and conjugates of immunoglobulins or their fragments can be used where appropriate so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

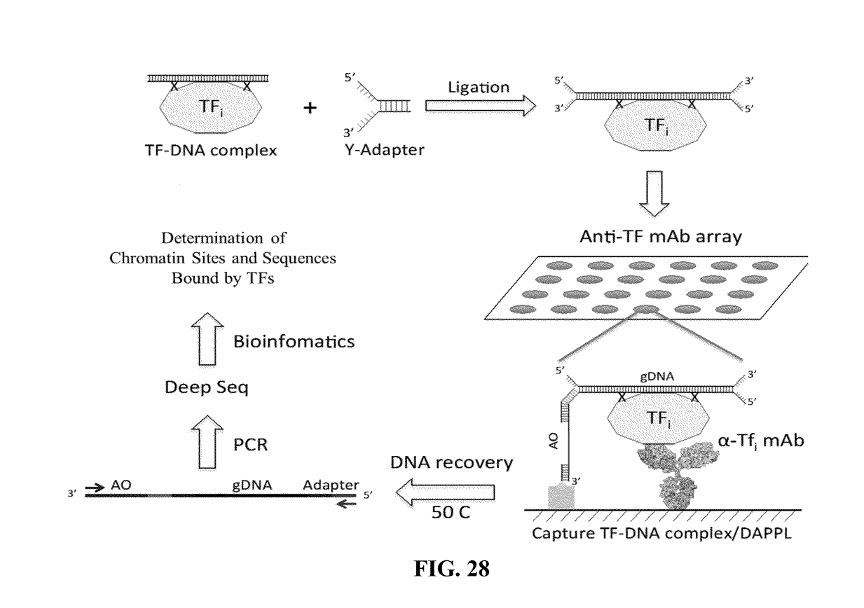

In some embodiments, a binding moiety is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide binding moiety is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a binding moiety is a polynucleotide comprising double stranded region and an end that is not double stranded (e.g., a 5′ or 3′ overhang region. In some embodiments, a binding moiety is a polynucleotide comprising double stranded region that is hybridized and a double stranded end comprising two non-hybridized single strands (e.g., two single stranded overhangs at an end such as a “Y-adapter” depicted in FIG. 28). In some embodiments, a binding moiety is a polynucleotide comprising a double stranded region that is hybridized and two double stranded ends each comprising two non-hybridized single strands (e.g., both ends comprise two single stranded overhangs, such as a polynucleotide comprising two “Y-adapters” as depicted in FIG. 28). In some embodiments, a binding moiety is a recombinant polynucleotide. In some embodiments, a binding moiety is a heterologous polynucleotide. For example, a binding moiety can comprise polynucleotides produced in bacterial (e.g., E. coli), yeast, mammalian, or insect cells (e.g., polynucleotides heterologous to the organisms). In some embodiments, a binding moiety is a polynucleotide containing a mutation, insertion, deletion, or polymorphism.

In some embodiments, a binding moiety is an aptamer. An aptamer is an isolated nucleic acid molecule that binds with high specificity and affinity to a target analyte, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is entirely fortuitous and plays no role whatsoever in the binding of an aptamer to its cognate target. Aptamers must also be differentiated from the naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand are short, isolated, non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any target of interest including small molecules, carbohydrates, peptides, etc. For most targets, even proteins, a naturally occurring nucleic acid sequence to which it binds does not exist. For those targets that do have such a sequence, e.g., nucleic acid-binding proteins, such sequences will differ from aptamers as a result of the relatively low binding affinity used in nature as compared to tightly binding aptamers. Aptamers are capable of specifically binding to selected targets and modulating the targets activity or binding interactions, e.g., through binding, aptamers may block their target's ability to function. The functional property of specific binding to a target is an inherent property an aptamer. A typical aptamer is 6-35 kDa in size (20-100 nucleotides), binds its target with micromolar to sub-nanomolar affinity, and may discriminate against closely related targets (e.g., aptamers may selectively bind related proteins from the same gene family). Aptamers are capable of using commonly seen intermolecular interactions such as hydrogen bonding, electrostatic complementarities, hydrophobic contacts, and steric exclusion to bind with a specific target. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides.

In some embodiments, a binding moiety is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a binding moiety is a cell. For example, a binding moiety can be an intact cell, a cell treated with a compound (e.g., a drug), a fixed cell, a lysed cell, or any combination thereof. In some embodiments, a binding moiety is a single cell. In some embodiments, a binding moiety is a plurality of cells.

In some embodiments, a binding moiety is a plurality of binding moieties, such as a mixture or library of binding moieties. In some embodiments, a binding moiety is a plurality of different binding moieties. For example, a binding moiety can comprise a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties. In some embodiments, a binding moiety is a plurality of different binding moieties that represents an entire, or portion of, a proteome of an organism. For example, a binding moiety can comprise a plurality of proteins representing at least about 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of an organism's proteome. For example, a binding moiety can comprise a plurality of antibodies that bind to at least about 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of the proteins of an organism's proteome. The proteome can be a bacterial, viral, fungal proteome. The proteome can be of an insect or mammal, such as a mouse, rat, rabbit, cat, dog, monkey, goat, or human. In some embodiments, the proteome is human. The proteome can be of an animal or a non-human animal, such as a bovine, avian, canine, equine, feline, ovine, porcine, or primate. The proteome can be of a mammal, such as a mouse, rat, rabbit, cat, dog monkey, or goat.

### Proximity Polynucleotides

A proximity polynucleotide is a region, molecule, domain, portion, fragment, or moiety of a proximity probe that can be coupled to an address polynucleotide when in proximity to the address polynucleotide. A proximity polynucleotide can be coupled directly or indirectly to a binding moiety. A proximity polynucleotide can be coupled covalently or non-covalently to a binding moiety. In preferred embodiments, a proximity polynucleotide does not substantially interact with a target analyte. In preferred embodiments, a proximity polynucleotide interacts with, or can be coupled to, an address polynucleotide when the proximity polynucleotide is in proximity to the address polynucleotide. In preferred embodiments, a proximity polynucleotide interacts with, or can be coupled to, an address polynucleotide when the binding moiety of a proximity probe binds to a target analyte in proximity to the address polynucleotide.

A proximity polynucleotide can comprise a plurality of proximity polynucleotides. The plurality of proximity polynucleotides can be comprised by a plurality of proximity probes. For example, a proximity polynucleotide can comprise a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 proximity polynucleotides. For example, a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 proximity polynucleotides can be comprised by a plurality of at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 proximity probes.

A proximity polynucleotide can comprise a proximity barcode sequence, a proximity linker sequence, a proximity primer binding sequence, a proximity spacer sequence, or any combination thereof. A proximity polynucleotide can be arranged in an order such that a proximity linker sequence is located at one end of the proximity polynucleotide. A proximity polynucleotide can be arranged in an order such that it contains a proximity barcode upstream of the proximity linker sequence. A proximity polynucleotide can comprise a proximity linker sequence between the proximity barcode and the proximity linker sequence. A proximity polynucleotide can be arranged in an order such that it contains a proximity primer binding sequence upstream of the proximity barcode. A proximity polynucleotide can comprise a proximity linker sequence between the proximity barcode and the proximity primer binding sequence. A proximity polynucleotide can comprise a proximity linker sequence between the proximity barcode and the proximity primer binding sequence. A proximity polynucleotide can be arranged in an order such that a proximity spacer sequence is located upstream or downstream of the proximity primer binding sequence. A proximity polynucleotide can be arranged in an order such that a proximity spacer sequence is located upstream of the proximity barcode sequence. A proximity polynucleotide can be arranged in an order such that a proximity spacer sequence is located at one end of the proximity polynucleotide, for example, an end of the proximity polynucleotide that does not contain the proximity linker sequence. For example, a proximity polynucleotide can be arranged in an order of the proximity linker sequence, the proximity barcode, the proximity primer binding sequence, and the proximity spacer sequence. For example, a proximity polynucleotide can be arranged in an order of the proximity linker sequence, the proximity barcode, the proximity primer binding sequence, and the proximity spacer sequence propagating toward the binding moiety. For example, a proximity polynucleotide can be arranged in the order of the proximity linker sequence, the proximity barcode, the proximity primer binding sequence, and the proximity spacer sequence from the 5′ end to the 3′ end. For example, a proximity polynucleotide can comprise a 5′ end proximity linker sequence, a unique proximity barcode sequence, a reverse proximity primer binding sequence, and a 3′ proximity spacer sequence attached to a binding moiety (e.g., via a primary amine group attached to the 3′ end) in that order. For example, a proximity polynucleotide attached to a binding moiety can be arranged, propagating toward the binding moiety, in the order of the proximity linker, the proximity barcode, the proximity primer binding site, and the proximity spacer.

### Proximity Polynucleotide Linker

A proximity polynucleotide can comprise a proximity linker sequence. A proximity linker sequence is a sequence or end of a proximity polynucleotide that can be coupled to an address polynucleotide, (e.g., an address linker sequence). For example, a proximity linker sequence can be indirectly hybridized to an address polynucleotide through use of a splint polynucleotide. For example, a proximity linker sequence can be hybridized to an address polynucleotide directly. For example, an end of a proximity linker sequence can be ligated to an end of an address polynucleotide. For example, 3′ end of a proximity polynucleotide comprising a proximity linker sequence can be ligated to a 5′ end of an address polynucleotide (e.g., an address linker sequence). A proximity linker sequence can be located at a terminus or an end of a proximity polynucleotide. For example, a proximity linker sequence can be a 3′ terminus or end of a proximity polynucleotide. A proximity linker sequence can be interposed between an end of a proximity polynucleotide and a proximity primer binding sequence of a proximity polynucleotide. A proximity linker sequence can be located downstream of a proximity primer binding sequence. For example, a proximity linker sequence can be located 3′ to a proximity primer binding sequence. A proximity linker sequence can be located downstream of a proximity barcode sequence of a proximity polynucleotide. For example, a proximity linker sequence can be located 3′ to a proximity barcode sequence. A proximity linker sequence can be located downstream of a proximity spacer sequence of a proximity polynucleotide. For example, a proximity linker sequence can be located 3′ to a proximity spacer sequence.

A proximity linker sequence can be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more consecutive nucleotides. A proximity linker sequence can be a sequence of known length.

A proximity linker sequence of each proximity polynucleotide of a plurality of proximity probes can be a unique or a same linker sequence. For example, any one proximity linker sequence of a plurality of proximity linker sequences can be a unique linker sequence. In preferred embodiments, each proximity linker sequence of a plurality of proximity linker sequences is the same sequence. For example, each proximity linker sequence of a plurality of proximity linker sequences can comprise a same sequence that hybridizes to a splint polynucleotide. For example, each proximity linker sequence of a plurality of proximity linker sequences can comprise a same sequence that hybridizes to a same sequence of a splint polynucleotide. For example, each address linker sequence of a plurality of address linker sequences can comprise a same sequence that hybridizes to a same sequence of a splint polynucleotide, wherein the splint polynucleotide can hybridize to a proximity linker sequence (thus, coupling the address polynucleotide and the proximity polynucleotide). For example, each proximity polynucleotide can comprise the same proximity linker sequence. A proximity linker sequence can be a universal sequence.

A proximity linker sequence can comprise a randomly assembled sequence of nucleotides. A proximity linker sequence can be a sequence of known length. A proximity linker sequence can be a known sequence. A proximity linker sequence can be a predefined sequence. A proximity linker sequence can be an unknown sequence of known length. A proximity linker sequence can be an known sequence of known length. In preferred embodiments, a proximity linker sequence is a universal sequence such that coupling of each proximity linker sequence of a plurality of proximity probes to an address polynucleotide can be carried out with a universal splint polynucleotide. Thus, a universal splint polynucleotide that hybridizes to each of the proximity linker sequences can be utilized to couple all proximity probes to an address polynucleotide in a single reaction, simultaneously, and/or without bias for the coupling reaction.

### Proximity Polynucleotide Barcode

A proximity polynucleotide can comprise a proximity barcode sequence or compliment thereof. A proximity barcode can allow for identification of a proximity probe comprising the proximity barcode. A proximity barcode can allow for identification of a binding moiety to which the proximity barcode is attached. A proximity barcode can be used to identify a binding moiety from a plurality of binding moieties that binds to a target analyte. A proximity barcode can be barcoded to a proximity probe exclusively. A proximity barcode can be barcoded to a binding moiety exclusively. Thus, a proximity barcode sequence can be barcoded to a specific binding moiety.

A proximity barcode can be a unique barcode sequence. For example, any one proximity barcode of a plurality of proximity barcodes can be a unique barcode sequence. The number of different barcode sequences theoretically possible can be directly dependent on the length of the barcode sequence. For example, if a DNA barcode with randomly assembled adenine, thymidine, guanosine and cytidine nucleotides can be used, the theoretical maximal number of barcode sequences possible can be 1,048,576 for a length of ten nucleotides, and can be 1,073,741,824 for a length of fifteen nucleotides. A proximity barcode sequence can comprise a sequence of at least 3, 4, 5, 6, 7, 8, 9, 10, 11, 12, 13, 14, 15, 16, 17, 18, 19, 20, 25, 30, 40, 45, or 50 or more consecutive nucleotides. A proximity polynucleotide can comprise two or more proximity barcode sequences or compliments thereof. A proximity barcode sequence can comprise a randomly assembled sequence of nucleotides. A proximity barcode sequence can be a degenerate sequence. A proximity barcode sequence can be a known sequence. A proximity barcode sequence can be a predefined sequence. In a preferred embodiment, a proximity barcode sequence is a known, unique sequence that is barcoded to a binding moiety to which it is coupled such that a signal containing the proximity barcode (e.g., a sequence read) or compliment thereof can be used to identify a binding moiety of a plurality of binding moieties that interacted with a target analyte of a plurality of target analytes.

### Proximity Polynucleotide Primer Binding Site

A proximity primer binding sequence can be used as a primer binding site for a reaction, such as amplification or sequencing. A proximity primer binding sequence can be a first primer binding sequence for a pair of primers used for a reaction, such as amplification or sequencing. For example, a proximity primer binding sequence can be a forward primer binding site. For example, a proximity primer binding site can be a reverse primer binding site. For example, a proximity primer binding site can be a forward primer binding site and an address primer binding sequence can be a reverse primer binding sequence. In some embodiments, a proximity primer binding sequence is a universal primer binding sequence.

A proximity primer binding sequence and an address primer binding sequence (e.g., of an address polynucleotide) can comprise melting temperatures that differ by no more than 6, 5, 4, 3, 2, or 1 degree Celsius. The nucleotide sequence of a proximity primer binding sequence and an address primer binding sequence of an address polynucleotide can differ such that a polynucleotide that hybridizes to the proximity primer binding sequence does not hybridize to the address primer binding sequence. The nucleotide sequence of a proximity primer binding sequence and an address primer binding sequence of an address polynucleotide can differ such that a polynucleotide that hybridizes to the address primer binding sequence does not hybridize to the proximity primer binding sequence.

A proximity primer binding sequence can be located upstream of an address barcode. For example, a proximity primer binding sequence can be 5′ to a proximity barcode. A proximity primer binding sequence can be located upstream of a proximity linker sequence. For example, a proximity primer binding sequence can be 5′ to a proximity linker sequence. A proximity primer binding sequence can be located upstream of an address linker sequence when the proximity polynucleotide is coupled to an address polynucleotide. For example, a proximity primer binding sequence can be 5′ to an address linker sequence when the proximity polynucleotide is coupled to an address polynucleotide. A proximity primer binding sequence can be located upstream of an address barcode sequence when the proximity polynucleotide is coupled to an address polynucleotide. For example, a proximity primer binding sequence can be 5′ to an address barcode sequence when the proximity polynucleotide is coupled to an address polynucleotide.

### Proximity Polynucleotide Spacer

A proximity polynucleotide can comprise a proximity spacer sequence. A proximity spacer sequence is a sequence used to increase the length of the proximity polynucleotide or to separate one or more of a proximity barcode, proximity linker, a proximity primer binding site, and a binding moiety from each other. In some embodiments, a proximity polynucleotide does not comprise a proximity spacer sequence. For example, a proximity polynucleotide can be coupled to a binding moiety at an end of the proximity polynucleotide comprising a proximity primer binding site.

In some embodiments, a proximity spacer sequence is attached to a binding moiety of a proximity probe. In some embodiments, a proximity spacer is located upstream of a proximity primer binding sequence. For example, a proximity spacer sequence can be located 5′ to a proximity primer binding sequence. In some embodiments, a proximity spacer is located downstream of a proximity primer binding sequence. For example, a proximity spacer sequence can be located 3′ to a proximity primer binding sequence. In some embodiments, a proximity spacer is located upstream of a proximity barcode. For example, a proximity spacer sequence can be located 5′ to a proximity barcode. In some embodiments, a proximity spacer is located downstream of a proximity barcode. For example, a proximity spacer sequence can be located 3′ to a proximity barcode. In some embodiments, a proximity spacer is located upstream of a proximity linker sequence. For example, a proximity spacer sequence can be located 5′ to a proximity linker sequence.

In some embodiments, a proximity spacer is interposed between a proximity primer binding sequence and a binding moiety of a proximity probe. For example, a proximity spacer sequence can be located 5′ to a proximity primer binding sequence and 5′ end of the proximity polynucleotide containing the proximity linker sequence can be attached to a binding moiety of a proximity polynucleotide. In some embodiments, a proximity spacer is interposed between a proximity primer binding sequence and a proximity barcode. For example, a proximity spacer sequence can be located 3′ to a proximity primer binding sequence and 5′ to a proximity barcode. In some embodiments, a proximity spacer is interposed between a proximity linker sequence and a proximity barcode. For example, a proximity spacer sequence can be located 3′ to a proximity barcode and 5′ to a proximity linker sequence.

A proximity spacer sequence can be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 250, 300, 400, 500 or more consecutive nucleotides. A proximity spacer sequence can comprise a randomly assembled sequence of nucleotides. A proximity spacer sequence can be a sequence of known length. A proximity spacer sequence can be a known sequence. A proximity spacer sequence can be a predefined sequence. A proximity spacer sequence can be an unknown sequence of known length. A proximity spacer sequence can be a known sequence of known length.

### Coupling of Proximity Polynucleotide to a Binding Moiety

The proximity probes employed in the methods and compositions described herein may be prepared using any convenient method. A binding moiety can be coupled directly or indirectly (e.g., via a linker) to a proximity polynucleotide. A binding moiety can be coupled covalently (e.g. via chemical cross-linking) or non-covalently (e.g., via streptavidin-biotin) to a proximity polynucleotide. The design and preparation of proximity probes is widely described in the art, for example various different binding moieties which may be used, the design of proximity polynucleotides for proximity ligation assays, and the coupling of such o proximity polynucleotides to the binding moieties to form the proximity probes. The details and principles described in the art may be applied to the design of the proximity probes for use in the methods of the invention (See, e.g., WO2007107743, and U.S. Pat. Nos. 7,306,904 and 6,878,515).

A direct coupling reaction between a proximity polynucleotide and a binding moiety may be utilized, for example, where each possesses a functional group (e.g., a substituent or chemical handle) capable of reacting with a functional group on the other. Functional groups may be present on the proximity polynucleotide or binding moiety, or introduced onto these components (e.g. via oxidation reactions, reduction reactions, cleavage reactions and the like). Methods for producing nucleic acid/polypeptide conjugates have been described (See, e.g. U.S. Pat. No. 5,733,523).

Functional groups of an antibody or a polypeptide that can be used for coupling to a proximity polynucleotide include, but are not limited to carbohydrates, thiol groups (HS—) of amino acids, amine groups (H₂N—) of amino acids, and carboxy groups of amino acids. For example, carbohydrate structures can be oxidized to aldehydes, and reacted with a H₂NNH group containing compound to form the functional group —C═NH—NH—. For example, thiol groups can be reacted with a thiol-reactive group to form a thioether or disulfide. For example, free thiol groups of proteins may be introduced into proteins by thiolation or splitting of disulfides in native cysteine residues. For example, an amino group (e.g., of an amino-terminus or an omega amino group of a lysine residue) may be reacted with an electrophilic group (e.g., an activated carboxy group) to form an amide group. For example, a carboxy group (e.g., a carboxy-terminus or a carboxy group of a diacidic alpha amino acid) may be activated and contacted with an amino group to form an amide group. Other exemplary functional groups include, e.g., SPDP, carbodiimide, glutaraldehyde, and the like.

In an exemplary embodiment, a proximity polynucleotide is covalently coupled to a binding moiety using a commercial kit (“All-in-One Antibody-Oligonucleotide Conjugation Kit”; Solulink, Inc.). For example, first, a 3′-amino-proximity polynucleotide can be derivatized with Sulfo-S-4FB. Second, a binding moiety can be modified with an S-HyNic group. Third, the HyNic-modified binding moiety can be reacted with the 4FB-modified proximity polynucleotide to yield a bis-arylhydrazone mediated proximity probe. Excess 4FB-modified proximity polynucleotide can be further removed via a magnetic affinity matrix. The overall binding moiety recovery can be at least about 95%, 96%, 97%, 98%, 99%, or 100% free of HyNic-modified binding moiety and 4FB-modified proximity polynucleotide. The bis-arylhydrazone bond can be stable to both heat (e.g., 94° C.) and pH (e.g., 3-10).

Where linking groups are employed, such linkers may be chosen to provide for covalent attachment or non-covalent attachment of the binding domain and proximity polynucleotide through the linking group. A variety of suitable linkers are known t in the art. In some embodiments, the linker is at least about 50 or 100 Daltons 100 Daltons. In some embodiments, the linker is at most about 300; 500; 1,000; 10,000, or 100,000 Daltons. A linker can comprise a functional group at either end with a reactive functionality capable of bonding to the proximity polynucleotide. A linker can comprise a functional group at either end with a reactive functionality capable of bonding to the binding moiety. Functional groups may be present on the proximity polynucleotide, binding moiety, and/or linker, or introduced onto these components (e.g. via oxidation reactions, reduction reactions, cleavage reactions and the like).

Exemplary linkers include polymers, aliphatic hydrocarbon chains, unsaturated hydrocarbon chains, polypeptides, polynucleotides, cyclic linkers, acyclic linkers, carbohydrates, ethers, polyamines, and others known in the art. Exemplary functional groups of linkers include nucleophilic functional groups (e.g., amines, amino groups hydroxy groups, sulfhydryl groups, amino groups, alcohols, thiols, and hydrazides), electrophilic functional groups (e.g., aldehydes, esters, vinyl ketones, epoxides, isocyanates, and maleimides), and functional groups capable of cycloaddition reactions, forming disulfide bonds, or binding to metals. For example, functional groups of linkers can be primary amines, secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, maleimides, azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl[4-azidophenyl]-1,3′-dithiopropionate, N-succinimidyl[4-iodoacetyl]aminobenzoate, glutaraldehyde, and succinimidyl-4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

In other embodiments, the proximity probes may be produced using in vitro protocols that yield nucleic acid-protein conjugates (e.g. molecules having nucleic acids covalently bonded to a protein), such as producing the binding domain in vitro from vectors which encode the proximity probe. Examples of such in vitro protocols of interest include: RepA based protocols (See, e.g., Fitzgerald et al., Drug Discov Today (2000) 5:253-58 and WO9837186), ribosome display based protocols (See, e.g., Hanes et al., PNAS (1997) 94:4937-42; Roberts et al., Curr Opin Chem Biol (1999) June; 3: 268-73; Schaffitzel et al., J Immunol Methods (1999) December 10; 231:119-35; and WO9854312), etc.

### Complexes

The methods provided herein comprise forming complexes. A complex refers to an association between at least two moieties (e.g. chemical or biochemical) that have an affinity for one another. The methods provided herein comprise forming a complex between a target analyte and a binding moiety. In some embodiments, the methods comprise forming a complex between a target analyte and a single binding moiety. In some embodiments, the methods comprise forming a complex between a target analyte and a complex of two or more binding moieties. In some embodiments, the methods comprise forming a complex between a target analyte and a complex of two or more binding moieties. In some embodiments, the methods comprise forming a complex between two or more target analytes and a complex of two or more binding moieties. In some embodiments, the methods comprise forming a complex between a first complex comprising a target analyte and another moiety (e.g., a polypeptide, polynucleotide, or small molecule) and a binding moiety. In some embodiments, the methods comprise forming a complex between a first complex comprising a target analyte and another moiety (e.g., a polypeptide, polynucleotide, or small molecule) and a second complex comprising two or more binding moieties. For example, complexes can be formed between a target analyte coupled to a solid support, and a proximity probe comprising a binding moiety and a proximity polynucleotide coupled to the binding moiety.

Complexes include a proximity probe bound to a target analyte. Complexes include a binding moiety of a proximity probe bound to a target analyte and a proximity polynucleotide of the proximity probe coupled to an address polynucleotide. Complexes include a binding moiety (e.g., a binding moiety of a proximity probe) bound to a target analyte.

For example, complexes can include antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

Complexes that may be excluded in some instances include complexes consisting of an address polynucleotide bound directly to a target analyte. Complexes that may be excluded in some instances include complexes consisting of an address polynucleotide bound directly to a binding moiety. Complexes that may be excluded in some instances include complexes consisting of a proximity polynucleotide bound directly to a target analyte.

In some instances, a complex comprises a polypeptide interacting with a single-stranded DNA. In some instances, a complex comprises a tagged protein interacting with a single-stranded DNA. In some instances, a complex comprises an antibody interacting with a single-stranded DNA. In some instances, a complex comprises a virus particle interacting with a single-stranded DNA. In some instances, a complex comprises a cell interacting with a single-stranded DNA. In some instances, a complex comprises a small molecule interacting with a single-stranded DNA. In some instances, a complex comprises polypeptide interacting with a double-stranded DNA. In some instances, a complex comprises a tagged protein interacting with a double-stranded DNA. In some instances, a complex comprises an antibody interacting with a double-stranded DNA. In some instances, a complex comprises a virus particle interacting with a double-stranded DNA. In some instances, a complex comprises a cell interacting with a double-stranded DNA. In some instances, a complex comprises a small molecule interacting with a double-stranded DNA. In some instances, a complex comprises a polypeptide interacting with a single-stranded RNA. In some instances, a complex comprises a tagged protein interacting with a single-stranded RNA. In some instances, a complex comprises an antibody interacting with a single-stranded RNA. In some instances, a complex comprises a virus particle interacting with a single-stranded RNA. In some instances, a complex comprises a cell interacting with a single-stranded RNA. In some instances, a complex comprises a small molecule interacting with a single-stranded RNA. In some instances, a complex comprises a polypeptide interacting with a double-stranded RNA. In some instances, a complex comprises a tagged protein interacting with a double-stranded RNA. In some instances, a complex comprises an antibody interacting with a double-stranded RNA. In some instances, a complex comprises a virus particle interacting with a double-stranded RNA. In some instances, a complex comprises a cell interacting with a double-stranded RNA. In some instances, a complex comprises a small molecule interacting with a double-stranded RNA. In some instances, a complex comprises a polypeptide interacting with a RNA-DNA hybrid. In some instances, a complex comprises a tagged protein interacting with a RNA-DNA hybrid. In some instances, a complex comprises an antibody interacting with a RNA-DNA hybrid. In some instances, a complex comprises a virus particle interacting with a RNA-DNA hybrid. In some instances, a complex comprises a cell interacting with a RNA-DNA hybrid. In some instances, a complex comprises a small molecule interacting with a RNA-DNA hybrid. In some instances, a complex comprises a small molecule interacting with a double-stranded RNA.

In some instances, a complex comprises a polypeptide interacting with a methylated polynucleotide. In some instances, a complex comprises a tagged protein interacting with a methylated polynucleotide. In some instances, a complex comprises an antibody interacting with a methylated polynucleotide. In some instances, a complex comprises a virus particle interacting with a methylated polynucleotide. In some instances, a complex comprises a cell interacting with a methylated polynucleotide. In some instances, a complex comprises a small molecule interacting with a methylated polynucleotide. In some instances, a complex comprises a polypeptide interacting with an unmethylated polynucleotide. In some instances, a complex comprises a tagged protein interacting with an unmethylated polynucleotide. In some instances, a complex comprises an antibody interacting with an unmethylated polynucleotide. In some instances, a complex comprises a virus particle interacting with an unmethylated polynucleotide. In some instances, a complex comprises a cell interacting with an unmethylated polynucleotide. In some instances, a complex comprises a small molecule interacting with an unmethylated polynucleotide.

In some instances, a complex comprises a polypeptide interacting with a polynucleotide-coupled small molecule. In some instances, a complex comprises a tagged protein interacting with a polynucleotide-coupled small molecule. In some instances, a complex comprises an antibody interacting with a polynucleotide-coupled small molecule. In some instances, a complex comprises a virus particle interacting with a polynucleotide-coupled small molecule. In some instances, a complex comprises a cell interacting with a polynucleotide-coupled small molecule.

In some instances, a complex comprises a polypeptide interacting with an aptamer. In some instances, a complex comprises a tagged protein interacting with an aptamer. In some instances, a complex comprises an antibody interacting with an aptamer. In some instances, a complex comprises a virus particle interacting with an aptamer. In some instances, a complex comprises a cell interacting with an aptamer. In some instances, a complex comprises a small molecule interacting with an aptamer.

In some instances, a complex comprises a polypeptide interacting with another polypeptide. In some instances, a complex comprises a tagged protein interacting with a polypeptide. In some instances, a complex comprises an antibody interacting with a polypeptide. In some instances, a complex comprises a virus particle interacting with a polypeptide. In some instances, a complex comprises a cell interacting with a polypeptide. In some instances, a complex comprises a small molecule interacting with a polypeptide. In some instances, a complex comprises a tagged protein interacting with an antibody. In some instances, a complex comprises an antibody interacting with another antibody. In some instances, a complex comprises a virus particle interacting with an antibody. In some instances, a complex comprises a cell interacting with an antibody. In some instances, a complex comprises a small molecule interacting with an antibody. In some instances, a complex comprises a tagged protein interacting with a virus particle. In some instances, a complex comprises a virus particle interacting with another virus particle. In some instances, a complex comprises a cell interacting with a virus particle. In some instances, a complex comprises a small molecule interacting with a virus particle. In some instances, a complex comprises a tagged protein interacting with a cell. In some instances, a complex comprises a cell interacting with another cell. In some instances, a complex comprises a small molecule interacting with a cell.

In some instances, a complex comprises one or more polypeptides bound to one or more other polypeptides, one or more polynucleotides (e.g. DNAs, RNAs aptamers), one or more tagged proteins, one or more antibodies, one or more virus particles, one or more cells, one or more small molecules, or any combination thereof. In some instances, a complex comprises one or more tagged proteins bound to one or more polynucleotides (e.g. DNAs, RNAs aptamers), one or more other tagged proteins, one or more antibodies, one or more virus particles, one or more cells, one or more small molecules, or any combination thereof. In some instances, a complex comprises one or more antibodies bound to one or more polynucleotides (e.g. DNAs, RNAs aptamers), one or more other antibodies, one or more virus particles, one or more cells, one or more small molecules, or any combination thereof. In some instances, a complex comprises one or more virus particles bound to one or more polynucleotides (e.g. DNAs, RNAs aptamers), one or more other virus particles, one or more cells, one or more small molecules, or any combination thereof. In some instances, a complex comprises one or more cells bound to one or more polynucleotides (e.g. DNAs, RNAs aptamers), one or more other cells, one or more small molecules, or any combination thereof. In some instances, a complex comprises one or more small molecules bound to one or more polynucleotides (e.g. DNAs, RNAs aptamers), one or more other small molecules, or any combination thereof.

### Coupling a Proximity Polynucleotide to an Address Polynucleotide

The methods disclosed herein can also include coupling a proximity polynucleotide in proximity to an address polynucleotide. The proximity linker sequence and the address linker sequence are generally of a length sufficient to allow coupling to each other. For example, the proximity linker sequence and the address linker sequence can be of a length to permit hybridization of the two polynucleotides. For example, the proximity linker sequence can be of a length to permit splint polynucleotide-mediated interactions with the address linker sequence (e.g., when a binding moiety to which the proximity linker sequence is coupled is bound to a target analyte (e.g., in proximity to the address polynucleotide). Proximity linker sequences and address linker sequences can be from about 8 up to about 1,000 nucleotides in length, about 8 to about 500 nucleotides in length, about 8 to about 250 nucleotides in length, about 8 to about 160 nucleotides in length, about 12 to about 150 nucleotides in length, about 14 to about 130 nucleotides in length, about 16 to about 110 nucleotides in length, about 8 to about 90 nucleotides in length, about 12 to about 80 nucleotides in length, about 14 to about 75 nucleotides in length, about 16 to about 70 nucleotides in length, about 16 to about 60 nucleotides in length, and so on. In certain representative embodiments, the proximity linker sequences and address linker sequences may range in length from about 10 to about 80 nucleotides in length, from about 12 to about 75 nucleotides in length, from about 14 to about 70 nucleotides in length, from about 34 to about 60 nucleotides in length, and any length between the stated ranges. In some embodiments, the proximity linker sequences and address linker sequences are not more than about 28, 29, 30, 31, 32, 33, 34, 35, 36, 37, 38, 39, 40, 41, 42, 44, 46, 50, 55, 60, 65, or 70 nucleotides in length.

The use of a splint polynucleotide in proximity ligation assays is known in the art. The splint polynucleotide may accordingly be viewed a “connector” polynucleotide which acts to connect or “hold together” the proximity linker sequence and the address linker sequence, such that they may interact, or may be coupled together. A splint polynucleotide is generally of a length sufficient to allow coupling of the proximity linker sequence and the address linker sequence to each other. The sequence of the splint polynucleotide may be chosen or selected with respect to the proximity linker sequence and the address linker sequence. The sequence of the proximity linker sequence and the address linker sequence may be chosen or selected with respect to a splint polynucleotide. Thus, these sequences are not critical as long as the proximity linker sequence and the address linker sequence may hybridize to the splint polynucleotide However, the proximity linker sequences and the address linker sequences should be chosen to avoid the occurrence of hybridization events other than between the proximity linker sequence and the address linker sequence with that of the splint polynucleotide. Once the proximity linker sequence and the address linker sequence are selected or identified, the splint polynucleotide sequence may be synthesized using any convenient method. In some instances, the splint polynucleotide can be a short single-stranded molecule complementary to an end of the address polynucleotide linker and an end of the proximity linker. Hence, the splint polynucleotide will bring the termini of the address polynucleotide linker and the proximity linker into position for a ligase to join the two ends. The splint can then be removed by using exonucleases e.g. exonuclease I and exonuclease III, to digest the splint polynucleotide. The splint polynucleotide can be at least 2 nucleotides, e.g., 2, 3, 4, 5, 6, 7, 8, 9, 10, 11, 12, 13, 14, 15, 16, 17, 18, 19, 20 or more nucleotides) in length.

In particular the splint polynucleotide hybridizes with the proximity linker sequences and the address linker sequences. A splint polynucleotide can hybridize (anneal) simultaneously or in the same reaction with each of a plurality of proximity linker sequences. A splint polynucleotide can hybridize simultaneously or in the same reaction with each of a plurality of address linker sequences. A splint polynucleotide can hybridize simultaneously or in the same reaction with each of a plurality of proximity linker sequences and each of a plurality of address linker sequences. The hybridization of the proximity linker sequences of each proximity probe and the address polynucleotide to each other increases the avidity of the proximity probe-target analyte complex upon binding to the target analyte. This avidity effect contributes to the sensitivity of the assays by supporting the formation of signal-generating proximity probe-target analyte complexes.

A proximity linker sequence and an address linker sequence can be coupled when in proximity to each other. A proximity linker sequence and an address linker sequence can be coupled using any method that permits amplification and/or detection of the address barcode and the proximity barcode such that the two barcodes are known to arise from a sample molecule or complex of molecules. In some embodiments, a proximity linker sequence and an address linker sequence are coupled by ligating the two polynucleotides to each other.

Ligation involves creating a phosphodiester bond between the 3′ hydroxyl of one nucleotide and the 5′ phosphate of another. In a ligation step, a suitable ligase and any reagents that are necessary and/or desirable are contacted to the solid support or spot on a solid support and maintained under conditions sufficient for ligation of the proximity linker sequence and address linker sequence to occur. Ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary (e.g. a splint polynucleotide). Any convenient ligase (e.g., temperature sensitive and thermostable ligases) may be employed, where representative ligases of interest include, but are not limited to bacteriophage T4 DNA ligase, Taq ligase, Tth ligase, Ampligase®, Pfu ligase, Thermus thermophilus ligase, Thermus acquaticus ligase, Pyrococcus ligase, bacteriophage T7 ligase, and E. coli ligase. Thermostable ligase may be obtained from thermophilic or hyperthermophilic organisms (e.g., prokaryotic, eukaryotic, or archael organisms). Certain RNA ligases may also be employed in the methods of the invention.

Ligation reaction conditions are well known to those of skill in the art. Ligation can be carried out at 4-45° C. in the presence of a ligase enzyme (e.g., a DNA ligase). For example, during ligation, the reaction mixture may be maintained at a temperature ranging from about 4° C. to about 50° C., or 20° C. to about 37° C.; and for a period of time ranging from about 5 seconds to about 16 hours, such as from about 1 minute to about 1 hour. In yet other embodiments, the reaction mixture may be maintained at a temperature ranging from about 35° C. to about 45° C., such as from about 37° C. to about 42° C. (e.g., at or about 38° C., 39° C., 40° C. or 41° C.), for a period of time ranging from about 5 seconds to about 16 hours, such as from about 1 minute to about 1 hour, including from about 2 minutes to about 8 hours. A ligation reaction mixture can include, for example, 50 mM Tris pH7.5, 10 mM MgCl₂, 10 mM DTT, 1 mM ATP, 25 mg/ml BSA, 0.25 units/ml RNase inhibitor, and T4 DNA ligase at 0.125 units/ml. A ligation reaction mixture can include, for example, 2.125 mM magnesium ion, 0.2 units/ml RNase inhibitor; and 0.125 units/ml DNA ligase.

It will be evident that the ligation conditions may depend on the ligase enzyme used in the methods of the invention. Hence, the above-described ligation conditions are merely a representative example and the parameters may be varied according to well-known protocols. For example, a ligase, namely Ampligase®, may be used at temperatures of greater than 50° C. However, it will be further understood that the alteration of one parameter (e.g. temperature) may require the modification of other conditions to ensure that other steps of the assay are not inhibited or disrupted, (e.g. binding of the proximity probe to the target analyte). Such manipulation of the methods is routine in the art.

In some instances, ligating comprises bringing an end of a proximity polynucleotide adjacent to an end of an address polynucleotide. In some instances, bringing an end of a proximity polynucleotide adjacent to an end of an address polynucleotide comprises hybridizing a splint polynucleotide to the proximity linker sequence and the address polynucleotide linker sequence. In some instances, ligating comprises hybridizing a splint polynucleotide to the proximity linker sequence and the address polynucleotide linker sequence. Ligation of a proximity polynucleotide to an address polynucleotide wherein the proximity polynucleotide and address polynucleotide are hybridized to a splint polynucleotide, can be achieved by contacting a ligating activity thereto (e.g. provided by a suitable nucleic acid ligase) and maintaining the mixture under conditions sufficient for ligation of the proximity linker sequence and address linker sequence to occur. For example, a proximity linker sequence and an address linker sequence can be coupled to each other by ligating an end of the proximity linker sequence to an end of the address linker sequence. For example, a proximity linker sequence and an address linker sequence can be coupled to each other by ligating a 5′ end of the address polynucleotide to a 3′ end of the proximity polynucleotide.

Thus, the methods provide for ligating a proximity polynucleotide to an address polynucleotide, wherein the address polynucleotide is coupled to a solid support and in proximity to a target analyte or in proximity to a proximity probe bound to the target analyte. A ligated product of the resulting ligation reaction between the proximity polynucleotide and the address polynucleotide, or an amplified product thereof, can then be detected and/or amplified.

In some instances, coupling comprises hybridizing an address linker sequence to a proximity linker sequence. Such a coupled product can be subjected to extension of one or both ends of the hybridized linker sequences. Such a coupled product containing one or both extended ends of the hybridized linker sequences can then be amplified as described herein (e.g., such that the amplified products contain the proximity barcode and the address barcode.

The new paired barcoded polynucleotide composition generated using the methods of the invention can serve multiple functions. For example, the paired barcoded polynucleotide allows for quantitative and/or qualitative detection of target analytes, binding moieties, and affinities and specificities between target analytes and binding, on multiplex and multiplex-on-multiplex formats. The paired barcoded polynucleotide can serve to pair or join a binding event between a single target analyte and a single binding moiety from a plurality of target analytes and a plurality of binding moieties. The paired barcoded polynucleotide barcodes the identity or location of the address polynucleotide to an array. The paired barcoded polynucleotide barcodes the identity or location of the target analyte on an array. The paired barcoded polynucleotide barcodes the identity of a binding moiety, a proximity probe, and/or a proximity polynucleotide. The paired barcoded polynucleotide barcodes the location of a binding event of a binding moiety and a target analyte.

Various proximity ligation assay formats (e.g., in solution) are described in WO0161037, WO9700446, WO0161037, WO03044231, WO05123963; U.S. Pat. No. 6,511,809; Fredriksson et al. (2002) Nat Biotech 20:473-477; and Gullberg et al. (2004) Proc Natl Acad Sci USA 101:8420-8424.

For example, rather than being ligated to each other, the nucleic acid domains of the proximity probes when in proximity may template the ligation of one or more added oligonucleotides to each other (which may be the nucleic acid domain of one or more proximity probes), including an intramolecular ligation to circularize an added linear oligonucleotide, for example based on the so-called padlock probe principle, wherein analogously to a padlock probe, the ends of the added linear oligonucleotide are brought into juxtaposition for ligation by hybridizing to a template, here a nucleic acid domain of the proximity probe (in the case of a padlock probe the target nucleic acid for the probe).

For example, nucleic acid domains may be joined to form a new nucleic acid sequence generally by means of a ligation reaction, which may be templated by a splint polynucleotide added to the reaction, the splint polynucleotide containing regions of complementarity for the ends of the respective polynucleotide domains of the barcoded proximity probe and the barcoded address polynucleotide.

In a further modification described in WO07/107743 the splint polynucleotide to template ligation of the nucleic acid domains of two proximity probes is carried on a third proximity probe.

Although pairs of proximity probes are generally used, modifications of the proximity-probe detection assay have been described, in e.g. WO01/61037, WO07/044903, WO09/012220, and WO05/123963, where three proximity probes are used to detect a single analyte molecule, the nucleic acid domain of the third probe possessing two free ends which can be joined (ligated) to the respective free ends of the nucleic acid domains of the first and second probes, such that it becomes sandwiched between them. In this embodiment, two species of splint polynucleotides are required to template ligation of each of the first and second probes' nucleic acid domains to that of the third.

In addition to modification to the proximity-probe detection assay, modifications of the structure of the proximity probes themselves have been described, in e.g. WO03/044231, where multivalent proximity probes are used. Such multivalent proximity probes comprise at least two, but as many as 100, analyte-binding domains conjugated to at least one, and preferably more than one, nucleic acid(s).

Coupling can comprise hybridizing an address linker sequence to a proximity linker sequence via enzymatic and non-enzymatic (e.g., chemical) methods. Examples of ligation reactions that are non-enzymatic include the non-enzymatic ligation techniques described in U.S. Pat. Nos. 5,780,613 and 5,476,930. In some embodiments, a ligase, for example a DNA ligase or RNA ligase is used for coupling. Ligation techniques comprise blunt-end ligation and sticky-end ligation. Ligation reactions may include DNA ligases such as DNA ligase I, DNA ligase III, DNA ligase IV, and T4 DNA ligase. Ligation reactions may include RNA ligases such as T4 RNA ligase I and T4 RNA ligase II. Multiple ligases, each having characterized reaction conditions, are known in the art, and include, without limitation NAD⁺-dependent ligases including tRNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof.

In some instances, ligation can be between polynucleotides having hybridizable sequences, such as complementary overhangs. In some instances, ligation can be between polynucleotides having. Generally, a 5′ phosphate is utilized in a ligation reaction. The 5′ phosphate can be provided by the target polynucleotide, the adaptor oligonucleotide, or both. 5′ phosphates can be added to or removed from polynucleotides to be joined, as needed. Methods for the addition or removal of 5′ phosphates are known in the art, and include without limitation enzymatic and chemical processes. Enzymes useful in the addition and/or removal of 5′ phosphates include kinases, phosphatases, and polymerases. In some embodiments, 3′ phosphates are removed prior to ligation.

In some embodiments, the coupling makes use of CLICK chemistry. Suitable methods to link various molecules using CLICK chemistry are known in the art (for CLICK chemistry linkage of oligonucleotides, see, e.g. El-Sagheer et al. (PNAS, 108:28, 11338-11343, 2011). Click chemistry may be performed in the presence of Cul.

In some embodiments, the coupling makes use of topoisomerase, e.g., a Vaccinia virus topoisomerase I. In some embodiments, the coupling makes use of restriction enzyme known in the art that produces blunt ends. For example, following the generation of blunt ends, a 3′ overhang can be added to the blunt ends. For example, a 3′ overhang can be added using terminal transferase in the presence of dNTPs. For example, a 3′ overhang can be added using a polymerase in the presence of dNTPs. The polymerase can be a polymerase lacking proof-reading activity. In some cases, the polymerase can be a Taq polymerase. After the addition of a 3′ overhang, topoisomerase I bonded to can ligate the polynucleotides. For example, coupling can comprise incubation with Vaccinia virus topoisomerase I using any method as provided herein, processing with a blunt end cutting restriction enzyme, incubation with an enzyme (e.g., Taq polymerase) that adds a residue to each blunt end, and ligation via the topoisomerase I.

In some cases a proximity polynucleotide and an address polynucleotide are subjected to end repair. End repair can include the generation of blunt ends, non-blunt ends (i.e. sticky or cohesive ends), or single base overhangs, such as the addition of a single dA nucleotide to the 3′-end of the double-stranded polynucleotide product by a polymerase lacking 3′-exonuclease activity. In some cases, end repair is performed to produce blunt ends wherein the ends contain 5′ phosphates and 3′ hydroxyls. End repair can be performed using any number of enzymes and/or methods known in the art. An overhang can comprise about, more than, less than, or at least 1, 2, 3, 4, 5, 6, 7, 8, 9, 10, 11, 12, 13, 14, 15, 16, 17, 18, 19, or 20 nucleotides. A sticky end refers to an end of a double stranded nucleic acid wherein the 5′ or the 3′ end has an extension of one or more nucleotides and which do not form a base pair. This is in contrast to a blunt end wherein the terminal 5′ polynucleotide forms a basepair with the 3′ terminal polynucleotide.

Blunt ends can be generated by the use of a single strand specific DNA exonuclease such as for example exonuclease 1, exonuclease 7 or a combination thereof to degrade overhanging single stranded ends. Alternatively, blunt ends can be generated by the use of a single stranded specific DNA endonuclease for example but not limited to mung bean endonuclease or S1 endonuclease. Alternatively, blunt ends can be generated by the use of a polymerase that comprises single stranded exonuclease activity such as for example T4 DNA polymerase, any other polymerase comprising single stranded exonuclease activity or a combination thereof to degrade the overhanging single stranded ends. In some cases, the polymerase comprising single stranded exonuclease activity can be incubated in a reaction mixture that does or does not comprise one or more dNTPs. In other cases, a combination of single stranded nucleic acid specific exonucleases and one or more polymerases can be used to generate blunt ends. In still other cases, products of an extension reaction can be made blunt ended by filling in the overhanging single stranded ends of the double stranded polynucleotides. For example, the polynucleotides can be incubated with a polymerase such as T4 DNA polymerase or Klenow polymerase or a combination thereof in the presence of one or more dNTPs to fill in single stranded portions of the double stranded polynucleotides. Alternatively, the polynucleotides can be made blunt by a combination of a single stranded overhang degradation reaction using exonucleases and/or polymerases, and a fill-in reaction using one or more polymerases in the presence of one or more dNTPs.

For example, a polymerase without terminal transferase activity or with proofreading activity can be used for coupling the address polynucleotide to the proximity polynucleotide. DNA polymerization with these DNA polymerase enzymes can result in double stranded DNA with blunt ends, without overhang or recessive end at the 3′ end. Enzymes within this class are for example Klenow polymerase and several polymerases which have polymerase activity below 95° C. such as pfu polymerase.

### Amplification of Coupled Products

The methods provided herein can comprise an amplification step. In some embodiments, a determining step comprises amplification. Amplification can be used in the methods described herein to increase the number of copies of a nucleic acid sequence, such as through the use of enzymes. For example, detection can comprise amplifying a sequence of a polynucleotide comprising an address barcode. For example, detection can comprise amplifying a sequence of a polynucleotide comprising a proximity barcode. For example, detection can comprise amplifying a sequence of a polynucleotide comprising an address barcode and a proximity barcode. For example, detection can comprise amplifying a coupled (e.g., ligated) polynucleotide containing the proximity barcode and the address barcode and/or complements thereof.

Provided herein are methods for which detecting comprises amplifying. For example, detection can comprise amplifying a sequence of a polynucleotide comprising an address barcode. For example, detection can comprise amplifying a sequence of a polynucleotide comprising a proximity barcode. For example, detection can comprise amplifying a sequence of a polynucleotide comprising an address barcode and a proximity barcode. For example, detection can comprise amplifying a sequence of a polynucleotide that is a ligated product, such as a ligated product containing the proximity barcode and the address barcode and their complementary sequence.

The methods described herein can be used to amplify coupled polynucleotides (e.g., an address polynucleotide coupled to a proximity polynucleotide). The methods described herein can employ amplification, such as to increase in the number of copies of a sequence and/or compliment thereof, of a target polynucleotide, such as a ligated product containing a proximity barcode and an address barcode and their complementary sequence.

Amplification may be performed using any method known in the art. A variety of amplification processes are known. One of the most commonly used is the polymerase chain reaction (PCR). The PCR process of Mullis is described in U.S. Pat. Nos. 4,683,195 and 4,683,202. Any type of PCR may be used. In general, the PCR amplification process involves an enzymatic chain reaction for preparing exponential quantities of a specific nucleic acid sequence. It requires a small amount of a sequence to initiate the chain reaction and polynucleotide primers that will hybridize to the sequence. In PCR, primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed. Amplification methods also include methods performed at a single temperature (isothermal).

Other means of amplifying nucleic acid that can be used in the methods include, for example, reverse transcription-PCR, real-time PCR, quantitative real-time PCR, digital PCR (dPCR), digital emulsion PCR (dePCR), clonal PCR, amplified fragment length polymorphism PCR (AFLP PCR), allele specific PCR, assembly PCR, asymmetric PCR (in which a great excess of primers for a chosen strand can be used), colony PCR, helicase-dependent amplification (HDA), Hot Start PCR, inverse PCR (IPCR), in situ PCR, long PCR (extension of DNA greater than about 5 kilobases), multiplex PCR, nested PCR (uses more than one pair of primers), single-cell PCR, touchdown PCR, loop-mediated isothermal PCR (LAMP), recombinase polymerase amplification (RPA), and nucleic acid sequence based amplification (NASBA). Other amplification methods include LCR (ligase chain reaction) which utilizes DNA ligase, and a probe consisting of two halves of a DNA segment that is complementary to the sequence of the DNA to be amplified, enzyme QB replicase and an RNA sequence template attached to a probe complementary to the DNA to be copied which is used to make a DNA template for exponential production of complementary RNA, strand displacement amplification (SDA), Qβ replicase amplification (QβRA), self-sustained replication (3SR), Branch DNA Amplification, Rolling Circle Amplification, Circle to Circle Amplification, SPIA amplification, Target Amplification by Capture and Ligation (TACL) amplification, and RACE amplification.

Amplification may be performed by amplifying a sequence of a polynucleotide, such as a ligated product, as a single amplification product (e.g., a single amplified amplicon). For example, a primer may be selected such that one amplified product can include all target sequences, such as an address barcode sequence and a proximity barcode sequence, contained in one ligated product.

Amplification may be performed by amplifying a sequence of a polynucleotide, such as a ligated product, that has a length of about 5,000 nucleotides or less. For example, the length of the ligated product may be a length of 4,500; 4,000; 3,500; 3,000; 3,000; 2,500; 2,000; 1,500; 1,000; 800; 600; 400; 200; or 100 nucleotides or less. Amplification may be performed by amplifying a sequence of a polynucleotide, such as a ligated product, that has a length of about 10 or more nucleotides. For example, the length of the ligated product may be a length of 4,500; 4,000; 3,500; 3,000; 3,000; 2,500; 2,000; 1,500; 1,000; 800; 600; 400; 200; or 100 nucleotides or more. For example, the length of the ligated product may be a length of from about 10-5,000; 10-4,500; 10-4,000; 10-3,500; 10-3,000; 10-3,000; 10-2,500; 10-2,000; 10-1,500; 10-1,000; 10-800; 10-600; 10-400; 10-200; 15-5,000; 15-4,500; 15-4,000; 15-3,500; 15-3,000; 15-3,000; 15-2,500; 15-2,000; 15-1,500; 15-1,000; 15-800; 15-600; 15-400; 15-200; 18-4,000; 18-3,500; 18-3,000; 18-3,000; 18-2,500; 18-2,000; 18-1,500; 18-1,000; 18-800; 18-600; 18-400; 18-200; 21-4,000; 21-2,000; or 21-1,000 nucleotides.

### Reverse Transcription

The information in RNA in a sample can be converted to cDNA by using reverse transcription. When the target nucleic acids to be amplified are RNAs, the methods may further comprise, producing a DNA (cDNA) complementary to the target nucleic acid by reverse-transcribing the target nucleic acid. Reverse-transcribing may be performed before or after forming a ligation product. It is known in the art that the reverse-transcribing produces a DNA complementary strand using an RNA strand using a reverse transcriptase.

Thus, the method may further include ligating an adaptor sequence to at least one of the 3′-terminus and the 5′-terminus of an RNA before reverse-transcribing the RNA, such that the resulting RNA ligation product or cDNA thereof contains a sequence complimentary to the RNA and a sequence complimentary to the adaptor sequence, which complimentary sequence also can be an adaptor sequence. The adaptor sequence may be specifically ligated to at least one of the 3′-terminal and the 5′-terminal of the nucleic acid.

Alternatively, the method may include reverse transcribing RNA, and subsequently attaching, such as ligating, one or more adaptor and primer (or primer binding) sequences to at least one of the 3′-terminal and the 5′-terminal of cDNA of nucleic acid after the reverse-transcribing. The adaptor sequence may be specifically ligated to at least one of the 3′-terminal and the 5′-terminal of the target nucleic acid. The ligating the adaptor sequence can be as described above.

### Primers

Amplification, reverse transcription, sequencing, and combinations thereof can be performed with one or more primers, or one or more primer sets. A primer is polynucleotide, the sequence of at least a portion of which is complementary to a segment of a template polynucleotide which to be amplified or replicated. A primer is capable of acting as a point of initiation of nucleic acid synthesis when placed under conditions in which synthesis of a primer extension product which is complementary to a nucleic acid strand is induced, e.g., in the presence of nucleotides and an agent for polymerization such as DNA polymerase, reverse transcriptase and the like, and at a suitable temperature and pH. The primer is preferably single stranded for maximum efficiency, but may alternatively be in double stranded form. If double stranded, the primer is first treated to separate it from its complementary strand before being used to prepare extension products. A primer must be sufficiently long to prime the synthesis of extension products in the presence of the agents for polymerization. The exact lengths of the primers will depend on many factors, including temperature and the source of primer.

The primers used herein are selected to be substantially complementary to the different strands of each specific sequence to be amplified, reverse transcribed, or sequenced, and preferably non-randomly hybridize with its respective template strand. Therefore, the primer sequence may or may not reflect the exact sequence of the template. Primers can be prepared using any suitable method, such as, for example, the phosphotriester or phosphodiester methods described in Narang et al., (1979) Meth Enzymol, 68:90; Brown et al., (1979) Meth Enzymol, 68:10; and U.S. Pat. Nos. 4,356,270; 4,458,066; 4,416,988; and 4,293,652. Exemplary reverse transcription primers include poly-A primers, random primers, sequence specific primers, and gene specific primers.

A primer for use in the methods described herein can be substantially complementary to an address polynucleotide primer binding sequence. An amplification primer for use in the methods described herein can be substantially complementary to a proximity polynucleotide primer binding sequence. For example, a primer pair can comprise a first primer substantially complementary to an address polynucleotide primer binding sequence and a second primer substantially complementary to a proximity polynucleotide primer binding sequence. Amplification of a polynucleotide comprising a proximity polynucleotide coupled to an address polynucleotide can be used to amplify the polynucleotide comprising a proximity polynucleotide coupled to an address polynucleotide such that amplified products produced contain the proximity barcode or compliment thereof. Amplification of a polynucleotide comprising a proximity polynucleotide coupled to an address polynucleotide can be used to amplify the polynucleotide comprising a proximity polynucleotide coupled to an address polynucleotide such that amplified products produced contain the address barcode or compliment thereof. Amplification of a polynucleotide comprising a proximity polynucleotide coupled to an address polynucleotide can be used to amplify the polynucleotide comprising a proximity polynucleotide coupled to an address polynucleotide such that amplified products produced contain both the proximity barcode or compliment thereof and the address barcode or compliment thereof.

A primer used in amplification can have any suitable sequence for amplification. In preferred embodiments, an amplification primer does not have a sequence complementary to a proximity barcode, such as an address barcode contained in a ligated product. In preferred embodiments, an amplification primer does not have a sequence complementary to an address barcode, such as an address barcode contained in a ligated product. In preferred embodiments, an amplification primer binds to a polynucleotide comprising a proximity polynucleotide coupled to an address polynucleotide at a region upstream of the address barcode. In preferred embodiments, an amplification primer binds to a polynucleotide comprising a proximity polynucleotide coupled to an address polynucleotide at a region upstream of the address barcode.

Amplification can be performed using a primer set comprising a first primer and a second primer. For example, amplification can be performed using a primer set comprising a forward primer and a reverse primer. For example, a forward primer can be complementary to a region of a ligated product that is upstream of a proximity barcode. For example, a reverse primer can be complementary to a region of a ligated product that is upstream of an address barcode. In preferred embodiments, an amplification primer set comprises a first primer (e.g., a forward primer) and a second primer (e.g., a reverse primer) that bind to a polynucleotide comprising a proximity polynucleotide coupled to an address polynucleotide, wherein the first primer binds to a region upstream of the address barcode, and wherein the second primer binds to a region upstream of the proximity barcode.

A primer can be a universal primer. A universal primer contains a unique amplification or sequencing priming region that is, for example, about 5, 7, 10, 13, 15, 17, 20, 22, or 25 nucleotides in length, and is present on each polynucleotide of a plurality of polynucleotides to be amplified. Thus, a universal primer can be used to amplify multiple polynucleotides simultaneously, in a single reaction, and/or with similar amplification efficiencies.

A primer can comprise a universal adaptor. For example, a primer can comprise a universal sequencing primer binding region such that amplified products contain the universal sequencing primer region.

### Detecting Coupled Products

The methods described herein can comprise detecting a product (or amplified product thereof) comprising an address polynucleotide coupled to a proximity polynucleotide. The detecting can comprise sequencing. Thus, provided herein are methods for sequencing ligation products and or the amplified ligation products as described above. For example, the detecting can comprise sequencing the proximity barcode and the address barcode of a product comprising an address polynucleotide coupled to a proximity polynucleotide. Thus, provided herein are methods for sequencing products (or amplified products thereof) comprising an address polynucleotide coupled to a proximity polynucleotide using one or more primers or primer pairs located upstream of the proximity barcode and address barcode. Thus, a sequence read can comprise an address barcode sequence and a proximity barcode sequence on the same sequence read. Any sequencing technique described herein or known to one skilled in the art can be used in the methods herein.

Sequencing methods include deep sequencing methods. The detecting can comprise deep sequencing (i.e., ultra-deep sequencing or next generation sequencing (NGS)) which is directed to an enhanced sequencing method enabling the rapid parallel sequencing of multiple nucleic acid sequences. (See, e.g., Bentley et al., Nature (2008), 456:53-59). Deep sequencing methods include sequencing nucleic acids to a depth that allows each base to be read hundreds of times, typically at least about 500, 1,000, or 1,500 times. In a typical deep sequencing protocol, nucleic acids (e.g. DNA fragments) are attached to the surface of a reaction platform (e.g., flow cell, microarray, and the like). In some embodiments, polynucleotides are amplified in situ and used as templates for synthetic sequencing (e.g., sequencing by synthesis) using a detectable label (e.g. fluorescent reversible terminator deoxyribonucleotide). Representative reversible terminator deoxyribonucleotides may include 3′-O-azidomethyl-2′-deoxynucleoside triphosphates of adenine, cytosine, guanine and thymine, each labeled with a different recognizable and removable fluorophore, optionally attached via a linker. Where fluorescent tags are employed, after each cycle of incorporation, the identity of the inserted based may be determined by excitation (e.g., laser-induced excitation) of the fluorophores and imaging of the resulting immobilized growing duplex nucleic acid. The fluorophore, and optionally linker, may be removed by methods known in the art, thereby regenerating a 3′ hydroxyl group ready for the next cycle of nucleotide addition.

Exemplary suitable deep sequencing methods include single molecule real time (SMRT™) sequencing (Pacific Biosciences), Ion Torrent sequencing, MiSeq sequencing, HiSeq sequencing, massively parallel signature sequencing (MPSS), sequencing by synthesis (SBS), SBS pyrosequencing (454 Life Sciences), SOLiD™ sequencing by ligation (Applied Biosystems), single-molecule synthesis (SMS) platforms (Helicos Biosciences), SOLEXA™ sequencing (Illumina), Nanopore sequencing, Chemical-Sensitive Field Effect Transistor (chemFET) array sequencing with an electron microscope, and two stage PCR techniques coupled with a pyrophosphate sequencing technique (Harris et al., (2008) Science 320:106-09; Margulies et al., (2005) Nature, 437, 376-80; Soni and Meller, (2007) Clin Chem 53; Moudrianakis and Beer, (1965) PNAS USA 53:564-71; and U.S. Pub. No. 20090026082).

A sequencing technique used in the methods of the provided invention generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1,000 reads per run, at least 5,000 reads per run, at least 10,000 reads per run, at least 50,000 reads per run, at least 100,000 reads per run, at least 500,000 reads per run, at least 1,000,000 reads per run, at least 2,000,000 reads per run, at least 3,000,000 reads per run, at least 4,000,000 reads per run at least 5,000,000 reads per runs at least 6,000,000 reads per run at least 7,000,000 reads per run at least 8,000,000 reads per runs at least 9,000,000 reads per run, or at least 10,000,000 reads per run.

### Methods of Use

The methods, kits, and compositions described herein can be used for numerous applications, including identification of binding partners, determination of affinities of binding moieties to target analytes, determination of specificities of binding moieties to target analytes, quantification of target analytes in a sample, quantification of binding events, identification of biomarkers of a disease or condition, drug discovery, molecular biology, immunology and toxicology. Arrays can be used for large scale binding assays in numerous diagnostic and screening applications. These methods of use include, but are not limited to, high-content, high-throughput assays for screening for binding moieties that interact with target analytes. Additional methods of use include medical diagnostic, proteomic, and biosensor assays. The multiplexed measurement of quantitative variation in levels of large numbers of target analytes allows the recognition of patterns defined by several to many different target analytes. The multiplexed identification of large numbers of interactions between target analytes and binding moieties allows for the recognition of binding and interaction patterns defined by several to many different interactions between target analytes and binding moieties.

The assays used with the arrays of the presently disclosed subject matter may be direct, noncompetitive assays or indirect, competitive assays. In the noncompetitive method, the affinity for a target analyte to a binding moiety can be determined directly. In this method, the target analyte can be directly exposed to a binding moiety. The binding moiety may be labeled or unlabeled. A label refers to a molecule that, when attached to another molecule provides or enhances a means of detecting the other molecule. A signal emitted from a label can allow detection of the molecule or complex to which it is attached, and/or the label itself. For example, a label can be a molecular species that elicits a physical or chemical response that can be observed or detected by the naked eye or by means of instrumentation such as, without limitation, scintillation counters, colorimeters, UV spectrophotometers and the like. Labels include but are not limited to, radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence detection, electrochemiluminescence detection, Raman energy detection, colorimetric detection, hybridization protection assays, and mass spectrometry.

If the binding moiety is labeled, the methods of detection could include fluorescence, luminescence, radioactivity, and the like. If the binding moiety is unlabeled, the detection of binding would be based on a change in some physical property of the target analyte. Such physical properties could include, for example, a refractive index or electrical impedance. The detection of binding of unlabeled binding moiety could include, for example, mass spectroscopy. In competitive methods, binding-site occupancy may be determined indirectly. In this method, the target analytes can be exposed to a solution containing a cognate labeled binding moiety and an unlabeled moiety. The labeled cognate binding moiety and the unlabeled moiety compete for the binding sites on the target analyte. The affinity of the unlabeled moiety for the target analyte relative to the labeled cognate binding moiety is determined by the decrease in the amount of binding of the labeled binding moiety. The detection of binding can also be carried out using sandwich assays, in which after the initial binding, the array is incubated with a second solution containing molecules such as labeled antibodies that have an affinity for the binding moiety bound to the target analyte, and the amount of binding is determined based on the amount of binding of the labeled antibodies to the binding moiety. The detection of binding can be carried out using a displacement assay in which after the initial binding of a labeled moiety, the array is incubated with a second solution containing unlabeled binding moiety. The binding capability and the amount of binding of the binding moiety are determined based on the decrease in number of the pre-bound labeled moieties in the target analytes.

The arrays of the presently disclosed subject matter may also be used in a method for screening for binding moieties, wherein a potential binding moiety candidate is screened directly for its ability to bind or otherwise interact with a plurality of target analytes on the array. Alternatively, a plurality of potential binding moieties may be screened in parallel for their ability to bind or otherwise interact with one or more types of target analytes on the array. The screening process may involve assaying for the interaction, such as binding, of at least one binding moiety of a sample with one or more target analytes on the array, both in the presence and absence of the potential binding moiety candidate. This allows for a potential binding moiety to be tested for its ability to act as an inhibitor of the interaction or interactions originally being assayed.

The arrays of the presently disclosed subject matter may also be used in a method for screening a plurality of target analytes for their ability to bind a particular binding moiety of a sample containing a plurality of binding moieties. For example, the sample can be contacted to an array comprising target analytes and the presence or amount of the particular binding moiety retained at each microspot can be detected, either directly or indirectly, or by sequencing. In some embodiments, the method further comprises characterizing the particular binding moiety retained on at least one microspot.

Also disclosed herein are methods for determining a quantity, amount, or concentration of a target analyte in a sample, wherein the determining comprises determining a number of sequence reads having a proximity barcode sequence corresponding to the binding moiety and an address barcode sequence corresponding to the target analyte, wherein the number of the sequence reads is proportional to the quantity, amount, or concentration of the target analyte in the sample. For example, the determining can comprise determining a number of sequence reads having a proximity barcode sequence corresponding to the binding moiety and an address barcode sequence corresponding to the target analyte and comparing the number of reads to a standard curve, such as a standard curve generated using a same method using a particular binding moiety known to interact with a particular target analyte, wherein the particular target analyte is present at one or more known concentrations.

Also disclosed herein are methods for determining a relative binding affinity of a binding moiety for a target analyte, wherein the determining comprises determining a number of sequence reads having a proximity barcode sequence corresponding to the binding moiety and an address barcode sequence corresponding to the target analyte.

Also disclosed herein is a method of determining a relative binding affinity of a binding moiety for a target analyte, wherein the determining comprises determining a number of sequence reads having a proximity barcode sequence corresponding to the binding moiety and an address barcode sequence corresponding to the target analyte, wherein the number of sequence reads is proportional to the relative binding affinity.

Also provided herein is a method of determining a relative binding affinity of a binding moiety for a target analyte, the method comprising amplifying coupled proximity polynucleotide and address polynucleotide products; measuring an amount of sequence reads having the proximity barcode sequence and the address barcode sequence from the amplified product; and determining a relative binding affinity of the binding moiety for the target analyte by using the measured amount.

The relative binding affinity of a binding moiety for a target analyte may be measured by measuring or counting the coupled product and/or amplified products thereof by using any suitable method known in the art. For example, the determining may be performed by standardizing an amount of sequence reads having the proximity barcode sequence and the address barcode sequence with respect to a predetermined value, for example, a threshold value, or comparing the amount of sequence reads having the proximity barcode sequence and the address barcode sequence with a standard value. For example, the determining may be performed by standardizing an amount of sequence reads having the proximity barcode sequence and the address barcode sequence with respect to a control, for example, an amount of sequence reads generated from a control reaction. The determining of a relative binding affinity of the binding moiety's binding to the target analyte may be used to determine whether an association between the target nucleic acid and various physiological conditions or diseases exists.

A method herein can include further determining a relative binding specificity of the binding moiety for the target analyte, wherein the determining can include determining the number of sequence reads having the same address barcode but different proximity barcodes. The number or quantity of sequence reads having a different target analyte barcode can be inversely proportional to the binding specificity. The relative binding specificity of the binding moiety for the target analyte may be measured according to coupled product and/or amplified products thereof using any method known in the art. The determining may be performed by standardizing the amount of sequence reads having a same address barcode but different proximity barcodes and/or a same address barcode and a same proximity barcode, with respect to a predetermined value, for example, a threshold value, or comparing the amount of sequence reads a same address barcode but different proximity barcodes and/or a same address barcode and a same proximity barcode with a standard value.

In addition to detecting a wide variety of analytes, the subject methods may also be used to screen for agents that modulate the interaction between a binding moiety of a proximity probe with a target analyte to which it binds. The term modulating includes both decreasing (e.g., inhibiting) and enhancing the interaction between the two molecules. The screening method may be an in vitro or in vivo format, where both formats are readily developed by those of skill in the art.

A variety of different agents may be screened by the above methods. Candidate agents encompass numerous chemical classes including, but not limited to, peptides, polynucleotides, and organic molecules (e.g., small organic compounds having a molecular weight of more than 50 and less than about 2,500 Daltons). Candidate agents can comprise functional groups for structural interaction with target analytes, such as hydrogen bonding, and can include at least one or at least two of an amine, carbonyl, hydroxyl or carboxyl group. The candidate agents can comprise cyclical carbon or heterocyclic structures and/or aromatic or polyaromatic structures substituted with one or more functional groups. Candidate agents can be biomolecules including peptides, saccharides, fatty acids, steroids, purines, pyrimidines, derivatives, structural analogs or combinations thereof. Candidate agents can be obtained from a wide variety of sources including libraries of synthetic or natural compounds. For example, numerous means are available for random and directed synthesis of a wide variety of organic compounds and biomolecules, including expression of randomized polynucleotides and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs. Agents identified find uses in a variety of methods, including methods of modulating the activity of a target analyte, and conditions related to the presence, activity, and/or interactions of a target analyte

Also disclosed herein are methods for determining or screening binding moieties, wherein a selected binding moiety is identified as monospecific. In some instances, at most about 0.01% of the screened binding moieties can be monospecific. For example, at most about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of the screened binding moieties can be identified as monospecific. Also disclosed herein are methods for determining affinities of a plurality of binding moieties for target analytes, wherein at least one of the binding moiety can have a binding affinity of at least 10⁻⁷M (K_{D}), such as at least 10⁻⁸M, 10⁻⁹M, 10⁻¹⁰M, 10⁻¹¹M, 10⁻¹²M, 10⁻¹³M, 10⁻¹⁴M, 10⁻¹⁵M, or 10⁻¹⁶M, for a target analyte.

Specific binding of a binding moiety to a target analyte can be validated or determined by various established methods known in the art and include ELISA, FACS, Western Blot, ImmunoBlot, MSD, BIAcore and SET; and these values can be compared to the corresponding binding affinities determined using the methods described herein. A binding moiety can be deemed to be a binding partner for a target analyte if the binding moiety is demonstrated to be able to bind to a specific target analyte at least 2-fold, 3-fold, 4-fold, 5-fold, 6-fold, 7-fold, 8-fold, 9-fold, 10-fold, 20-fold, 50-fold, 100-fold, 500-fold, or 1,000-fold over background or a negative control reaction. For example, a binding moiety can be deemed to be a binding partner for a target analyte if the number of sequence reads containing an address barcode sequence corresponding to that target analyte and a proximity barcode corresponding to the binding moiety is at least about 2-fold, 3-fold, 4-fold, 5-fold, 6-fold, 7-fold, 8-fold, 9-fold, 10-fold, 20-fold, 50-fold, 100-fold, 500-fold, or 1,000-fold higher than the number of sequence reads containing an address barcode sequence that does not correspond to that target analyte and a proximity barcode corresponding to the binding moiety.

A binding moiety can be deemed monospecific for a target analyte if the binding moiety is demonstrated to be able to bind to a specific target analyte at least 2-fold, 3-fold, 4-fold, 5-fold, 6-fold, 7-fold, 8-fold, 9-fold, 10-fold, 20-fold, 50-fold, 100-fold, 500-fold, or 1,000-fold more than the binding moiety binds to any other target analyte of a plurality of target analytes. For example, a binding moiety can be deemed monospecific for a target analyte if the binding moiety is demonstrated to be able to bind to a specific target analyte at least 2-fold, 3-fold, 4-fold, 5-fold, 6-fold, 7-fold, 8-fold, 9-fold, 10-fold, 20-fold, 50-fold, 100-fold, 500-fold, or 1,000-fold more than the binding moiety binds to any other target analyte of a plurality of at least about 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,00 target analytes. For example, a binding moiety can be deemed monospecific for a target analyte if the number of sequence reads containing an address barcode sequence corresponding to that target analyte and a proximity barcode corresponding to the binding moiety is at least about 2-fold, 3-fold, 4-fold, 5-fold, 6-fold, 7-fold, 8-fold, 9-fold, 10-fold, 20-fold, 50-fold, 100-fold, 500-fold, or 1,000-fold higher the number of sequence reads containing an address barcode sequence corresponding to any other target analyte of a plurality of target analytes and a proximity barcode corresponding to the binding moiety. For example, a binding moiety can be deemed monospecific for a target analyte if the number of sequence reads containing an address barcode sequence corresponding to that target analyte and a proximity barcode corresponding to the binding moiety is at least about 2-fold, 3-fold, 4-fold, 5-fold, 6-fold, 7-fold, 8-fold, 9-fold, 10-fold, 20-fold, 50-fold, 100-fold, 500-fold, or 1,000-fold higher the number of sequence reads containing an address barcode sequence corresponding to any other target analyte of a plurality of at least about 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,00 target analytes and a proximity barcode corresponding to the binding moiety.

### Diagnostics

The methods and apparatus disclosed herein can be used to screen for various diseases or conditions, including an alteration in the state of the body or of some of the organs, interrupting or disturbing the performance of the functions and/or causing symptoms such as discomfort, dysfunction, distress, or even death to the person afflicted or those in contact with a person. A disease or condition can also include a distemper, ailing, ailment, malady, disorder, sickness, illness, complain, interdisposition and/or affectation.

For example, samples containing binding moieties from a diseased animal can be simultaneously screened for the binding moieties' ability to interact with target analytes on an array. These interactions can be compared to those of samples from individuals that are not in a disease state, not presenting symptoms of persons in the disease state, or presenting symptoms of the disease state. For example, the levels of target analytes in samples from a diseased animal can be simultaneously determined. These levels can be compared to those of samples from individuals that are not in a disease state, not presenting symptoms of persons in the disease state, or presenting symptoms of the disease state.

The methods, kits, and compositions described herein can be used in medical diagnostics, drug discovery, molecular biology, immunology and toxicology. Arrays can be used for large scale binding assays in numerous diagnostic and screening applications. The multiplexed measurement of quantitative variation in levels of large numbers of target analytes (e.g. proteins) allows the recognition of patterns defined by several to many different target analytes. The multiplexed identification of large numbers of interactions between target analytes and binding moieties allows for the recognition of binding and interaction patterns defined by several to many different interactions between target analytes and binding moieties. Many physiological parameters and disease-specific patterns can be simultaneously assessed. One embodiment involves the separation, identification and characterization of proteins present in a biological sample. For example, by comparison of disease and control samples, it is possible to identify disease specific target analytes. These target analytes can be used as targets for drug development or as molecular markers of disease.

For many diagnostic and investigative purposes, it can be useful to determine the level of a target analyte. For many diagnostic and investigative purposes, it can be useful to determine the binding specificity and strength of the binding moiety. This application can be important for the discovery and diagnosis of clinically useful markers that correlate with a particular diagnosis or prognosis. For example, by monitoring a range of antibody or T-cell receptor specificities in parallel, one may determine the levels and kinetics of antibodies during the course of autoimmune disease, during infection, through graft rejection, etc. Alternatively, novel markers and interactions between markers associated with a disease of interest can be developed by comparing normal and diseased samples, or by comparing clinical samples at different stages of a disease.

Detection a level of one or more target analyte or detection of interactions between binding moieties and target analytes can lead to a medical diagnosis. For example, the identity of a pathogenic microorganism can be established unambiguously by binding a sample of the unknown pathogen to an array containing many types of antibodies specific for known pathogenic antigens.

The sample can be a sample from a subject with a condition or disease. For example, a sample can be a diseased tissue or cell, such as a breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell. The sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. The disease or condition can also be a perinatal or pregnancy related condition (e.g. preeclampsia or preterm birth), or a metabolic disease or condition, such as a metabolic disease or condition associated with iron metabolism.

### Supports/Substrates

The present disclosure provides substrates and methods of making substrates. The nature and geometry of a support or substrate can depend upon a variety of factors, including the type of array (e.g., one-dimensional, two-dimensional or three-dimensional) and the mode of coupling the address polynucleotide, target analyte, or other moiety (e.g., covalently or non-covalently). Generally, a substrate can be composed of any material which will permit coupling of an address polynucleotide and/or a target analyte, which will not melt or otherwise substantially degrade under the conditions used to hybridize and/or denature nucleic acids. A substrate can be composed of any material which will permit coupling of an address polynucleotide, a target analyte, and/or other moiety at one or more discrete regions and/or discrete locations within the discrete regions. A substrate can be composed of any material which permit washing or physical or chemical manipulation without dislodging an address polynucleotide or target moiety from the solid support.

Substrates can be fabricated by the transfer of target analyte and or address polynucleotide onto the solid surface in an organized high-density format followed by coupling the target analyte and/or address polynucleotide thereto. The techniques for fabrication of a substrate of the invention include, but are not limited to, photolithography, ink jet and contact printing, liquid dispensing and piezoelectrics. The patterns and dimensions of arrays are to be determined by each specific application. The sizes of each target analyte spots may be easily controlled by the users.

A method of making a solid substrate can comprise contacting or coupling an address polynucleotide to a first discrete location of a discrete region on a solid support, and contacting or coupling a target analyte to a second discrete location of the discrete region on the solid support, wherein the target analyte is in proximity to the address polynucleotide. The coupling can include any of the coupling methods described herein or otherwise known in the art.

A method of making an array can comprise contacting or coupling a first address polynucleotide to a first discrete location of a first discrete region on a solid support, and contacting or coupling a first target analyte to a second discrete location of the first discrete region on the solid support, wherein the first target analyte is in proximity to the first address polynucleotide; and contacting or coupling a second address polynucleotide to a first discrete location of a second discrete region on the solid support, and contacting or coupling a second target analyte to a second discrete location of the second discrete region on the solid support, wherein the second target analyte is in proximity to the second address polynucleotide. In preferred embodiments, the first address polynucleotide is not in proximity to the second target analyte. In preferred embodiments, the second address polynucleotide is not in proximity to the first target analyte. In preferred embodiments, the first discrete region is not in proximity to the second discrete region.

A substrate may take a variety of configurations ranging from simple to complex, depending on the intended use of the array. Thus, a substrate can have an overall slide or plate configuration, such as a rectangular or disc configuration. A standard microplate configuration can be used. In some embodiments, the surface may be smooth or substantially planar, or have irregularities, such as depressions or elevations. For example, the substrates of the presently disclosed subject matter can include at least one surface on which a pattern of recombinant virion microspots can be coupled or deposited. In some instances, a substrate may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

A support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A solid support as described above can be formed of any suitable material, including metals, metal oxides, semiconductors, polymers (particularly organic polymers in any suitable form including woven, nonwoven, molded, extruded, cast, etc.), silicon, silicon oxide, and composites thereof.

A number of materials (e.g., polymers) suitable for use as substrates (e.g., solid substrates) in the instant invention have been described in the art. Suitable materials for use as substrates include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, silicon oxide or other inorganic or semiconductor material (e.g., silicon nitride), compound semiconductors (e.g., gallium arsenide, and indium gallium arsenide), and combinations thereof.

Examples of well-known solid supports include polypropylene, polystyrene, polyethylene, dextran, nylon, amylases, glass, natural and modified celluloses (e.g., nitrocellulose), polyacrylamides, agaroses and magnetite. In some instances, the solid support can be silica or glass because of its great chemical resistance against solvents, its mechanical stability, its low intrinsic fluorescence properties, and its flexibility of being readily functionalized. In one embodiment, the substrate is glass, particularly glass coated with nitrocellulose, more particularly a nitrocellulose-coated slide (e.g., FAST slides).

A substrate may be modified with one or more different layers of compounds or coatings that serve to modify the properties of the surface in a desirable manner. For example, a substrate may further comprise a coating material on the whole or a portion of the surface of the substrate. In some embodiments, a coating material enhances the affinity of the target analyte, and address polynucleotide, or another moiety (e.g., a functional group) for the substrate. For example, the coating material can be nitrocellulose, silane, thiol, disulfide, or a polymer. When the material is a thiol, the substrate may comprise a gold-coated surface and/or the thiol comprises hydrophobic and hydrophilic moieties. When the coating material is a silane, the substrate comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of a thioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the substrate. The attachment of the monolayer to the substrate may also be achieved by non-covalent interactions or by covalent reactions.

After attachment to the substrate, the coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Exemplary activated functional groups of the coating on the substrate for covalent coupling to terminal amino groups include anhydrides, N-hydroxysuccinimide esters or other common activated esters or acid halides, Exemplary activated functional groups of the coating on the substrate include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

A substrate can contain a linker (e.g., to indirectly couple a moiety to the substrate). In one embodiment, a linker has one terminal functional group, a spacer region and a target analyte adhering region. The terminal functional groups for reacting with functional groups on an activated coating include halogen, amino, hydroxyl, or thiol groups. In some instances, a terminal functional group is selected from the group consisting of a carboxylic acid, halogen, amine, thiol, alkene, acrylate, anhydride, ester, acid halide, isocyanate, hydrazine, maleimide and hydroxyl group. The spacer region may include, but is not limited to, polyethers, polypeptides, polyamides, polyamines, polyesters, polysaccharides, polyols, multiple charged species or any other combinations thereof. Exemplary spacer regions include polymers of ethylene glycols, peptides, glycerol, ethanolamine, serine, inositol, etc. The spacer region may be hydrophilic in nature. The spacer region may be hydrophobic in nature. In some instances, the spacer has n oxyethylene groups, where n is between 2 and 25. In some instances, a region of a linker that adheres to an address polynucleotide, target analyte, or other moiety is hydrophobic or amphiphilic with straight or branched chain alkyl, alkynyl, alkenyl, aryl, arylalkyl, heteroalkyl, heteroalkynyl, heteroalkenyl, heteroaryl, or heteroarylalkyl. In some instances, a region of a linker that adheres to an address polynucleotide, target analyte, or other moiety comprises a C₁₀-C₂₅ straight or branched chain alkyl or heteroalkyl hydrophobic tail. In some instances, a linker comprises a terminal functional group on one end, a spacer, a target analyte adhering region, and a hydrophilic group on another end. The hydrophilic group at one end of the linker may be a single group or a straight or branched chain of multiple hydrophilic groups (e.g., a single hydroxyl group or a chain of multiple ethylene glycol units).

A support or substrate can be an array. In some embodiment a solid support comprises an array. An array of the invention can comprise an ordered spatial arrangement of two or more discrete regions. Address, spot, microspot, and discrete region are terms used interchangeably and refer to a particular position, such as on an array. An array can comprise target analytes located at known or unknown discrete regions. An array can comprise address polynucleotides located at known or unknown discrete regions.

Each of two or more discrete regions can comprise an address polynucleotide. Each of two or more discrete regions can comprise a target analyte. Each of two or more discrete regions can comprise an address polynucleotide and a target analyte. The two or more discrete regions of an array can comprise two or more first discrete locations and two or more second discrete locations. Each first discrete location can comprise a coupled address polynucleotide. Each second discrete location can comprise a target analyte. An address polynucleotide in a discrete region can be in proximity to the target analyte within the same discrete region. An address polynucleotide in a discrete region can be barcoded to the target analyte within the same discrete region. An address polynucleotide can be used to identify the target polynucleotide in the same region.

For example, an array can comprise a first discrete region comprising a first address polynucleotide and a first target analyte, and a second discrete region comprising a second address polynucleotide and a second target analyte. For example, an array can comprise a first discrete region comprising a first address polynucleotide at a first discrete location within the first discrete region and a first target analyte at a second discrete location within the first discrete region, and a second discrete region comprising a second address polynucleotide at a first discrete location within the second discrete region and a second target analyte at a second discrete location within the second discrete region.

Row and column arrangements of arrays can be selected due to the relative simplicity in making such arrangements. The spatial arrangement can, however, be essentially any form selected by the user, and optionally, in a pattern. Microspots of an array may be any convenient shape, including circular, ellipsoid, oval, annular, or some other analogously curved shape, where the shape may, in certain embodiments, be a result of the particular method employed to produce the array. The microspots may be arranged in any convenient pattern across or over the surface of the array, such as in rows and columns so as to form a grid, in a circular pattern, and the like, where generally the pattern of spots will be present in the form of a grid across the surface of the substrate.

An array can comprise an ordered spatial arrangement of two or more target analytes, two or more address polynucleotides, or a combination thereof, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 target analytes. An array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for a target analyte. The target analytes can be linked to the array by the antibodies. Thus, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 target analytes linked to the array by at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for the target analytes.

For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 address polynucleotides. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 target analytes and at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 address polynucleotides.

An array can comprise an ordered spatial arrangement of two or more same or different target analytes, two or more same or different address polynucleotides, or a combination thereof, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different target analytes. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different address polynucleotides. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different target analytes and at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different address polynucleotides.

An array can be a high-density array. A high-density array can comprise tens, hundreds, thousands, tens-of-thousands or hundreds-of-thousands of target analytes and/or address polynucleotides. The density of microspots of an array may be at least about 1/cm² or at least about 10/cm², up to about 1,000/cm² or up to about 500/cm². In certain embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct antibodies per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm². In some embodiments, target analytes, address polynucleotides, linkers, or another moiety in each discrete region are present in a defined amount (e.g., between about 0.1 femtomoles and 100 nanomoles). For example, an array can comprise at least about 2 target analytes and/or address polynucleotides per cm². For example, an array can comprise at least about 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more target analytes and/or address polynucleotides. For example, an array can be a high-density protein array comprising at least about 10 target analytes and/or address polynucleotides per cm². For example, an array can comprise at least about 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more target analytes and/or address polynucleotides per cm².

### Kits

Also provided are kits that find use in practicing the subject methods, as mentioned above. A kit can include one or more of the compositions described herein. In some embodiments, a kit includes at least one proximity probe. A kit can include at least one proximity polynucleotide. A kit can include at least one address polynucleotide. A kit can include at least one target analyte. A kit can include at least one binding moiety. A kit can include at least one splint polynucleotide. A kit can include at least one proximity polynucleotide, at least one target analyte, at least one binding moiety, at least one address polynucleotide, at least one splint polynucleotide, or any combination thereof. The binding moiety may already be coupled to the proximity polynucleotide and a proximity probe is provided in the kit. The binding moiety may not already coupled to the proximity polynucleotide in the kit. A kit can include a reagent for coupling at least one proximity polynucleotide and at least one binding moiety.

A kit can include a solid support. In some embodiments, the solid support is already functionalized with at least one address polynucleotide and/or at least one target analyte. In some embodiments, the solid support is not functionalized with at least one address polynucleotide and/or at least one target analyte. A kit can include a reagent for coupling at least one address polynucleotide to the solid support. A kit can include a reagent for coupling at least one target analyte to the solid support.

A kit can include one or more reagents for performing amplification, including suitable primers, enzymes, nucleobases, and other reagents such as PCR amplification reagents (e.g., nucleotides, buffers, cations, etc.), and the like. Additional reagents that are required or desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc.

As indicated above, certain protocols will employ two or more different sets of such probes for simultaneous detection of two or more target analytes in a sample (e.g., in multiplex and/or high throughput formats). In some embodiments a kit includes two or more distinct sets of proximity probes, proximity polynucleotides, binding moieties, address polynucleotides, and/or target analytes.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

### OTHER EMBODIMENTS

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims. While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Some inventive embodiments herein contemplate numerical ranges. When ranges are present, the ranges include the range endpoints. Additionally, every sub range and value within the rage is present as if explicitly written out. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed.

### EXAMPLES

### Example 1

### Coupling of Proximity Polynucleotide to a Proximity Probe

A proximity polynucleotide is covalently crosslinked to a binding moiety, (e.g., a mAb) using a commercial kit (Solulink, Inc.). First, a 3′-amino-oligo (proximity polynucleotide) is derivatized with Sulfo-S-4FB. Second, mAb proteins are modified with S-HyNic groups. Third, the HyNic-modified mAb is reacted with the 4FB-modified proximity polynucleotide to yield a bis-arylhydrazone mediated conjugate. Excess 4FB-modified proximity polynucleotide can be further removed via magnetic affinity matrix. The overall yield of the antibody-proximity polynucleotide conjugate is 30-50% based on the mAb recovery and is >95% free from HyNic-modified mAb and 4FB-modified polynucleotides. The bis-arylhydrazone bond is stable to heat (e.g., 94° C.) and pH (e.g., 3-10).

### Example 2

### Annotating an Address of a Target Analyte

To annotate the addresses of each protein in a plurality of proteins printed on an array, such as the HuProt array, address polynucleotides are designed and individually synthesized with primary amine groups attached to their 5′-ends and arrayed in 384-well titer dishes. An aliquot of the arrayed address polynucleotide is then added to the protein source plates also arrayed in the 384-well format. These mixtures of address polynucleotide are spotted together with the target analyte (purified human proteins) onto a derivatized glass slide that can form covalent bonds with the primary amine groups presented in both the target analyte and address polynucleotide. Thus, each printed target analyte is co-immobilized with an address polynucleotide comprising a unique barcode to form a barcoded array.

### Example 3

### Binding Assays

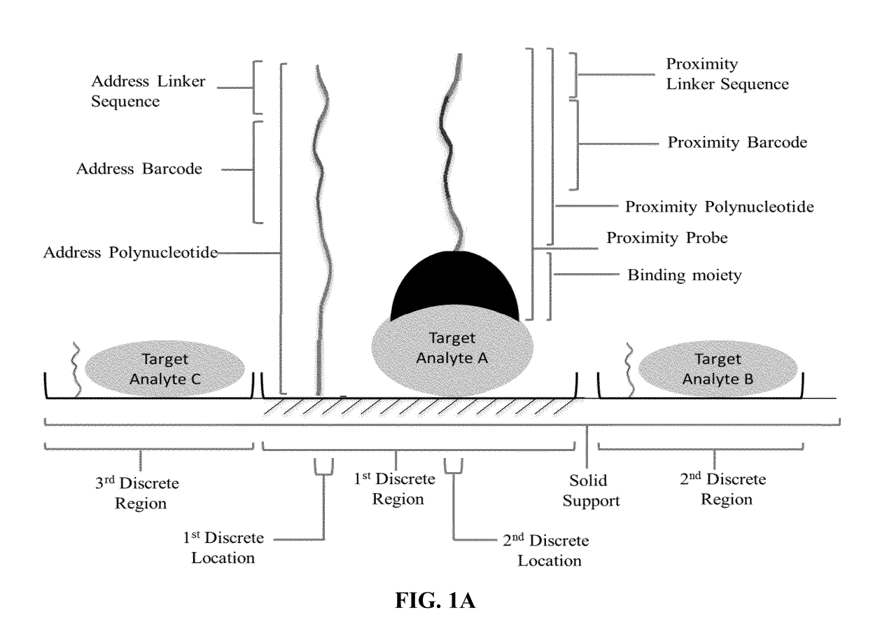

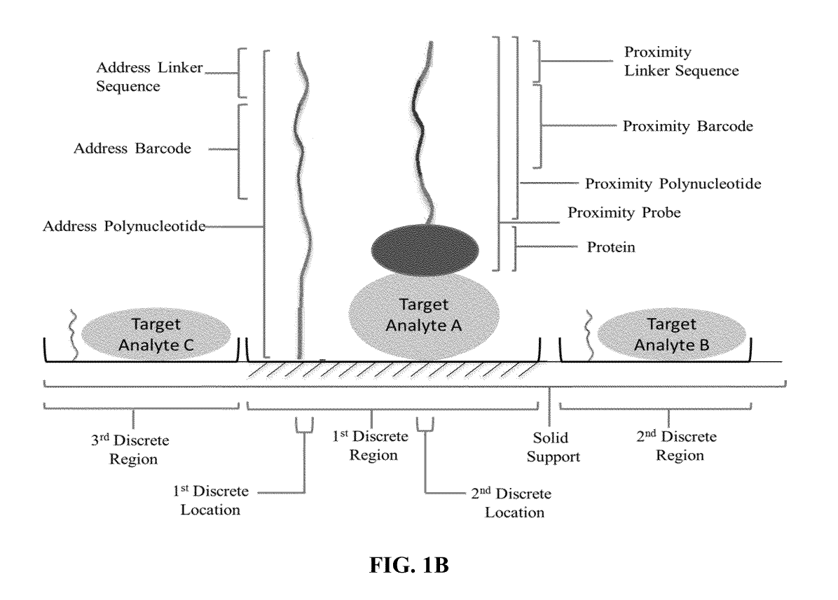

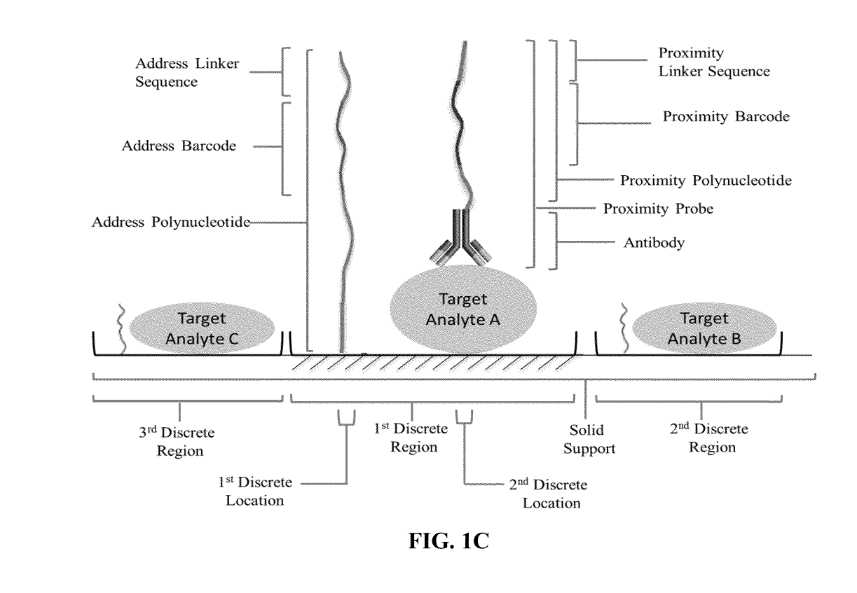

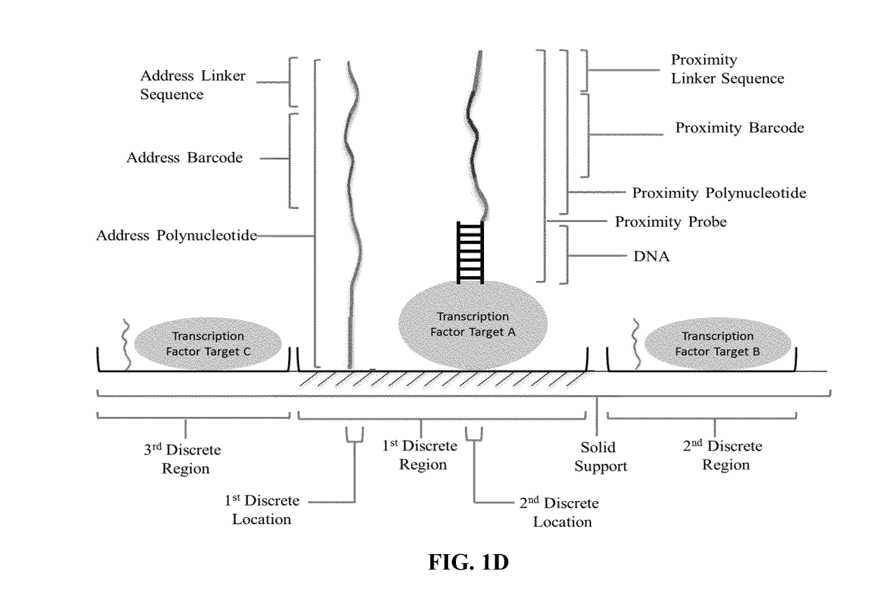

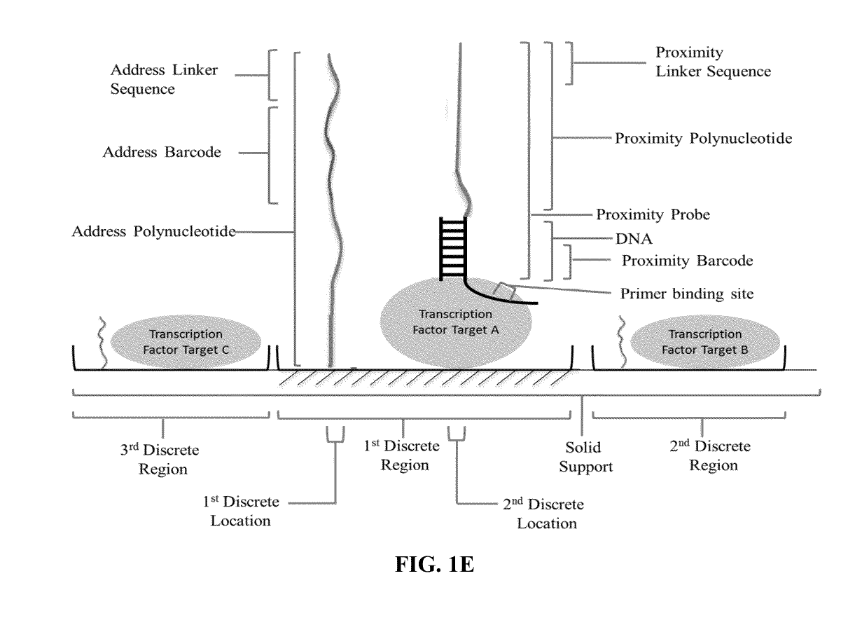

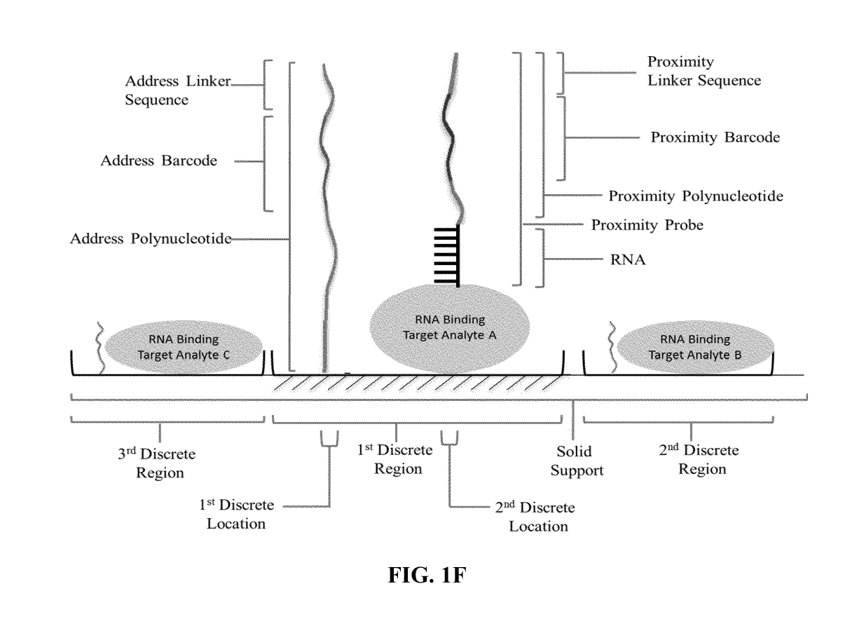

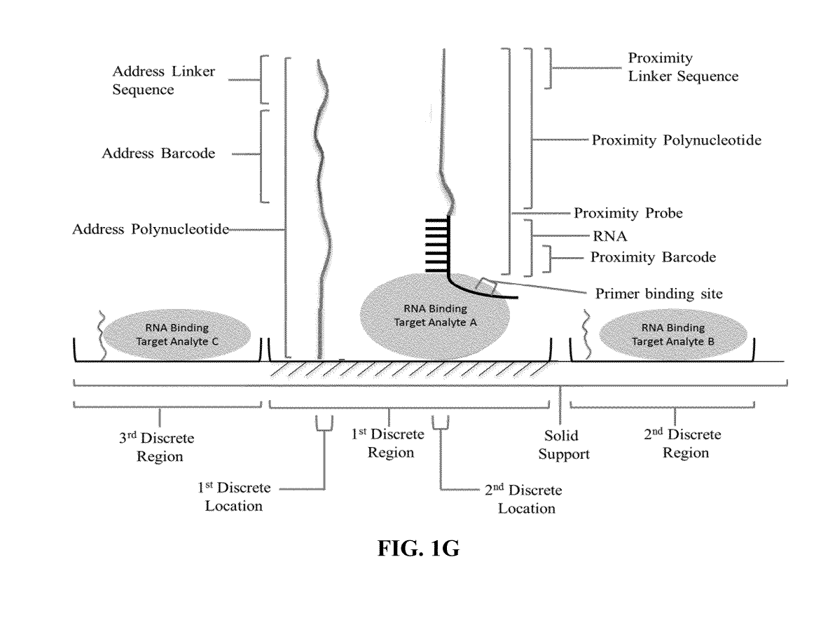

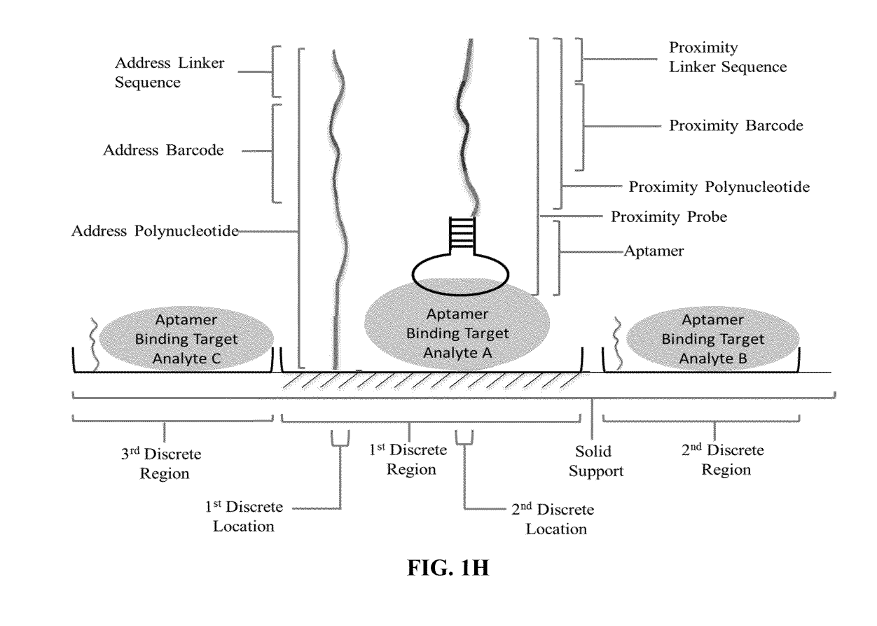

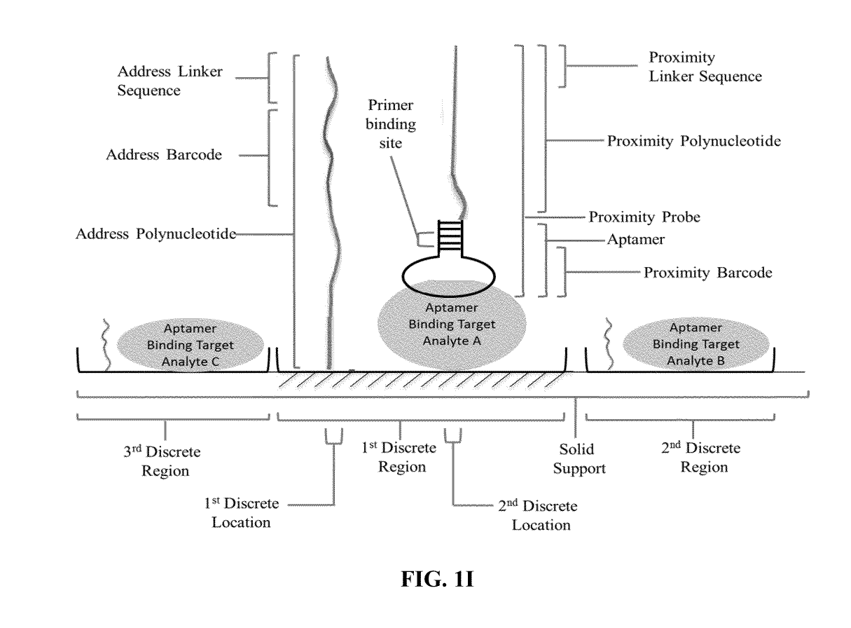

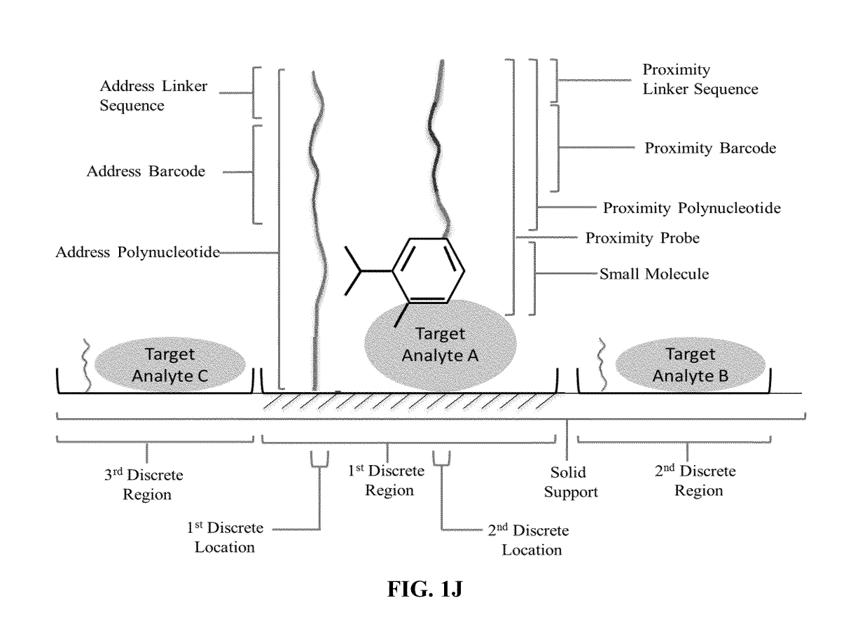

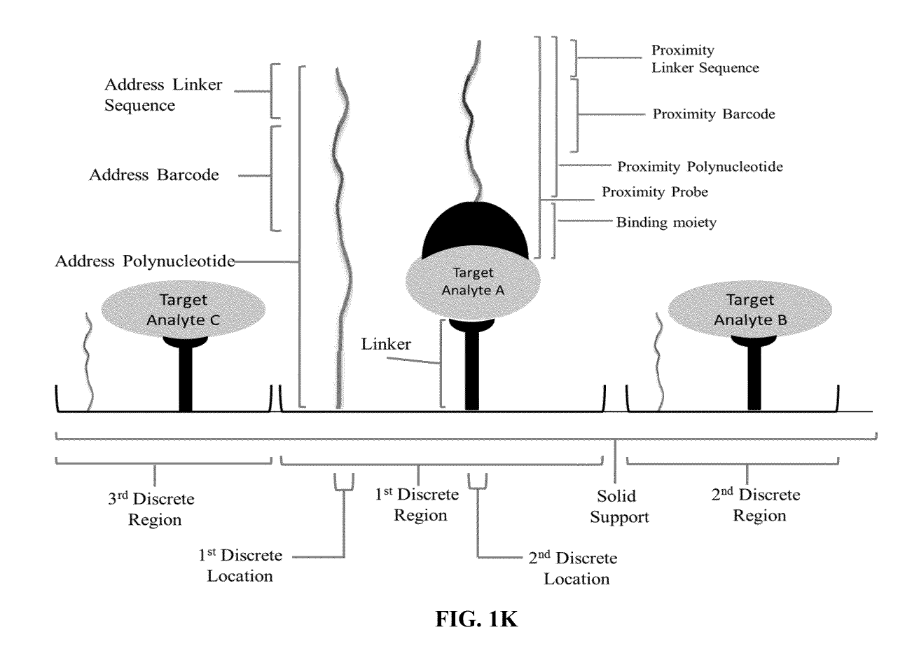

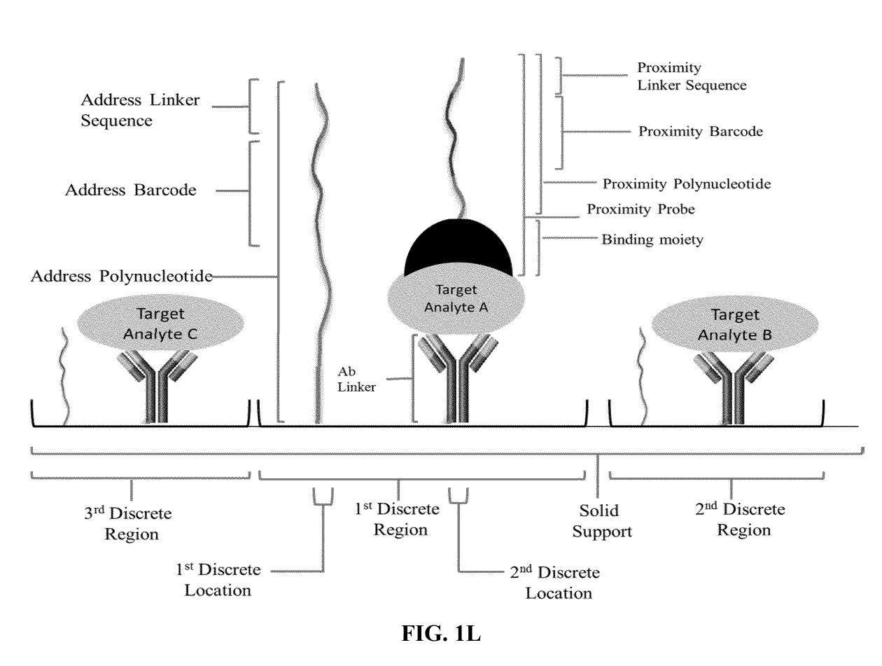

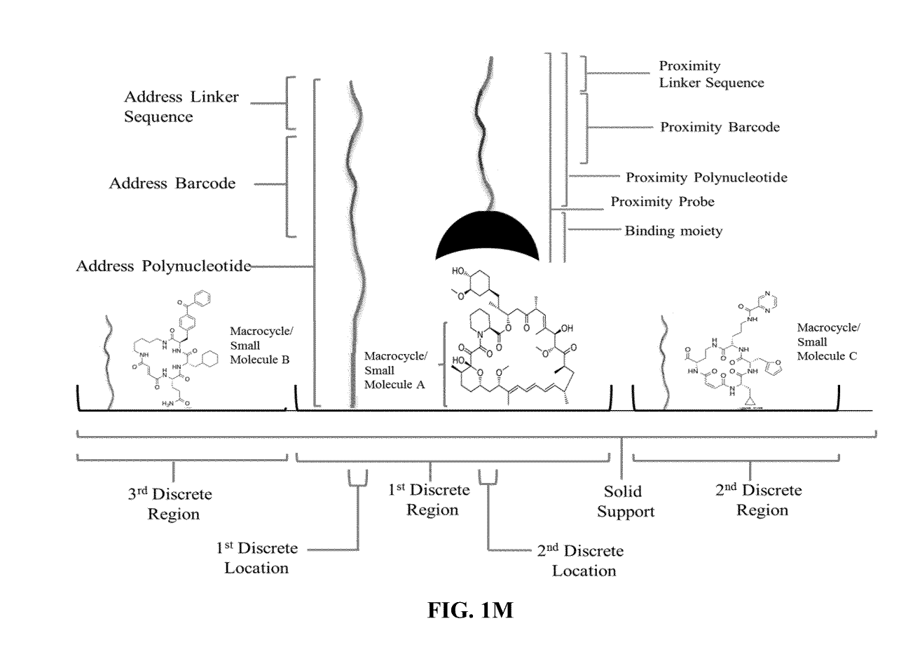

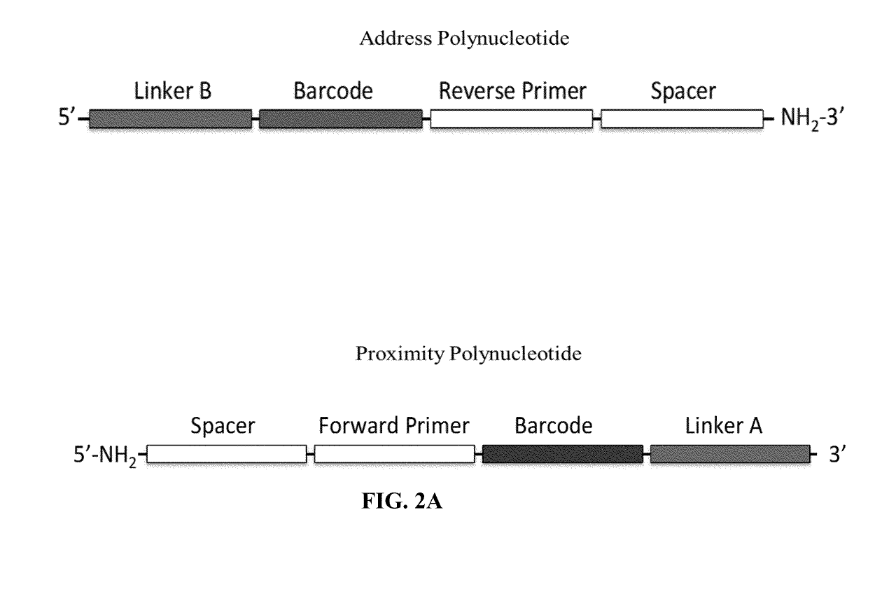

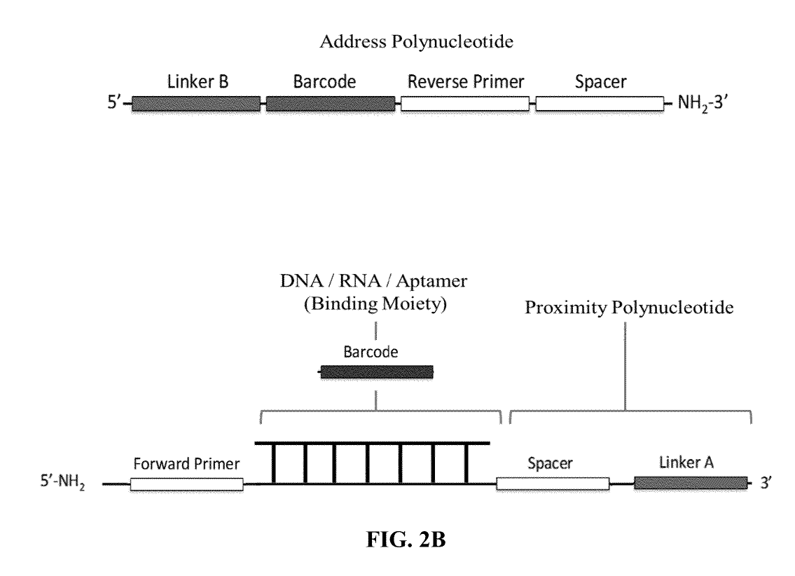

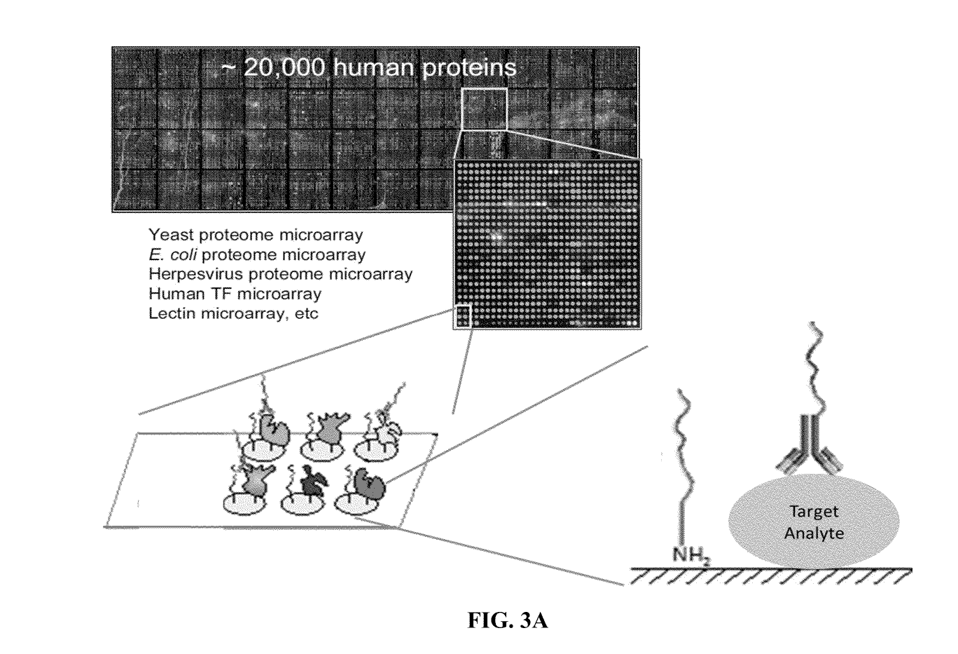

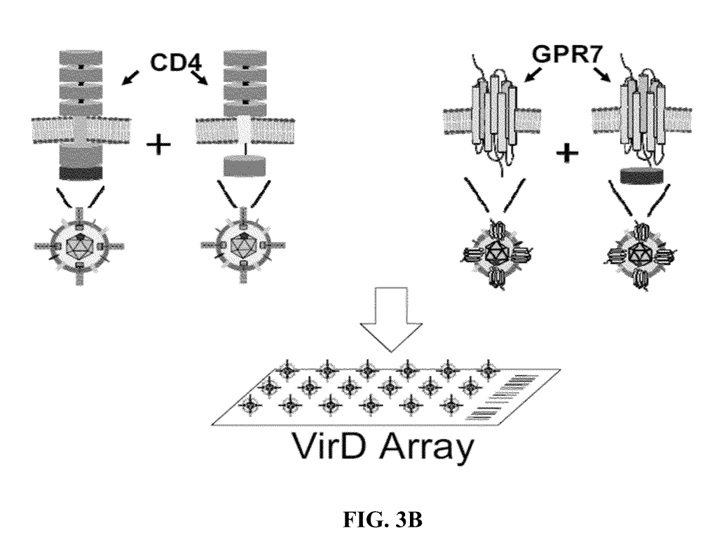

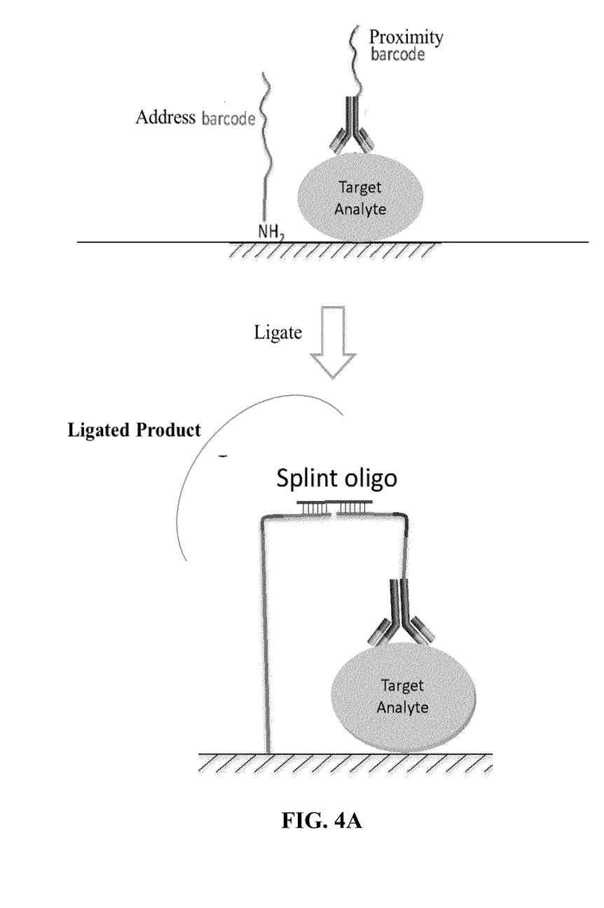

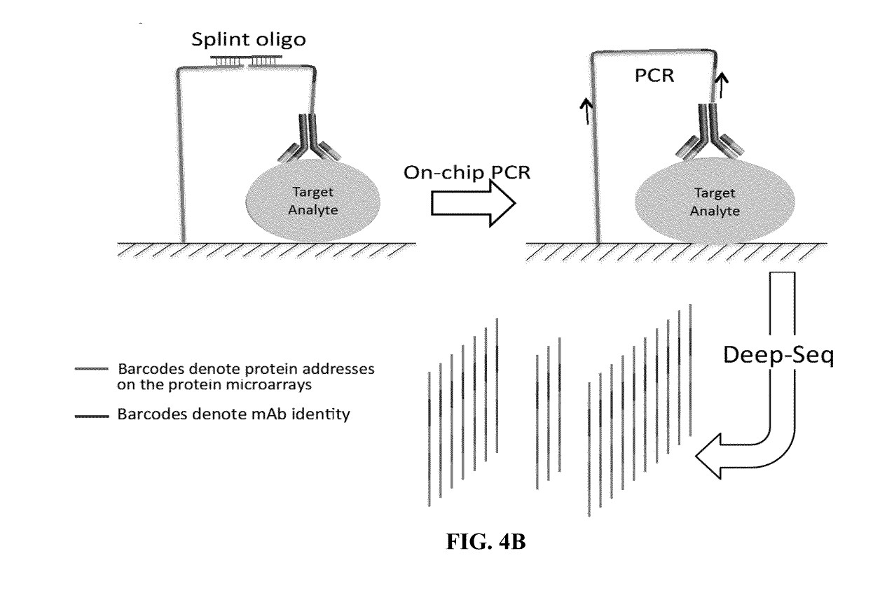

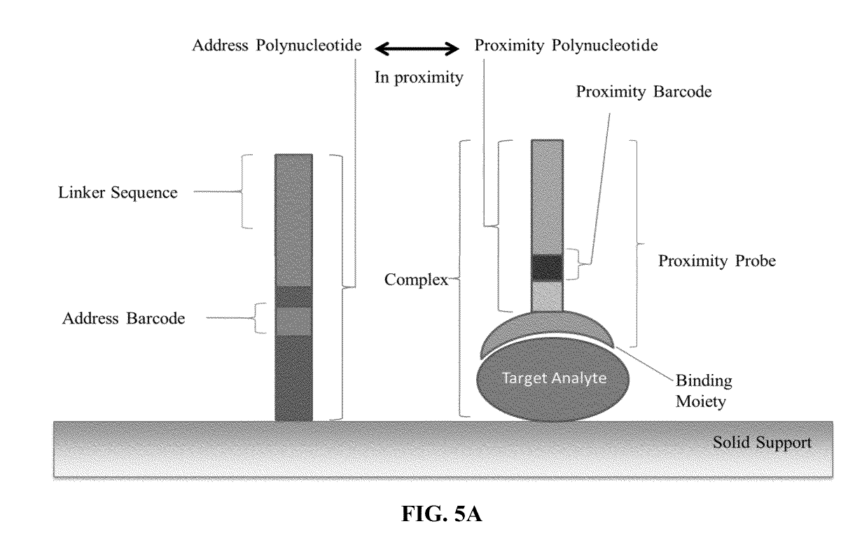

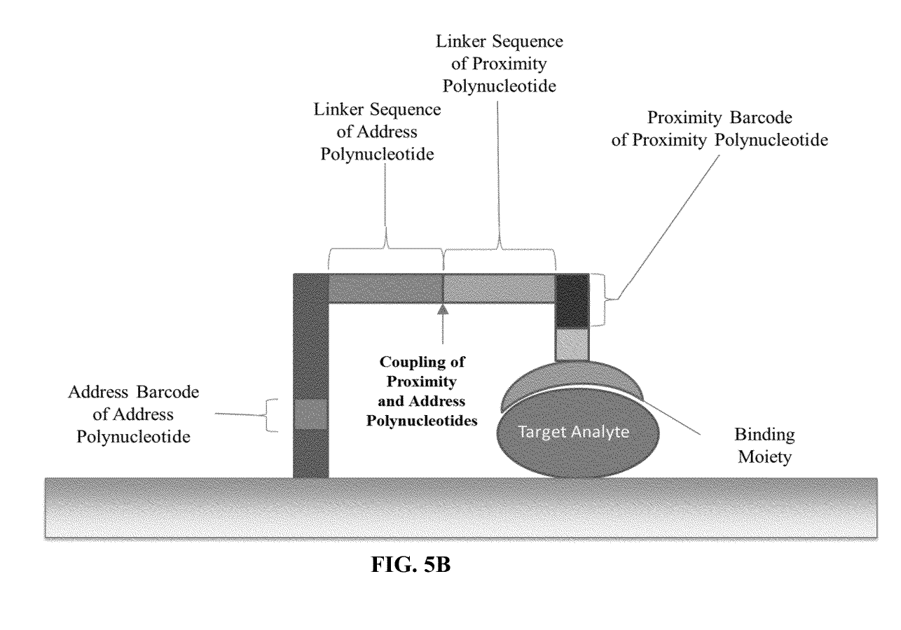

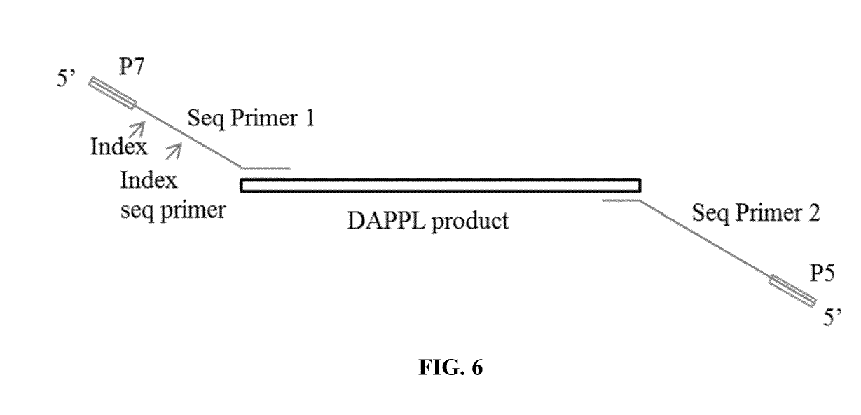

To perform multiplexed mAb-antigen binding assays on the barcoded arrays, hundreds to thousands of mAb-proximity polynucleotide conjugates are mixed and added to an address polynucleotide-barcoded array. After 2 hr. incubation at room temperature (RT), non-specific interactions are washed off with three 15-min washes in Tris-buffered saline Tween (TBST) (FIG. 1B). Next, a connector polynucleotide (splint polynucleotide) is added to the array that can hybridize to both Linkers A and B. When a mAb is bound on a protein spot, the splint polynucleotide brings together the proximity polynucleotide conjugated to the mAb and the address polynucleotide attached to the glass surface, leaving a nick between the two polynucleotides. DNA ligase is then added to covalently ligate the address polynucleotide and the proximity polynucleotide. The ligation products are then PCR-amplified using universal primer pairs, followed by deep-sequencing to determine the sequences of the ligated products.

When a mAb recognizes a particular protein on a barcoded HuProt array, its barcode and the protein address barcode appear in the same sequence. By counting the number of the reads for the same sequences, the relative strength of the mAb can be determined. By counting the number of different address polynucleotide barcodes coexisting with a given mAb's proximity polynucleotide barcode, binding specificity of the mAb can be determined.

### Example 4

### Single Cell Protein-Sequencing Via DAPPL

Hundreds to thousands of single cells are sorted into 384-well titer dishes. After a cell lysis reaction in each well, each single cell lysate receives a small amount of address polynucleotide (tethered with streptavidin). The resulting mixtures are transferred to a 384-well ELISA plate to allow immobilization of the total cell lysate proteins and address polynucleotides to the bottom of the well. After incubation at RT, bovine serum albumin (BSA) is added to each well to block further absorption of proteins. Meanwhile, a group of antibodies (e.g., 100) that each specifically recognizes a particular protein is tethered with a unique probe polynucleotide either via Cys or Lys residues of the antibodies. Probe polynucleotide-tethered antibodies are mixed and added to the ELISA plate. After incubation at RT, the ELISA plate is washed extensively. Next, the splint polynucleotide is added to each well to carry out the proximity ligation. After the ligation reaction, ligated products are PCR amplified, pooled, and subjected to deep-sequencing. Identity of each single cell is represented by the address polynucleotides; identity of proteins is revealed by the proximity polynucleotide. Similar to other DAPPL data, the number of each probe polynucleotide counts serves as the proxy of the protein concentration in each single cell detected by the corresponding antibodies.

### Example 5

### Single Cell Chromatin Immunoprecipitation Coupled with Deep-Sequencing (ChIP-seq) Via DAPPL

Single cells in the number of 100s are sorted into 384-well titer dishes. After cell lysis, each cell is briefly treated with DNase. Meanwhile, each ChIP-grade antibodies raised against transcription factors is individually mixed with an address polynucleotide and printed into each well of an ELISA plate. After incubation at RT, each well is blocked with BSA, and rinsed with phosphate buffered saline (PBS) buffer. DNase-treated single cell lysates are transferred into the antibody coated ELISA plates to allow immunoprecipitation (e.g., ChIP). After each well is extensively washed, splint polynucleotide is added to each well, followed by proximity ligation reactions. The ligation products are amplified with PCR reactions, pooled, and subjected to deep-sequencing. Identity of each antibody and the corresponding single cell is represented by the address polynucleotides; DNA sequences chromatin immunoprecipitated by each antibody are revealed by sequencing from the opposite end of the PCR products. Similar to other DAPPL data, the number or counts of each immunoprecipitated sequence reveals the binding sites by each transcription factor that the corresponding antibodies recognize.

### Example 6

### Multiplex ChIP-seq

A multiplexed chromatin-precipitation coupled deep-sequencing method is for simultaneous detection of transcription factor binding sites in chromatin. The principle of this idea is illustrated in FIG. 28. First, multiple monoclonal antibodies against hundreds to thousands of human transcription factors are spotted onto a nitrocellulose-coated slide (e.g., anti-TF mAb array) with address polynucleotides. Second, cells of interest are crosslinked, sheared, and ligated to a Y-shaped DNA adapter. Third, the ligated chromatin preps are incubated on the anti-TF mAb array to allow capture of the chromatin-DNA complexes by the mAbs spotted on the array. Fourth, after overnight incubation, the array is washed thoroughly, and the splint polynucleotide is added to facilitate ligation between captured genomic DNA fragments to the address polynucleotides at each mAb spot. Fifth, after recovery of the ligated DNA products from the array, they are PCR-amplified and subjected to deep-sequencing. Finally, a bioinformatics analysis is used to deconvolute the sequencing data.

### Example 7

### Single Cell Protein-Sequencing Via DAPPL

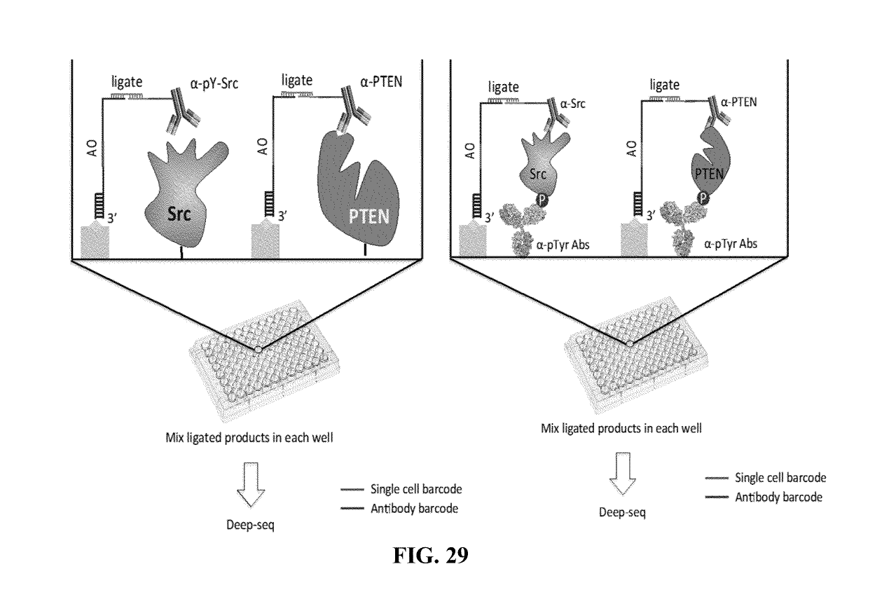

Hundreds to thousands of single cells are sorted into 384-well titer dishes. After a cell lysis reaction in each well, each single cell lysate receives a small amount of address polynucleotide (tethered with streptavidin). The resulting mixtures are transferred to a 384-well ELISA plate to allow immobilization of the total cell lysate proteins and address polynucleotides to the bottom of the well (FIG. 29; left panel). After incubation at RT, BSA is added to each well to block further absorption of proteins. Meanwhile, a group of antibodies (e.g., 100) that each specifically recognizes a particular protein is tethered with a unique proximity polynucleotide via either Cys or Lys residues of the antibodies. The proximity polynucleotide-tethered antibodies are mixed and added to the ELISA plate. After incubation at RT, the ELISA plate is washed extensively. Next, the splint polynucleotide is added to each well to carry out the proximity ligation. After the ligation reaction, ligated products are PCR amplified, pooled, and subjected to deep-sequencing. Identity of each single cell is represented by the address polynucleotides; identity of proteins is revealed by the proximity polynucleotides. Similar to other DAPPL data, the number of each probe polynucleotide counts serves as a measure of the protein concentration in each single cell detected by the corresponding antibodies.

Alternatively, a DAPPL-based approach to enable quantitative detection of Tyr phosphorylome in a single cell is performed. As illustrated in FIG. 29 (right panel), each FACS-sorted single cell is collected in a 96-well, plated, lysed, mixed with a particular address polynucleotide, and transferred to another 96-well plate, in which each well is already coated with anti-pTyr antibodies (e.g., 4G10 and pTyr100) and an address polynucleotide. After overnight incubation, a mixture of proximity polynucleotide-tethered antibodies that each recognizes a particular protein is added to each well and allowed to incubate at RT for 2 hr. The plate is then washed and a splint polynucleotide is added to each well, followed by ligation reactions. Ligated DNA products are collected from each well, mixed, and subjected to deep-sequencing. Address polynucleotides (AO) serve to identify a single cell and barcodes on the proximity-polynucleotides tethered to the mAbs serve to identify a particular protein.

### Example 8

### Single Cell ChIP-seq Via DAPPL

Hundreds to thousands of single cells are sorted into 384-well titer dishes. After cell lysis, each cell is briefly treated with DNase. Meanwhile, individual ChIP-grade antibodies raised against transcription factors are individually mixed with an address polynucleotide and printed into each well of an ELISA plate. After incubation at RT, each well is blocked with BSA, and rinsed with PBS buffer. DNase-treated single cell lysates are transferred into the antibodies coated ELISA plates to allow immunoprecipitation (e.g., ChIP). After each well is extensively washed, splint polynucleotide is added to each well, followed by proximity ligation reactions. The ligation products are amplified with PCR reactions, pooled, and subjected to deep-sequencing. Identity of each antibody and the corresponding single cell are represented by the address polynucleotides; DNA sequences immunoprecipitated by each antibody are revealed by sequencing from the opposite end of the PCR products. Similar to other DAPPL data, the number or counts of each immunoprecipitated sequence reveal the binding sites by each transcription factor that the corresponding antibodies recognize.

### Example 9

### Single Cell Quantification of Histone PTMs

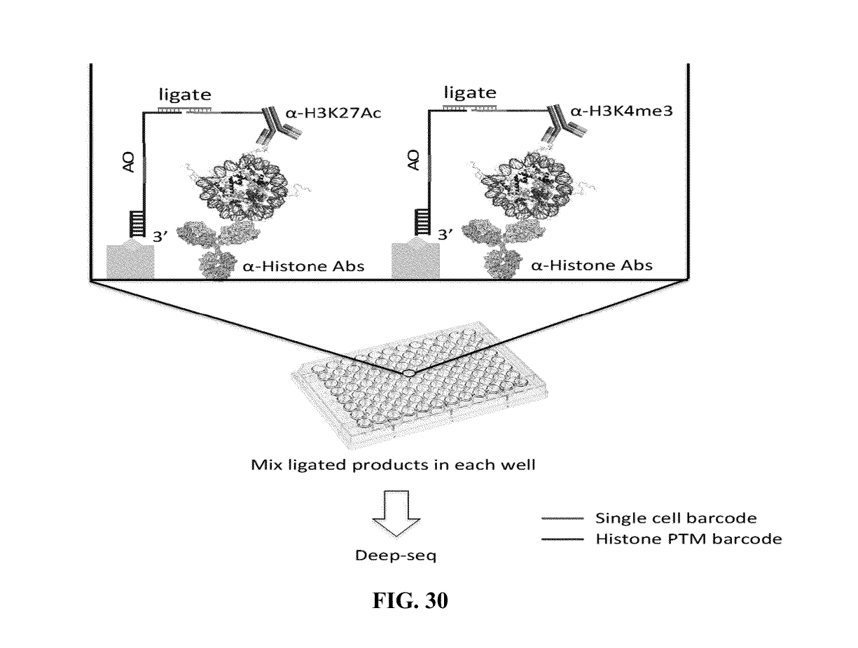

A DAPPL-based approach to enable quantitative detection of histone PTM abundance in a single cell is performed. As illustrated in FIG. 30, a generic anti-histone antibody is first used to coat each well with a particular address polynucleotide in a 96-well dish. Each FACS-sorted single cell is collected in another 96-well plate, lysed, and transferred to histone antibody-coated plates. After incubation and several washes, a mixture of anti-histone mark mAbs (e.g., anti-H3K27Ac and—H3K4me3), each tethered with a particular proximity polynucleotide, is added to each well. After overnight incubation, the plate is washed and a splint polynucleotide is added to each well, followed by ligation reactions. Ligated DNA products are collected from each well, mixed, and subjected to deep-sequencing. Address polynucleotides (AO) serve to identify a single cell and barcodes on the proximity polynucleotides tethered to the mAbs serve to identify a particular histone mark.

### Example 10

### DNA/RNA Aptamer Screens

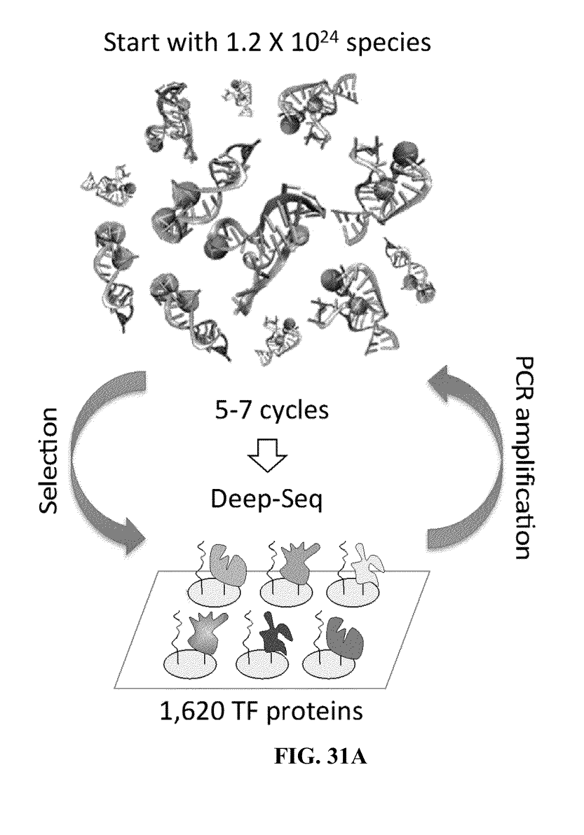

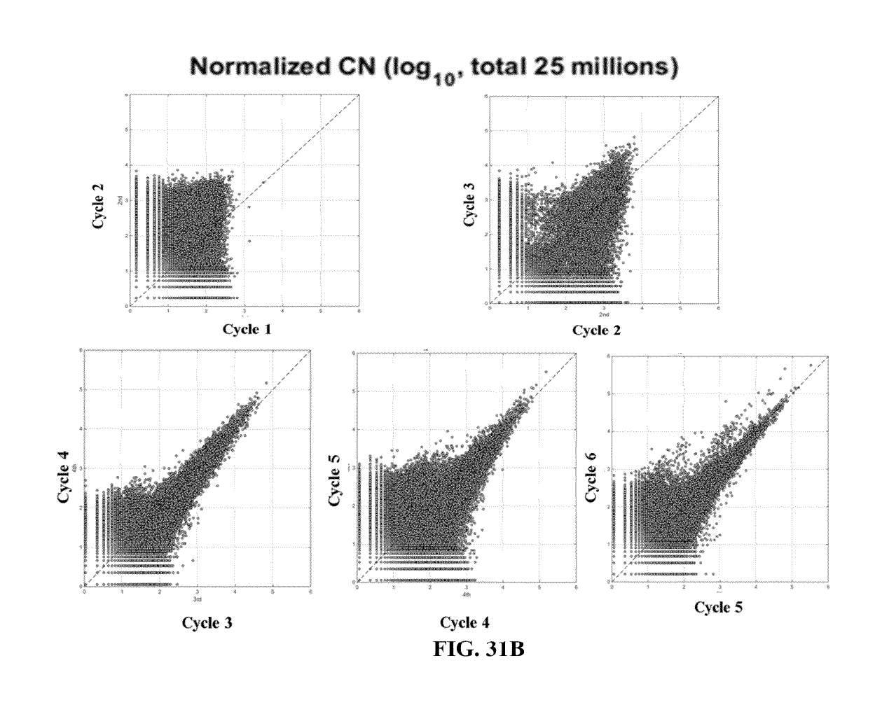

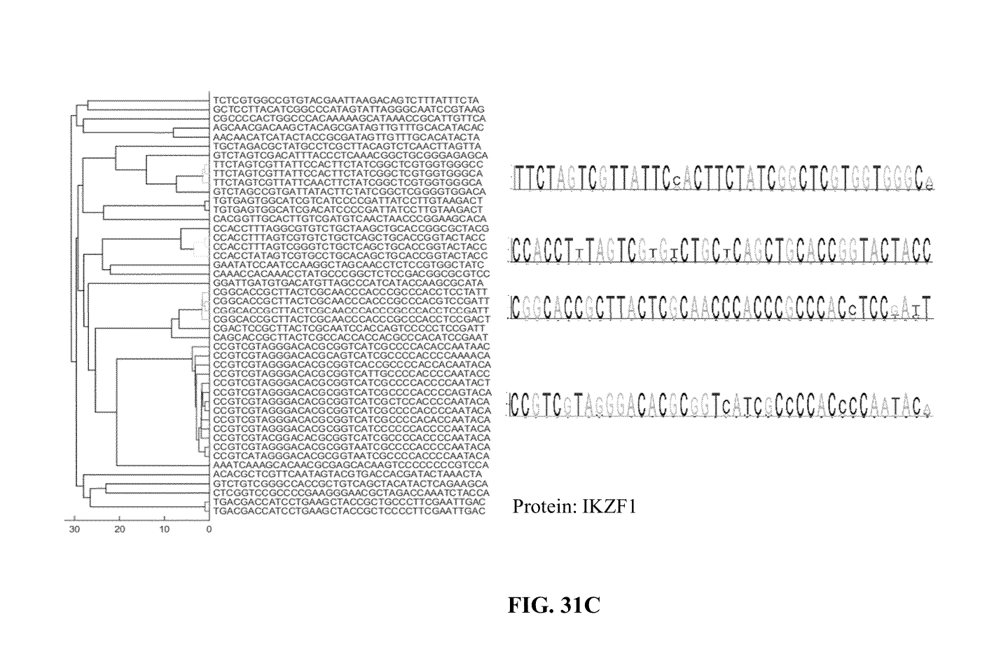

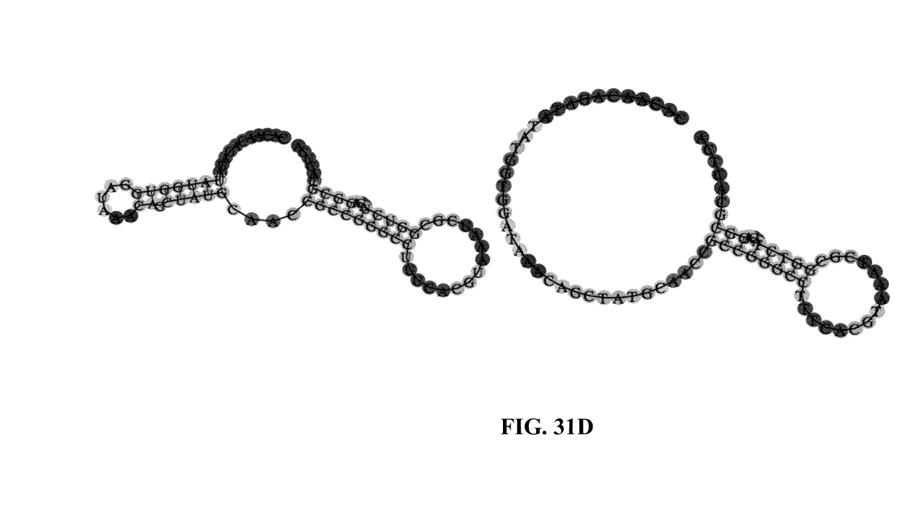

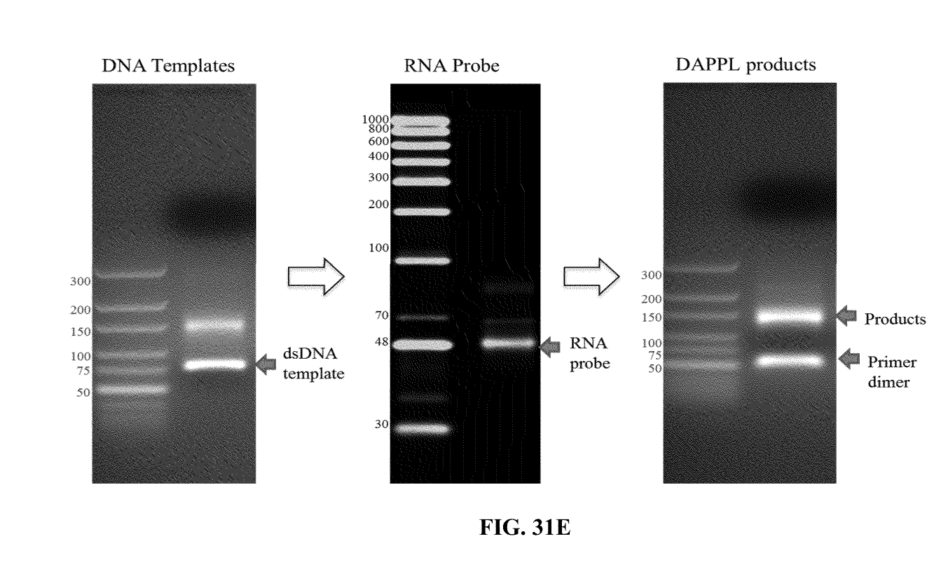

A DAPPL approach is applied to select DNA/RNA aptamers that can recognize human transcription factors and protein kinases with mono-specificity and high affinity. To screen DNA aptamers, 20- and 40-mer random DNA polynucleotides with fixed flanking sequences on both sides are used. They are heated at 98° C. for 10 min and slowly cooled down to allow formation of secondary or tertiary structures (FIG. 31A). The mixture of DNA aptamers is incubated on the human TF array at 4° C. overnight. After several washes, the bound DNA aptamers are recovered from the slide and PCR-amplified. Next, asymmetric PCR is performed using an aliquot of the PCR products to regenerate DNA aptamers, which go through the same procedure 4 times. At the DNA aptamer incubation step on the TF array in cycle 5, the bound DNA aptamers are ligated to the address polynucleotides spotted on the TF array and an aliquot of the recovered ligation products is deep-sequenced. The same procedure is continued for two more cycles, and at the ends of cycles 6 and 7, the ligated products are deep-sequenced. By comparing the deep-sequencing data obtained at cycles 5, 6, and 7, mono-specific DNA aptamers of high affinity can be selected. The same approach is applied to identify aptamers on HuProt arrays. A summary of the sequencing data can be seen in the table below.

| | | Reads with | | # of | # of |
|---|---|---|---|---|---|
| | Total reads | correct | # of unique | unique | unique |
| | (S > 0.8) | AO (MM < 2**) | interactions | AO | 40mers |
| 1^{st} | 18,522,417 | 17,254,637 | 5,013,079 | 1,632 | 3,796,493 |
| 2^{nd} | 15,952,482 | 14,236,488 | 1,936,440 | 1,632 | 1,600,056 |
| 3^{rd} | 25,816,916 | 23,011,582 | 2,734,733 | 1,627 | 1,924,468 |
| 4^{th} | 23,891,027 | 21,892,598 | 2,312,094 | 1,629 | 2,156,529 |
| 5^{th} | 25,852,913 | 21,978,068 | 2,599,546 | 1,604 | 1,964,519 |
| 6^{th} | 16,989,728 | 15,464,832 | 1,492,780 | 1,621 | 1,349,077 |

Meanwhile, RNA aptamers are screened against human proteins kinases (>500 proteins, including some splice variants). The procedure is almost the same as above, except a step of in vitro transcription after PCR amplification is added to convert double-stranded DNA templates to RNA aptamers. Similarly, recovered RNA aptamers at the end of cycles 5, 6, and 7 are deep-sequenced to reveal their identity.

The ability of DNA aptamers to perform Western analysis (WB), immunoprecipitation (IP), chromatin precipitation (ChIP), and/or immunohistochemistry analysis (IHC), is performed by selecting a small random set (e.g., 20-40) of identified aptamers and synthesis of them with a biotin moiety attached to their 5′- or 3′-ends. Using HRP-conjugated streptavidin, these DNA aptamers are tested in at least 6 cell lines. A single band in WB analysis with a particular aptamer is optimal. Cell lines transfected with FLAG-tagged target constructs are used to perform aptamer-assisted IP assay. The success of IP is detected with anti-FLAG WB. Similarly, the success of aptamer-assisted ChIP is determined by comparison between the immunoprecipitated peaks with those identified with antibody-based approaches. A significant overlap between the two approaches is expected to be observed.

To determine which RNA aptamers can effectively inhibit autophosphorylation activity of their targets, autophosphorylation assays with γ-³²P-ATP on a kinase array in the presence or absence of the mixture of identified RNA aptamers is performed. First, purified kinase proteins are spotted on an epoxy surface to form a kinase array. After blocking the surface with BSA, the immobilized kinases are preincubated with a mixture of identified RNA aptamers at different concentrations for 1 hr. at RT. Kinase reaction buffer containing γ-³²P-ATP are added to the kinase array and the autophosphorylation reactions are carried out for 20 min at 30° C. The autophosphorylation signals are detected by exposure of the kinase array to a piece of X-ray film. As a positive control, a kinase array without the RNA aptamer treatment is carried out in parallel, signals of which are used as positive controls. Those RNA aptamers that can significantly reduce autophosphorylation signals are selected for further in vivo validation. Kinases that are well-studied with known downstream substrates are selected for the in vivo validation. Their corresponding RNA aptamers are overexpressed in cell lines by transfecting constructs carrying the cDNAs of the RNA aptamers. The kinase's autophosphorylation level and the phosphorylation level of the kinase's downstream targets are expected to be reduced as detected with a phospho-specific antibody, when an RNA aptamer effectively inhibits its target kinase in cells.

Three rounds of screening of 12-mer RNA libraries against an array of over 2000 annotated RNA-binding proteins have been performed using the rDAPPL approach.

### Example 11

### DNA-Templated Macrocycle Screens

A DAPPL approach is also applied for detection of small molecule and protein interactions. A protein array with Src and IDE as positive controls is generated, and 10 other random proteins as negative controls. Each protein is spotted with a unique address polynucleotide. Two DNA-templated macrocycles (small molecules) are first tested individually on the array at a wide range of concentration (e.g., pM to attoM). Interactions with their expected targets (e.g., Src and IDE) are confirmed by Sanger sequencing the PCR products of the ligated products. The two small molecules are tested again if successful in the context of a compound mixture.

### Example 12

### Synthetic V_{H} and V_{L} Single Domain Screens

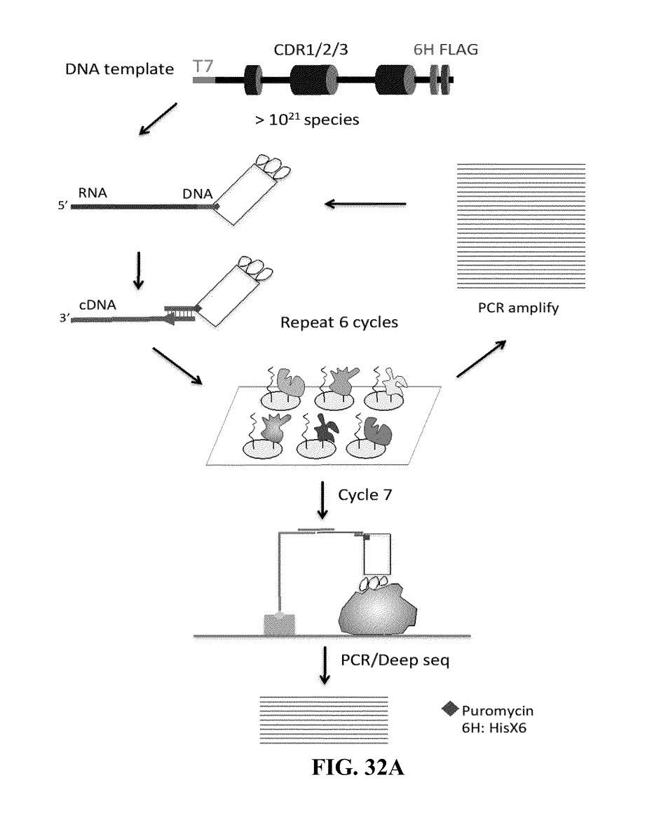

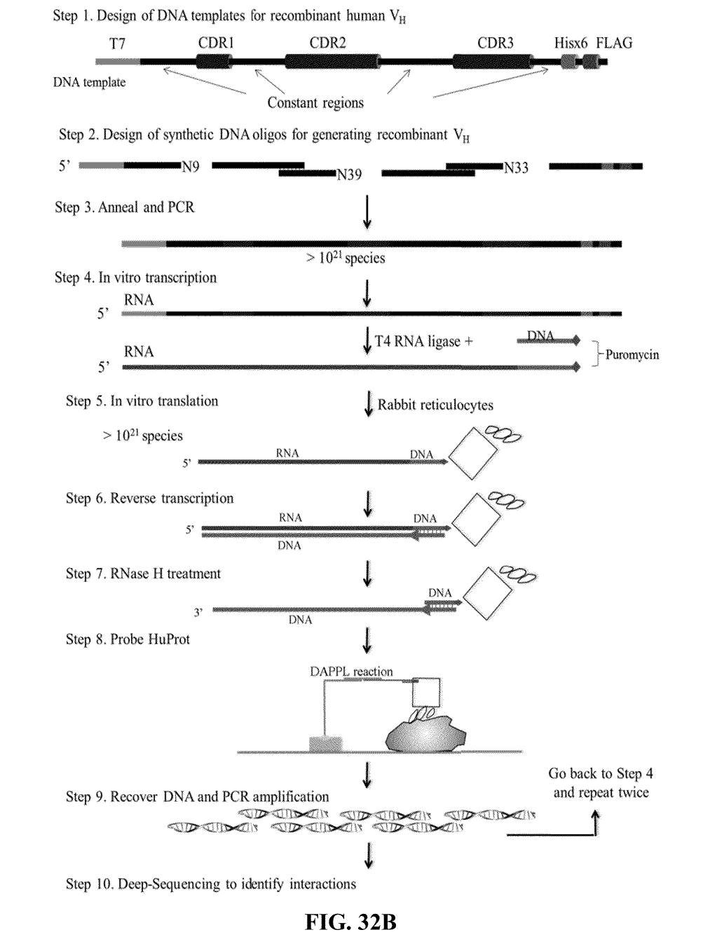

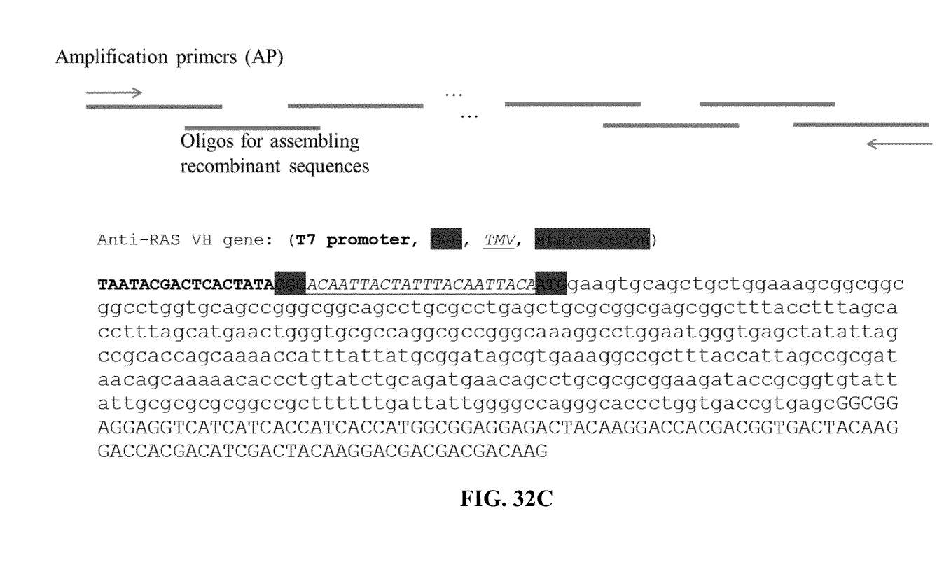

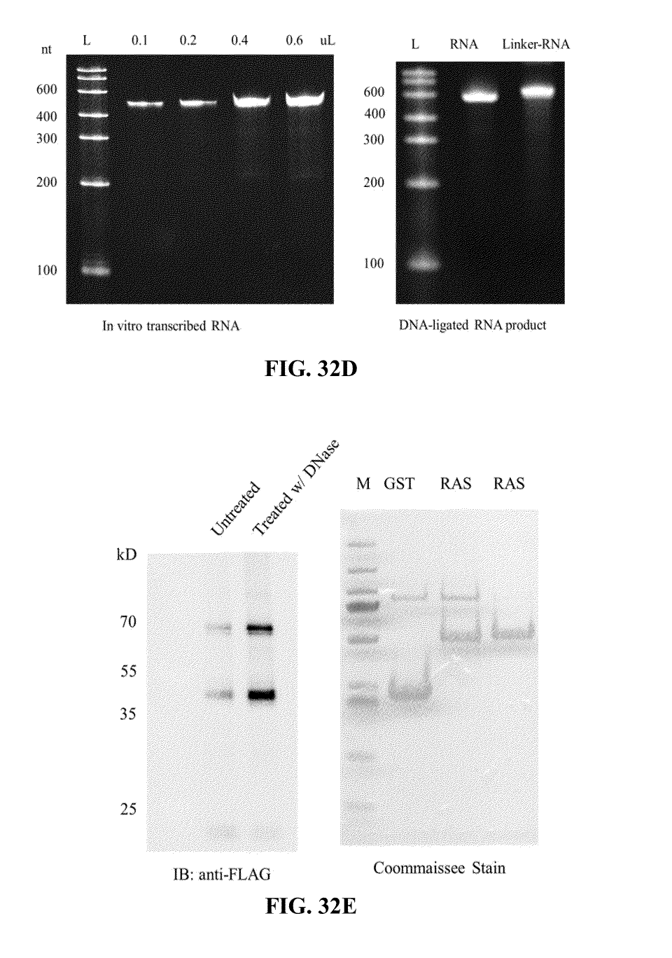

A DAPPL approach is used to identify synthetic heavy chain variable (V_{H}) and light chain variable (V_{L}) single domains that can specifically recognize human proteins with high affinity. First, a pool of DNA polynucleotides that will be used as templates to produce single domains using the mRNA display approach (FIG. 32) is generated. As illustrated in FIG. 32, a T7 promoter sequence is added to a 5′-end of the polynucleotide, followed by the V_{H} or V_{L} sequence containing compliment determining regions (CDR1, CDR2, and CDR3). A polynucleotide sequence encoding His×6 (SEQ ID NO: 5) and FLAG epitopes is added to the 3′-end of the polynucleotide. Next, using a standard protocol, a protein pool of either V_{H} or V_{L} single domains is generated that are tethered with its coding RNA sequence via a puromycin moiety. Reverse transcription is applied to create the cDNA strands. This mixture is incubated on the human protein microarray. After stringent washes, the captured single domains are recovered from the microarray and the tethered cDNA is PCR-amplified. The PCR products serve as the templates for the next round of screen. This screen will be performed for 6 cycles. At the incubation step in cycle 7, the cDNAs tethered to the single domains are ligated to the address polynucleotide spotted together with each protein on the array, followed by PCR amplification. The PCR products are deep-sequenced to determine which protein on the array is recognized by which single domain.

### Example 13

### ChIP-omix Approach to Comprehensively Map TF Binding Sites to Chromatin

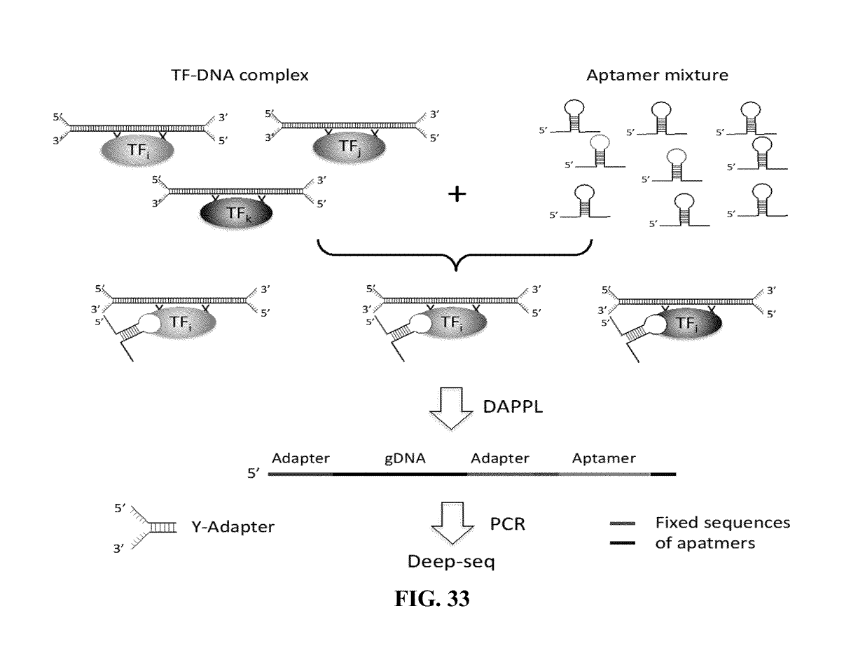

The above mentioned aptamers are used to perform a comprehensive ChIP assay against all human TFs at once with a mixture of their corresponding aptamers. As illustrated in FIG. 33, a set of oligos that each encode a particular aptamer sequence flanked by two flexed sequence tags is generated first. To facilitate purification of the aptamer-TF complexes, a biotin moiety is added to either the 5′- or 3′-end of the aptamers. Equal amounts of all the synthesized aptamers are mixed together to various final concentrations (e.g., 10 nM to 1 μM). Meanwhile, standard chromatin preparation are carried out in selected cell lines (e.g., ENCODE cell lines). After shearing the chromatin, the shared genomic DNAs are end-repaired and ligated to a Y-shaped DNA adapter. Next, the aptamer mixture is added to the chromatin preps and allowed to incubate overnight. To avoid cross-complex ligation, the mixture is diluted at least 10-fold and a splint polynucleotide is added to facilitate ligation between the aptamer ends (e.g., 5′- or 3′-fixed sequences) and the single-stranded sequences of the Y-shaped adapters. After the ligation reaction, streptavidin-coated beads are added to the mixture to capture the aptamer-TF-DNA complexes via the biotin moiety on the aptamers. The beads are washed under stringent conditions and the bound DNA ligation products are recovered by boiling the beads. Next, the ligation products are PCR-amplifed and subjected to Hi-seq analysis to identify chromatin locations to which each TF bind.

Because the biotin moiety can be attached to either the 5′- or 3′-ends of the DNA aptamers, the ChIP-omix assays are performed in both labeling orientations. Given the complexity of the chromatin distribution of the TFs as a whole, a single run of Hi-seq (e.g., 300 M reads) may not be sufficient to fully cover all the possibilities. If this is the case, more Hi-seq runs are used to generate up to 3 billion reads. The ChIP peaks identified with the ChIP-omix approachare expected to show a significant overlap with those identified with the traditional antibody-based ChIP-seq approach.

### Example 14

### WB-Omix Approach to Globally Quantify Proteins of Human Proteome in Cells and Tissues

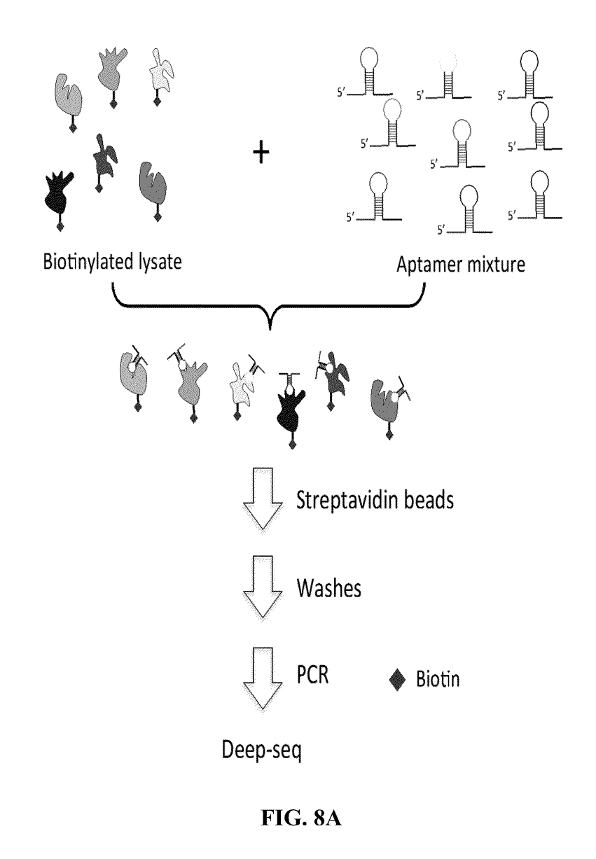

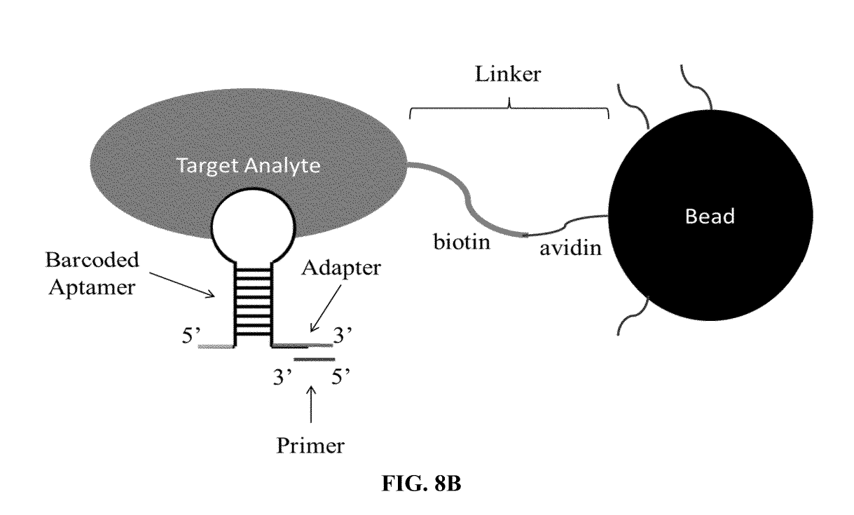

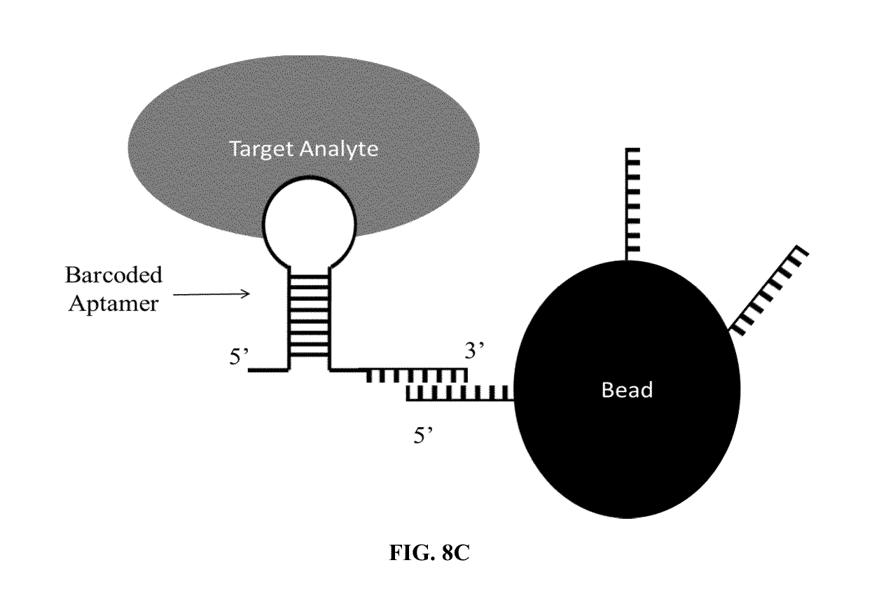

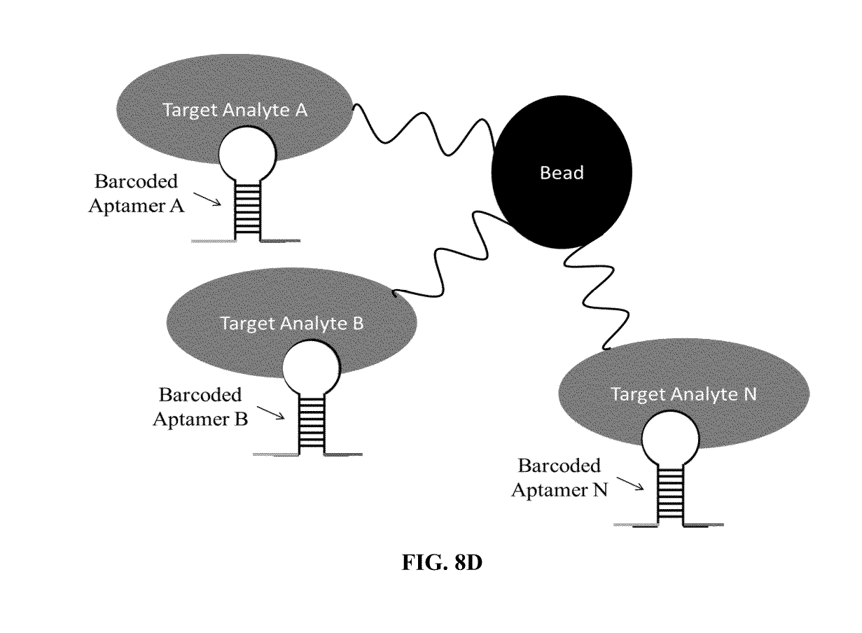

The identified DNA/RNA aptamers are used for proteome-wide detection of protein abundance inside a cell or tissue (FIG. 8). First, total protein lysates obtained from cultured cell lines or primary tissues are biotinylated. Second, a mixture of hundreds to thousands of aptamers are mixed and added to the lysates. After incubation at RT, the aptamer-protein complexes are purified using streptavidin beads, followed by stringent washes. After recovery of the aptamers captured by proteins, they are PCR-amplified and deep-sequenced. Alternatively, the identity of captured aptamers can be revealed by hybridiuzing recovered aptamer, end-labeled with a fluorphore (e.g., Cy5), to a DNA polynucleotide array that encodes the complementary sequences of the entire aptamer set used in this assay. The number of reads per aptamer serve as a determination of a relative abundance of the targeted proteins. As a positive control, a foreign protein (e.g., GFP) with a V5-tag is spiked into the lysate at a known concentration (e.g., 1 nM). An aptamer recognizing V5 epitope is included in the aptamer mixture to serve as a normalization control.

### Example 15

### IP-Omix Approach to Comprehensively Map Protein-Protein Interactomes

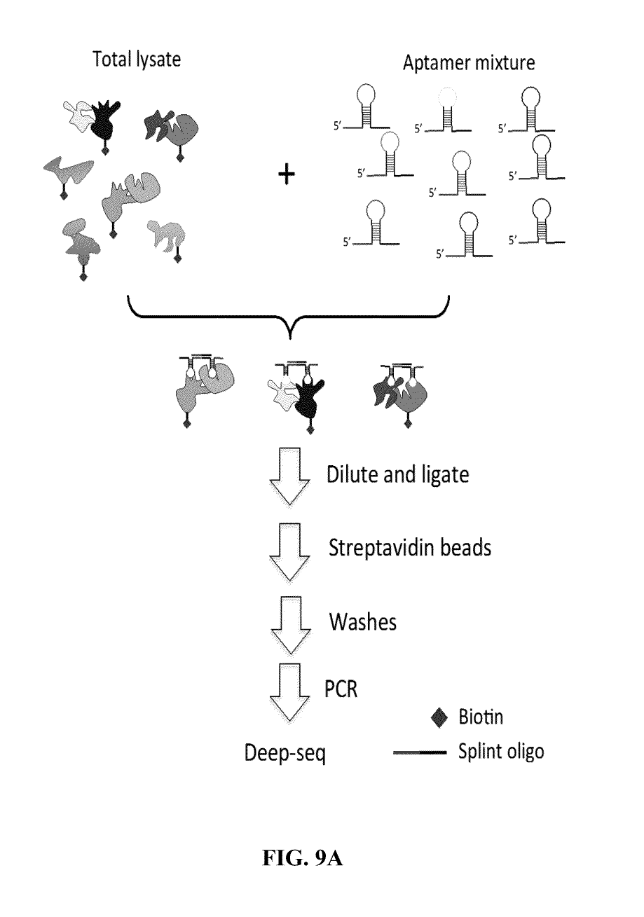

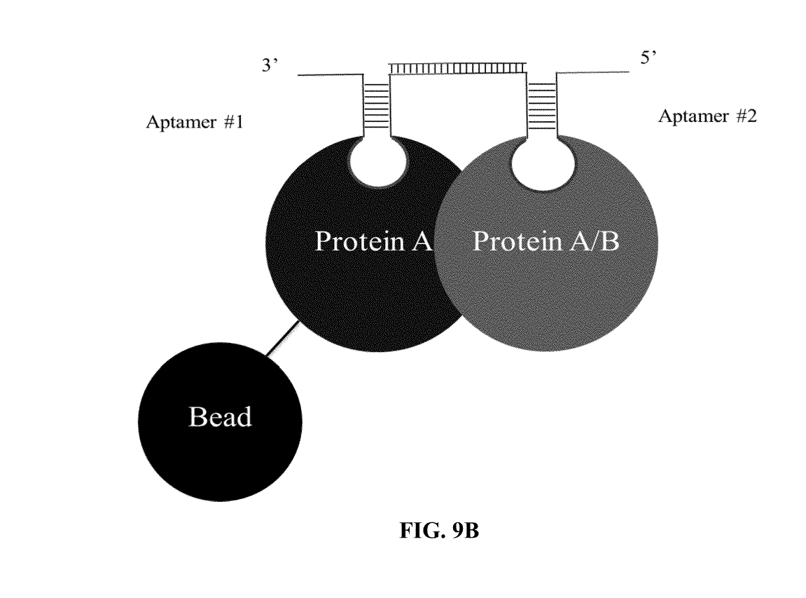

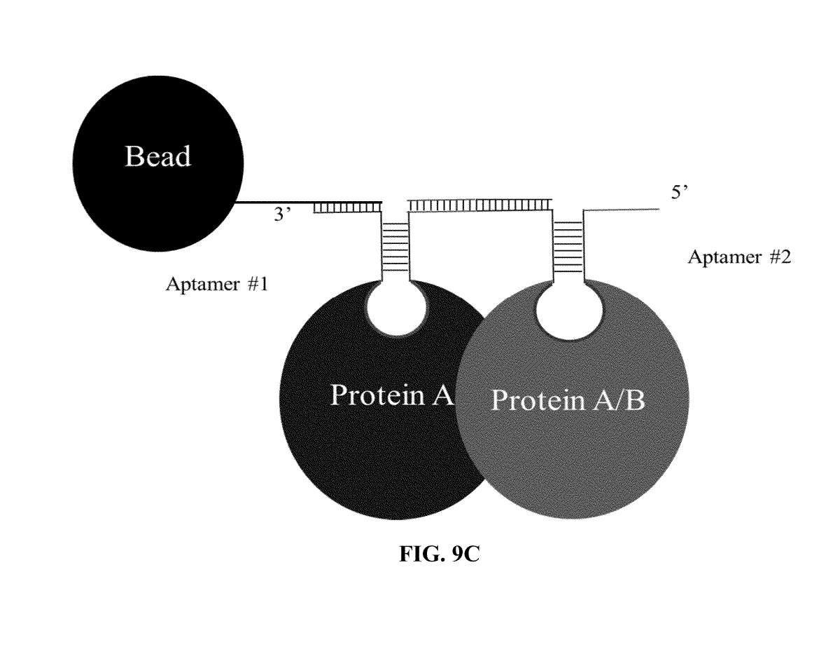

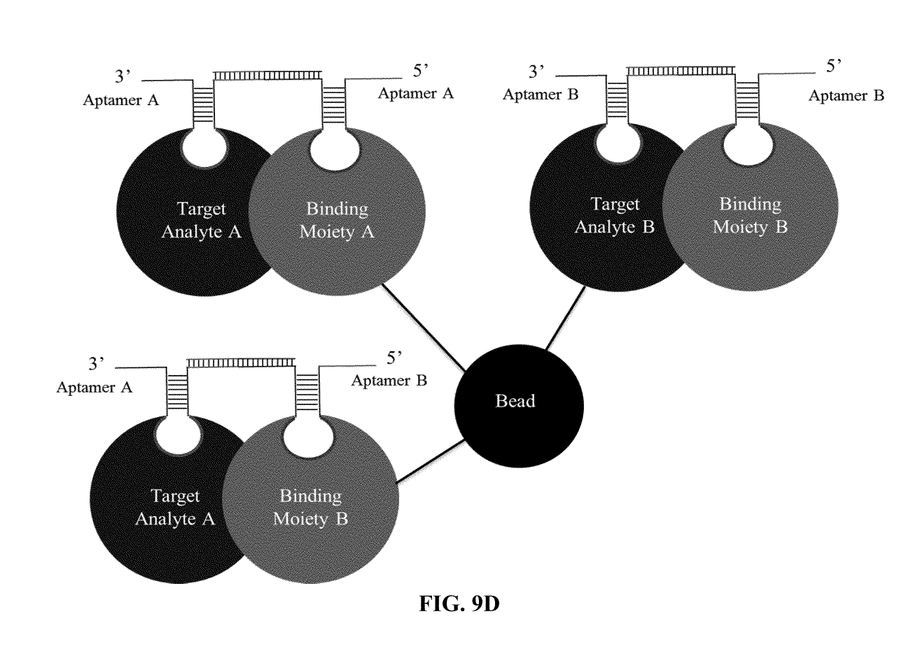

The previously identified aptamers are used to determine protein-protein interactions by testing all possible combinations in a cell line and/or tissue. As illustrated in FIG. 9A, the an exemplary IP-omix method utilizes a mixture of DNA/RNA aptamers that each recognizes a unique human protein to examine all possible combinations of protein-protein interactions inside a cell or tissue. First, total protein lysates obtained using standard protocols from cultured cell lines or primary tissues are lightly biotinylated. Second, a mixture of hundreds to thousands of aptamers are mixed and added to the lysates. When two proteins form a complex inside the cell, their corresponding aptamers are brought to proximity. After diluting the lystate-aptamer mixture to prevent cross-complex ligation, a splint polynucleotide is added to the mixture and allowed to aneal to the 5′- and 3′-ends of the fixed sequences on the aptamers. After ligation reaction, the two aptamers bound to the same protein complex form an aptamer dimer To remove aptamer dimers formed between free aptamers (e.g., those that are not bound to a protein), biotinylated protein complexes are purified with streptavidin beads and washed under stringent conditions. The ligated aptamer dimers are then recovered from the beads and PCR-amplified. The PCR products are deep-sequenced to identify all possible aptamer pairs. To validate the identified protein complexes, standard co-IP experiments are performed using a random set of candidate protein pairs (e.g., 20-40 pairs). For example, to validate interactions between proteins X and Y, cells are co-transfected with X and Y expression constructs each tagged with a different epitope (e.g., V5 and FLAG). Using the standard IP-WB analysis, in vivo interactions between selected candidate pairs are expected to be validated. Recovery of many literature-reported interactions is also determined.

### Example 16

### Peptide Ligand Screens for Human GPCRs on VirD Arrays

The DAPPL and VirD approaches are used to identify ligands for human transmembrane proteins. Peptide ligands for 128 orphan GPCRs are identified by performing high-throughput screening against a 10-mer random peptide library in a microarray format (e.g., a human GPCR VirD array). Among the 288 well-annotated GPCRs (International Union of Basic and Clinical Pharmacology, a.k.a., IUPHAR), Class A is the largest with 79 orphans, such as those for somatostatin, relaxin, prokineticin, and peptide ligands. Therefore, these 79 orphan GPCRsre included. Because of the high-throughput nature of the VirD technology, the 49 orphans in other classes and 12 well characterized GPCRs with known peptide ligands are included as positive controls. Therefore, a total of 140 GPCRs are expressed in recombinant virions. Full-length GPCR ORFs are selects from a human ORFeome collection to generate recombinant viruses. Individually purified virions are spotted on a glass slide to form an orphan GPCR VirD array. In parallel, a peptide library, comprised of random 10-mer peptide sequences is constructed using the mRNA-display method. Assuming a random distribution, a 10-mer peptide pool contains >1×10¹³ peptide species, much more complex than phage- or bacteria-display libraries. A pool of DNA oligo templates is synthesized, each encoding a 30-mer random nucleotide sequence flanked by an upstream T7:Kozak sequence to facilitate in vitro transcription/translation, and by a downstream reverse T3 sequence to facilitate ligation to a puromycin-tethered single-stranded DNA oligo (FIG. 8). Using a standard mRNA-display protocol, these oligos are then converted into a random 10-mer peptide pool in which each peptide is conjugated with its coding sequence (FIG. 8; steps 1-4). To screen for peptides that can be captured by orphan GPCRs, the pool of the peptide conjugates is incubated on the GPCR VirD arrays, followed by a stringent washing step (FIG. 8; step 5). The captured mRNA-peptide conjugates are recovered and their coding sequences PCR-amplified (FIG. 8; Step 6). To enrich for peptides with high affinity to their orphan GPCRs, the above selection procedure is repeated 5 times. After step 4 in cycle 6, the cDNA-mRNA-peptide conjugate are treated with RNase to remove the RNA moiety. After incubation on the GPCR VirD array in which each recombinant virion is spotted with a unique address polynucleotide, the captured cDNA-peptide conjugates are ligated to the address polynucleotides. After recovery of the ligation products from the VirD array, they are PCR-amplified and a fraction of the products are deep-sequenced (FIG. 8; step 7). A fraction of the remaining product serves as the template for cycle 7 screen using the same procedure as cycle 6. Bioinformatics analysis sithen performed to identify statistically enriched peptide-GPCR interactions. The selection is performed against 128 orphan GPCRs in parallel.

The GPCR ligands identified above are confirmed with a different system. The activation of many GPCRs, particularly those coupled with the G_{q}-PLC pathway, leads to an increase in intracellular Ca²⁺ level. A heterologous cell-based Ca²⁺ imaging assay is employed for further characterization of these identified peptide ligands. At least 5 positive orphan GPCRs (e.g., Mrgs) coming out of the VirD array assays are validated employing Ca²⁺ imaging assays. Heterologous cells, which do not express endogenously a GPCR-of-interest are used. The parental cells without GPCR expression are included in the experiments as negative controls. Judged by increase or decrease (e.g., 20%) in fluorescence intensity as compared to the baseline level, agonist versus antagonist ligands are identified, respectively. As a control, validated ligands are counter-screened in heterologous cells expressing unrelated GPCR, and in parental cells to ensure target specificity.

### Example 17

### Aptamer Screens on VirD Arrays

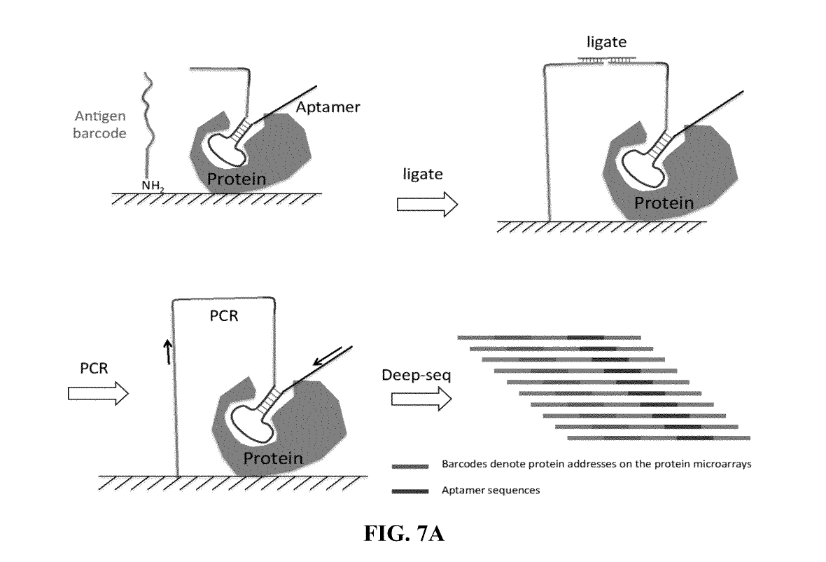

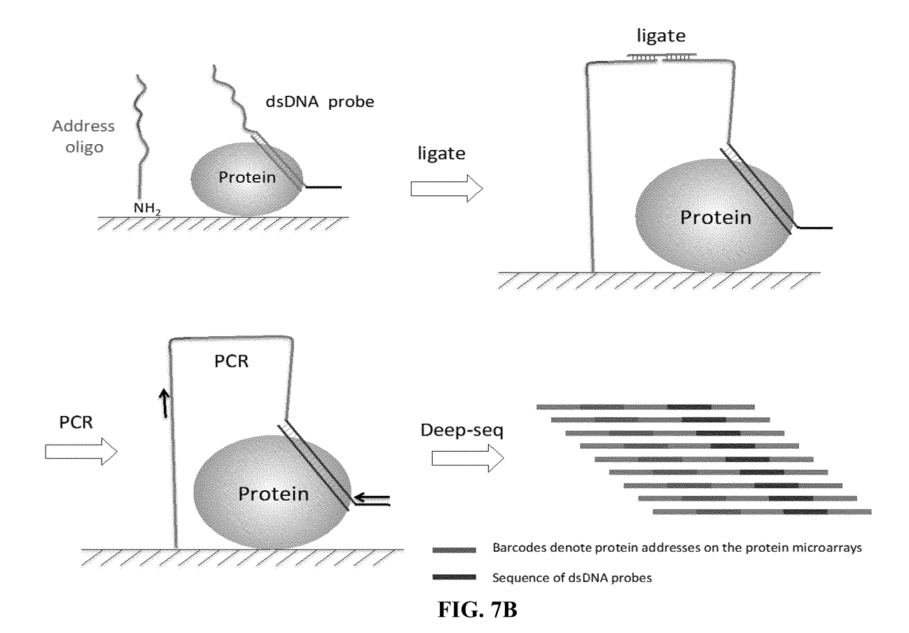

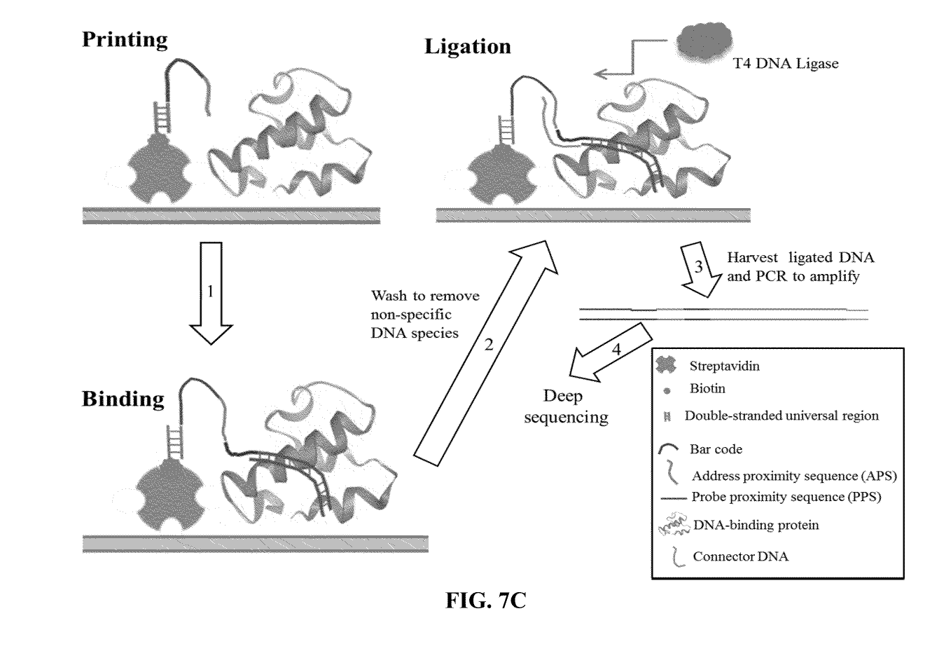

The DAPPL and VirD approaches are employed to select DNA/RNA aptamers that can recognize human transmembrane proteins with mono-specificity and high affinity. To screen the aptamer pools, 20- and 40-mer random DNA polynucleotides are generated with fixed flanking sequences on both sides. They are heated at 98° C. for 10 min and slowly cooled down to allow formation of secondary or tertiary structures (FIGS. 7A and 7B). Meanwhile, a VirD array comprised of human GPCRs (e.g., ˜300 non-odorant GPCRs), 300 ion channels, and 100 members of immunoglobulin superfamily is constructed. The mixture of DNA aptamers is incubated on the human transmembrane VirD array (˜700 recombinant virions) at 4° C. overnight. After several washes, the bound DNA aptamers are recovered from the slide and PCR-amplified. Next, asymmetric PCR is performed using an aliquot of the PCR products to regenerate DNA aptamers, which goes through the same procedure 4 times. At the DNA aptamer incubation step on the VirD array in cycle 5, the bound DNA aptamers are ligated to the address polynucleotides spotted on the VirD array and an aliquot of the recovered ligation products is deep-sequenced. The same procedure continues for two more cycles, and at the ends of cycles 6 and 7 the ligated products are deep-sequenced. By comparing the deep-sequencing data obtained at cycles 5, 6, and 7, mono-specific DNA aptamers of high affinity are selected.

### Example 18

### Functional Aptamer Screens Against Human Receptor Tyrosine Kinases on Virion Arrays

DAPPL-assisted virion (VirD) technology is employed to identify aptamers that can recognize conformational epitopes in the ecto-domains of 58 receptor tyrosine kinases (RTKs). First, a virion-displayed RTK VirD array is generated. A mixture of DNA aptamers is incubated on the human RTK VirD array at 4° C. overnight. After several washes, the bound DNA aptamers are recovered from the slide and PCR-amplified. Next, asymmetric PCR is performed using an aliquot of the PCR products to regenerate DNA aptamers, which goes through the same procedure 4 times. At the DNA aptamer incubation step on the VirD array in cycle 5, the bound DNA aptamers are ligated to the address polynucleotides spotted on the VirD array and an aliquot of the recovered ligation products is deep-sequenced. The same procedure continues for two more cycles, and at the ends of cycles 6 and 7, the ligated products are deep-sequenced. By comparing the deep-sequencing data obtained at cycles 5, 6, and 7, mono-specific DNA aptamers of high affinity are selected.

To determine whether a positive aptamer activates or inhibits its target RTK, a cell-based system is employed. If a given aptamer can block the corresponding RTK signaling, pretreatment of cells with this aptamer (assuming the target RTK is expressed) prior to adding the canonical ligand abolishes/reduces autophosphorylation signals of the RTK as judged by Western Blot (WB) analysis with antibodies that specifically recognize the autophosphorylated form of the RTK. On the other hand, if an aptamer can activate the RTK signaling, incubation with this aptamer in the absence of the canonical ligand is sufficient to induce autophosphorylation of its target RTK, as judged with the same WB analysis. Both types of aptamers are of great value. Alternatively, when a RTK of interest is not readily expressed in a cell, or antibodies that recognize its activated form are not commercially available, cells transfected with a C-terminally V5-tagged (or FLAG) RTK construct are employed to go through a similar immunoprecipitation-coupled WB analysis to evaluate functionality of the aptamers.

### Example 19

### Identify Small Molecule Inhibitors Against Ion Channels

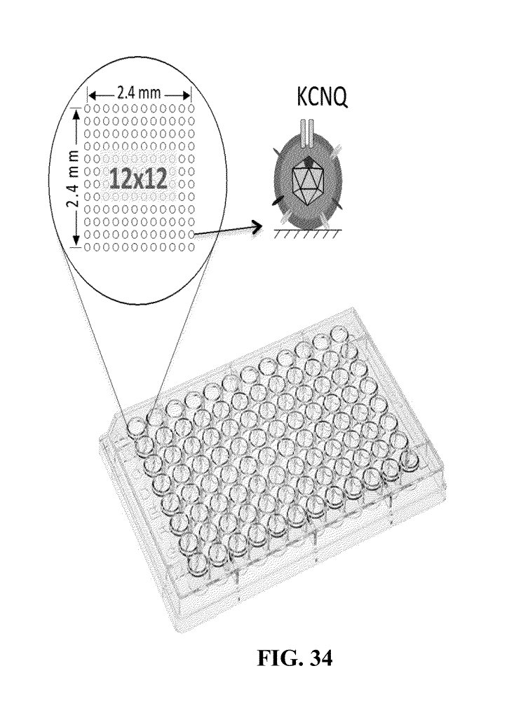

A highly multiplexed platform for inhibitor screens against human ion channels is also employed. Because the VirD technology offers a cell-free system, multiple ion channels can be simultaneously screened against a compound library, allowing for both specific target screen and simultaneous counter-screens against all other ion channels. As the viral envelope is almost identical to plasma membrane, ion channels displayed on virions are functional. 10 sodium and 55 (40 voltage-gated and 15 inwardly rectifying) potassium channels are used. Opening of these channels can be readily detected by a high-content imaging system using fluorescent dyes as a reporter. Such a screen scheme has established using several high-content, automated imaging systems, such as BD Pathway Imager. First, a robotic microarrayer (NanoPrint, Arraylt) is used to spot a total of 65 virion-displayed ion channels in duplicate at the bottom of wells in a 96-well plate (FIG. 34); WT virions are included as negative controls. After virions in the plates are loaded with the dyes (e.g., ANG-2 for sodium channel imaging), 3,280 compounds (Sigma LOPAC and Microsource Spectrum) at 10 μM are robotically added to each well. After loading the plates onto a BD Pathway Imager to establish a baseline, stimulus buffer is added, and fluorescence signals are detected continuously for 180 sec. Signals obtained from WT virions serve as baseline of fluorescent signal detection. Compared to the WT virions, a compound that causes a signal reduction >3 standard deviations in activity is scored as a hit. Z-scores are calculated for each interaction. To avoid potential side effects, those hits that only specifically inhibit a single channel in the assays are selected for further validation.

To confirm the activity of the hits and to estimate their potency, high-throughput planar array electrophysiology using the IonWorks Quattro system (Molecular Devices) is used. Using a standard protocol, 50 stable cell lines (e.g., HEK293 or CHO) that each overexpresses one of the 65 ion channels upon induction (15 are already available) are obtained or created. For those without previous validation data, the optimal conditions for channel recording are first identified using the single-hole mode on the IonWorks Quattro. Under the optimized condition, population patch clamp (PPC) electrophysiology is performed using the IonWorks Quattro by testing each candidate compound at 8 different concentrations (e.g., 100 μM to 10 nM) in quadruplicate. Given different biophysical properties, the sodium and potassium channels are assayed separately. On the basis of these analyses, potency and efficacy values of the tested hits are calculated as IC₅₀ and minimum activity using origin 6, respectively. Those compounds that have IC₅₀ values below 5-10 μM and are at least 10-fold more specific than any other channel are considered as validated.

### Example 20

### Protein-DNA Interactions

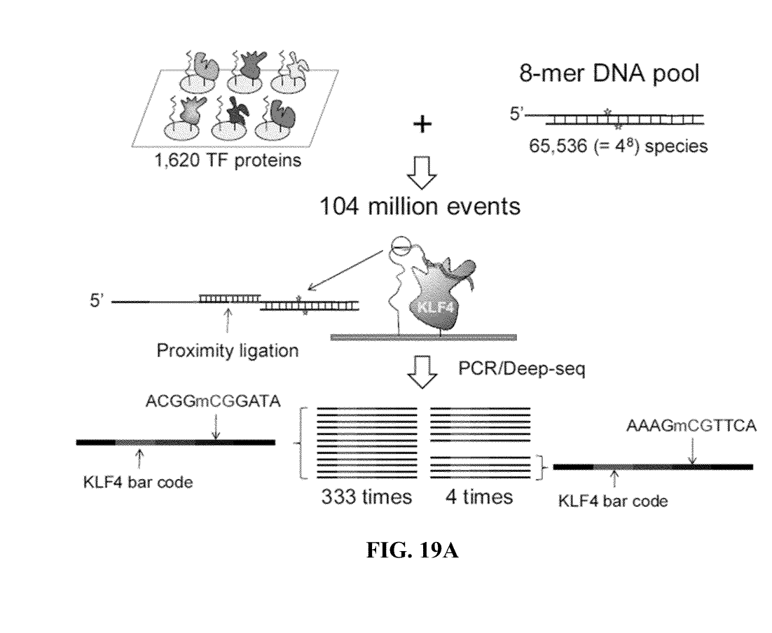

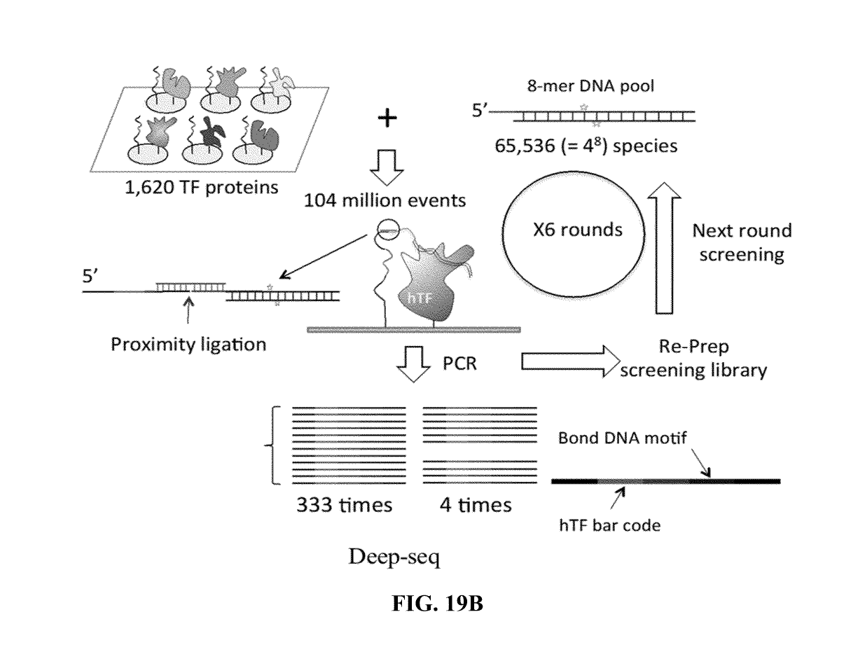

A DAPPL approach was applied to establish a comprehensive screen for protein-DNA interactions. As illustrated in FIG. 19A and FIG. 19B, 1,600 human TF proteins were each mixed with a unique address polynucleotide and spotted to form a TF protein microarray. Meanwhile, a pool of DNA polynucleotides was synthesized that each contains eight random nucleotides with a CpG in the middle and two fixed sequences on both sides. After a double-stranded DNA pool was made with a Klenow reaction, bacterial enzyme SssI is used to methylate the CpG, followed by treatment with a T4 DNA polymerase to generate a 5′-overhang. This pool of 65,536 species was then incubated on the TF array, washed, and the captured DNA fragments were ligated to the address polynucleotides. In each ligated sequence, a mCpG containing 8-mer sequence is now connected to an address oligo representing the TF protein that has captured this 8-mer in the binding assay. Therefore, it is an “all-to-all” screening approach. In other words, 4⁸ (i.e., 65,536) 8-mer species can be screened simultaneously against 1,620 TFs in a single experiment, representing 1×10⁹ combinations. The ligated products were then recovered from the slide and PCR-amplified. This pool of DNA was then re-screened using the same protocol for a total of six cycles, and the products of cycles 4-6 were deep-sequenced. Bioinformatics analyses were performed to determine whether there was any enrichment of consensus motifs for each TF protein.

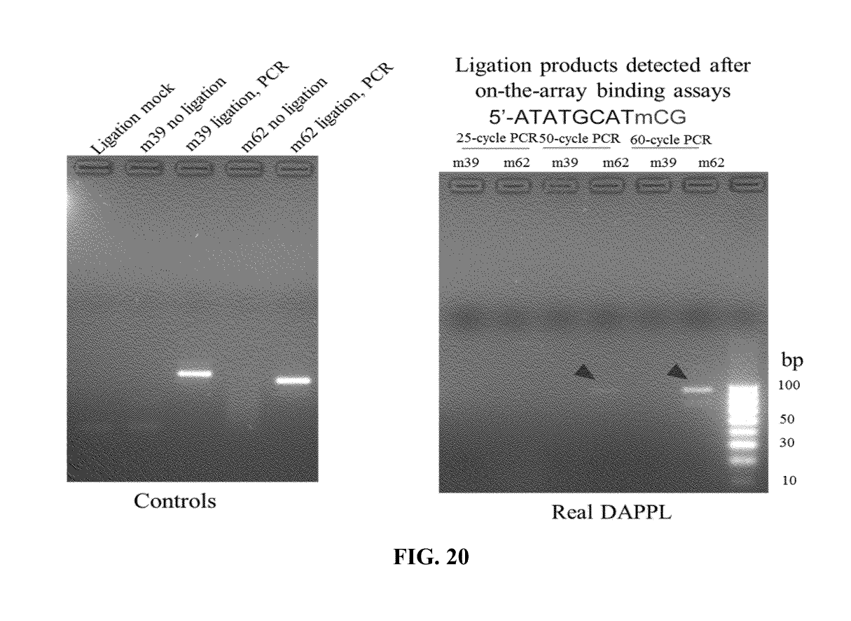

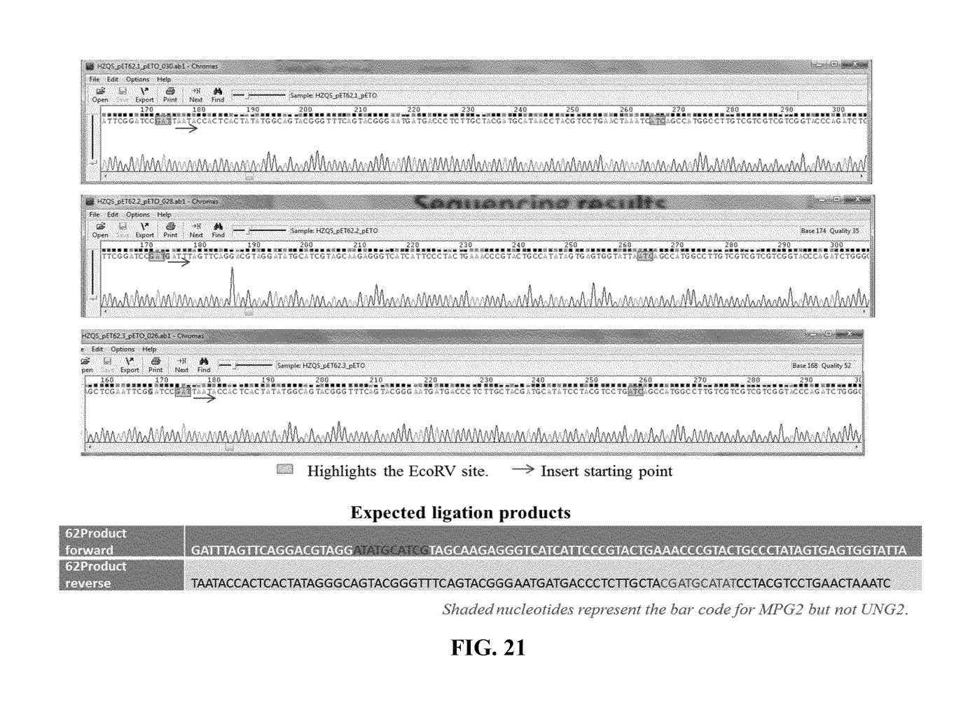

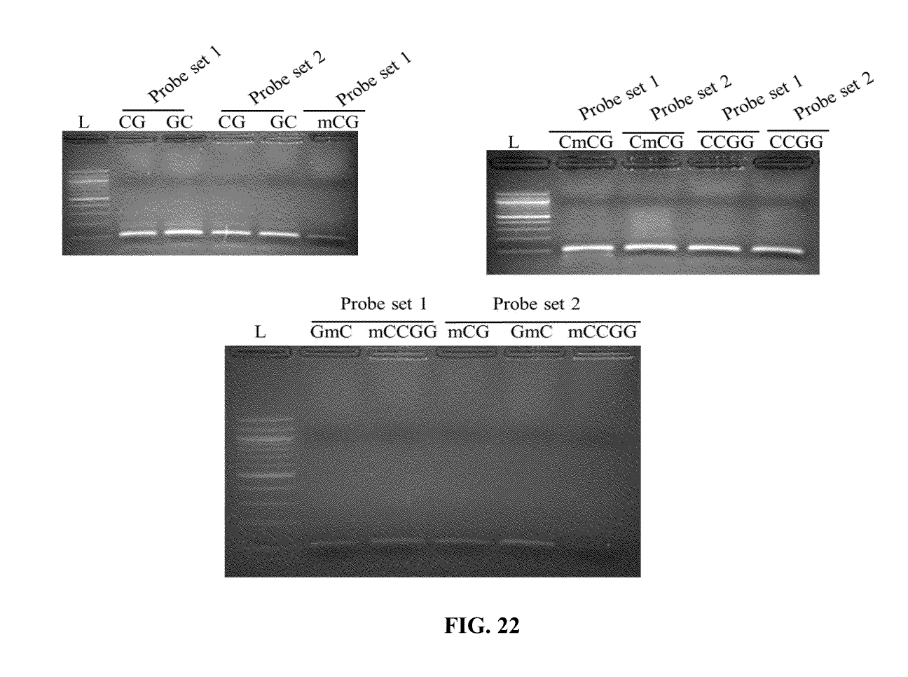

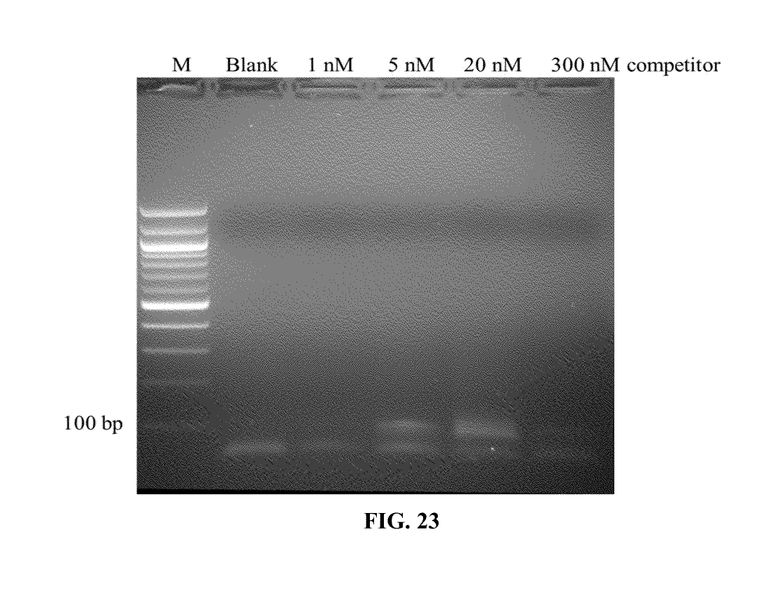

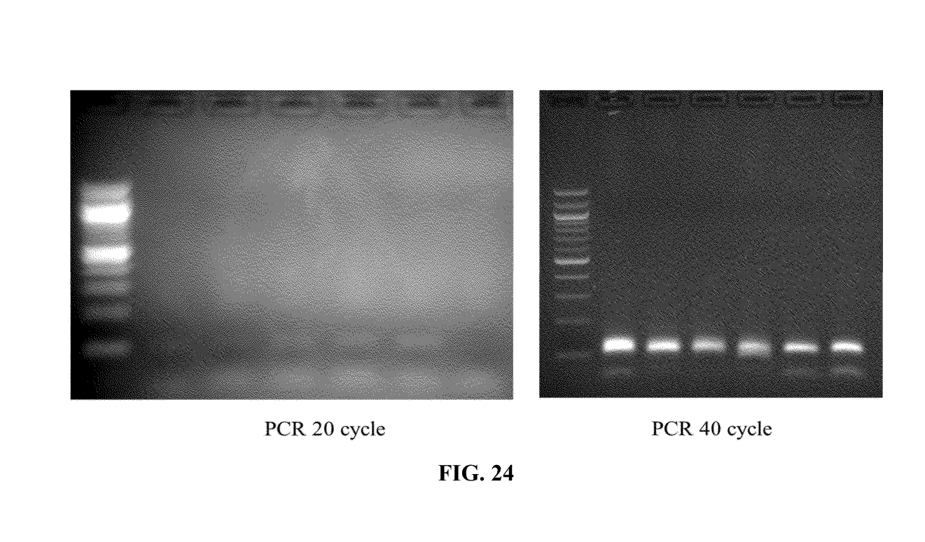

As shown in FIG. 20, when a particular DNA motif (e.g., M62) was used on a protein microarray comprised of two human TF proteins and BSA, the expected ligation products were successfully recovered using a PCR reaction only from the expected TF spot, but not from the other TF and BSA spots. After cloning the PCR fragments, Sanger sequencing was performed to determine the PCR-amplified sequences. Six of six sequenced clones gave the expected sequences from the ligation on the array. One example is shown in FIG. 21.

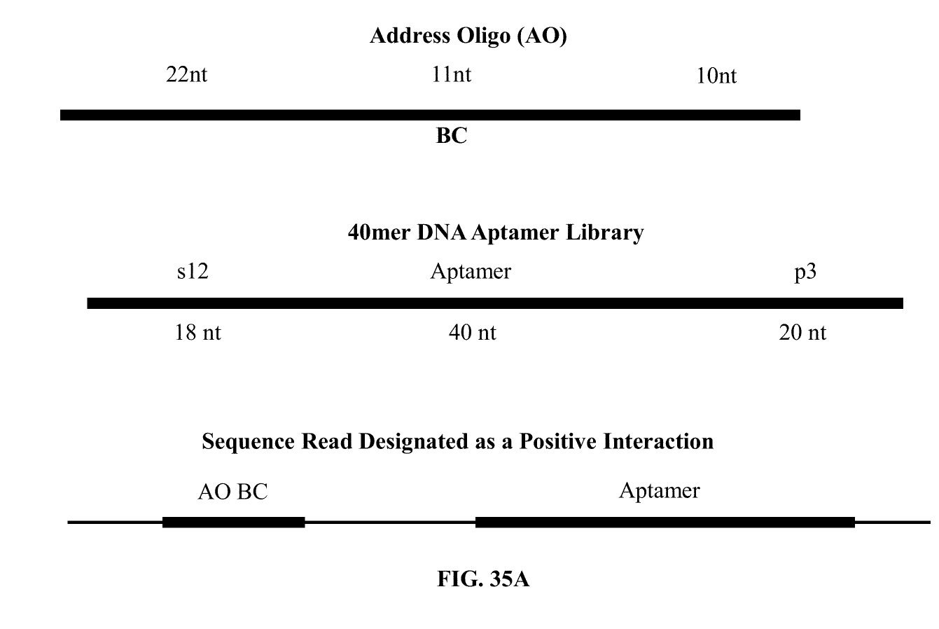

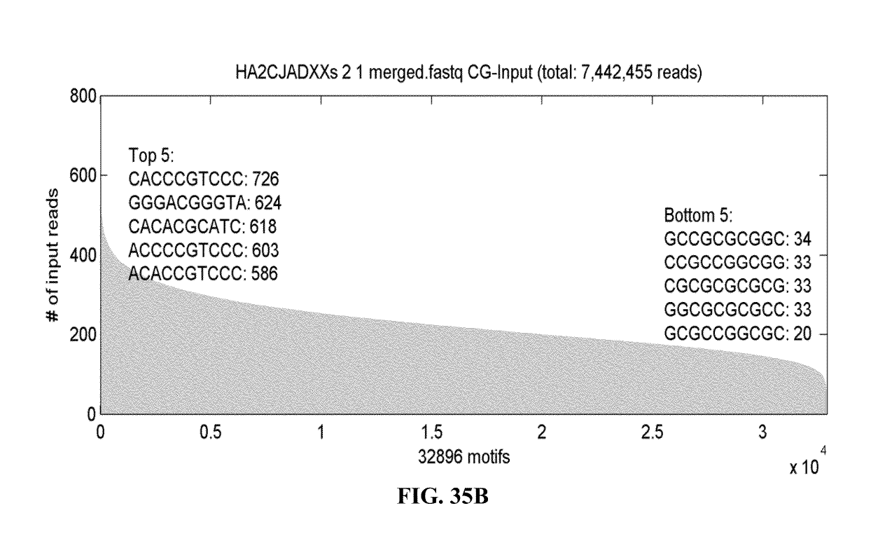

Address oligos and DNA aptamer libraries were also utilized in a DAPPL approach to screen for protein-DNA interactions using a 40 mer DNA aptamer library as shown in FIG. 35A. Address oligos were designed such that there were at least 3 nt differences between all other address oligos and were designed to avoid self-annealing. A total of 22,168 address oligos were designed and screen against 1,632 TF proteins. For some of these experiments, address oligos that differed by at least 4 nts were utilized. The initial complexity of the DNA aptamer library was about 1×10¹⁹. A computer algorithm was designed to extract sequences with an address oligo (AO) and a 40-mer DNA aptamer sequence with fixed regions as shown in FIG. 35A (bottom).

### Example 21

### Global Mapping of Interactions Between DNaseI Hypersensitive Sites (DHS) and Nuclear Proteins

Global mapping of genome interactions revealed a complex 3-D architecture of the nucleus, which is also subjected to dynamic reorganization upon changes in the microenvironments, such as matrix-compliance. Although recent technology advances, such as Hi-C, have revealed most interactions happen between enhancers in the open chromatins, the molecular mechanism of maintenance and reorganization of the nuclear 3-D architecture remains largely unknown. An attractive hypothesis is that nuclear proteins, such transcription factors and co-factors (e.g., CTCF), play a crucial role in 3-D architecture maintenance and dynamics. However, no high-throughput assays or methods have been reported to globally identify the protein component involved in the formation of the 3-D architecture. To remedy this technology hurdle, the methods described herein are utilized for highly multiplex screens for protein-DNA interactions between DNaseI hypersensitive sites (DHS) and nuclear proteins.

Enhancers are highly enriched in DHSs. DHS-nuclear protein interactions are comprehensively profiled using a DAPPL approach. To capture dynamic changes of the 4-D nucleome, different DHS pools are obtained during a time course of matrix-compliance-induced morphological changes of mouse embryonic stem (ES) cells. Each DHS pool is separated in an ultra-centrifuge in order to recover DHS species around 150 bps. The recovered DHSs are end-fixed and ligated to a Y-shaped adapter DNA. Each DHS pool is incubated on a human protein microarray containing ˜4,200 nuclear proteins each spotted with a unique address polynucleotide. After washing, a splint polynucleotide is added to the array that anneals to the constant region at one end of the address oligo and to the single-stranded sequence of the adapter. After ligation reaction on the array, the ligated DNA is recovered, PCR-amplified, and deep-sequenced using Hi-Seq. Bioinformatics analysis of the sequences is used to determine which DHS sequence is recognized by which nuclear protein(s). The resulting DHS-protein interaction networks obtained at different time points in the process of matrix-compliance-induced morphological changes is compiled together, and global DHS-protein interaction network with a temporal resolution is generated. Selected predictions (e.g., important TF protein candidates) made from these networks are examined using traditional methodologies.

### Example 22

### Global Mapping of Interactions Between Nucleosomes and Nuclear Proteins

A similar DAPPL approach is applied to comprehensively profile interactions between nuclear proteins and individual DNA-bound nucleosomes in open chromatin obtained in a similar time course described above in Example 21.

To capture dynamic changes of the 4-D nucleome, different nucleosome pools are obtained during a time course of matrix-compliance-induced morphological changes of mouse ES cells. Each nucleosome pool is separated to recover nucleosome species. After coupling a proximity polynucleotide is coupled to the nucleosomes, each nucleosome pool is incubated on a human protein microarray containing ˜4,200 nuclear proteins each spotted with a unique address polynucleotide. After washing, a splint polynucleotide is added to the array that anneals to the constant region at one end of the address oligo and to the single-stranded sequence of the adapter. After ligation reaction on the array, the ligated DNA is recovered, PCR-amplified, and deep-sequenced using Hi-seq. Bioinformatics analysis of the sequences is used to determine which nucleosomes interact with which nuclear protein(s). The resulting nucleosome-protein interaction networks obtained at different time points in the process of matrix-compliance-induced morphological changes is compiled together, and global nucleosome-protein interaction network with a temporal resolution is generated. Selected predictions made from these networks are examined using traditional methodologies.

### Example 23

### Protein-RNA Interactions

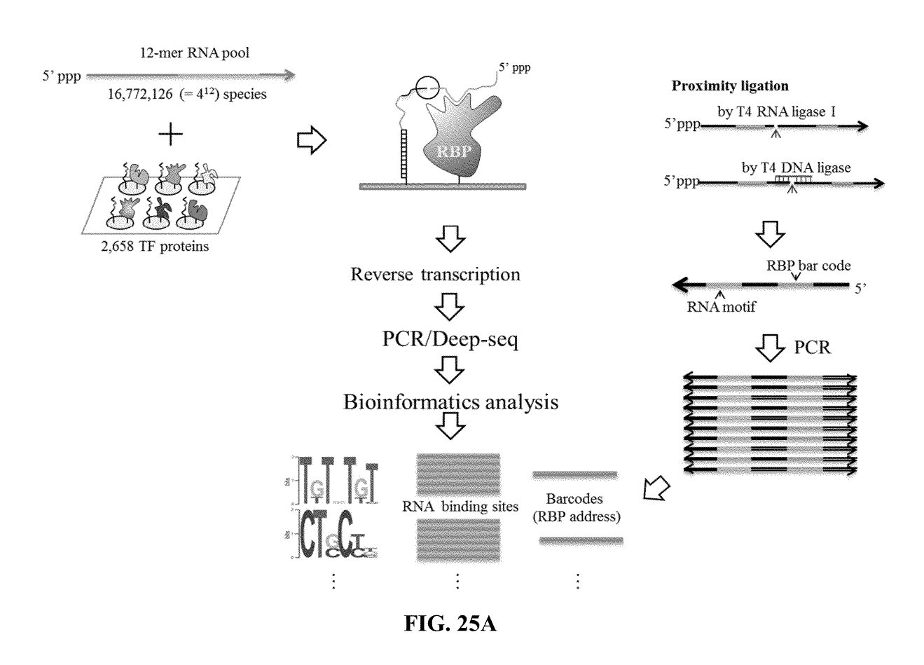

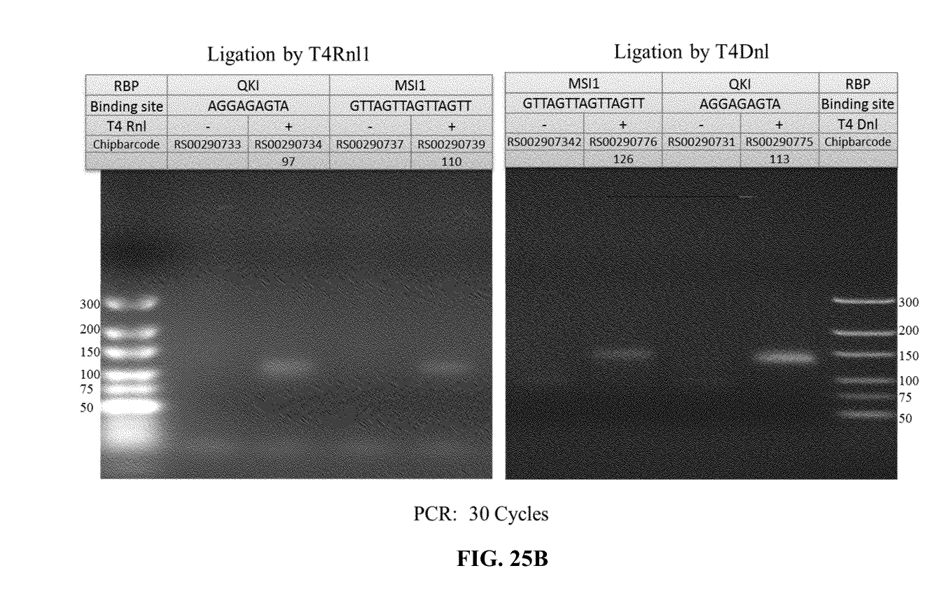

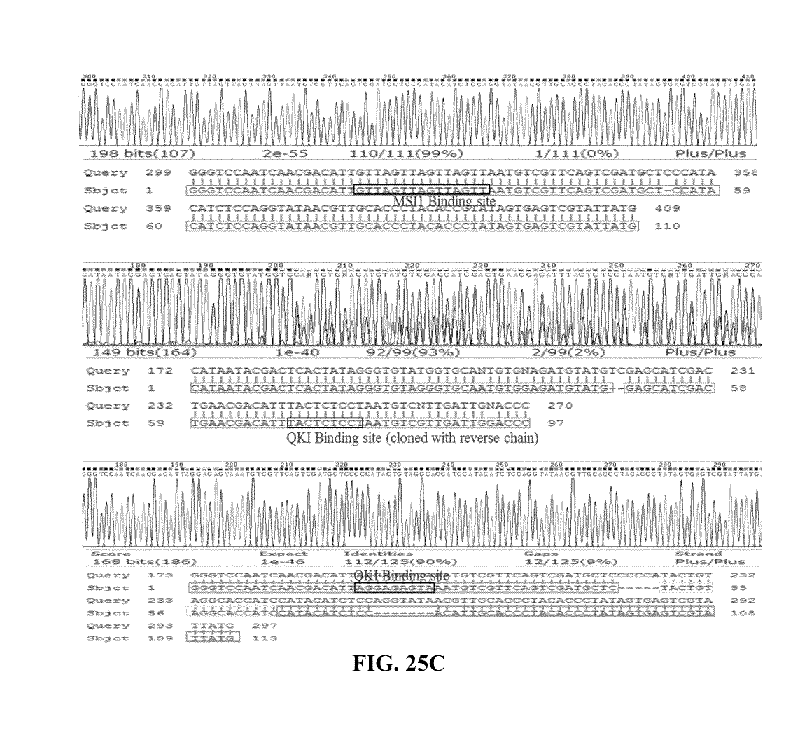

A comprehensive screening was performed with random 12-mer RNA sequences on a protein microarray, comprised of ˜1,600 TF and ˜1,000 annotated RNA-binding proteins. A DNA polynucleotide pool was synthesized that each contains a 12-mer random sequence with a T7 sequence and a fixed sequence to its 5′- and 3′-ends. After ds-DNA templates were created, in vitro transcription was performed to generate the RNA molecules with a complexity of ˜16 million. This mixture of RNAs was incubated on the protein microarray and, after stringent washes, the captured RNA molecules were ligated to the free 5′-end of the address polynucleotide with the T4 DNA ligase, which can ligate single-stranded DNA and RNA fragments (FIG. 25A). Next, a reverse transcription was performed to generate the complementary DNA strand of the ligated DNA-RNA fragments, followed by PCR-amplification with a primer pair that adds back the T7 primer sequences to the ds-DNA templates. The recovered DNA templates were subjected to the same screening process for 5 more cycles and the ligated products from cycles 4 to 6 were deep-sequenced. Similar bioinformatics analyses were performed to determine whether statistically significant consensus RNA motifs were obtained for the RNA-binding proteins.

As shown in FIG. 25B, the recovered ligation products between the RNA probe and the address polynucleotides from two RNA-binding proteins, MSI1 and QK1PCR amplification, could be readily PCR amplified as demonstrated by probing separately for their known RNA sequences. Sanger sequencing confirmed the expected ligation sequences (FIG. 25C).

### Example 24

### PTM-Omix Approach to Globally Quantify Posttranslationally Modified (PTM) Human Proteome in Cells and Tissues

Identified DNA/RNA aptamers can be used to enable proteome-wide detection of posttranslationally modified human proteome inside a cell or tissue. Total protein lysates are obtained using standard protocols from cultured cell lines or primary tissues and are lightly biotinylated. A mixture of hundreds to thousands of aptamers that each specifically recognized a PTM-modified proteins are mixed and added to the lysates. After incubation at RT, the aptamer-protein complexes will be purified using streptavidin beads, followed by stringent washes. After recovery of the aptamers captured by proteins, they are PCR-amplified and deep-sequenced. The number of reads per aptamer serves as the proxy for the relative abundance of the PTM-modified proteins. As a positive control, a foreign protein (e.g., e.g., GFP) with a V5-tag is spiked into the lysate at a known concentration (e.g., 1 nM). An aptamer recognizing V5 epitope is included in the aptamer mixture to serve as a normalization control.

### Example 25

### Aptamer-Based Perturbation in Cells and Tissues

RNA aptamers that each encodes specific inhibition activity against a particular enzyme (e.g., protein kinases, phosphatases, (de)acetyltransferases, deubiquitinases, etc.) are cloned into an inducible mammalian expression constructed and transfected to human cell lines. Upon induction, the encoded RNA aptamers are expressed and targeted to their corresponding enzyme target, and result in inhibition of the enzyme activity. Each DNA/RNA aptamer with specific inhibition activity against a particular enzyme is packaged into viruses and transfected into human cell lines or tissues to inhibit one or many enzymes of interest.

### Example 26

### Aptamer-Based Scaffolds to Dictate Protein-Protein and Enzyme-Substrate Interactions in Cells and Tissues

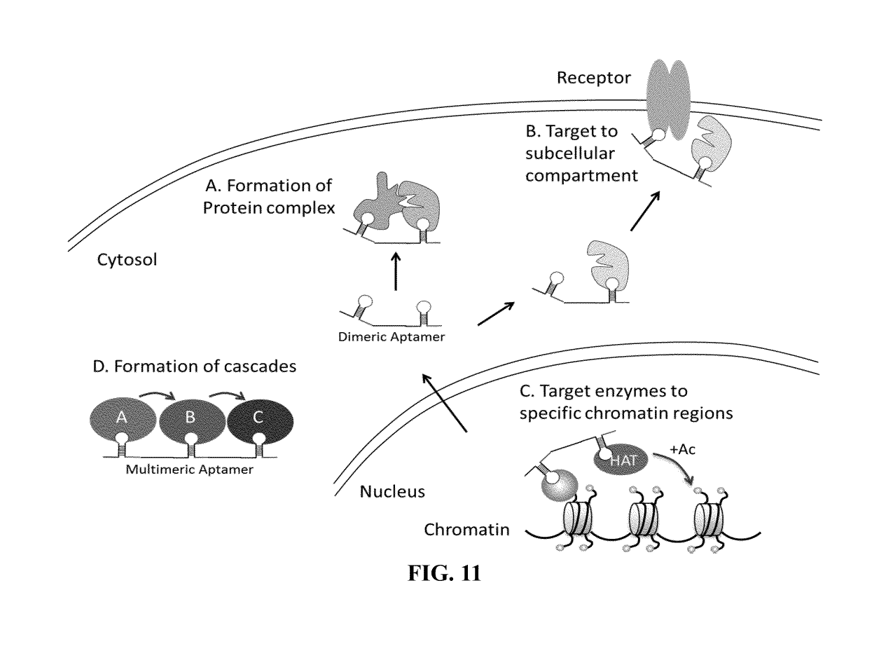

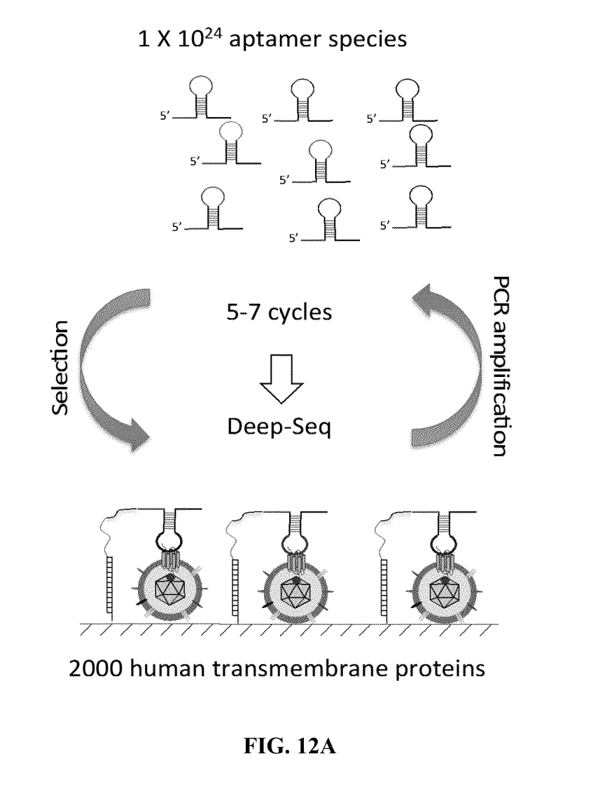

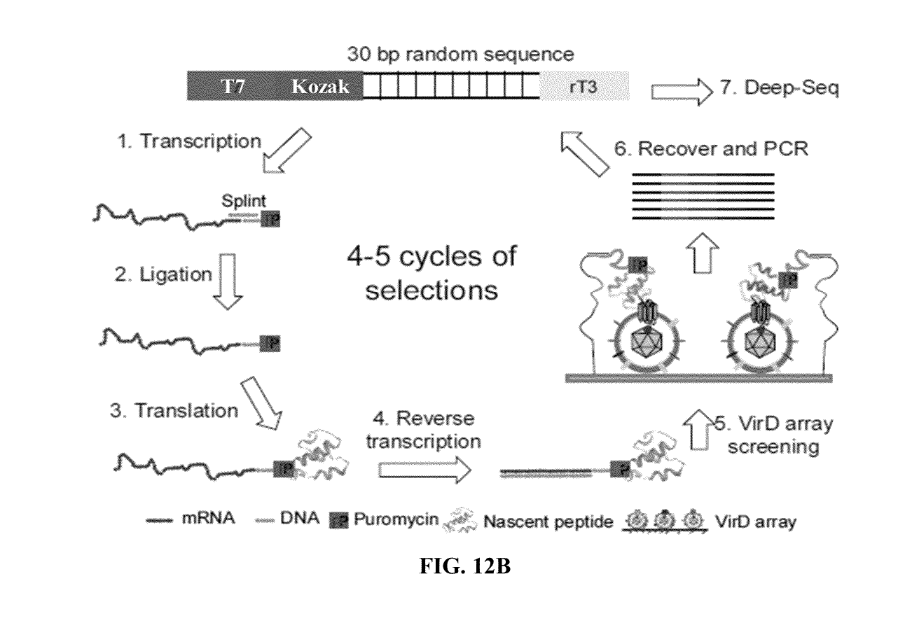

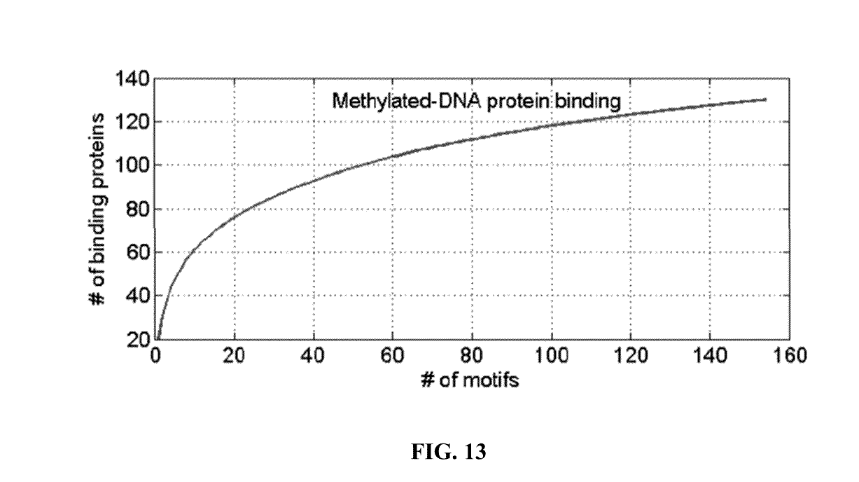

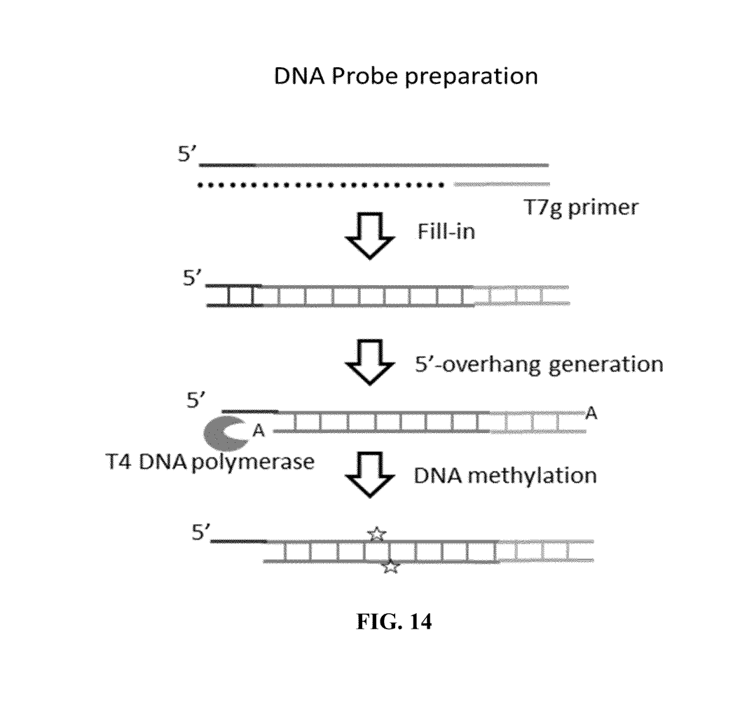

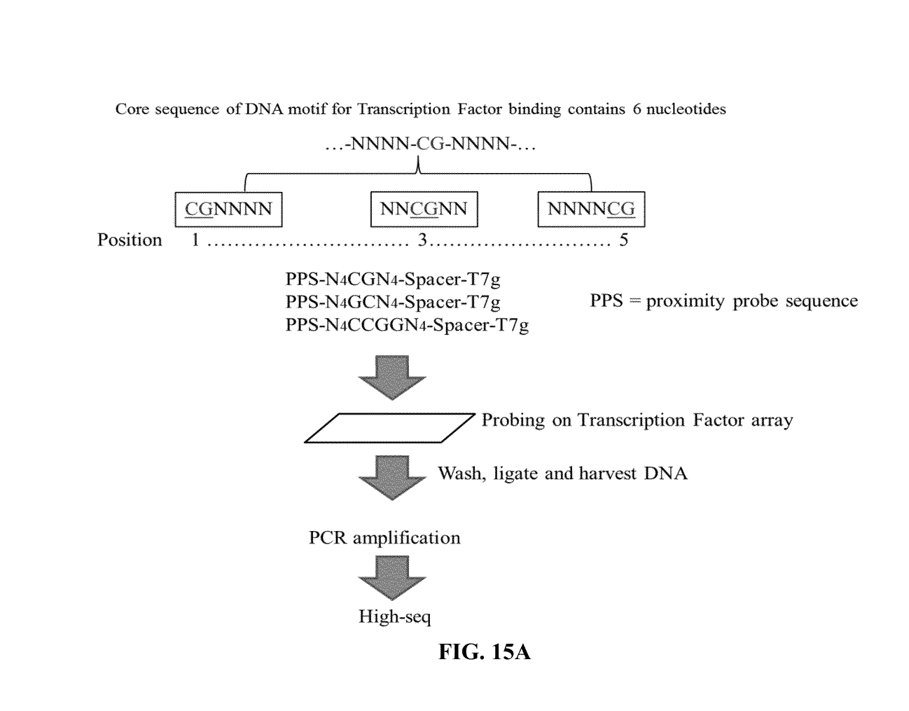

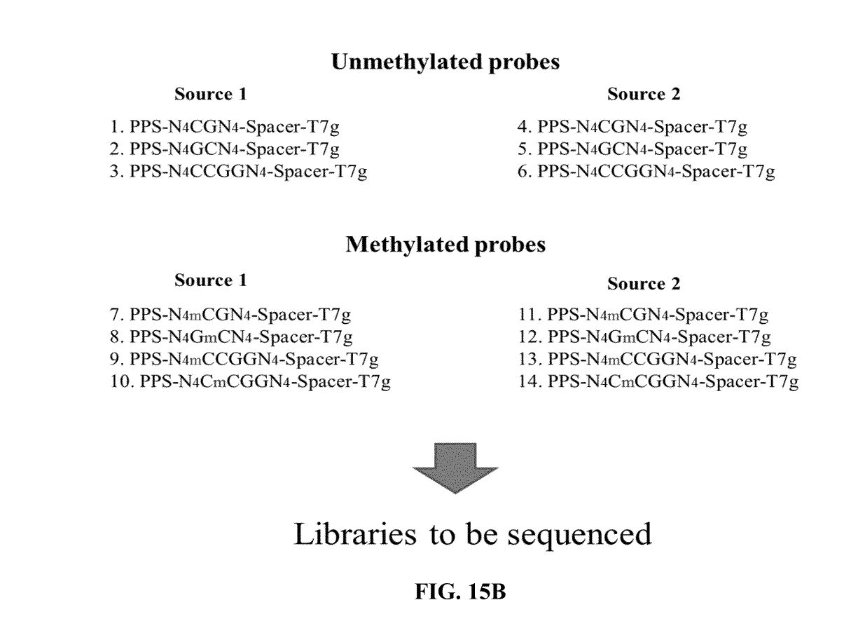

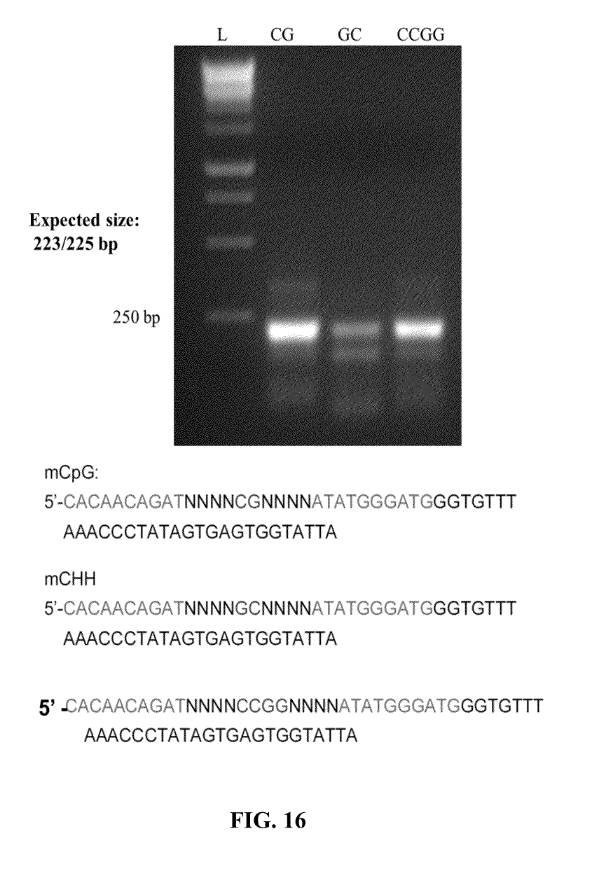

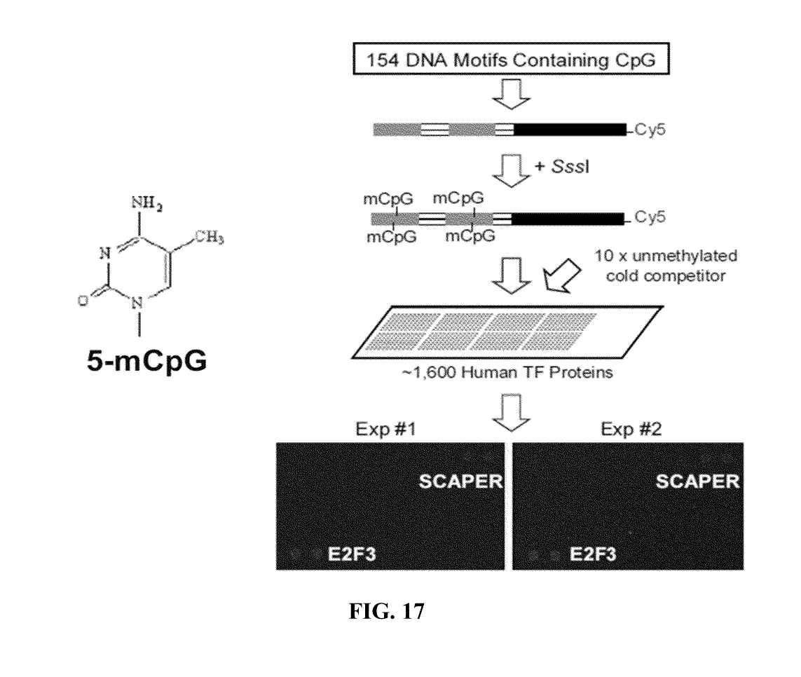

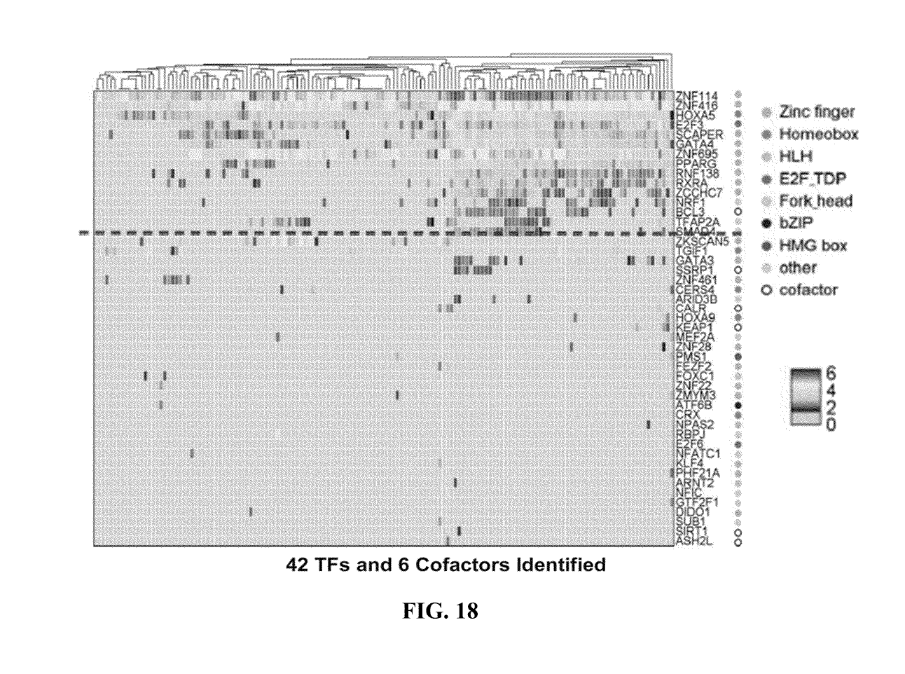

Two aptamers (either DNA or RNA) that each specifically recognizes a particular human protein are connected via a polynucleotide link as a single molecule to create a dimeric or multimeric aptamer scaffold, a tailor-made molecular scaffold that can be used to dictate formation of protein homo- or heterodimers. When expressed in cells or tissue via induction or transient transfection, the aptamer scaffold brings the two desired proteins into proximity and facilitates homo- or heterodimeric protein complex formation or promotes enzyme-substrate interactions (FIG. 11). When one of the aptamers of the dimeric or multimeric aptamer scaffold targets to a particular subcellular compartment via specific interactions with a protein that is known to localize to that location, such an aptamer scaffold can act as a molecular chaperon to deliver its cargo, namely the protein that interacts with the other aptamer of the dimeric aptamer scaffold, to the desired subcellular compartment, such as the mitochondria, Golgi, lipid rafts, to name a few (FIG. 11). Furthermore, when one of the aptamers recognizes a given chromatin-associated protein, such as transcription factors, co-factors, cohesins, etc., this aptamer scaffold can be used to deliver chromatin modification enzymes, such as histone acetylases, deacetylases, methylases, demethylases, and DNA methylases, to particular chromatin regions and modify the local histones or DNAs (FIG. 11). When 2 or more aptamers of a dimeric or multimeric aptamer scaffold aptamers encode specific binding activities and are connected together to form a multimeric scaffold, a desired protein complex is dictated to form inside a cell (FIG. 11). For example, when a group of metabolic enzymes is brought together in the order of the metabolism cascade via this type of multimeric aptamer, it may greatly enhance the production of the desired metabolites. In another example, a series of protein kinases in the same signaling cascade, such as MAP kinases, are brought together to form a multi-member kinase sink that may greatly amplify the phosphorylation signals in cells. In this case, the multimeric aptamer serves as a signaling dock to facilitate signal transduction.

### Example 27

### Aptamer-Based Biomarker Identification

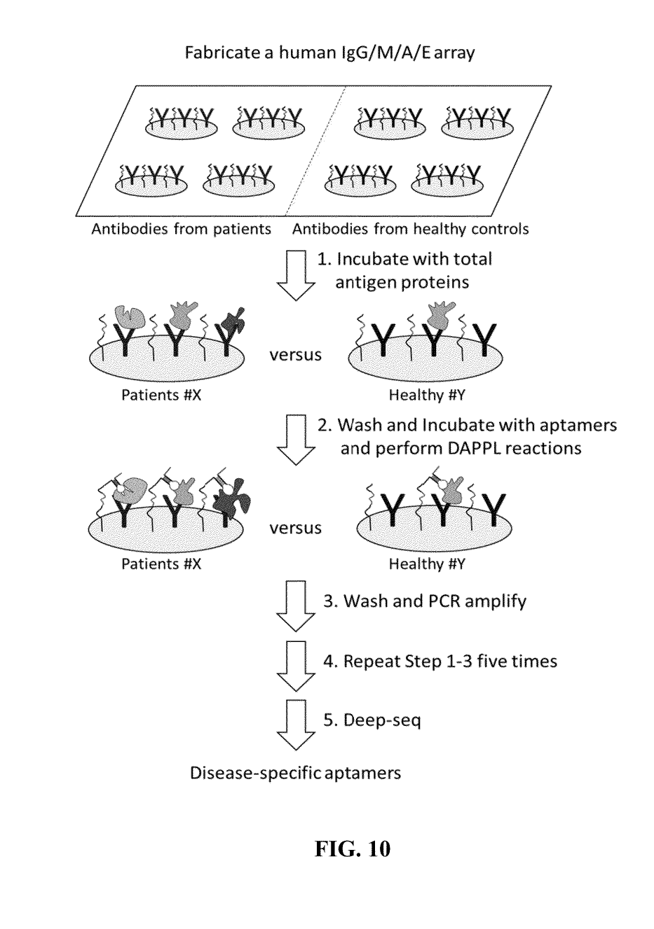

A DAPPL approach is applied to identify aptamers that can distinguish patients from healthy subjects. Briefly, immunoglobulins, such as IgG/IgM/IgA/IgE, are isolated from serum samples collected from a cohort of patients and healthy controls (e.g., >30 subject in each category), using Protein A/G or L conjugated beads. After a stringent wash step to remove non-specific proteins, the captured immunoglobulins are eluted at low pH (e.g., glycine-HCl, pH 2). Each immunoglobulin mixture of a particular subject is mixed with a unique address polynucleotide and spotted multiple times to form an autoantibody array (FIG. 10). When the goal is to identify biomarkers for autoimmune diseases, such as RA, PBC, SLE, etc., total protein lysates isolated from either human cell lines or relative tissues will be incubated on the array at RT for 1-3 hr, followed by stringent washes (FIG. 10).

Meanwhile, a mixture of DNA/RNA aptamers with fixed sequences flanking the variable regions (e.g., 20-60 mer in length) will be pre-incubated with a mixture of commercial human IgG/IgM/IgA/IgE at RT for 1 hr, followed by adding Protein A/G or L conjugated beads to deplete those aptamers that can directly recognize these immunoglobulin. The depleted aptamer pool are added to the array and incubated in the presence of a mixture of human IgG/IgM/IgA/IgE for 1-3 hr at RT, in order to further eliminate aptamers that can recognize human immunoglobulin (FIG. 10). This step allows formation of a sandwich-like complex, comprised of autoantibodies immobilized on the surface, captured antigens in the middle, and aptamers on top. After stringent washes (e.g., 300 mM NaCl with 0.1% Tween 100 at pH 7), the captured aptamers are recovered and PCR-amplified. Using the PCR products as templates, the recovered aptamers are regenerated and probed to the same autoantibody array. The same procedure can be repeated for at least 4-6 times. At the ends of cycles 4-6, DAPPL reactions are carried out to ligate address polynucleotides to the captured aptamer sequences and the products are deep-sequenced. In each sequence read, the address polynucleotide represents the identity of the patient or healthy subject tested; whereas the aptamer sequence(s) ligated to the address polynucleotide indicates the autoantigen(s) that it recognizes. Because in each sequence read an aptamer sequence to ligated to an address polynucleotide, representing a subject identity (e.g., an IBD patient or healthy subject), its sensitivity and specificity are readily known. Follow-up bioinformatics analysis identifies those aptamers that are statistically enriched in the patient cohort and they are re-tested against an additional cohort for validation purposes.

For human autoimmune diseases, such as RA, AIH, PBC, SLE, etc., each identified aptamer is individually synthesized, pooled, and probed to the HuProt® array. After ligation between the captured aptamers and the address polynucleotides takes place on the HuProt® array, the ligation products are recovered, PCR-amplified, and deep-sequenced. Alternatively, the identity of captured aptamers can be revealed by hybridiuzing recovered aptamer, end-labeled with a fluorphore (e.g., Cy5), to a DNA polynucleotide array that encodes the complementary sequences of the entire aptamer set used in this assay. In the case that a given aptamer fails to recognize any protein on the HuProt® array, presumably due to a lack of proper protein posttranslational modifications, this aptamer is resynthesized with an affinity tag (e.g., biotin) used to pull down protein(s) directly from total proteins extracted from cell lines or tissues. The identity of the captured proteins can be revealed by mass spectrometry (e.g., MS/MS). In the case of identification of cancer antigens, total proteins extracted from tumors of cancer patients are used to incubate with the autoantibody arrays instead of total protein lysates.

For diseases caused by microbiome in the guts, such as inflammatory bowel disease (IBD), the mixture of total proteins extracted from the microbiome of an IBD patient cohort is incubated on the autoantibody arrays, comprised of purified IgG/IgM/IgA/IgE from IBD patients and healthy controls. To identify the antigen identity, the identified disease-specific aptamer(s) is synthesized with an affinity tag (e.g., biotin) and used to pull down the candidate antigens from the total proteins extracted from the microbiome of IBD patients. The identity of the antigens can be revealed by mass spectrometry (e.g., MS/MS). A fraction of immunoglobulin in IBD patients may tightly bind to human proteins (e.g., autoantigens) and therefore, some aptamers may unavoidably recognize these human autoantigens. Because these autoantigens are also valuable in IBD diagnosis or prognosis, all the identified aptamers are probed to the HuProt® arrays to determine the autoantigens using the same approach as described above.

### Example 28

### Alternative Aptamer-Protein Screening Methodology

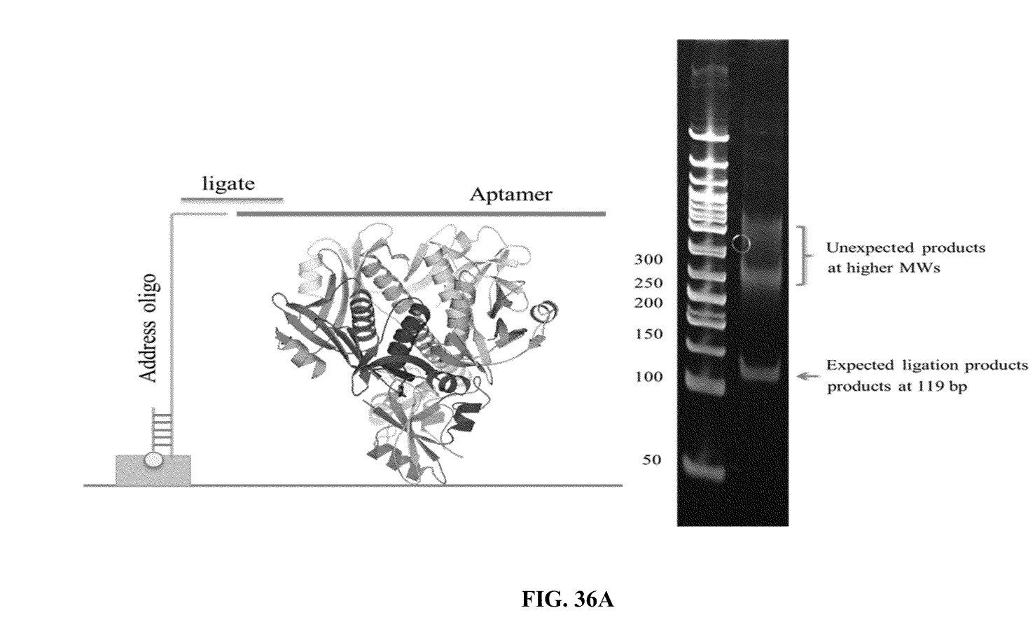

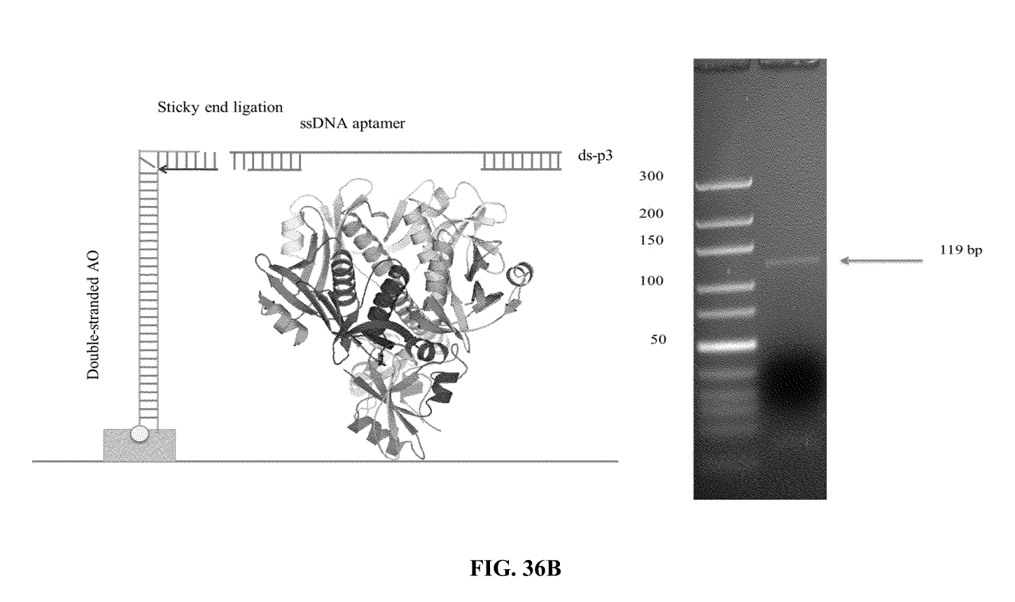

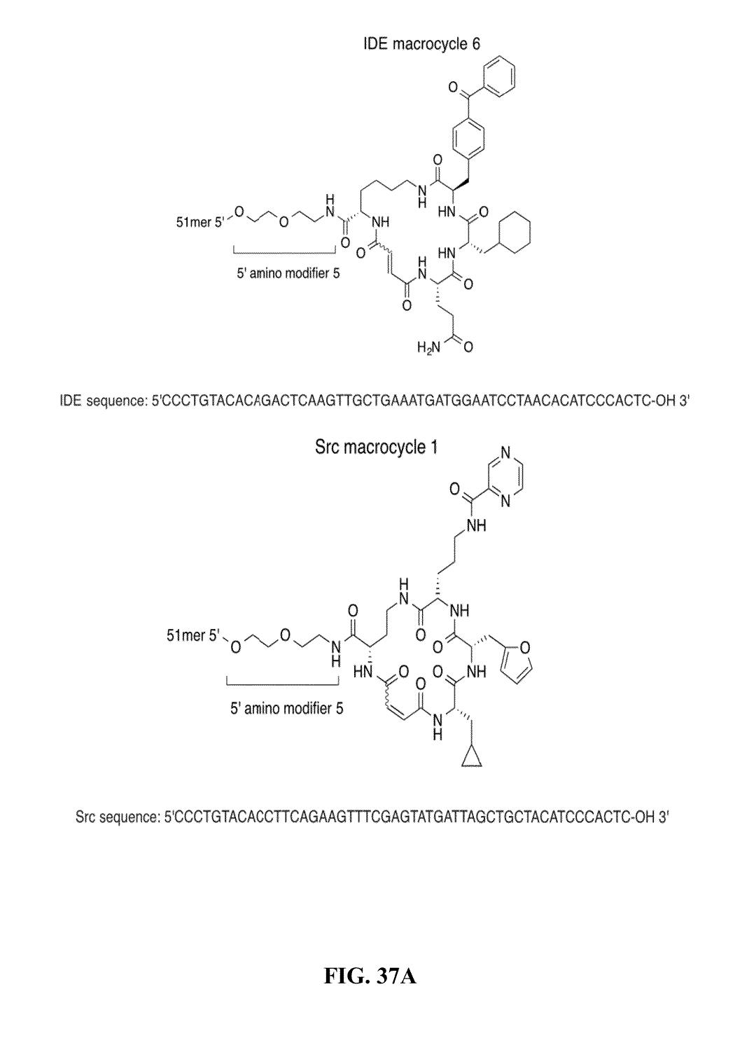

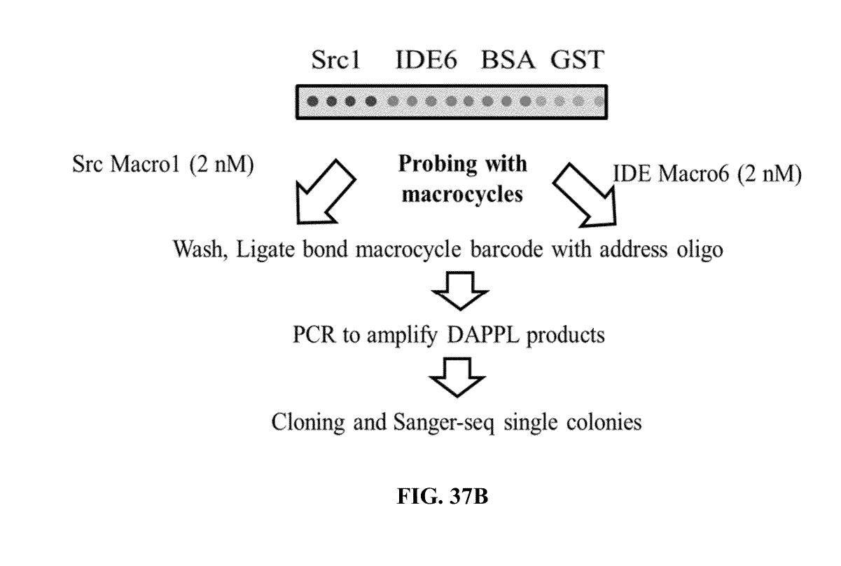

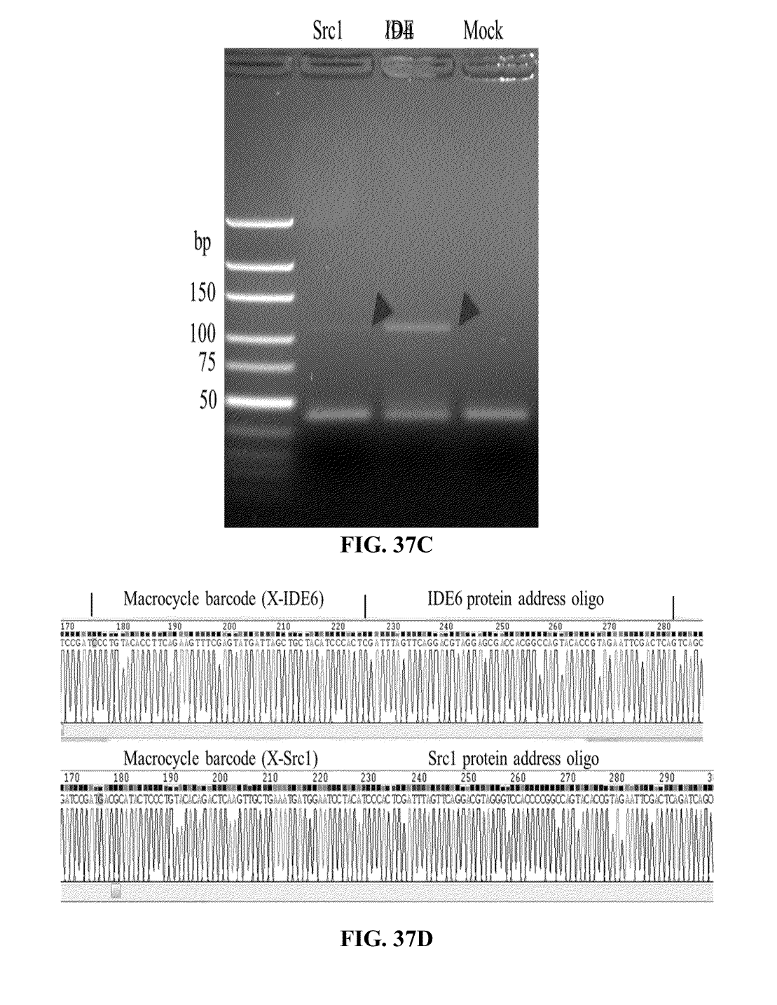

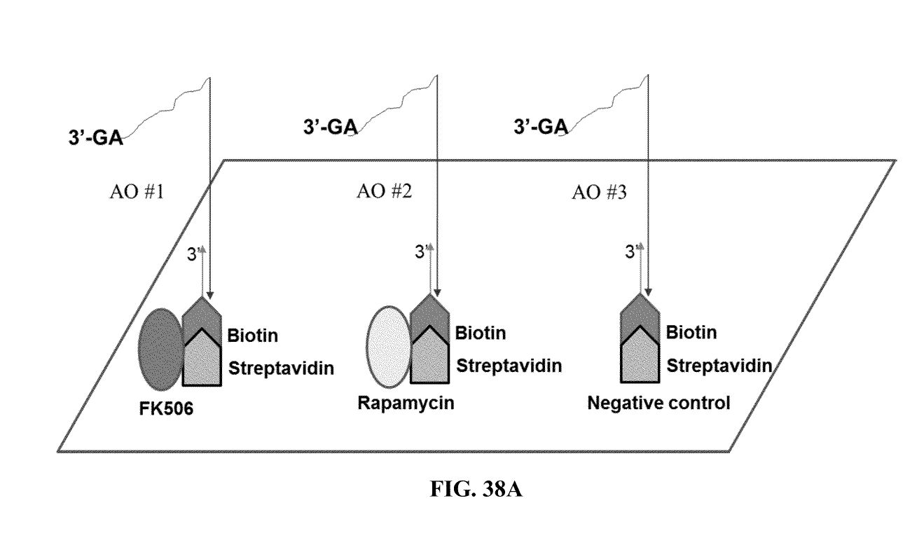

Address oligos were first converted to double-stranded polynucleotides using Klenow polymerase (3′→5′ exo⁻) (an N-terminal truncation of DNA Polymerase I which retains polymerase activity, but has lost 5′→3′ exonuclease activity and has mutations (D355A, E357A) which abolish 3′→5′ exonuclease activity). The Klenow polymerase was added with a primer (FIG. 36B) that anneals to the free end of the address polynucleotide, which left a 5′ sticky end. Two sequence-specific primers were annealed to the 5′- and 3′-fixed regions of the DNA aptamer library (FIG. 36B), generating another 3′-sticky end. After washes, a captured aptamer was ligated to the address oligonucleotide via sticky end ligation. Amplification and sequencing was then performed. Thus, in some embodiments, the above described methodology may be preferred over the methodology depicted in (FIG. 36A) which uses a splint oligo, rather than sticky ends, for ligation.

### Example 29

### Antibody-Protein and Protein-Protein Screens

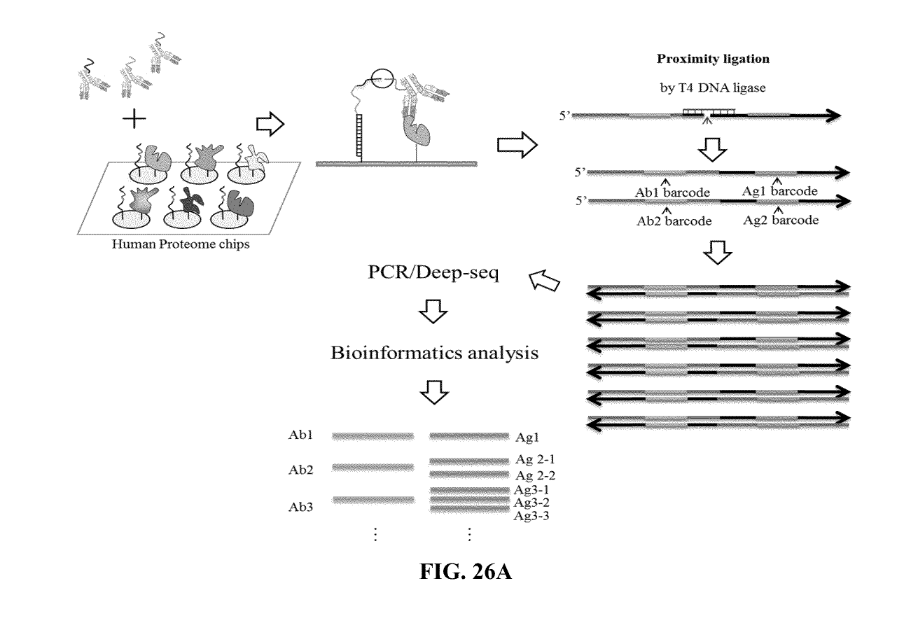

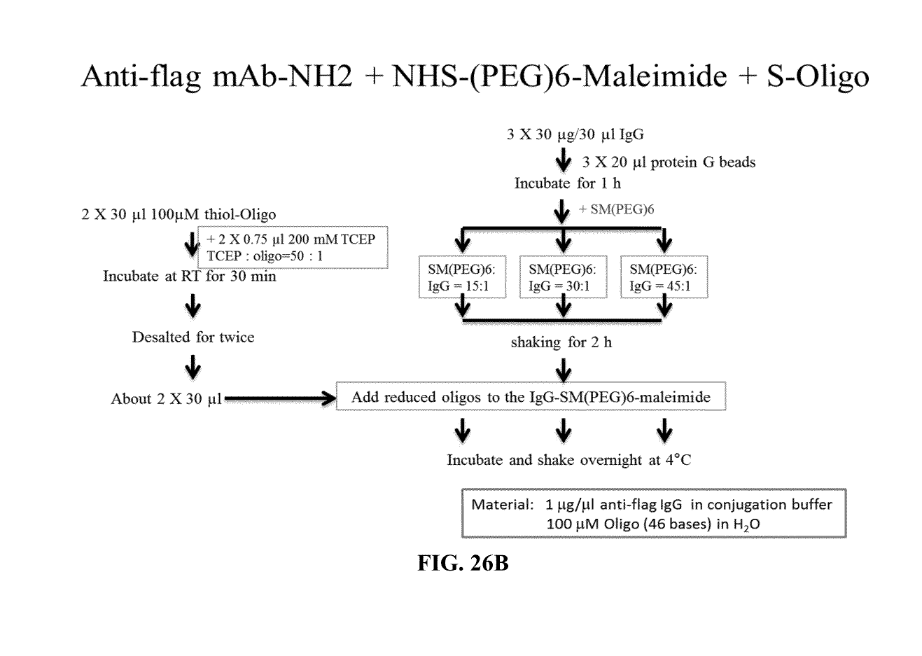

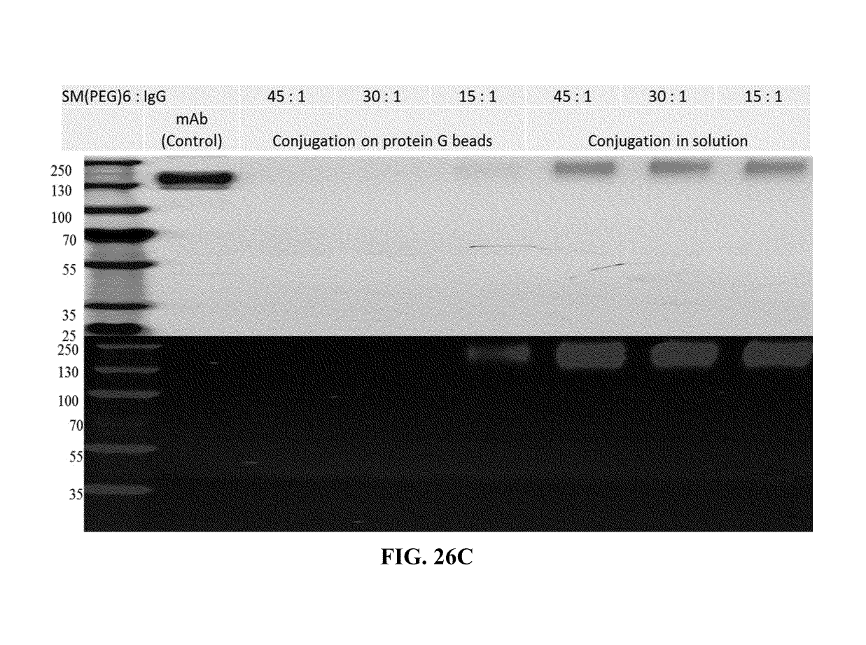

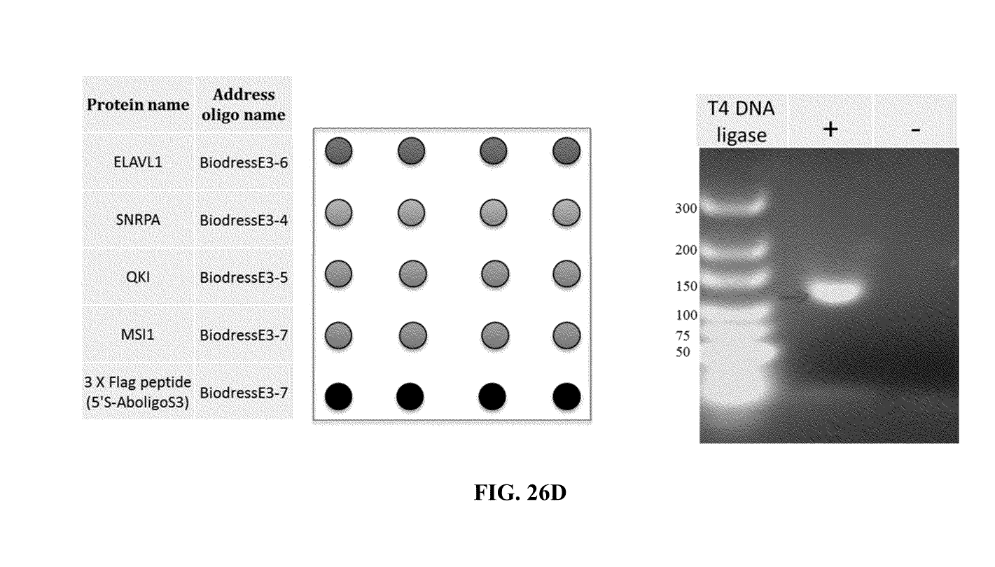

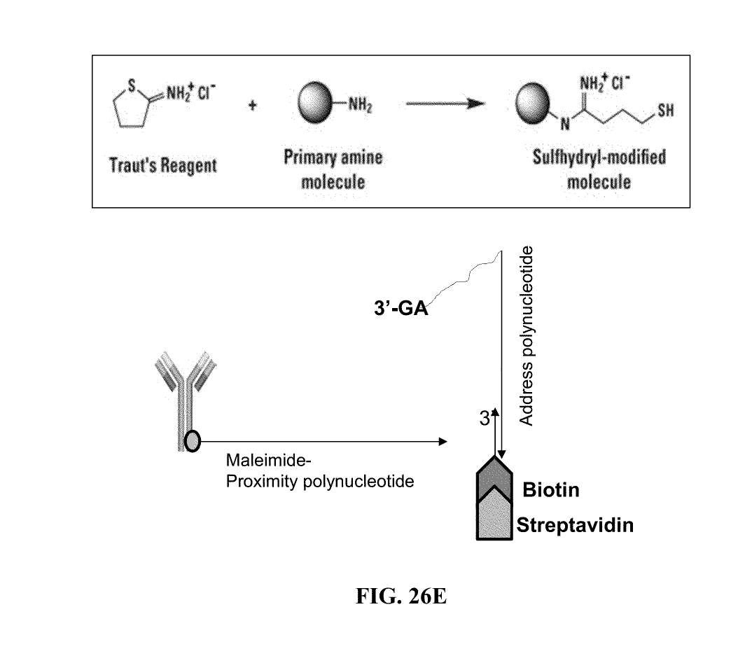

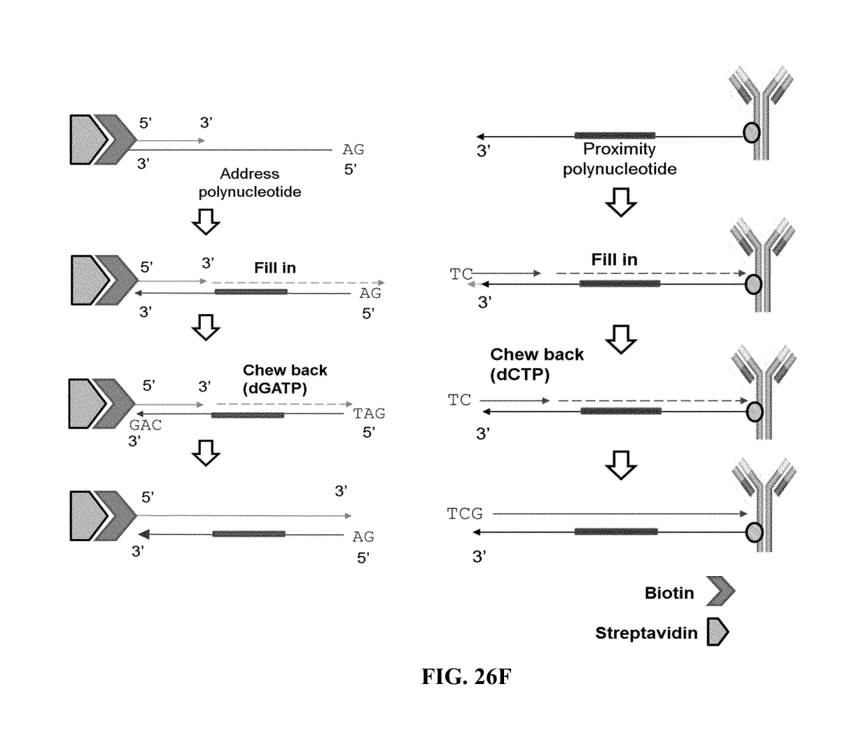

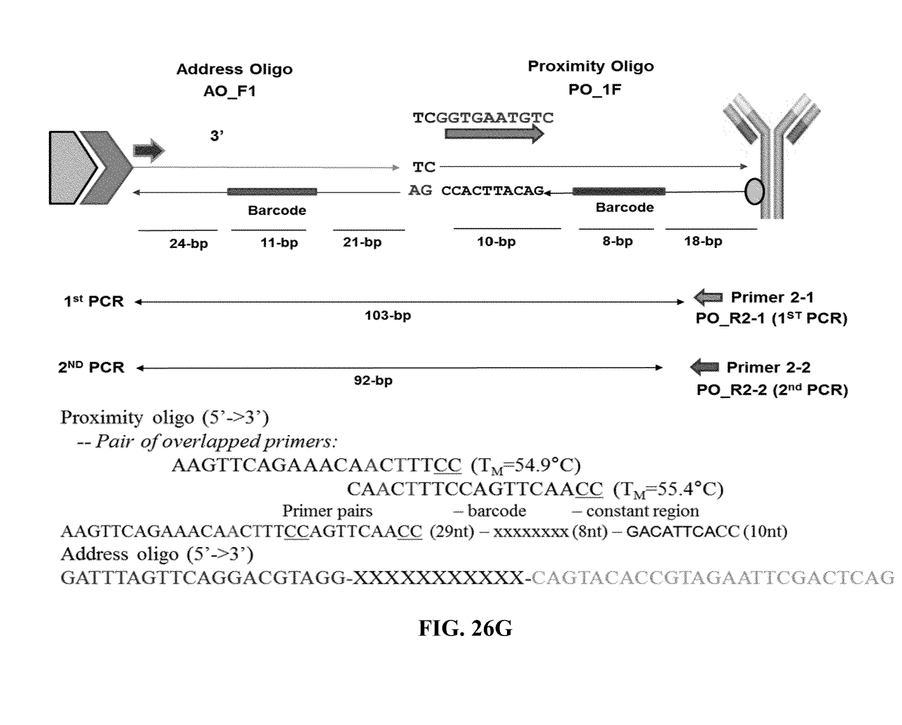

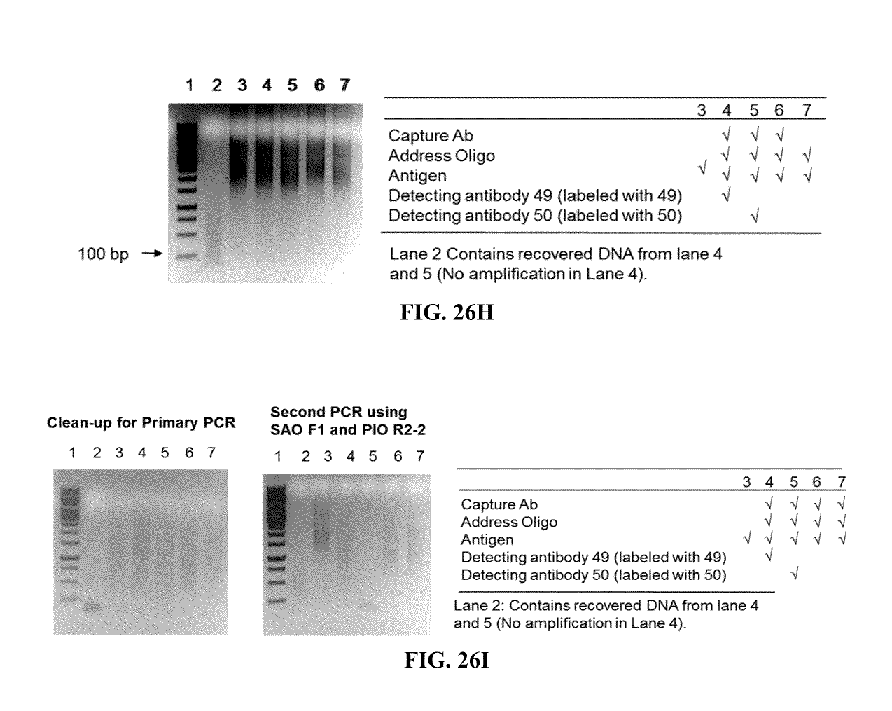

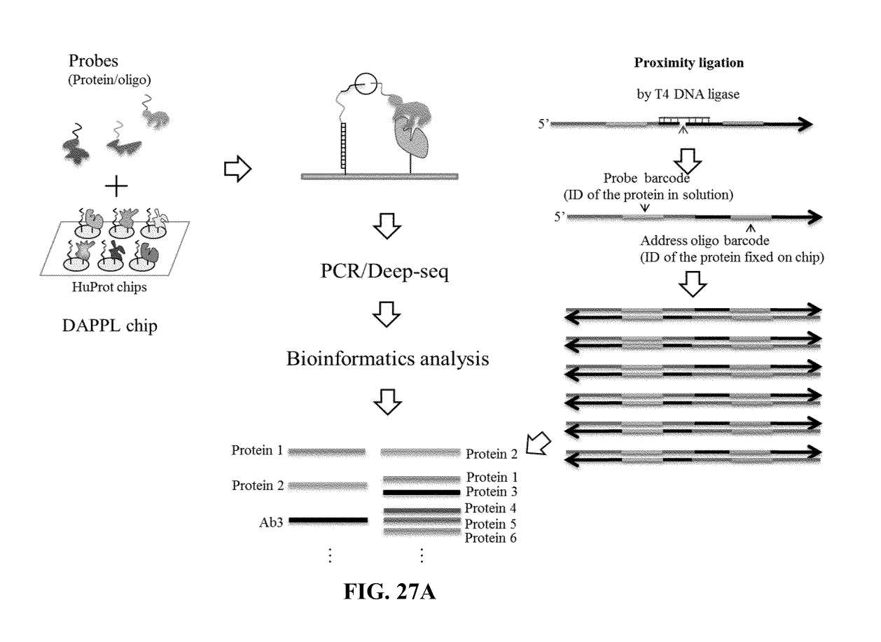

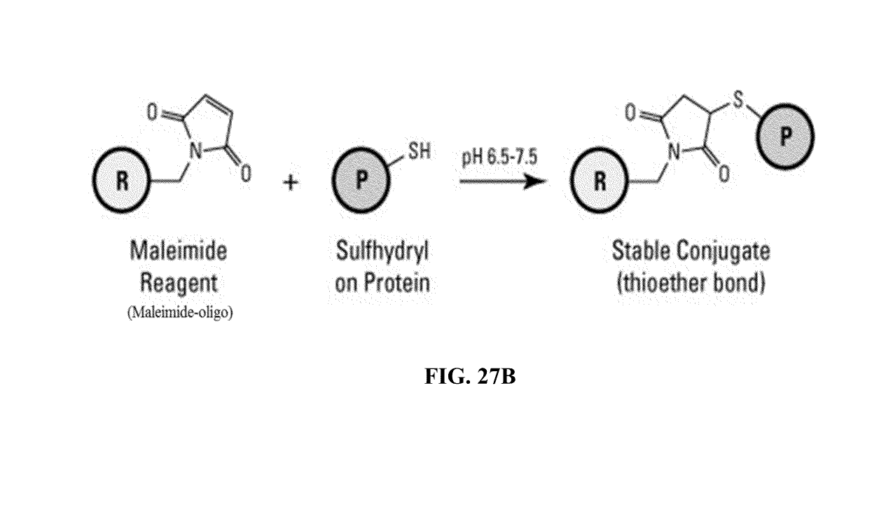

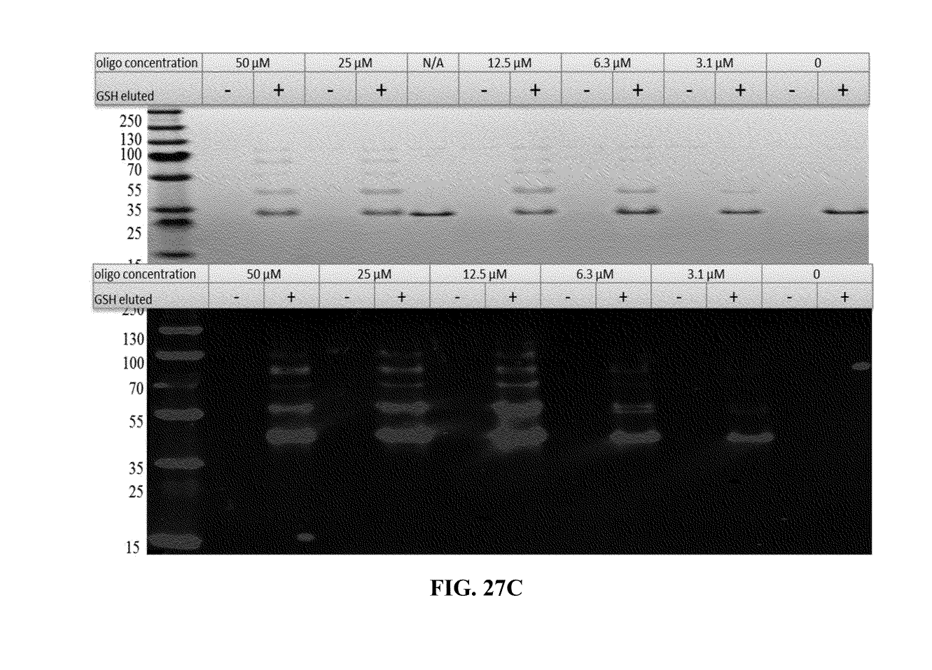

A DAPPL approach is applied to identify high affinity antibodies (FIG. 26A) or protein (FIG. 27A) binding partners that interact with protein targets of interest. Briefly, an ELISA plate was prepared by coating the surface of a 384 well-PCR plate with a 0.01% poly-L-Lysine solution and incubating for 20 min. The plate was then rinsed with water and allowed to dry. 10 μl of a biotinylated address oligo mixture was mixed with streptavidin (1.7 μM) and capture antibody (500 ng) and incubated for 1 hr (FIG. 26E, bottom). The Klenow polymerase was added with a primer (FIG. 26F, left panel, right to left arrow; and FIG. 26G) that anneals to the free end of the address polynucleotide (FIG. 26F, right panel; and FIG. 26G), which left a 5′ sticky end. Two sequence-specific primers were annealed to the 5′- and 3′-fixed regions of the DNA aptamer library

The “chew-back” reaction was mediated by T4 DNA polymerase to generate cohesive ends to the AG overhang. 20 μl of IL-10 antigen (100 pg) was added and incubated for 2 hrs and washed with 1×PBST for 5 min twice. 20 μl of Oligo-labeled detection Antibody was incubated for 2 hrs and washed with 1×PBST twice, then rinsed with water for 5 min. Ligation was carried out at RT for 1 hr and washed with 1×PBST for 10 min twice, followed by a water wash. The plate was then twice heated for 20 min. Ligated products were harvested and transferred to a PCR tube (around 20 μl). A 1^{ST} PCR reaction was performed (30 cycles); PCR products were separated by gel electrophoresis, and then purified. A 2^{nd} PCR reaction was then performed (35 cycles) using inner nested primers. PCR products were separated by gel electrophoresis, purified, ligated, and transformed into bacteria. DNA from the transformed bacteria was then sequenced and analyzed.

### Example 30

### DAPPL-PPI Screens

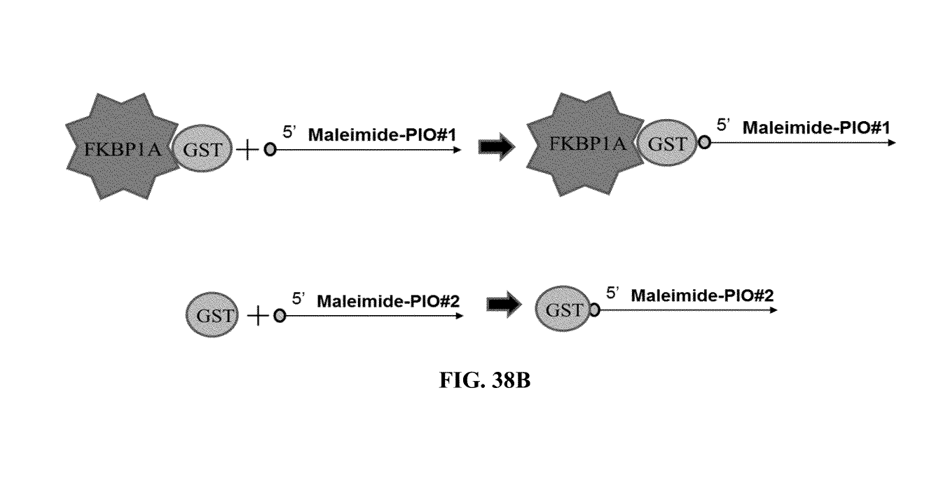

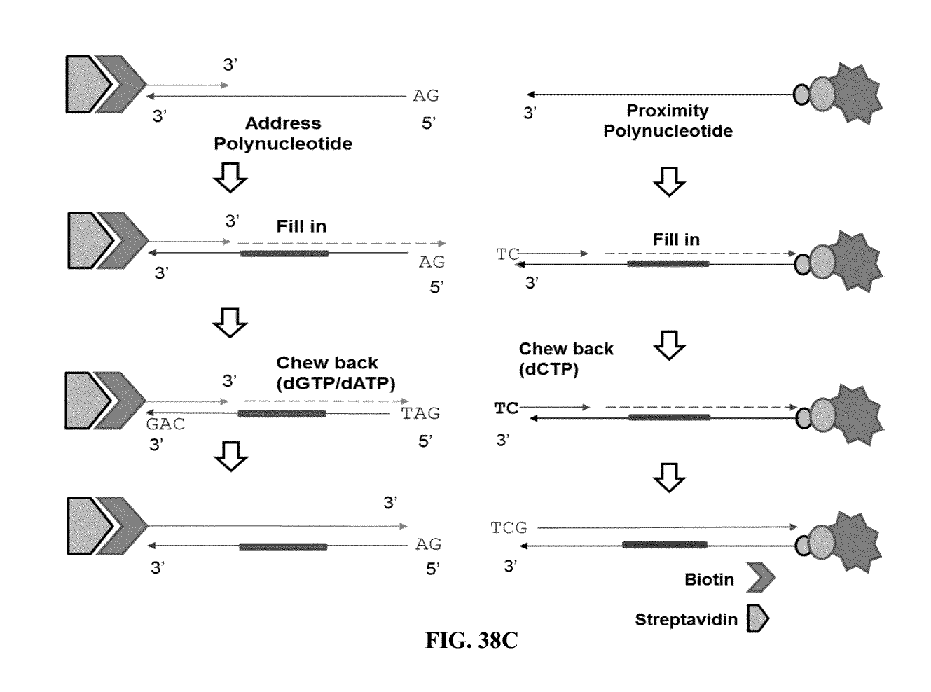

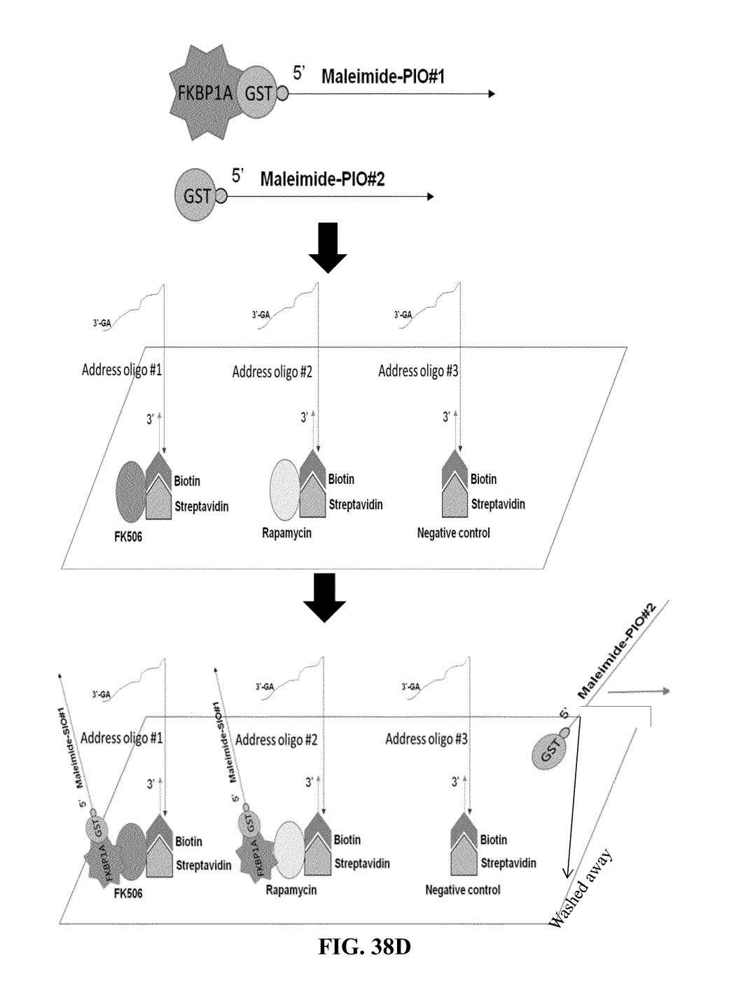

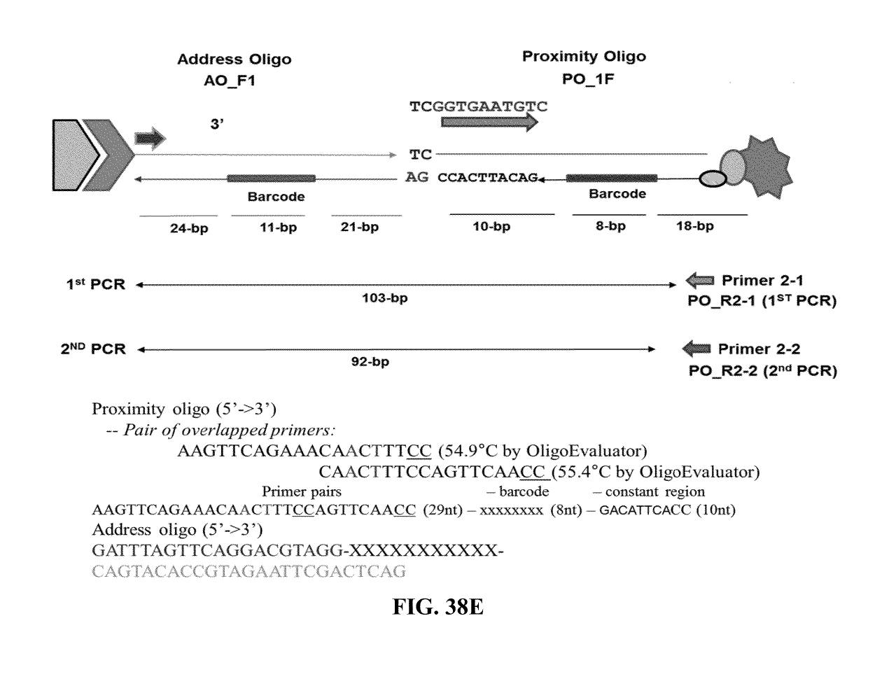

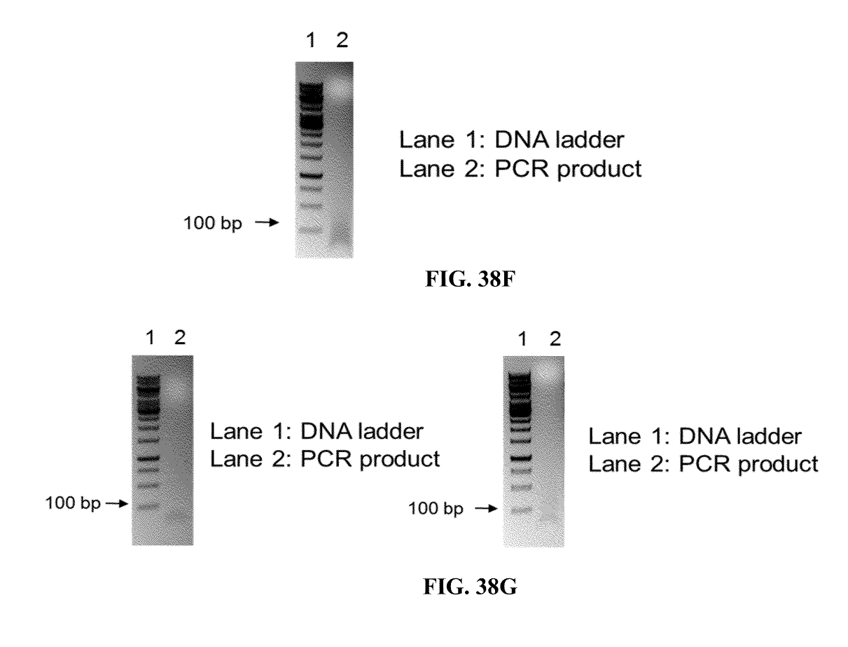

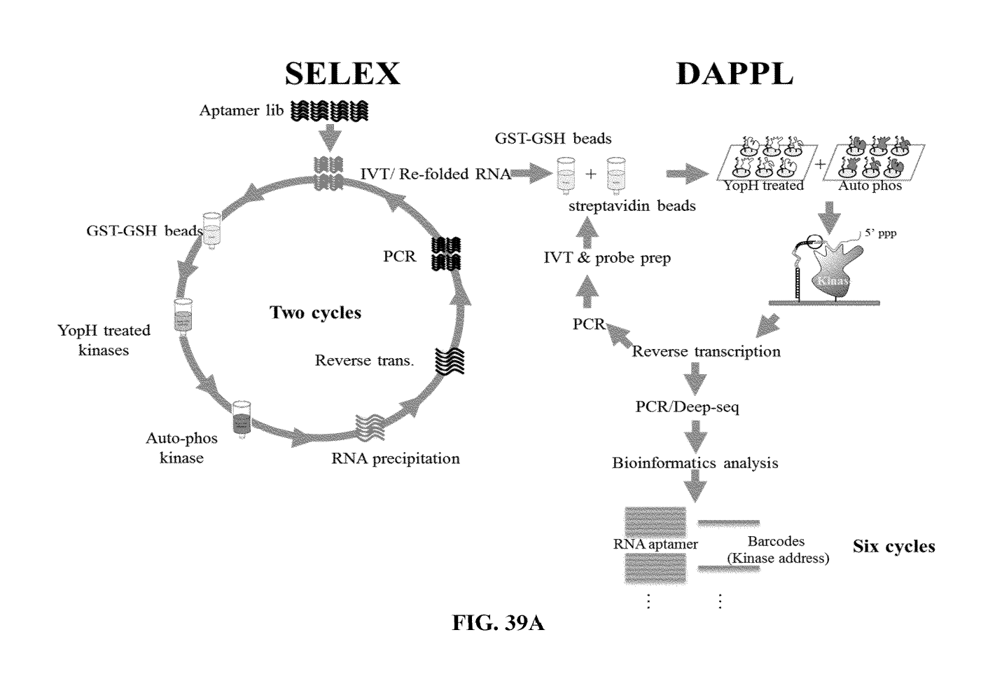

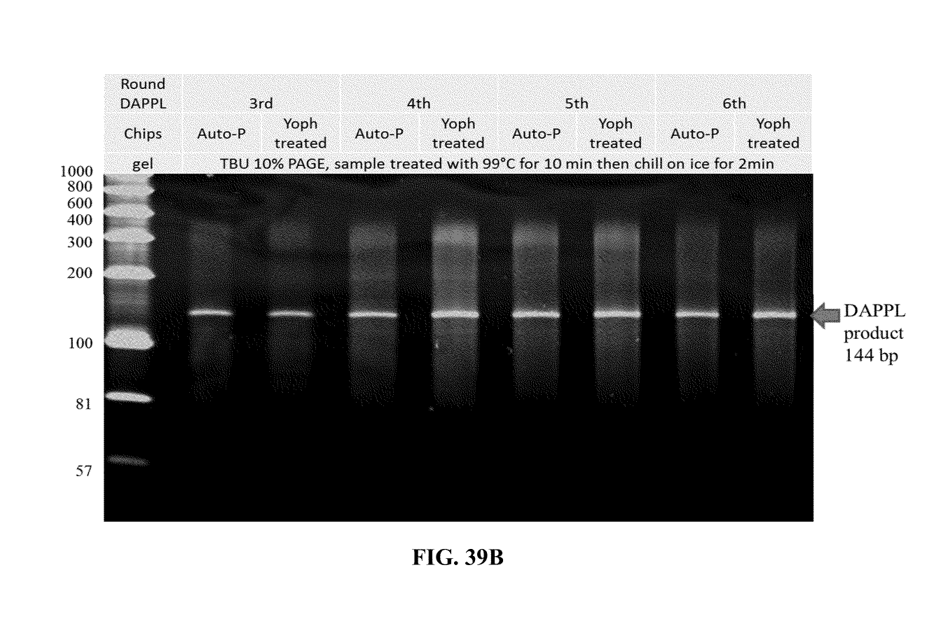

A DAPPL approach is applied to identify high affinity macrocycles or protein (FIG. 38A) binding partners that interact with protein targets of interest. FKBP1A is a protein known to bind FK506 and rapamycin. GST-tagged FKBP1A and GST (negative control) were labeled with different barcoded oligos. FK506 and rapamycin were printed on an array with different address oligos along with a negative control (printing buffer). GST-tagged FKBP1A and GST were probed on the array for peptidyl-prolyl isomerase (PPI) activity. DAPPL methods were carried out after binding PCR amplification of the DAPPL product was then performed. The PCR products were then cloned into E. coli and verified by sequencing

The array was blocked with 5% BSA in PBS for 1 hour. The address oligos on the array and the barcode oligos on FKBP1A and GST were then filled in by Klenow enzyme to generate double stranded DNA. The “chew-back” reaction was mediated by T4 DNA polymerase to generate cohesive ends to the AG overhang. A mixture of FKBP1A and GST was incubated on the array for 1 hour. The array was then washed with 1×TBST for 10 mins 3 times then dried. Ligation was carried out at RT for 1 hr and washed twice with 1×TBST+10 mM EDTA for 10 min followed by a water wash. The nitrocellulose membrane on the array was harvested and transferred to a 1.5 ml tube. 30 μl of ddH₂O was added and boiled for 10 min. The tube was spun and the supernatant was transferred to a new tube (PCR template). A 1^{ST} PCR reaction was performed (30 cycles); PCR products were separated by gel electrophoresis, and then purified. A 2^{nd} PCR reaction was then performed (35 cycles) using inner nested primers. PCR products were separated by gel electrophoresis, purified, ligated, and transformed into bacteria. DNA from the transformed bacteria was then, mini-prepped, sequenced and analyzed.

### Example 31

### Phospho-Specific Aptamer Screens

RNA aptamer screening was performed against human kinases to identify phospho-specific RNA aptamers potential for application as therapies. RNA aptamers can be induced to express in cells and phospho-specific RNA aptamers can serve as a unique set of molecular tools for dissecting protein kinase functions in cells. Briefly, a library of DNA or RNA aptamers is incubated on an array of containing protein kinases of interest that have been autophosphorylated, treated with kinases, or treated with phosphatases. Bound aptamers are then recovered and amplified. Asymmetric amplification of the amplified products is then performed to regenerate the DNA aptamers. In the case of using an RNA aptamer library, in vitro transcription is then performed to regenerate the RNA aptamers. This process is repeated for 4 cycles. During the 5^{th} cycle, bound aptamers are ligated to address polynucleotides, amplified, and sequenced to identify phospho-specific RNA aptamers. This process is repeated for a 6^{th} and 7^{th} cycle. Sequencing data from cycles 5, 6, and 7 can be compared to identify high affinity phospho-specific RNA aptamers.

1.-485. (canceled)

486. A composition comprising one or more solid supports coupled to a plurality of target analytes and a plurality of address polynucleotides, wherein each address polynucleotide of the plurality is barcoded to a target analyte and in proximity to the target analyte to which it is barcoded; and wherein each target analyte of the plurality is different and does not base pair with the address polynucleotide to which it is barcoded.

487. The composition of claim 486, wherein the one or more solid supports is an array.

488. The composition of claim 486, wherein the one or more solid supports is a plurality of beads.

489. The composition of claim 488, wherein each bead of the plurality comprises a different target analyte and a uniquely barcoded address polynucleotide.

490. The composition of claim 486, wherein the target analyte is selected from the group consisting of a polypeptide, a small molecule, and a polynucleotide.

491. The composition of claim 486, wherein the address polynucleotide, the target analyte, or both, are coupled to the one or more solid supports by a linker.

492. The composition of claim 491, wherein the address polynucleotide is coupled to the one or more solid supports by a first linker and the target analyte is coupled to the one or more solid supports by a second linker, wherein the first and second linker are a same linker.

493. The composition of claim 492, wherein the same linker is GST.

494. The composition of claim 492, wherein the same linker is glutathione, wherein the glutathione is conjugated to the one or more solid supports.

495. The composition of claim 486, further comprising a proximity probe comprising a binding moiety coupled to a proximity polynucleotide, wherein the binding moiety is bound to a target analyte and the proximity polynucleotide is barcoded to the binding moiety.

496. The composition of claim 495, wherein the proximity polynucleotide is hybridized or ligated to an address polynucleotide that is barcoded to the target analyte to which the binding moiety is bound.

497. The composition of claim 486, wherein the target analyte to which an address polynucleotide is barcoded is known.

498. A method comprising contacting a proximity probe to one or more solid supports coupled to a plurality of target analytes and a plurality of address polynucleotides, wherein each address polynucleotide of the plurality is barcoded to a target analyte and in proximity to a target analyte to which it is barcoded, wherein each target analyte of the plurality is different and does not base pair to the address polynucleotide to which it is barcoded, and wherein the proximity probe comprises a binding moiety coupled to a proximity polynucleotide that is barcoded to the binding moiety.

499. The method of claim 498, wherein the method further comprises binding the proximity probe to a target analyte and coupling the proximity polynucleotide to an address polynucleotide, thereby forming a coupled product.

500. The method of claim 499, wherein the coupling comprises ligating the address polynucleotide to the proximity polynucleotide.

501. The method of claim 499, wherein the coupling comprises hybridizing the address polynucleotide to the proximity polynucleotide.

502. The method of claim 499, wherein the coupling comprises hybridizing a splint polynucleotide to the proximity polynucleotide and the address polynucleotide.

503. The method of claim 499, wherein the method further comprises amplifying the coupled product.

504. The method of claim 503, wherein the amplifying comprises amplifying a plurality of the coupled products simultaneously or in a single reaction.

505. The method of claim 499, wherein the method further comprises detecting the coupled product or an amplified product thereof.

506. The method of claim 505, wherein the detecting comprises sequencing.

507. The method of claim 499, wherein the proximity probe comprises a plurality of proximity probes, wherein the binding moiety of each proximity probe of the plurality is different.

508. The method of claim 498, wherein the method further comprises identifying a target analyte as a specific binding partner of a binding moiety.

509. A method comprising coupling a plurality of address polynucleotides and a plurality of target analytes to one or more solid supports; wherein each address polynucleotide of the plurality is barcoded to a target analyte and in proximity to a target analyte to which it is barcoded, and wherein each target analyte of the plurality is different and does not base pair with an address polynucleotide to which it is barcoded.


SEQUENCE LISTING (94 sequences)

SEQ ID NO: 1 - DNA, 12 bp, Artificial Sequence
  Description of Artificial Sequence Synthetic probe
nnnnccggnnnn

SEQ ID NO: 2 - DNA, 12 bp, Artificial Sequence
  Description of Artificial Sequence Synthetic probe
nnnnccggnnnn

SEQ ID NO: 3 - DNA, 12 bp, Artificial Sequence
  Description of Artificial Sequence Synthetic probe
nnnnccggnnnn

SEQ ID NO: 4 - DNA, 10 bp, Artificial Sequence
  Description of Artificial Sequence Synthetic probe
acggcggata

SEQ ID NO: 5 - protein, 6 aa, Artificial Sequence
  Description of Artificial Sequence Synthetic 6xHis tag
HHHHHH

SEQ ID NO: 6 - DNA, 60 bp, Artificial Sequence
  Description of Artificial Sequence Synthetic oligonucleotide
cacaacagatnnnncgnnnnatatgggatgggtgtttaaaccctatagtgagtggtatta

SEQ ID NO: 7 - DNA, 60 bp, Artificial Sequence
  Description of Artificial Sequence Synthetic oligonucleotide
cacaacagatnnnngcnnnnatatgggatgggtgtttaaaccctatagtgagtggtatta

SEQ ID NO: 8 - DNA, 62 bp, Artificial Sequence
  Description of Artificial Sequence Synthetic oligonucleotide
cacaacagatnnnnccggnnnnatatgggatgggtgtttaaaccctatagtgagtggtatta

SEQ ID NO: 9 - DNA, 10 bp, Artificial Sequence
  Description of Artificial Sequence Synthetic probe
aaagcgttca

SEQ ID NO: 10 - DNA, 10 bp, Artificial Sequence
  Description of Artificial Sequence Synthetic probe
atatgcatcg

SEQ ID NO: 11 - DNA, 143 bp, Artificial Sequence
  Description of Artificial Sequence Synthetic polynucleotide
attcggatccgattaataccactcactatatggcagtacgggtttcagtacgggaatgatgaccctcttgctacgatgcataacctacgtcctgaactaaatcatcagccatggccttgtcgtcgtcgtcggtacccagatct

SEQ ID NO: 12 - DNA, 144 bp, Artificial Sequence
  Description of Artificial Sequence Synthetic polynucleotide
ttcggatccgatgatttagttcaggacgtaggatatgcatcgtagcaagagggtcatcattccctactgaaacccgtactgccatatagtgagtggtattaatcagccatggccttgtcgtcgtcgtcggtacccagatctggg

SEQ ID NO: 13 - DNA, 143 bp, Artificial Sequence
  Description of Artificial Sequence Synthetic polynucleotide
gctcgaattcggatccgattaataccactcactatatggcagtacgggtttcagtacgggaatgatgaccctcttgctacgatgcatatcctacgtcctgatcagccatggccttgtcgtcgtcgtcggtacccagatctggg

SEQ ID NO: 14 - DNA, 90 bp, Artificial Sequence
  Description of Artificial Sequence Synthetic oligonucleotide
gatttagttcaggacgtaggatatgcatcgtagcaagagggtcatcattcccgtactgaaacccgtactgccctatagtgagtggtatta

SEQ ID NO: 15 - DNA, 90 bp, Artificial Sequence
  Description of Artificial Sequence Synthetic oligonucleotide
ctaaatcaagtcctgcatcctatacgtagcatcgttctcccagtagtaagggcatgactttgggcatgacgggatatcactcaccataat

SEQ ID NO: 16 - DNA, 15 bp, Artificial Sequence
  Description of Artificial Sequence Synthetic oligonucleotide
gttagttagttagtt

SEQ ID NO: 17 - DNA, 113 bp, Artificial Sequence
  Description of Artificial Sequence Synthetic polynucleotide
gggtccaatcaacgacattgttagttagttagttaatgtcgttcagtcgatgctcccatacatctccaggtataacgttgcaccctacaccctatagtgagtcgtattatgat

SEQ ID NO: 18 - DNA, 111 bp, Artificial Sequence
  Description of Artificial Sequence Synthetic polynucleotide
gggtccaatcaacgacattgttagttagttagttaatgtcgttcagtcgatgctcccatacatctccaggtataacgttgcaccctacaccctatagtgagtcgtattatg

SEQ ID NO: 19 - DNA, 110 bp, Artificial Sequence
  Description of Artificial Sequence Synthetic polynucleotide
gggtccaatcaacgacattgttagttagttagttaatgtcgttcagtcgatgctccatacatctccaggtataacgttgcaccctacaccctatagtgagtcgtattatg

SEQ ID NO: 20 - DNA, 100 bp, Artificial Sequence
  Description of Artificial Sequence Synthetic polynucleotide
cataatacgactcactatagggtgtatggtgcantgtgnagatgtatgtcgagcatcgactgaacgacatttactctcctaatgtcnttgattgnaccca

SEQ ID NO: 21 - DNA, 99 bp, Artificial Sequence
  Description of Artificial Sequence Synthetic oligonucleotide
cataatacgactcactatagggtgtatggtgcantgtgnagatgtatgtcgagcatcgactgaacgacatttactctcctaatgtcnttgattgnaccc

SEQ ID NO: 22 - DNA, 97 bp, Artificial Sequence
  Description of Artificial Sequence Synthetic oligonucleotide
cataatacgactcactatagggtgtagggtgcaatgtggagatgtatggagcatcgactgaacgacatttactctcctaatgtcgttgattggaccc

SEQ ID NO: 23 - DNA, 125 bp, Artificial Sequence
  Description of Artificial Sequence Synthetic polynucleotide
gggtccaatcaacgacattaggagagtaaatgtcgttcagtcgatgctcccccatactgtaggcaccatccatacatctccaggtataacgttgcaccctacaccctatagtgagtcgtattatg

SEQ ID NO: 24 - DNA, 125 bp, Artificial Sequence; identical to SEQ ID NO: 23
  Description of Artificial Sequence Synthetic polynucleotide

SEQ ID NO: 25 - DNA, 113 bp, Artificial Sequence
  Description of Artificial Sequence Synthetic polynucleotide
gggtccaatcaacgacattaggagagtaaatgtcgttcagtcgatgctctactgtaggcaccatccatacatctccacattgcaccctacaccctatagtgagtcgtattatg

SEQ ID NO: 26 - DNA, 12 bp, Artificial Sequence
  Description of Artificial Sequence Synthetic probe
ctgtaagtggct

SEQ ID NO: 27 - DNA, 12 bp, Artificial Sequence
  Description of Artificial Sequence Synthetic probe
agccacttacag

SEQ ID NO: 28 - DNA, 20 bp, Artificial Sequence
  Description of Artificial Sequence Synthetic primer
aagttcagaaacaactttcc

SEQ ID NO: 29 - DNA, 18 bp, Artificial Sequence
  Description of Artificial Sequence Synthetic primer
caactttccagttcaacc

SEQ ID NO: 30 - DNA, 47 bp, Artificial Sequence
  Description of Artificial Sequence Synthetic oligonucleotide
aagttcagaaacaactttccagttcaaccnnnnnnnngacattcacc

SEQ ID NO: 31 - DNA, 56 bp, Artificial Sequence
  Description of Artificial Sequence Synthetic oligonucleotide
gatttagttcaggacgtaggnnnnnnnnnnncagtacaccgtagaattcgactcag

SEQ ID NO: 32 - DNA, 40 bp, Artificial Sequence
  Description of Artificial Sequence Synthetic oligonucleotide
tctcgtggccgtgtacgaattaagacagtctttatttcta

SEQ ID NO: 33 - DNA, 40 bp, Artificial Sequence
  Description of Artificial Sequence Synthetic oligonucleotide
gctccttacatcggcccatagtattagggcaatccgtaag

SEQ ID NO: 34 - DNA, 40 bp, Artificial Sequence
  Description of Artificial Sequence Synthetic oligonucleotide
cgccccactggcccacaaaaagcataaaccgcattgttca

SEQ ID NO: 35 - DNA, 40 bp, Artificial Sequence
  Description of Artificial Sequence Synthetic oligonucleotide
agcaacgacaagctacagcgatagttgtttgcacatacac

SEQ ID NO: 36 - DNA, 40 bp, Artificial Sequence
  Description of Artificial Sequence Synthetic oligonucleotide
aacaacatcatactaccgcgatagttgtttgcacatacta

SEQ ID NO: 37 - DNA, 40 bp, Artificial Sequence
  Description of Artificial Sequence Synthetic oligonucleotide
tgctagacgctatgcctcgcttacagtctcaacttagtta

SEQ ID NO: 38 - DNA, 40 bp, Artificial Sequence
  Description of Artificial Sequence Synthetic oligonucleotide
gtctagtcgacatttaccctcaaacggctgcgggagagca

SEQ ID NO: 39 - DNA, 40 bp, Artificial Sequence
  Description of Artificial Sequence Synthetic oligonucleotide
ttctagtcgttattccacttctatcggctcgtggtgggcc

SEQ ID NO: 40 - DNA, 40 bp, Artificial Sequence
  Description of Artificial Sequence Synthetic oligonucleotide
ttctagtcgttattccacttctatcggctcgtggtgggca

SEQ ID NO: 41 - DNA, 40 bp, Artificial Sequence
  Description of Artificial Sequence Synthetic oligonucleotide
ttctagtcgttattcaacttctatcggctcgtggtgggca

SEQ ID NO: 42 - DNA, 40 bp, Artificial Sequence
  Description of Artificial Sequence Synthetic oligonucleotide
gtctagccgtgattatacttctatcggctcggggtggaca

SEQ ID NO: 43 - DNA, 40 bp, Artificial Sequence
  Description of Artificial Sequence Synthetic oligonucleotide
tgtgagtggcatcgtcatccccgattatccttgtaagact

SEQ ID NO: 44 - DNA, 40 bp, Artificial Sequence
  Description of Artificial Sequence Synthetic oligonucleotide
tgtgagtggcatcgacatccccgattatccttgtaagact

SEQ ID NO: 45 - DNA, 40 bp, Artificial Sequence
  Description of Artificial Sequence Synthetic oligonucleotide
cacggttgcacttgtcgatgtcaactaacccggaagcaca

SEQ ID NO: 46 - DNA, 40 bp, Artificial Sequence
  Description of Artificial Sequence Synthetic oligonucleotide
ccacctttaggcgtgtctgctaagctgcaccggcgctacg

SEQ ID NO: 47 - DNA, 40 bp, Artificial Sequence
  Description of Artificial Sequence Synthetic oligonucleotide
ccacctttagtcgtgtctgctcagctgcaccggtactacc

SEQ ID NO: 48 - DNA, 40 bp, Artificial Sequence
  Description of Artificial Sequence Synthetic oligonucleotide
ccacctttagtcgggtctgctcagctgcaccggtactacc

SEQ ID NO: 49 - DNA, 40 bp, Artificial Sequence
  Description of Artificial Sequence Synthetic oligonucleotide
ccacctatagtcgtgcctgcacagctgcaccggtactacc

SEQ ID NO: 50 - DNA, 40 bp, Artificial Sequence
  Description of Artificial Sequence Synthetic oligonucleotide
gaatatccaatccaaggctagcaacctctccgtggctatc

SEQ ID NO: 51 - DNA, 40 bp, Artificial Sequence
  Description of Artificial Sequence Synthetic oligonucleotide
caaaccacaaacctatgcccggctctccgacggcgcgtcc

SEQ ID NO: 52 - DNA, 40 bp, Artificial Sequence
  Description of Artificial Sequence Synthetic oligonucleotide
ggattgatgtgacatgttagcccatcataccaagcgcata

SEQ ID NO: 53 - DNA, 40 bp, Artificial Sequence
  Description of Artificial Sequence Synthetic oligonucleotide
cggcaccgcttactcgcaacccacccgcccacctcctatt

SEQ ID NO: 54 - DNA, 40 bp, Artificial Sequence
  Description of Artificial Sequence Synthetic oligonucleotide
cggcaccgcttactcgcaacccacccgcccacgtccgatt

SEQ ID NO: 55 - DNA, 40 bp, Artificial Sequence
  Description of Artificial Sequence Synthetic oligonucleotide
cggcaccgcttactcgcaacccacccgcccacctccgatt

SEQ ID NO: 56 - DNA, 40 bp, Artificial Sequence
  Description of Artificial Sequence Synthetic oligonucleotide
cggcaccgcttactcgcaacccacccgcccacctccgact

SEQ ID NO: 57 - DNA, 40 bp, Artificial Sequence
  Description of Artificial Sequence Synthetic oligonucleotide
cgactccgcttactcgcaatccaccagtccccctccgatt

SEQ ID NO: 58 - DNA, 40 bp, Artificial Sequence
  Description of Artificial Sequence Synthetic oligonucleotide
cagcaccgcttactcgccaccaccacgcccacatccgaat

SEQ ID NO: 59 - DNA, 40 bp, Artificial Sequence
  Description of Artificial Sequence Synthetic oligonucleotide
ccgtcgtagggacacgcggtcatcgccccacaccaataac

SEQ ID NO: 60 - DNA, 40 bp, Artificial Sequence
  Description of Artificial Sequence Synthetic oligonucleotide
ccgtcgtagggacacgcagtcatcgccccaccccaaaaca

SEQ ID NO: 61 - DNA, 40 bp, Artificial Sequence
  Description of Artificial Sequence Synthetic oligonucleotide
ccgtcgtagggacacgcggtcaccgccccaccacaataca

SEQ ID NO: 62 - DNA, 40 bp, Artificial Sequence
  Description of Artificial Sequence Synthetic oligonucleotide
ccgtcgtagggacacgcggtcattgccccaccccaatacc

SEQ ID NO: 63 - DNA, 40 bp, Artificial Sequence
  Description of Artificial Sequence Synthetic oligonucleotide
ccgtcgtagggacacgcggtcatcgccccaccccaatact

SEQ ID NO: 64 - DNA, 40 bp, Artificial Sequence
  Description of Artificial Sequence Synthetic oligonucleotide
ccgtcgtagggacacgcggtcatcgccccaccccagtaca

SEQ ID NO: 65 - DNA, 40 bp, Artificial Sequence
  Description of Artificial Sequence Synthetic oligonucleotide
ccgtcgtagggacacgcggtcatcgctccaccccaataca

SEQ ID NO: 66 - DNA, 40 bp, Artificial Sequence
  Description of Artificial Sequence Synthetic oligonucleotide
ccgtcgtagggacacgcggtcatcgccccaccccaataca

SEQ ID NO: 67 - DNA, 40 bp, Artificial Sequence
  Description of Artificial Sequence Synthetic oligonucleotide
ccgtcgtagggacacgcggtcatcgccccacaccaataca

SEQ ID NO: 68 - DNA, 40 bp, Artificial Sequence
  Description of Artificial Sequence Synthetic oligonucleotide
ccgtcgtagggacacgcggtcatccccccaccccaataca

SEQ ID NO: 69 - DNA, 40 bp, Artificial Sequence
  Description of Artificial Sequence Synthetic oligonucleotide
ccgtcgtacggacacgcggtcatcgccccaccccaataca

SEQ ID NO: 70 - DNA, 40 bp, Artificial Sequence
  Description of Artificial Sequence Synthetic oligonucleotide
ccgtcgtagggacacgcggtaatcgccccaccccaataca

SEQ ID NO: 71 - DNA, 40 bp, Artificial Sequence
  Description of Artificial Sequence Synthetic oligonucleotide
ccgtcatagggacacgcggtaatcgccccaccccaataca

SEQ ID NO: 72 - DNA, 40 bp, Artificial Sequence
  Description of Artificial Sequence Synthetic oligonucleotide
aaatcaaagcacaacgcgagcacaagtccccccccgtcca

SEQ ID NO: 73 - DNA, 40 bp, Artificial Sequence
  Description of Artificial Sequence Synthetic oligonucleotide
acacgctcgttcaatagtacgtgaccacgatactaaacta

SEQ ID NO: 74 - DNA, 40 bp, Artificial Sequence
  Description of Artificial Sequence Synthetic oligonucleotide
gtctgtcgggccaccgctgtcagctacatactcagaagca

SEQ ID NO: 75 - DNA, 40 bp, Artificial Sequence
  Description of Artificial Sequence Synthetic oligonucleotide
ctcggtccgccccgaagggaacgctagaccaaatctacca

SEQ ID NO: 76 - DNA, 40 bp, Artificial Sequence
  Description of Artificial Sequence Synthetic oligonucleotide
tgacgaccatcctgaagctaccgctgcccttcgaattgac

SEQ ID NO: 77 - DNA, 40 bp, Artificial Sequence
  Description of Artificial Sequence Synthetic oligonucleotide
tgacgaccatcctgaagctaccgctccccttcgaattgac

SEQ ID NO: 78 - RNA, 78 nt, Artificial Sequence
  Description of Artificial Sequence Synthetic oligonucleotide
cacaacagauauaugguggauaaacagcuaugcaaccgccgggccuuucacguaaaucgcggucuacaggcgcacuga

SEQ ID NO: 79 - DNA, 78 bp, Artificial Sequence
  Description of Artificial Sequence Synthetic oligonucleotide
cacaacagatatatggtggataaacagctatgcaaccgccgggcctttcacgtaaatcgcggtctacaggcgcactca

SEQ ID NO: 80 - DNA, 492 bp, Artificial Sequence
  Description of Artificial Sequence Synthetic polynucleotide
taatacgactcactatagggacaattactatttacaattacaatggaagtgcagctgctggaaagcggcggcggcctggtgcagccgggcggcagcctgcgcctgagctgcgcggcgagcggctttacctttagcacctttagcatgaactgggtgcgccaggcgccgggcaaaggcctggaatgggtgagctatattagccgcaccagcaaaaccatttattatgcggatagcgtgaaaggccgctttaccattagccgcgataacagcaaaaacaccctgtatctgcagatgaacagcctgcgcgcggaagataccgcggtgtattattgcgcgcgcggccgcttttttgattattggggccagggcaccctggtgaccgtgagcggcggaggaggtcatcatcaccatcaccatggcggaggagactacaaggaccacgacggtgactacaaggaccacgacatcgactacaaggacgacgacgacaag

SEQ ID NO: 81 - DNA, 10 bp, Artificial Sequence
  Description of Artificial Sequence Synthetic oligonucleotide
cacccgtccc

SEQ ID NO: 82 - DNA, 10 bp, Artificial Sequence
  Description of Artificial Sequence Synthetic oligonucleotide
gggacgggta

SEQ ID NO: 83 - DNA, 10 bp, Artificial Sequence
  Description of Artificial Sequence Synthetic oligonucleotide
cacacgcatc

SEQ ID NO: 84 - DNA, 10 bp, Artificial Sequence
  Description of Artificial Sequence Synthetic oligonucleotide
accccgtccc

SEQ ID NO: 85 - DNA, 10 bp, Artificial Sequence
  Description of Artificial Sequence Synthetic oligonucleotide
acaccgtccc

SEQ ID NO: 86 - DNA, 10 bp, Artificial Sequence
  Description of Artificial Sequence Synthetic oligonucleotide
gccgcgcggc

SEQ ID NO: 87 - DNA, 10 bp, Artificial Sequence
  Description of Artificial Sequence Synthetic oligonucleotide
ccgccggcgg

SEQ ID NO: 88 - DNA, 10 bp, Artificial Sequence
  Description of Artificial Sequence Synthetic oligonucleotide
cgcgcgcgcg

SEQ ID NO: 89 - DNA, 10 bp, Artificial Sequence
  Description of Artificial Sequence Synthetic oligonucleotide
ggcgcgcgcc

SEQ ID NO: 90 - DNA, 10 bp, Artificial Sequence
  Description of Artificial Sequence Synthetic oligonucleotide
gcgccggcgc

SEQ ID NO: 91 - DNA, 54 bp, Artificial Sequence
  Description of Artificial Sequence Synthetic oligonucleotide
ccctgtacacagactcaagttgctgaaatgatggaatcctaacacatcccactc

SEQ ID NO: 92 - DNA, 51 bp, Artificial Sequence
  Description of Artificial Sequence Synthetic oligonucleotide
ccctgtacaccttcagaagtttcgagtatgattagctgctacatcccactc

SEQ ID NO: 93 - DNA, 119 bp, Artificial Sequence
  Description of Artificial Sequence Synthetic polynucleotide
tccgatccctgtacaccttcagaagtttcgagtatgattagctgctacatcccactcgatttagttcaggacgtaggagcgaccacggccagtacaccgtagaattcgactcagtcagc

SEQ ID NO: 94 - DNA, 132 bp, Artificial Sequence
  Description of Artificial Sequence Synthetic polynucleotide
gatccgatgacgcatactccctgtacacagactcaagttgctgaaatgatggaatcctacatcccactcgatttagttcaggacgtagggtccaccccggccagtacaccgtagaattcgactcagatcagc